SEQUENCE LISTING (8 sequences)

SEQ ID NO: 1 - DNA, 163954 bp, Homo sapiens
tgaactgaggcgaactccgccgccaagtgagtgaggaggaggaggaggagaaggaggaggaggaagaggaggagcgcagtcggagcgcggcggcagcggcagaggcgccgcggcggggaccagcccagagagaccccccgagcccgcggcacaggcggcggcggcgctgagcgggctcgaccagccgagcgagcggcggccccggctcctcctcgtccggcgccagcagcccgcgccccgcgcccgcactcggccagccgagacccgcctcccgcccgcccgccggcctcgtgagggacgcgccgagccgggcggccgagagcggcggcggctgggagcgcgtcgggcccgggcggaaagtgcctgcggggggcgggcgagcgcgcggtgccggccgccccggggctcggcgcgggagccagagcgggagccggcgcggagcgggacgccgggtcccgagcggcccgcggccggggctcgcccccgcccctccctcctctccggcggcggcggcggcggcggcgggcggagtgagctgcggagcctggaatatggtcggggaaatggaaacgaaggagaagccgaagcccaccccagattacctgatgcagctgatgaacgacaagaagctcatgagcagcctgcccaacttctgcgggatcttcaaccacctcgagcggctgctggacgaaggtgagcgtctccagggccccggccccggcccgacccccgccggggcggcccctttccccgcttgggatggtggggagggcgggaaggtcacggccgggcgggaccgagcgccggggggactgggaggccaggagggcgcacaaaggaggtgccgggccgggcttgcggcgggcgggcgggccggggtggacccgccggtgccggacgccccctgcccggccgggaccccgcgccgctggcgtccaagttggcgcgttgcggagggctgcggggtgagccctgcggcccctctagcttcggcggtcacatttccgagaggtcggcgggggctgcgcggactcggtggtcctcgccgagccctcccgagagcaggcccacgccgcgccgccggcctggaggacgcgtgtgcccggcgcgggccccgctcctccgctcggcctcccgagcctgctctgcgcggacccccaccgccccgcgttcgggagctctcccctcccgtgaggactaaagcggggtttcgtggttggggagttggtgcagctgttcaatactaagtggaagtcagtgtggggctcgttagtttaaagaaattccgtaggagaccgaaattatgctggcgtagccgacaaggaggactaagtgacggcgaaaagcacttttttcccttttgtttggggaagcgagaactgttagggagttgaacttcacttttcgggggtggggggggggggggtggagtgcgaaataacgcgatgacagatcggagcgcagcctgagttagaagaacttgggcggagggtttgctcccggggacgcagccgcctgagtttgtaggacagggtttcgagacgtttggaaagggcgaaataaaattgagccaaagtttccaaaaagctgtatggagcgctgtggggagtgaagaatgacaagaagattaatgttgaccaactttggaatttccttaagaagttctcatagcagtacctgaagtattaatttctagtagctgtgggtttctgtggacgcctagagaaaaaaaagaccacaaaaaaagtctgtttcgaaaatcgactggccaggagtttgtttgtttttttttcctgcttagaggcaccaccggaggactgtgtaaaggaaactgaaaactacaggttgaagagatgaacaatggagtttagatttttcgcaagaatttcgagattttaaaaatagagtcggggaacaggagtttgaagcttggggagttggaatgtcactagagaacaaaaggccggaaatgtttccatgataaaaaatggttaaagaaaaatcccaggagttctgttgtcttgaaagtcatagaaacaacttaaaagtagaaaatggttaggtgttacggctttctcgtcgttttgcttccgtatgttaaacatacagcactcaccaacgttgaaatgggcattttccgctgttggaacagcaaatctgttaatttcaagttgctccagagaaaacctgagctgtccctttaaggggttctgcttttcctatttgtgttggcatttgaaagattgtggaggcagaaaactttctggaatgtcaaaaaaaaaaaaaaaatgctggaactcttaaattgagtatttccaaatgtgagcgctaaggggcgccagttattttttaaccatggaaatggttggaacaaaatgtgtaacatactcaatctgactgacctatattaaatttagttgaaacttaagttatagattatttgatttgtcataatgtgtttatatagtgttttcgaacattgaaaatactttccttaaaattattggcacaactttatgacgtttcaaaataacactcttactgtcttctccttggcatattttggttaagttaaagttttagtagtattagtaacaatttctggtgaaagtttgttttcttgagtgcagttgtttagaaggaatcctttatcgacgataaaatgaaaataggccttggcaatatttagggttaattaaaattaaattgcattcctgaaatgtcgaattcaaagactataggagattccaaaaaaaagtttgaaaatactttcaccttatgtatactatttaaaaagctgcatacttttgcgttctttagttttaaaaggcttcttgtgcttttatgttgatctaaaatctctaaatttagagctgattccttgttatcaagtttccaaccttaggctttaataatttttaatctttcaaatccagaaatttcaattatgtcattgtctctgacaaaggcttatttctctctggttggaaatagtactcgtttgcgttttaaatgtaagagaccactatgatatctcctagataatgtttgtttccccttccccttttaatgtggtttactccatctcttgtcttgctatctttagttttagccttgtcaattccccagtgtaaagattgcttctgtgtggcatatagtatgtttaaaatgggcaatataatgacgaggaagtttgattgttttttatcagaccagtacaaacatgcaggaaaaagggctattccttagtgtggaataagcggcttcctgtaattgttagccttgttataaacttaatttgacttaagaaaaatattggtttctgaagtccatcacatttttttttctggatgatttaattgtgaaatttggcatcttgtgctttacccctttgtggtttgtcattgtccaaaacaaattgatcatgacttttgtttctggttaggcattacttttttttttttgcctataatgcttggctgaaaaattacttttaaatttagataaaatttagtaaaaattttactttagtgattggtttgatgaagttaacatcgtgcttagagtagatgtattttctgataattaggaattttaaatgagattttaaattttttattttaaaatatttttcaaagaataagctttttaaaaatatgcttgtcagcctaagaatttccatttggctagttacaaatttgaggattaagtgtgctggttttcccagttgatgaagacttggatgttttagaatgagtattttaattccttgccaagttaacctttagtctgtatcttgactgtggacagtccttttgttttactgcttgtttttgttttgttttgttttttgctgttgctggtggtgggggtgtgtgtgccagctcttccactgaagtatcttcaacacggttgttttttaaagttgtgaatacattttaggttgggcccatcataaaatataaaggacataaacttgagtttgaaaccttttattgaagatagagatgattataggattacacataaaacctaggtcttaaactgctctacctttagagagacgttttcaactgaacccttagagcagttaatatatggtttttaaaaaaacagacttttcttgtcatgagaatgaaacaacagtagttgaatgcaaattgttataataaagttttgtaatttagaatcgagaaggtgaagagtacggtctattagacttaacaatcctgtattaggggaaaatggagaactagtattttaaaagctgattttgagttttcaattttttaggtggaggtgggcttaaagctgctgatagggaaataaaaaatgtgtaatgaggaagaacagtcaatctttttatcgtggatatcagaaaaattagtgtgtggctcagaaaaagagagcagatggattagaatgccgtattactgatttgaaaatatattttcattttctgatggtctttttgtgtttacagtaaactttctgtctgacctcagtttagtttataccctgcctgctccctctcctttaaatatatgagtaaaaatatttcgttaaaacttgcttaaatagttgctattttttcatgttaatgtttattttcacttcttctgcctctgttattggatgtatcataaggttttcttaccataatttttttttttcctgagaattcatttacaaaagccttgacttaagaattatatggtggttggtattgagaggactcttgctatacagatttcttacaaaaaggttttctttttgtctgaagaagcaattactgttgtcagtaatcatcatctgtaacaaacagattttaaggtgaaataatctgttctttagaaactaagatattgcttttgtacacagtccttgaattagttgtagcaagaaggaagctactatttataatcacttctttccattatcttttctcacccaggaggtgtcagcagctatatattggcagtcacggaaaagatcagttgtatcattatggggcatttattaaacattcattctgtacttttaaagctaaattcccctgtaggtggaacgtgattcctttggaaactgtatccagtttacatgttttgtagtaaaagttgtatccgactgtaacacagttgagttgatagaaataggtctctttgctagtcacatttacgagtgtattttgggatgaacactttttcttaaatgactgagaatgtcaagttaaatcaagttaatttgacttcttaaatgctttatgcacattatatttctgttaatgcttcactagtcactgtcgctgtcctggagctgattgaaattggtcagtttcccataagagcaaatggtttttggtttcctttttcagggaaatagatttctgtgatgaaagaagtgaagtataataaaaccctcatgctttgacctggccttcaccgaagcatgattcagattctttttttgaggccaatatacttagacatgtaagggcttttcttttcttttcttttttttttttttttttggtggtttttttctttccttttttttttttatggtttggtttggtttttatatcaagggaattacaccagtgtgatcataggagcaactttttataaactggaactacagtttaatttacttgagaaattcggtgtagctaatacatttgtttgatttatttgtttgatgttgcagttgtaagcaaaacttgcccttctctgttggagatggaaaatgctgtgacattgttggtttttgtttgttgataacttaaaaggcagcccactgagcaactttctgctattggtatgtcagctgttatgaacatatccaaggttctattttaaggccaagggatggggagtggtgagtgggcctatgttaggaggtcacacagatatataccagctttttatttacatgatcaacgggacttcatttttttctccagtccctcctgtgtttgacattaagacagcttaatgtgaattttaagtgcaataatgtgttgtttacccttaatgataacttttagggaatatttatacccctttaaaaaagttttgggcccacaatagctagattatttcgtgttttacagttatattttttacttaacagaaaatcacaactaacaataaacttttatggtgaatatagtttgttatcttagtgggactgttaagaaatttgtttataaccccagtctgtgttaggtactaccttctatggagtgatcattatagttagcattggaatattattacctgtctgccctcttaaaattactttcttgaaggtgcctaccttagagcctggcatacacttctcagatgtttgttgaattaaacatgttgacatcttacctccttttttgatttctagtttctcaaagatagggtatgtgcacatggtttaaaaaaaaatgaaaatctgaaagtgacattagaaatattgtctaataattttaagaaatgaagcagttgtgtgattgttgatagttttgtgcccttagaggaaaaggcacttatatgttttcttaaaaacaacttaaaaacatgtcatgttatttgtagtacttgctgccaaggtgcttaattttactttactgtcattgttgggaaggagatttaggaagataaaattgctttacttaaatagtatagtttcttgtcagtccttataccaaagggtatcaattcataagcagagaaaaactaccagtgctcatctcagctcttggaaaattttggcatttatggaattatataggtaaactccccaccccgttagttacccttttaaaagtccccacatatatagttcttgtactccatgcagggcacaatagataggaatgatttcttgtccctaagtgctgctttggtgtgttcaggataacgactgtatttgattgagtgattttagtttaaaagaaagctgagtataatcatatgtggctgattgtcatttaacacaaatagctatgtttttaaacacaggtgtgtcttttaattttaaacagtgccattacttagaaaactgataaattgaagttttcacttagctgaaactaagctcttttatttcttttttcttttttttttttcagttggagtctcgctctgtcacccaggctggaatgcagtggcgtgctctcggctcactgcaacctctgcctcctgggttcaaacagttctcctccctcagcctcccgagtagctgggattacaggcgcccgccaccacgcccggctaatttttgtatttttagtagagacggggtttcaccatgttggccaggctgatcttgaactcttgaccccatgatctgaccgcctcggctcttgaccccatgatctgaccgcctcggcctcccaaagtgctgggattacaggcgtgagccaccaagctctaaactgtttattactgtgctttagcagtaacagtgtttgaaaatctgtgatgaatttttaaaaaatatattgcagaagtgatttatctagaattcttacaattagcaagaaaaggactgcacttaatgttttcgtcttcagtttcttgatgcggaaactctagtttgaaaatccggcaatcctatcaagtctaacaaaataaattatgttcataaagttgcttcatgtattagtctaatatttttgtagaactaaattttgaggctggggaaatgaatcgattgtaagatttatatcacattatttattataagcagaatgtgtcatattctgcttagcagataagcagatattgaagtatcggaaagaacataattagtcggatgtggtggtgtgtgcctgtagtcccagctacttgggacgctaaggagcgaggattgcttgagcccaggaggtcaaggctgcagtgttatgatcatgctactgcgctccaccttgggcaacagtgagaccctgtcttaaaaaataaaacatcaaaatgcagttacatccataaagtggctccacataatggtcacaatgtaagtaaagggcatgtgtgtgcaggcatttatatctgtaagtgaagacatgcagtgaatgtgagctgggctttgtgagtactggggctggttaagttccagctgagaaaggttggccccgggattgaataagtagctggccttaagtaagcacctcgcttttgctttagtgcaggagaggtcattgtagagactgggcctgatctcctggtcagtagcaaatcattggggagcccttggtttttaagctgattttatagtttgaaatatataggaaaaaacaaacaaaaaataatacattttaagagtagaattagggaaatgcatagtaagaaaacaaacatgcaattttaaaatttgaaaccgagcctggtgaggtggctcatgcctgtaatcccagcactttgggaggccgaggcaggtggatcacgaggtcaagatttcgagactagcctggccaatatggtgaaaccctgtctctgctaaaaattagccgggcatggtagagtgtgcctacagtgccagctacttgggaggctgaagcagaagaatcgcttgaacctggtaggcggcggttgcagggagaggagattgcgccactgcactccagcctgggtgacagagcgagattccatctcaaaaacaaataaaatttggaaccttttttgtaaactcttcaatgtcatagttttacagggtctcaaacttactttgcagattatatcagagaattgtgttttagagctgcaggggacctgagagttgggagtatatgttcatttcataggtgcaaagacaagtgggcagcagagctagagcaagacactcacttctgccctctgttccattttgtggcgtgcctctccacaaggggaactggagggaaaaacctggaatatttccatatgctaaaaatctgctgctgttgcctcaagttgtgaaggtaaagaagacaaaccaaatgattttttgctaattctgtgatatgctggttttgaacaaatgatggtcagattcttagaactagaatttatttttaacgataactttctaattaagaggctaaaagtgagaaagccatttttagaggatttcagctcattcacttctttgtaagcaaaggctagagcctcagttttttgaataatgtaaaaattgttgctgctattatttttactactcaagaggaaacatttttggctgggtgatttaatagagaagtatgctattgctaagtgattcaagttaggaatgtatacttgtatcagttttagatcagcctggggtaaacagcccaatgatatatcaggtagataactgaagagtgacacccaggaggtggatggttatgaattgaatggaaacggtgctgcaccagatagccaccaggtagctactatatcgtattcctgatgccagcatctgggggatgattgctcatttcaaataattcaaacaaattaaatgtaagccacagttctttgctatgttacagcaattatatccctttgatacagttgttacatggcaggtgaatacatttttttaagaaaaaaaaatttcaaactgggatcaaaaaaattaccctgaaaccgcatctaattgtctattgtcgctgattccaccgtggttgcagtgatgccattatcatgagagtattcaagttttaactctgtatgtgtgtgcctgcacatgcgtgagccagcataaagatgaaatgaatttatgtttttgtgcatatgctaatgagaccctgtgatgaagtggtaagcattcttgtactatagacctgggatctaatcatgactccgcaattaactggtttggtgatcttgggtgagataactaatgtctctgaacctcaagcttcttcatctgtgaatgaaattatcaactgccctgccaacctcaaatccaaatactctgaagttgatgggtattcagaagagcctctcatactataaagtagcagcatcactgcttggtttttaattttttttttttttttaattttgtgtgagtctttctctgttgccaaagcactggatcttggctcactgcaacctctgtctcccaggttcaagtgattctcctgcctcagcctcccgagtagctgggattacaggcgtgcacctccacacccggctaattgctgtattttagtagagacagggtttcaccatgttagccaggctggtctccaactcctgacctcaggtaatatgcctgcctcagcctcccaaagttctgggattacaggtgttagccactgcccccggcctgcgtcatctgctttctatcaacattttaaaatcgcttatgataggataaacaattaaataagcaaaacagcttttagagaatgagaaaagaaaaagaaagatgatgtatccatgtaggttcagctagtttttatgtgcatgagagtcactactaacattctggtagtttagaggatgtcactttgagctgaactatctagttatgtttatttgagaagtggaacctgagttgatattaacagctggtatgatttaatgaggtgaatgggaaggtggaggatgttccaggagaagggaaggtgcagcattgccagcaccatggtgatgggcaaggcttaggaaataggtacggatttagttagttggaatggcaaatgcatagatagaagtagtgggaatagatagagactacagaaagcagaccaggttagggctagattgtggtgggccttgaaggccagaatacatagtctgaattttattctgaaggttgtagggaatctttaaggcttggattatttgggggtgtcagaatgaactctttgtgcataaactgagaatgtatacaatatatatgggtttaaagtagacattggatgaagtgtgaagagtcaaactattatatacgtggctaaccctttaggaaaattttttgagtagtgatattttctcaaaatagtattaataaaaatatttatattgagttttctgtattttaaactccttaaagattttttaaattttgttttaactagaaacaagtctttgttttccttacctacaggttagtgtattcatcctgaaggattgagaacatgtggggctcatttgtttagctgtcagagcccaatttttagttagagaattgagcttttatttctatatgagctaaagacaattgaagagactcttggagaaaatcttaggaattcttgaattaatttgccttgaagaaaattttgtcccaatttcagagatcctttaaatatgttttaccaagtttgatgcgggcttttaaaatttaattgtattaatttttgtgtttattttaggtaaatgagagatatcttgacattctatccctagagagatacatatttgtgtatctttaaaagttaacacttccttacatagtgacattacactgagcaagatggattaatcctacagataacgtaataaccagtccatattcagatttctgtagtggatcccctgatattctttatagctggattgttcaaaccagaatcttactcaatactgctcattaaatctggttgttacgcctttttaaatctagaaccatctctttttttttttttttcaaatgacattgatttaacttgttcagggcttgttcctttatgccctgtgtatctattaacattggaagctagttctaaagtaggttcaagaatagcatcttccactggagtacttcataggtgctgctgtgtactttgttatcttacatcaggaggcacagtaactggctctgccatcagataaggaaagattgatacctggattagagtggtgatagatggatccttgtactttaaaggtagttttttttgttttgttttgttttgtttccccacttggaaccagaaggtgccccatcgaccaaaggtgccccgtcaacattcacacctggttttgaccagttttttgcctgaatcagtttcatcagagcttgcaccatgatgatactttaattctgccattctttctgcaattacttggcatcctttgaaaggtacagctttctgaccccaacacggaaatacattgcgattttgaagtgagtttctaatggggcaagccagatatatgctgctgcttattattacttggttctcaaagtaaagtgttggtttgctagttgcttcaaatggagaccaatgaattttttctctcttttctaatcatctttgtagactcatggatttcatagattcaatgtacttaagtcaactataattaattttgatgttaaaattattacaactttagccattgataattaaactggctcctgtgtctgttgaactgctgcttgctttctggggcacaaggcttcccttgtgccttgtctgccttgcctgttctggagctacacttggccatttctccaataggaatggtatgaaatacttgaatttaggtgttaaacatgttcattactctcaggatgtgattacttctgagccattttctatggacagtattgactttaaaattcattcattcatttttatattttcaattcaattttttaacgttactgttaattttatactcgtaccttttttttttttttttttttttgctggccacagctaaactaatgttgttcttgatggtgacatacttatttgctttattctacaatattaaggaaaatactttcaaaataacaaaattaataatattactaatagcagtaaaactatctttaaaatttttaaatttgttttagacatgttaccaataatatagccagtgacccatttttctggttgatgacaattattttccttttttggtgggggggtcctcattctgtgtatactaagttttaatttctactgaatcttgtttttgactttttgatgctgtcatttattagctgtgttcttttgttgtacttactgcttctattagcttttaccttatgctcagcagacatggaaaatacacatatattttaatgtcaggtgcgtgcgtgtgtgtgcctgcatgtgtgtgcgcgcgtgtgtgtgtgatggcaatatgatccagaaagataacttacaaggagatggttgtttatactagtgctttgtatcttaagcacgaagggtagggaaaaaagaatatgacaggcaaaatgtttcaatttagtgaattcttctgaataatatcagtaaaaatatatatagatctttaaagcttttaaagtcatatgaagtaggggaaacctcataggtagaaagtaagtcttaaaattgtatttcttagtagttgcaaaatctctagtgcaaacttgttgtcattaagaacttttagtagtaaggaacttatgttccagcagatggggatgttagtacacaaaagagagcactcagtgattaaaatgtgttgagaaataacccttttgccagaataggaaaatgttctcctttaaattgtaagggaagagttgctcaggaatgtgacaaataacagttacctaattgagtattttcattggcatgtgaaaaagttgtcatagttctttaaagaacttgtctttttaagagactatatgttggtatgttgaccatattaaatgtaactagtttagtacatcagattgattgcagctattttctgaaatagtttctgcctttacaactcagttgtttgaagggtttttgttttggtgggtgactgagtcacatatattgagaggtatcctgtttgtacgttttctttgtaattataaagtctttaggcagtttatttctgtttattaaaataaatcagaagaaaatgtgtcatagtttactatatgttatatgaagagaaagttcttttggggatcatagttttatcatagtaataaaaaattatcagcagtaaaaaagatagtcttatctcacttttattatagtttatagcattacgacatactgttagcttttttagtaggtaaatactacatagattgctttaacatatgctttgcaattgttgaaatactgctcatttaagttccttcttagatgtgattcagaagtgtactttaatgtatttaagttatattttcctagttcagttatattgagctttttttttttttaaactgtcaaggcaatttaaaatactcttgccttcagtgacttgaaattgaactctgcacaggagagaagaatatgtcttgtttctatatagagaaccaaggaggaagagattgactttgccacctttgtaactgataataacttgcattttagtccctgctccccagccccacttttttccttttctgttattcctaaaatgtagagaattggagatcgatccacagattttcagttcatctttggcagtagcatggtttgtaacttttttcattcctggaagaagcagtggtgagggcacagagatagcgtagcacattgtctttgaaccttaatgggaattacttcttttcagggaaatatagcaatggccaattttgtctatacattttaagattgtttcttatagttgttagagattttatgagaagcttgaatttctttcagtaatcctaaagaactgttataaagaacttatttatcagaagaaatattccttattgggaaaaactaaaatatatcaattaaaaagttaaaagtactcatcatgcactgatagctgctcttaatgtttccttgtagtccccccccaccagtgtttatggatgtagcaaaattggaattacggtttttaaaaaaaattatatcatgagcaaaatttttccttgccatttcatattctttgcaaatataatgtttatagtaatatatttcatgctgtcgaggtatattataatttacttaactaatctgttggacttgccactttgctttcaggaaatgtgtgtaatgatagcattaagtggctttagaagttacttttttaaaagccttaagttacaaaatactgtaactgggaattatgaataccaaactcttatttttgcaatgcagttaatctgaaataagcattttatgttgcattgcagtagaaactggttgttaggatgcaacgtgattttcgaataatgtcagagttgcatatttcatgtattagtttttagtgacttaacatacttaattgaaacttttcattctattctaggctgtttggttgtatgctattaatatgtgtgcgatgttatgagggacacactaaagcttcaaattcattccttcaagttatctcagtagttggaaagaatatgaaaataagagtaacaaaggaaatagaaacataccaagaagactaaagactggtgctaaagagggtgtttttggctgaggaggctatggaatgtttcaggagaggaggtaaaatttcatctggctcttaaaggacaggtaggattgaagtggagaacaggtaagggagtggcgttctagacacagggaacggtgttcttgaatgtgggaacattctgagtaggaaggtgtaaataattctctattagagagagctttttatacctttggtcttagctttttcctttatctgcctccatatttctgtttttagtccagattttcttatctctatcctgtacaggcattacattatagagtgatatattaatgtggtagattgctgtgattgctgctgtccttctcttcccttcattttcagatgttcttttttcttgactttgactctctttaggtttttggagaagaggggtatcttactgcctaacttttaattttaactattgtttatctgtatgcttaatttggctttttattttttttgttttcctagtgcatttggattttacttagcttttattatgaagccatgtgtttcagtttttttctgtatgtgtaggtgattgggcttatgaattgatcaggagcatggaggaataggtacatctggattagcgagtgtgtgaattcttgagctgatgggcctaagctctaaaggatagattagttgtcagcatattctggtctagatagtatggcatagtttttaccagtatgctttcttttccccatcttcccagtttgctttttactctaatggcttgaaaaagtataggttaggttggaagtgagagaacaaaaatctgctaatttaactctgaacaaggaaggctttaggctgatgtttgaaaagtgtgttccatggaatactggtctcctgaggtagtctgagaaaagaagggtcctgtagtccaggaagttctgcatactccttttgtgaattcaggatgtgttgccatattaagtctccaggatttgcagtcaagaaagttaacttggtttaactgagcattttctgaaattacttggttaaggaaatacggtttttctttcttttattcttggtactagttctgaggatatgttaagggaaattttaggggattgttaaagatctgtggatccttgggtggaggcaggtttaaacaataattgtttgggaatcttaattttgaatgagagtgctggctcaagtcaaatcaaagtaccccttggaaaaaaaaaagttatattattccacttaggttgggcttcaggtttcaattgaaaggaagcaagaaaactgtccttgtctggctttcattaaataacaattcagtcaaatgatttcagtaatgaattttgtctaactcaaatgcatttgttatttaaataaaaattaagatgtgctgttttttaaaaatgtaattctctaaataaaaaaaattaacatagaatcctgccagcttaaacatcaaactgaaacttactttaaaataggtatatacagagaaatatgctcatgttgtaactcatgcctggtttacatctacattttttaaatttgggaatgactgaattgtgaggtgaagaggaattagtaaattttggaaagaaaaatacttatctttgttaggtcatttaaacaaacattaagatgcaattttgtgagatctgtcagtaacaccggttgttttctgtgaacttagattttaatctattaatagaatatattatctcccctcattcagttatatcataaagttttggtgacctttgtagcctttataaaagggaagtctaagggtggatagtagaatagcattttatttggaaggccctaaagatttttgtaaaaaaaaaaaaacaaaaaactgttcttattaaaaaaaaaaaaaaaccctgtaattctgcaattttaaagcgggtgtggatttgagagatcgtagacccattcttcattatatatgtaaatgctgttcctttacaaaggatatgaatacattttatattttggaaactgctggtttcaggcatgttagatcatttggctcttgggacagaaatatatttaagttacattattaacaagtaagagtacctttgggcctgatcccctctaagcagaataatgtcatgactgataattgtgtaatgtggctaccttctgttgtgaatagtcttaattaatttaattctgttttgttgctggaagtatctacccagggtattttaaattcttcctccaaataagaatacaatcaagcatctaatgcacagtcatattatcattaaattgatgttaaaccaaagacaaaggctctcctaggtagtgtccattagcattttccagcagggtgaatctaggtctaggaacaattcactgaatatttgcctaagggttttcactttccatgttttacacaatataaataatttaaaatcccccccctttttttttttttaaagagatggggtttcaccctgtctaggctagagggcagtggtgctatcatagctcactgcatcctctaactcctgggctcaagcgatcctcctgcctcagcctccagagaggtaggactgcaggggtgagccaccatgcctggataattaaaaaaaattttttttttggtaagagacagggtctcacgatattgcccagcctggtcttaaacttaaggcctcaagtgatcctcctgtgtcaggtgtcagccagtaatttaaaatgttttgttaagagtagtctctctctttccccactttgttatagatctgagtcatttgctttggaataatgtgataaaagtctaagataaaggattgctagactaatatttagacatgtttgtgttgatgttctgtacaggttgtagttttataggctagaatagactaaattttgaagggtgattagttaagaatagactaaattttgaagggtgatctgaaaagactttatattgttcttttgtaaatcttgagtaaaagaaaataaccaaacagggtagtgagtagagagataaatgaatcccttaagagaatgatagtaataaaggcaggtggaagtaagatcagagttagaaaatcttagatttgttttaggttataggaaaattgatttattttacatgttggtaaaaggcacaagaggcttctttttacattttactttatcaaagtgaaactttttcatttgatttttaaattggaacacattaactgaatggcataggatatatcatagtgatgttgtttaggtacatggtaactgcagataggtctcaagttgaccacatttgaatgttacattattactttgtggatatttaaagacagatggcttgaagcctgagaagggggaaggagtgaaagtataggagcaagtggtggtaatttaagaagatcctcaagtagaagtctgattttgtgatttcagagtctctttaattgttaatgttactattattaaataacaggtacatatatcctaacagtataaatatgaaccagaaaaatagtataagaatgaaccacaggccgggcacggtggctcatgcctgtaatcccagcattttgggagaccgaggtgggcagatcacgaggtcaggagtttgaggccagcctggccaacatagtgaaatcccatctctactaaaaatatacaaaaattagccgggtgtggtggcacgtgcctgtagtcccagctactggggaggctgaggtgggagaatcgcttgaacctgggaggcagaggttgcagtgagctgagaccacgccattgcactccagcctgggcaacagagtgagactcctctcataaaaaaaaaagaatgaaccacgaaaggtagcatgccttgcacttttaaaatgctacctataaggttaaatattttgttatgaacttaaaatgtgtacatacacatttatataactcaacaattgaaatgaaagtttcatgaaataatacttgtctgtactgcctgtgatgcagtatactatttttctattctgtttcatttttaaaaaggttggttgtgattgataatattgatttagtggcttgtgtgctgtgcagtcttagcaaaaccgatgtcgattcctgctgtaatttagttttcaacaaacttttttttttaatccagctctttgctgggtgctggggggatacaaagatggataagacatagtctagtgctcaaggagccacacaatggcatggagagtaatgaaaatagaagctgcaatgctgtaactgccctaacagaggcatgcccagcctgagatggaaggggccacatcatttcctagcaggaagatggcagtttcttctaggttcacagattactactgcgtataatctgataatccctcttatgacgggcttaccttgatgggaaagttagttttttacctagattttataaaaactaaacaataatcaagtttacttgaaaataagatatttttgaacttttagcttactggccaagtttgttaaaaaataaatattaatcttggcactttgggaggctgaggcgggcggatcatgaggtcaggagatcgagaccatcctggctaacacagtgaaaccccctctctactaaaaatacaaaaaattagccgggcgtggtggtgggcgcctgtaatcccagctacttgggatgttgaggcaggaaaatggtgtgaacccaggaggcggagcttgcagtgagccaggatcgtgccactgcactccagcctgggcaacagtgtgagactctcaaaaaaaaaattaataaataagataaatgttaattatatagataatctttaaaaaaattaaattttacaaatgagtaatattttcctaaatatgtcataggaactagctttaatattattattggagaaatacttctttcctgactttttcccccctcagtattgtgacttattaagaaaggtaaagtcaggtagaagtttaatataaaaatatatttgattatttgtacagatggtaaattaaactgtaaaataaaccattaacttttaaataatggaataaaagatgggtacttaattatccaacacttaaaaatgatttttttaaaaaattgcaacttgtgtatattctgtatattgactttttatctcacagtgaattcacaataatactgaatctagacgttaatagattagttgcatattatccaaaaaacataaagcatgacatttctatagaatttagtttctgtaagttacgcaattgttacaggtttttcctgtgaaggataggctgctacctcaggttgaagaagctaatgttggagtgaatgtggtggttgccttagaatgattttgccggcagcagtgaaaaatgttttccttatagttcttgtgcagcaggccatggagtaaagcacatttttataataaaggtgatgaatttaatcgcaaactggcagcctaatggaaattgcagaccttgatactgggagaaaattgcctataaagcaactcttgagagaacaggctttaaatttatccacccattttgacattgaggagaacattggaatcatagggtattttgcttggcctgcttagttcagatgaatgacacttgagtattctatttaagtgttagagatggtttaaagttaagttttatacattaaagcctttagcacatgcatttttgtattttataaagtatttaaaattttgctggtttttaacattttagtagatgatatgcctaatgaaatccatttgccacgtttctagtcatatgccgtgtatttcatgtttctgctgtatatatgaggaactgcaatcttaaaggatatagaatattgtgggcttataattaacactactgacagaatgtttgtcatataaatgatcagtctttactattttctattgtatttaagccatttatattataatatttaaacacagaatggtagaaaatttctattacaggaaaatctgggtttttgtcattttttactctaagtgactaatgtttgtaatttcttaggaataaccaaacccttgattcagtacacactatttaaagtgtttaatccagaatttaggtgcattgtagtgtgtattttgagatgcctgttctgtcccgcagtatacatgcttgttaacagatagctaatgtccaagttcagagaattatttgtggctattggaaacatttacccatccaagcaaggtaaatgtttaaaatgtagaaatgctgatgtaaaatataaaaataacatttctcattatacttattgaaactactggggtttaaaggtaaagaagtgttaacttctagaaggttattcgattttgcttccatacacaaccctgattcttttgactgtagttttgggcagcatggtacattacataataagggtatagctgtggtagatctttgttctctttaggtatagaattggagaagtttaagtttaaagcaagtatgagagcttaatattttgatggagaaagcaagaaaaacgttttacaaattctctattcagggaacaacttactgaggtggtcatgagactgaaggtatgaaaaggaagaaatggcaaggtgcggtggctcacacctgtaatcccagcactttgggaggccgaggtgggctgatcacttgaggccaggagttcaagaccagccaggccaacatggtgaaactccatctctactaaaactacaaaaattagctgggcgtggtggtgtgtgcctgtagcaggagaatcttttgaacctgggaggccgaggttgcagtgagccaagatcactccattgctttcaagcctgggcgacagagcgagactccgtctcaaaaaaaaaaaaaaaaaaaaaaaagggaagaagaaatagaagtattccttttcttgctaaagcaagaaccagtaaattgtatatttttcattgaaagtagttccatctaaattaagagcctgactttttggagaatgattacattcataggagaagagcatgtaatgttggagatacatttgaatgtttagccatctctcaatttccatggagaggagagttcaaaataaatcaactctttaattcccttatcggtgacatagatacttggtcatttgttttatttggctgaaggaagctgtggcaagttgagaattcagaggtaaagcctttgtgagagagatgatgtagtttaattattttaactctagttctcctgatccaggaaagctttatttttactttcagatgaaaattaaaatttgttgcaatttaacgtgtaacacgaagctagaataaggtatatacataagctatatatgtatataatttaaatgatacaagtgatgtatgtatacatatagtacttatgttaaaattttactgtttttctgtaacaggctataaatttagtgtatcgattatcaaagtataattagctgaatataattatttgtttatgagatgatactaaattcagttcccatagatactggtggtgatttactttttaaaaagagtttttcttgttatgtgcttaaatccacacccgaaatgtagccataatttagttcaagattgtttatgccacagtctggaaaacacctgaagtaattgagatttttgagtggttgtgaatggattttagaatcatgtgttgaacagtgacatcactaaattagccatagtaatgttactgtttcacattgagggtttcaggtgtgatgcttttcagttttttgtttttgtttagataactattttagggtttgagttatagttatgcaaatttacattacggtaatgcaaatataccttccaaacttgtgtcaatatgtattagacgctaaaatgccctcccctcccatctctgtttccctcaaattattcatttgtggttaaactaattttaaactccggaaaagtggttaagccaaaaaaggtcagatcctggaagaaaaagctctgaccttttaaaaagagtgttggaaaagtatttgacggtcattttcataagctaaatgtactgtgaggtttgtgtttgagtagcttttaaggttttctggattatgtggttgaattttaaggaggaacttagtaaaaatgagttgggaaaattaacctgttacattctaccttccttttgctatgtatagtagatctacatgtgagaagtcatttgcaaacagcatttgttaggtgtcttttctaagtattttttatgcctacaaagtctgggaaaagaccattttcagatttcagataatatttcagataaataatgatcagatcagtagatactgtctttcagatctatcagaacacagtatttccagaatataattttacatcacagtcatcatttataatgacattgatgaccagtactattattattttaaagagaaagatatatatacacatacacataaagtcatgcattgcttaaagacagtgatagattctgataaatgcatcattatgctatttccttttgcgaacatcatagggtgtacttgcataaatctagatggtatagcctactacacacctcagctgtatggtatagccttttttgttcctaggctgcaaacctgtacagcatgctactgtactgaatactgtaggcagttataacacaatggtatttgtgtaaacgtagaaacattctaaacatagaaaaggtacagtaaaaatgtggtataaaagataaaaaatggtatacctgtataaagcacttaccataaatggggcttgcaggactggaagttgctctcggtgagtcagtgagtgagtggtgagtgaatgtgtgaaagtcctaggatactactgtggatttaataaacattgtattcttaggctgccctgaatttataaaagatatttttcttaagtaataaattaaccttaacatactataactttataaacttctaaattttttaaacctttgactacaattacagtttaaaatacaaatacattatacagctgtacagaaatattttctttatatccttattctataagctcttttctgttttcgaacttttctatttaatttttacttctgagtcctttttgttgaagacaaacacacacattaacctaggcctgcacagggtccagatcatcagtttcatcttctttcacctccacagcttggcccactgccaggtcttcatgggcaagggctgtatggcttaccagtgtgtctccagcacagtgtctgataccacagaaagataggaactactgcattaataatagcagattcttgagcatgtattacatgacagacacctacaaatatttgttgttcaagcgaaagaatttattctatcaaactcagaattttcgctctagaataatccttcgtggtttttttttgttttttttttttttcgggggggtgggggacggagtctcactctgttgcccaggctggattgcagtggcacgatctcggctcaatgcaccctccgcctcctggtttcaagtgattctcctgcctcagtctcccgagtagctgggattacaggtgcccgccaccacacatggctaatttttttttttgtattcttagtagagatggggtttcaccgtgttggccaggctgttctcaaactgctgacctcaggtgatccacttatctcggcctcccaaagtgttgagattacaggcatgagccaccgcgtcctgaatcctttttttttttttttttgaaacagagtctcactctgttgcccaggctggagtacagtggcttggctcactgcaacctccatctccagggtgtgagcaattctcatgtctcagcctcccgagtagcaggtaatacaagtgtgtgccaccacgcccagctaatttgtgtatttttagtaaagattgagttttgccatgttgaccaggctgatctcgaactcctgatctcaagtgatctgcctgccttggcctcccaaagtgctggaattacagacgtgagctactgtgcctggcctagaataatcttagaagtcctcatggatgagtaaatatggatggtgaaaagcttaaatgagaatgtcttgtctgattttcattccacttttaatttgaatataccatttcctttcccccctcattgtatatgtctctacttttttgttctttactttattcatagttctaatttctactcactgccccatcttttccttcttctctatctagctcttaccatctgtcatcactgcttttcccactatcatgatcactcactctagcagcaaccaaatttaactttggggatggagcagtagtgctggagaggccaaatggtatgagtaaaagaatacagcttttggagtcagctagattttcatctttatgtggtctttggtgaaagtacttaatccccaaatctctctttcctaactatgaaatgaaggtgaaaatattacttacctgtaagggaatctacctgataataatggaataaaatgctgacttccaccacagagttcttggggaggctttttatgtggaaagacattagacttcattactctgaattctggatttgtatctcctagccatatagactgaatgactgtagaatccaacataatgcttttaatattggcataacttccttagtttaagttatatgatgagatgtagaaactcaataaaaggtaaaattattctttgtgttatgaagtatgtttagaggtttcttttaagaggtcttattttgctactctgcttcaactatagtttttgttcatttagtttgttatagaactgggaccatgtctgtatttgcacatgactatattcctagggccttggccagtgcctggtacagagtaggtgtaaaatacatagttgttgaatgcatatacgaaaaaaatcattatattgtatcattggtaagacagttttttttttttaaaacaggttatactttattcaggtcagttcaattttgtaaagtttttacttttaggaaattaaaaagcttttattttattatgttctgaatatatttttactaaatatactttgagtacagggttaattaggactgtgcaggctatgaatatagggtttatgataaagctcagtaggagtggatgattgcagtccttttgtgcaatcctggcaggagcagccttttccttattggctcggtttttttcttaaagccaagactaagcacatatttagatgcctgtatattttattttctcatctgatgtaagatacactggaagaatttagcattgatgtaatgaacatatgttattagcaaagtgttctaaacttctcaagtccagaataaagagaccaagccatttgcagaagagcactgtgcatttatattactttaaagggaagaggagtaagctagctagaattgtcaagcagatgccatattgatgcttgagtgaagttacaagaaatatttttggagctttgcgtgtttttgtgctaatagtcctaatatttgttcaaaaggtgttttttctccaaggatgtagatgttacaaatatagcaaaaggcaaaaacaaagctatctgccctctcagctaacattctaatgtcaacaggcaataatcaataaacaagtacagtaatgtattaggaaatatgaattatagaaataaaattgaagggtaaaggggatctggagtgttcctatggattgaggcaagttgctgtttaaaaataggaagatcaaggttggactccttgaaaaactggagcagaaatctgaaggaggtgttagtgtgagccatactgctctttggggaaaagctttccaaggcagggaaagagttactgcaaaaccttgagggcagggctagcaattccttgtgtggacttgtagccagggacggaggttaggttggttaaggacatttgatcagaagggcaaaaaaggtgagggcaaggggagcctgtgtggatgctgtaggctattgtttatctttgtaaggacctgggcttttataccaactgagatgggaaatcattagagcagtggttctgggcatccttttagggaatctgtgaaatcagactatctttgtgataataagatgttatttgccattttcaccaccatttacatacgaatgtacagtggagttttccagaggctgcgtgctatgtgatattgcaacagattgaattcaagagaagagagaatctagctgtctttattaaaacagacatttaaaagattgcaaaagtataaaactgaccttttcagtaatttgtttagttggaaaataggttattttctgtaaaatgtgcatttttggttaatatataataagtttactagtattttaaaatgaaaaaataatttaaacatttttagttttaatttctagcgtggtaagttttattagacataactttcatgaacagggatctctttgggctcctccaattaatttttagagagtggagtcctgaaaccaaaaagtttgaacagaggagtgacatctcacttggatttcagcaggctaactccaaagaataaagaatagactgaccaaggtagtggtggaagcaggtagaccggttaggttaagattaggtaggaaatgattgtggttagagctgtaatgttagggcttgtgatggtgaagtgaaattggggatatagtctgaaagttgagtacacagtatttgctagtggatggaatatgaagtgtgagaatggacttgacattgtgtcattgcccgagatcaggaggaacaaattttggggaaggcttaggagtttagtcttgaatgtatcagataggttgagaagggagttggtgataggatttcattgtggagggttggtctggaatgtccattaccagggtttttgttggattgtattagtaatatatattttttaaatatagtatttagcttctaatcagttatacttttacaatctcaacacagagggctgtattctgcattgaaaaactgattaaatttgctttccaagtatgttattatatgatagaatgtacacttgaagaggcagtaaaaaagtaataaatgtagatttgccagtagcatgacatgacttagattttctgaatctttgtgaagtaaaccaactgtggtttaattaaaggagatcatattgcttaccatattaaaataatgttttcaataccaattactttgacatttgaaggcactgtgtaatatgttggtcttgttacagaccaagatatttttgctgaattttatttttcgaattcaatgtcagtgcttgttgtagttccttttggaatattgtgtatagtagattgtttcttaagcctttaaaaaactcttcaccgggtacagtggctcacgcctgtaatcccagcactttgggaggccaaggcgggtggattacgaggtcaggagttcaaaaccagcctggccaagatggtgaaatgctgtctctgctaaaaatacaaaaattagtcggtgtggtggcgggtgcctgtattcccagctacttgggagactgaggcagataatggcttgaatccgggaggcagaggttgcagtgagccaagattgcgccactacattccagcctgggcgagactccgtctcaaaacaataacaacaacaacaacaacaactcctcttggctttttcataatatgagagaattctcttgcaacacctgtgaatgtcttttgacatgtattttctttcctaaaaatgtcatgaagtgacagacatgtatttgctttgtttatggtattaaggtgatactgaggtaaacctatgctacatgattataaaccttaggttctttctgtattgtacttaggtgaaattttgaatacaatcgaaaaggttaattaaaaatcaaataaattattacctaggaaaaagcaatttacccctacctcctacacagacgtaactccttcagaatttttggggcatcggtttcattttcagtctgatttaccaaattccttttaaagccctaagggatttggagcttcaagagggcgaaatacataggtacagacctttccccacttcttcaagagcaagtgtagaggaatctaattacatgaggatttggtgttttccgaggatgtaatttaaagtaaattctggtggccagtagtagtttaagacattttagaatatatagttctggcaggagtaggatagtctggcctggagttctcaaccagtcattgagtatatacagcccttgcctgtagggggtcagtacagaaggagcacaaaatggaattctctgtaagaagacaaagagatgcattttagggcagatggaagccataaatgtgatgtggtctaatgtgctcatttcgcagatgaagaaaatgagacccagaaggttcagtttgaaggcacacagctcagctaaatcatgctggcgaaacaggagtaaaatccaggttgtgtaaactcaaggccagtgctttttgccttaccacatgtattaggaagataggctttttatagaaaggtcattgttttggattgtaatctttctccaccttctttaaggaaagttcaaagtgtgtgtgttttcttttaaagcagtatattaaagaaaatctaaaacgtatagataaaatttacaaagcttttgcagggaaagtcatctgtagttgaggttaacattagatcttttatctgtggatatttattttggagaagagtaacgaatatattaagcacttatatttaacatatattttggatgattagtaatgattgcttatttgaagaaagcaaatatgtataattaaaattttaacaacattatttaaactgtagaagataccattttgagtgatttttttccctcagttttctttatcttttttcacagctttcatactttgtcacattatttccccccttccctcattagaataaataaccccaaagtccaaagcaaacactttttaattattattattattatttttgagacagggtcctgctctgtcacccaggcgagtgcagcggagtgatcatggaccactgcagcctctacttcctgggctcaagcgatctttccacctcagtcttgagtagctgggactacaggcacaccaccacacctggctaatttttgtggagatgggatttgccatgttgcccaggctcactttttaaattgtaattctttattttgaaataaatttagatttacataacagtggaaaaaaataatgtggatgttaactcatataatcatagtatagttatgggaagcaggaaatgagtattgatacagtgctatcactaatctgcaaatcttactcaaattttacctgttttcctcctcactctgttggtttttctggtccaggttttcctactctgttggtttttctggtccaggttttcctactctgttggtttttctggtccaggattccacattgtatttagtagtgatgtctgtttcgtttcctccaatctgtggccatttcacattctttcatgaccttgacacttttgaagaatgctagttattttataaaaatgttcttgaatttgggtttgtttgatgtgttctcatgattataatgaggaccatttttggcaaaacaatcacacacaagcgctgttgtgcccatctcagtggatcatgctgtggagtatatgtcttttttttttttttttttagttggagtcttgctccgttgcccaggctggagtgcaggggcaccatctcggctcactccaggctccgcctcccgggttcatgccattctcctacctcagcctcttgagtagctgggactacaggcgcccgccaccacgcccagctaattttttgtatttttagtagagaagggatttcactgtgttagccaggatggtctcgatctcctgacctcctcatccgtccacctcggcctcccaaagtgctgggattacaggcgtgagccatcgagcccgaccgttgtggggtatatgtcttaatactggtaatattaactttgattagttggttaaggcgatgtctgtcagatttctccactgtagtttctccttttttccttagtaattaataaatgccttacaggggagatactctgaaaccgtacagctatcctttcatatttttgctaactaatttagcattcattgaatgatttttgtttgcattgatttttactaattaaaattctttaaggaagagctgtcccctttcccccttgtatttatttattcagttatttatatcaatcttttcttatgggttgtaattcattactatcactatataagtaaaaacttttaagaaaagtttctgaaaagttaatacattgagataattcaaaattcagagactcgtaagagtacattttcttctatccctgttatcttgggtacccgtttccattgctaagagtttatttttattctttcagaaatattaaaggattgggaaaagagttgttaataataattgctggattctcaggtatttctttttacctggattctgatgatatcaaactaacttgctgttgcatcttcttcggatattcattaaaatgttacttaactagttcgccattagatttaaatttacatttcagtacagtgactattatatatagatggggactggcttcccctgagtatcctaagaaacgacatgaccagaagaccacattttctattaaataaataatagttaaatattaacgtgtaaaagtaaacaggtttaaaagtaaactcttttttggtaagttgaaacaaaagtcagtttaaatgagaaaggctgcctatccaattaagtaactggtttttcaaaagtgaatgaaggccctccccaccaagtcccccgctctgttctgttcttttcagtgacctattttactttctgtgtctactggcagtatccgtttattcattcaggatgtcactttaggaacctagacctggagttcatctacagaactgtgttctgttaaaatggagtgaaagaaaagggctaaaaagactttctttcacacgctattaaaacagtgaacatactggtctacacttattatagtacagccagcaatacatcaaaaggtggcttattccattttatttccattctctaattaccgtttcttaaaatgaattttaagagtttcttatgccaggaaatcctctatgaaactcttaagtaaagctggttagactagctgtgacttggtccagtttcttttatttttttgatttaccttttctttatttttattgatagatattttacatatttatgaggtgcacgtgattttttttttttttttttttgctacatacatagaatgtgtaatgattaagtcagagtatttgaggtctccatcaccttgagtatttattatttctgtatgttggggataattcaagttctctcttctagctactttgaaatatacaatgtattagttaactgtagtcactctaatctgctgttgaacagtaaaattcatacctttatctaattatatgtttgtacctattaacatacttctcttcatctcttctcctactcatccatccttcccagcctctggtatctataattctactctgcctttatgagctaacttttgtagcttccctgtatgtgtgattgaggacataacgatatttgttaattctttaatcactgagcattagttgttttttctttttattttgaggggacagtaaaaagggtattgtaatatatcctccttcagattctggtgaagaccagttgagtcatgtaaaaaataactagatcgctttatttttagagttgggaggcaatgaattataacatctttattatgctactgtttttatataggcagtcccatgcttgcagtgttttgatgtaagcttttctgctttctactctcctaataggtgattattgtaagaaaaatttatataataacatgaaagcgaaatagtcctcgtaaattcatctcccagagatgattactgttaatagttgtatcttttttgactttgttgtgtctaccaaaaaaatgtatacttaaatataaatttgatcatacagtagaggttcttttctataaccccaggaacgtgtgtgcaacttactgttaggtacttctgtaatacagaactgctgctgagaacaatcaggaaaatcgaattaacattgttacaaagtttttgctgttttgtatttttgcatgtgaaatttatcgccagttgtggacaattgttagttttggtagtacaaagaggagttatgattaatttaattttgtagtttttgtttttggacttaggattgtgaagtacagagtacaatttaacccttggaataaggttccatctagggaattactaatagcttgggttttttttttttttttcttgccagtttgaaagcagttactttgaaattaaatcagatttatttggttgttagagtaagcaaacatatttcacttaatagtctgtactctaggctaggcgcagtggctcacgcctgtaatcccagcactttgtgaggccaaggcgggtggatcacctgaggtcaggagttcgagaccagcctggccgacatggtgaaaccccgtgtctactaaaaaaaaaaataataataaattagctgaccatggtggcgggcacctgtaatcccagctactcggggaggccgaggcaggagaatcgcttgaacccaggaggcggaggttgcagtgagctaagattgtgccattgcactccagccttggtgaaaagagtgaaactccatctcaaaaataaataagtaaataaataatactctagtaacttgctttttaatctagttgctctctgtaccttgtgaacttggcctcttaaaaagagcacagtgaagttaaattccatgggtggttctcagctcaaaaataactttaaaactttctctgtatttagtcaagccttaattaaagtttatttctttttacagaagagattgttgttaaccctaaattcataaggttacacaccagccacctctttatgaagtgacagttcttcagttttaaatagattactaatattattgttttaagatttaatgagtattatctaaataaaaacgagtaatttgttattaagacctaaaataactaaatcttactttgctgtttgtgttagtgagctactttaatgtctagcaagaggtagggattgcagacagtgtggaaatactggagcctatttaagcttcagatgaaagcagtcattttaacttacctcataaaatgtattttatgtatatcaaattatgtatgtgtttatatatacttgtcgttttactgacaactaagattttccataagttaatggaaaatgttaaaaagcacagttatggtcaaatgacacagtgaccttttcattcctttattagctcctgctctagtacaatgataaggcagattttgttgatggattcaaatgtacaaagaaacagcagatagatacccagtgaaaaaatatatatgtctataacatcagcattgcaagttgagcaattgaatcagctaccacaacactaagtataagatgcattttatgtgagtttttcttcagtgttttaaaactgttttgtttttttacaaatcctgaaagtaattctttggtgcattttggctttgaattttacttttcattaatattttgttgcttatgattgagttagtaaatagtttttgtgtttcatttcctaattttgattttcttagtttaaatttgctgaaaaatttctaaaccagaaagtaatatagagctgtatttgattctttggctttccaacattgcagtattgattttatacacagccttagctatgtatttatatattttatgaaatgactgctcaatgctgtaatttagatttctcaccccatatttttttattctcttaacagtttttcctgttcttaattccagtgttctagttttccagtgattgatctgttagttaaatccaataattcatatatatgtaattggatttaacatatgtgtgtataacatatatgtataacatatatatacacacacatatatatgtatatatgtgtatatatatatgtatttcttttttgagatggagtctcaccctgtcacccagactgtagtgcagtggcatgatctcagcttactgcagcctctgtctcccgagtttaagcaattctcctgcctcagcctcccgagtagctgggatgacaggtgcctgccaccacacctggccaatttttgtatttttagtagagatggggttttgccatgttggccaggctggtcttgaactcctaacttcaagtgatctgcccacctcggcctcccaaattgttgggattacaggcgtgatccaccacaaccggcccagtaattcagatattaaaaggatatgtacaaaagtagcagagtgcacgtacaataatttggatacagtaaaagagtagtggtaatggaaaagccaaagacaaacatgagctgaggctgttgaaacatgaaacctaagaattactttaaaaccttttagtgaaatccatagttgggtgaattaaggggaaagaaaaaagaataaaggtcacataaaacagaactgtgcagtgacatcggagcagaattaactgacagttgagccttagtttggaaaagaaaaaaaatcaagataattaaagcagagaaatgtgacgaggcagtaagtagtaacaggttttcaggcctaaacaaaaaatagttttcagcgtagtgaaattcgaaaaaaaaaaagtaaattcaaaaaattgtaggaaatgggaggagcctaaatgctgtaaaggcaaatactccccaaagctgaaaaatgtatatgctaaatttgatggcataacctgtagattcctaagcagattatttcctatttaaactggtggctccagaaggaggttctggtcccattgtagcttgggatttcatcaagtagtagataaagatgtgcctgtattttgggatctgggccaaagaaatctgtgctctataaaattacggttggattcttgactcggagactattcatccttaaagaataaagaatatcagctgatagattaatatcagtctgctgaagggatatgtgtagcagaaaataaccaggaaaaaaattgaaatcttctgttggtgtctaaacaaacagttgtggaattatggaatggtagagacttgtcttaacaccattagcaaaatgtcagcagtgtgctgtgtttgccaaggaagtaaagatattcttaaacatcatcacctcttaaacagtgattatggatggggcagagtttgggactagaatttgatagcttcagttggcttatccattgatcattatggaatgttgtgtacagtatagggttttgcatattaaaagggacctagacagatcagagtgcatctaaacaagtgggagggtataaaaccatgtcattggtttagggaagaggacacttagatattttagaatttttccaatgtttatggggctgctgagttgtggccaatggtagaaattttaggagagcagattttggtttattaaaacaaatttataatatttaagatctttagagatgtctaaaaatttctcttgatactggggatatttagatagaaaatacatgcctggggttcatgttgggagattttttgtactatgcgtgattaagcaaacggttctgaacttcagttctctgaacaactgctgcgggttgttgaaggtagtgcagttggtggtggggcatttgagtgttgtaatgtttgatacagcaaaatagtgtgtacagcatattagttatgaagcattcctatgtttcatgagaacacctgtgaagctattacttacaaaaggactaaagcatttcccaaacccgtactcgttgcatgtttccatagtagtttggctataattgtatcctcctgctaggataacattcccagaagctctgattgccacgacttacaaatggaaagttttaaagacaaaacttttaaaaacaggattatggagaaaaagaataaatggggctcttgcatagtaagattggaaaccagtggatttagttcattcattgaataagtatatgaagcacagacacagagaggttgtaagaaagagacttgtctaaaatcacagttactgttagaactggaatcagaacttgctctgactcgagtgttctgaataacaccagaatgctgatgattttctacttttttcccctgtttataaaggtagtacatagttggccaaactcaacagtgagtaagatttttaagtccttgaggactggaactatgtcttagttaccttttcattcccatcattactatcttatacattaatttgtgttctgttatgaaattgagtgatatatcttagagactgaaaaaaatcgtggtagaaagacttcttatctgtcttccttattagaataagttatttgagagctggaaccatgacttgtttaactttaatgctccagaaacctagctctttcttgcctttgtttacccccaccaagtatttattgagggccagctgtgttccaggcactgttcaaggacctgcggacacactgggtaacagaatctcatggaattttgtgttattaatcaaggaatcacataggtatttataaaaatacaacttaagtggtaaatggcttgtatcatctaaccagatagatgcttatatatgaggactgtttgttttatacattttctttttatttatttttttagacagttttttgctcttgttgcccgggctggagtgcaatggcaccatctcggctcactgaaatctccgcctcctgagtacaagcgattctcctgcctcagcctcctgagtagctgggattacaggcgtctgccaccatgcccagctaatttttgtatttttagtagagacggggtttcactatgttggctaggctgctcttgaactcctgacctcaagtggtctgcctgccttggcctcccaaagtgctgggattacaggcatgagccactgcgcccggcacgtttatttaattttttaatctagtcattttatctgaattttctgataacttgcaatagattcttttgttttttttttttctaagtttgataaatttgtttatctggaaaatcagtattttttcttccctagatttattatattactatggttacttataatagtagatttttcaatattgaactattcttgcactcagggtataaattctactatatcttagtgcatttttctttttctatttatttcagatgtttgcatctatatttttaagcaaattttatctatagttttctttcatgctgctattctcaggctttgatattagtgtggtgctaactgcagtaagaataggtagtttccgaagaagatatttaaataataattatatggaaagtgcttttttagtccagagacctggctaaaggatgtatgtgtgattttaattagattaaatttttctaatgtcccttggttaattacttgctgtaattatgtaaatgatgtaaaggagtttaaattgttgcttggttgttgaaaacttgatttgagaattgctgtcatatgctaatttaagataaacaccaagttgatactggacattattaggaaagatttaattatgaagctagaagtataaaaagaaccttaggaaataataaaattctgcccttatcatgtagtcaaatccaaggtaatctctagatttttgttgattttatttgctattcagagaaaagcatttcgtaagctttgacaaacaaattgttatttaagagataaaatttgtaatatgaatattggtatcactgaagcagaaaatgtattacagtgccttttttaattgattaagattttataactgagtaagagtacctttgatcctgcctttttgctgtgttggattttatttaaccgaaactgatttaaataactagttagaaaaattaaagagtgccttcatatttgatagttcctaaggaactatcaataaattgaacttccccatgtttggaaagcatacaaaatacatttaaaaaatacattttatttttaaatactaatggatgcaagcctttctcccaaggaggacacagtctgtaggatagagagacattacatctgcatctggctaaccttaataatgacaacccttattgggcagtatgttccaaaggacaggagcctgtcttctggtgcatactagaagcttaataatttttgaaaagatccattttctttatgggactcggctcattaaaatagaattctcctacttttaaaaaaaatgtctatgtatatccaagaacagtaatacaggtctttaaaaacaggtcattgattttaggtattgctacttccttttttatatttaatatttcaatcattaacccagtgtagcttgatctgttaccctctttcccttgaaacttctctgatcaccagtggcttagtggtcaaatccaaggtaccatcttttgctgtttatcttacattcttttgtagtattcgatactgttggtcaccctgtccttgaatctgtcatttcttagcttctataacacctctcttctctagttctcctgcctacctccatttcttcttggtttggatcttcttttccttaccccttcctttttctcaagtggattccgtccttctttctcccattacttgctctaagacatctcttctgatagctttaaccattaaatttgtatatgaaactcagacttctgctgaacttgcagtctgttgtcttttgaacatctctgtttggatattccaaagatatctaaaaataatggaagtggtcccgtttaaatatccttcggacaaattagttctgaagtacaaaagaggctttggtattagttttgtctgacaggcattcttatagttgtttactgaagctatgatttggaatgactggcctcgtttagacttggaaaaattagatgaactaacaagtccatattataccggttaggaaggagctcgggctttgaagttaatttaataagatatgttatttgattcgtataaaaattggtttgtagtagaaattaattaaggctatagtcttgtttaaacatgggcttaatccagagaatgatagaataggccaggagctccttatttgagttttaatgctttatcaatgaaaccctcccctgccctctcttttttcctctggactagcaagaatattttttttgtctaacacatttaaaatttttacttttaacagaaattagcagagtacggaaagacatgtacaatgacacattaaatggcagtacagagaaaaggagtgcagaattgcctgatgctgtgggacctattgttcagttacaagagaaactttatgtgcctgtaaaagaatacccagatgtaagtatattttgttgttttattcaattttgttctgcttcacatatgttgggaagagatatatttggtggtaaatacttaagcattattagccactttaaaaaaatgcagtcatcaactgaagtgtgaataatttggcatgataatttaaaaaatttctctctaaatatgtttgcatttgattgctgctatgtcactttggaaatgcacatggtatagactcttgggtttcaaaactggatgaggaattgtggaaagcactatggcctgactgacacagagcaccaagcacaacattgctcatctttaatctgaccttcaagaagccacagcttgggccacagcattctttctgtaaaatgaaggtgttggaccataagtttcctttcagctctaaaatttttattgaaaccactgattgtaatctgattttcccccatcttagtccattgttgaactattctctataagcatcttgttgtgaatagcctccttgtcctatatctgttaatttctaaataaatccagattctttgctttttgttacaaagccctatatgatacggtccattctatttctctggcctcatcttaaatgattttccctcttctttaccacactgtagtcacattgaccctcttttggcttttagaataagtcaagtctattcccagtcttagggaccccatgttagttgtgctctgatctctactggctggctgcttcttgtcattaagaatttagctcaaatgtcacttcagagaggcttcccctgacctttttatttaaaagccatctatctagctactcactgtttcacgttgtctttttttttctttgcaatagcacttataatcgggcatttttctggtttattttctctctctctgtctcccctgaagaatataaggtgtacataaaataagaaccttgtctttgttattcactgctgtatccctcataatattggcacataataggtaacaatacttgttgaatgaaccaagaaaagatgatggctttttgctaagtttcagaggacttttatgaaagcagatgaaaaaaggagttattataatatcaaaaatatgatgtgctctatgattctttagttatatggacagaatactagtctgtagatgagaaatgtcttaagttgaattaataggaaaaagctttgatattttacagttacattacaatttgcaaaaattgatctgatacttttttttaaaggttatatacatttactctggttttataggtatttttggttgcaaatacacaaatagtcttggataatgtaggaaaagaggagttggttggaagattattgagtaacacagaggattaaaggaatagttggaaaacaaagtgccatgttaggctaggaatcagggcatctgagaacttgagcagcaggagtttgtggagcttctcggttgagagccttgctgacataacttggcattggtgcctcccagactttatctttaaaattcagattcttagaagagtgaatgtgttttgggcctttctgactggggaagtgaggttatctaccattacctagacaggaccactgtgagcctaccctaggtattaggattattcctagagatgggaaattgtgatttgggcagctgttccaattggggtctactgctgttaggtttttcactatcctggaaaggatagcaactgtctgttttgcttatttaaacgttaagggataatgtttctgcagtgggcattaacatagaatatgtaagaccacaagaccttgatagagaatataatgttaatctgtctggttccatctcttaaaaaaagaaatcctattcaaggaaggggtatttatgcattctttaaaagacatgggtttcatacagtcttctaagtcaggacttgagtctagagggcacctgttcactatattcctttccttcctcctccagttgtgacttcttgccacggtgaactactattaccgactggtgtgttggagcatggcatttgtcttgatcggtgattctcaaaacacagtccttggaccatagtatctacgctttctgggaactgtaaatgttcaagcccctttccatacttattgaatcagaaactctggggtaagggcgaggcctagcaatttgcctgttaagccctgtagataattccgatgcatgctgaagtatgagaatcactcctctaatttttttttttttttttggagaggggacagggtcagccaggctgacaggtaggtgatcatgtacctggtcattttgccaggcatcgtttcaggaggcctgaaacagtaagtggtgaagtgatgaattaaacagtgtcactggtcttgtggagtggacatttcagtgacaaagacaaacagtacacaaatgaatctatagctgtaatcagtgttatggagaaacataaagtggggtagacagatgaatagtgactgtgtatggaaggaggtattcatttttaagggtaatcagggaagaaatctgttacaatatgatgggaatggagagacaggaacagaatgatgggaggaaggcatgaaaaccatctgggagaaaaacattctaggcagagtgaacagcaagtacagctgactcaggcaggaatgtgcttgggaagtgttgtaagatgtgaggttagagagttagcacaggggcagtgtcatgccgtggtcaaaagtaggaactcagattcagacttctagctagttttgctgcttgttttgtgactttggtactacacttgatcttaaccaaaaggccaggaagtgatgatgttttgtgactttaagcaaattacttttcattcctggtgttcgcttttctcatttctgtaatggagatattaaacctctcacaagattatgaagactaaagagttaatatgagtgaagcagttggaaaacatctttatgctatgtgtcacaagctctatgagggcaggaatctttttgtctttcagtgctgtatttctagtgcttagaatagtgtctgataaatggtgtattcacaataagtatttgttgcacatatttgtatgaaactcattctttcaaatattgttaaacttttctttttctttaaacctgttgtcttttgcatttgcacatagcaccttacttacagaagtcagtgttaagttagagatgtgttttggacttggcagtgcagatctctgaaactgatatgagaagaaagtctcagactcaaggtctgagaaatgaagtagtgctatagtctgtagccctttgcaacgtttttgaagtttggcctcatgatgtgagaatgagaggctaatatgaataggccactgaaaatgtattaaaattttcacagaattaatatttattaataaagcagacttctattgcatctctactgtgtaccaagcacatcctaggtaagaatatttaattcttttgaaaaaaactctttgatatagtaaagacaataggtttcttagtcaggcctaggttttgaattctggcttatcctcttattggctgtatttgtcaacagttgccacaataatgctccatgataaaacactctaaaactgggtggcttaaaaataataatcatttatttctgtgctcatgggtctgtatgttggctgagggtctactggttagggtgaagcctagcttgattgaggctaactgggtgggtttagatcttctccatttatgttcctgctgggcccaggctaaagcgggcagtggctgcttagggcatgctcttatggcaggccactggaatgcaagatccaagccaaactgcataagcaggtataaggcgtctgctcttgttgctttccattagcccagtgattctcacctggtggcagttttacctatggggacatttggcaatactagagatacttttggtgtctaaacttgggagagggggtactattggcatctagtgggtggagtccagggatactgttaaacatcctgtaatgcacagggcagtcagcctccgtgcacctccagccccccaacaaagaattacctggacccaaatgtcagtagtgctgaggttgagaaaccctgcgttagtgataccaagtcacttcatttacagagagaaggggactgaaataaatatttgccgaaaaataattgagatgatcacattggctttgagcatatgcttgctttttagaatctgtttctttgtctatgaagttataaggttcacttcataggattgttgggaggagtaaaggtaacaaatgtgtgacatctgatagagcaggcactctgtggtttttgtgattacacataagaaaaataatcctgttcttgaaggagttcagtcttgtgtgaaggataaacgattaaattattgaagactttaagtttctcatgcattcatgaaactacagacatgaaagaacattgaagttatatttagaccatcaaattgtttatttggctggtcaggttttcttgtttgaatccatggagatgtgttataaatgattatgaatagagttaactgatttatttaagtaaaaagtgttatattttgaatgagtcagtaataattttgtcattaaatttgcctagttatcaatttcaagttgttagaaattgttctataggatctgccagagtagtagacttgataaatattggcaaggtctagctgttgatgatactagtcgtcctgttaaatacatgaacagattagattggtaaactgcttcctttggacatgatgattaaattgtagtgagacttcttggagttttatgtgaatataattctgattttctgatgtttttaaaatgggatttaattttgactaatagctttgaaaggcatctcttgggtctaggccatcatgaatacccatattgtcaaaggttttgtaagacagacaaatgtcttttcaaggattgtgttttcagcctgtaggatcccagcctggttgctatagttagtagtttagattcataatgttattcacagtacactccactgtctctcttataataatacatgttgatgatggaacacgagttgtaagttctctttctgtttcattttcctgggtttgcaaatcagcttaggctcgtcatagcttctgagttagttagcatagggttgttgtttgtcagcagtcttgagaagttggtgtcaaattgggtgttgagctggcgtttgttttagagggattatggggagaggggccaattcagtaagttatctttccttaaagtctttgttcctttgaccattgcggccttcttattgttgaaggatcacgaacttcaggagtcttgatgtacactactttcactttcattcttctgaataacaaatgatattaaatcaaaatttaatgatattcaaagtctcctttttcaatacccaggttaacacttgatttttgcctgaatatgatcatattttgtgagtgaaaataataatgctttaaaattacgatctacacatctgtctttgatagtttgtacagaaatctatttaaaggtagtttttctcaaaaggttagtacatatttgtattcagtggagttgtgaagtgattcagattacatattaaagttttaatttagcagtgtgatttgataatatagggcatatctttatgttttaggtagggaaaacttaatgtataagttcaactagatcggtttagtaaaagccaatttatgactctttgttttgttttgaagtttcctctttatttcactctgtaagggtatatttagggcctgttgtgtaccaaatatttcataggctttttatggtttgggatataccacatttcttagaatgttagtaaataattacagaaagggttacaggacagtctgaaaacaatacattttgtttagctgatatttttccaacgtttaacatttctttaaataataaggcctctctgagttttaaaaatgcaggcactttcttttataaataatatattttagtatttgataggaactagtgcttagaagaacaaaatgaaagatggggttgttagggaagaattattcagattagacagaaacgtttgtctatggtttatctaaaaagagtttcatgttaggatctactctgttaaaattactgcttcaaaggacttatggataatttgatatctactcataggtgttcaatctccatatctcttaagattcttgctgaaatagccaaagtatgtaatatggaaatcgaggagaaatctgcttctcagtgctatccatattccaggaacattgagctgtttctgataaagattaaaggaaagctcaaggtgatgatctactttcagcccaatttgagagagcagtgtctccgggagtaaaatggctaataacagaatcttcctcatcctaacccctcagaattgtcacacatgaaatgggctatgaagcagtcaaaggttatactgcttgaactcttccactcatttcagttcctgtttataagtaggtgacctaaatttattttgtaagttaatgagactatgttttcttttgatttcagtttgggcggaaaagtaccttagtttttgagatcacgtgcctttactttagggatgttgaggttgagtttttcagctactagcttttgagataaattcagttaatcatttcattgggaaaaaatacatatatatttaagtaatttttgttttgttttatcttagagacaaggtttcactgtgttgcccaggctggtcttgaactcctgggctcaagcaatcttcctgcctcagcctcccaaagtgctgggattacaggcgggagccactgcgcccagccagaaaacatattttatgtcttaataaacccaggatttacaacaaggtagcttacaaataaattggggatttcttgaaatcttgttttgtctgccatagaacactgcttttcccaagtccaaaatcagtgtcatcaataagcagcctgtctatcataaacttgggcatctacttgtttcatattggtataatattttctttattatttggtagggaattgattgactttgaaaacttctatcctgtattgtttggggttacagaacaatgattgttttgattagtttctttgtacttttaagattgcatataaatagtataaatttgccatgttaaggacagattaaaacaaaactcttaaaatttgaaaagtagttagaacttgaacactaaaaaggatatctgaatataaaacagcaaaattttggtatcatttcttcatcctggaaatgtttgtgtgtggtctgtgctaggtaacaggacacaggaatatgaaaagatgtaattgctctcatggtactgatatagccacaatcctggtatggaataatgagagcttgaactcaggcaatggcagttgaaatgaaaagcacaaattaaatagggagaattttaagatttctttatcttttacatgtgaaggctaagaatgagggaagggccttgctggtattgccattcgttgagaataaagatgtatgtgatgattattaaagtggtgacaatcagtaaaacatatgaaaatattgagaaaccaagatgtgattatctgaaatgatacagtaatgatatgtttgggatatatgtaggtggggaagaaatgataaaaacacaactggactaattctgaaagtaaacttggatttgaatagagtttctataaactgagtactaaacattttgaaagattaacaataatgagaaaactaaccatgaatttttcttgttatcagggtatatgttaaggtcattagaagagaggcattatactgtattaagtgtctgaactctagaatcaacctaggtgttctaggatttgggctactgatgtaacctcacatgtattagatgtaggtggtagtggtaagcactgtgcagtggtgtttcgaggattaagtgacctaatgcatgtaaaatacccagcccagaagttggctcatacttgaacactcaaaacatactgcagcatactcaacaccaaagaaatgttgaataacgtagtccttgcactcgtgatacttcgatttgacaccagcagcaaaggggaaaaattaataaccagttataaccaaaatttgaaatctgtggtatgcgaattaggatatcctaaattacggtgtatcattctttgagtactgatataactggataataagctgaaaccattcaaaacaaattcacgtgaggaagatagattatcactctagatgctaagtaacaattagaaaaaagattatttaaccaattaataaaaatacgaaggcaataaaggtcaacaagtgttttatgacaagagcagaagacagtatttgtgagagtatgagatccaatgcacaaggccttttgaaagatcccaaactactttccaagcctttattttgctgctacccatgttttctagccacctaagcaattcactcttctgtaaaaatggcaggtacttttaatttttcttccctgtaacacttcttttcttatttcttccttcccaccaaacatgctcagctccccctcatcccacaatattgagctgtagtgctccttttgctatttgtatgtgctttgactcctacagacagttgtttccccttagagtactttgtacccacctcttaagctgttatcacatggtatgcagtgatctgcctgattcttgcaggtcattctctgaagtcaggaactcgctcttagtcttttattttttagaaatctatgatatctcgcgcaatacatagcccatagaagaaactatgtagtgaaattacatagtgatttttcattctcgcagtaaaaatttcctgggattttttttttttaatgggtgggaaactacctggaaatagtggatgccagagtcttaaacagaaggaatgaaataataaagatcagagcagaaataaatgaaatagaggccaggaatacaacagaaaagttcaacaaaacagagttggtttttgaaaaggtaaacaaaattgacagacctttagttggactaaccaagagaaaaaaaagtcaaaatacatgaaggagacattacaactgataccatagaaataataagatcataaaagactactctgagtaaatatatgccaaaaaattgaacaaactagaagaaatggataaattcctaaaacatacaacctatcaggaccacatcatgaaggaacagaaagtctgaacagacctataatgagtgaggagattgaatcactaagtcaaaacctcccaacaaatagaagcccaagagcagacagcttcacgggcaaattcctctgaacatctaaagacgaattaccaccaacccttctcaaacttccaaaaaactgagcaggagagaacacttcccagattcattttatgaggccagcattacccttaaccaaagccagataaggacattgtaagaaaataaagttacaggccaatatccttgatgaacagttgcaaaaatcctcaacaaaatactagcaaactgaattgaatagcacattaaaaggagcgtacaccatgatcaagtgagatttttcctctgaatgtaaaaatggttcaacatatgcaaatcattaactgagctacaccaaattaacagaatgaaaaataaaaaccataagatcatcttaaaagatgcaaaaaaggcatttgacaaaatgtaacatcctttcacaatgaaaattctcaacaagtaaggtatagaacagtatgccacaacataataaaggctatgtgtgacaggcccacagctgacatcatacttaatggtgaaaagctgaaagcttttcctctgaggtcaggaagaagacccttgctacttccattaaatatggtaagttctagccagaacagttaggccaagaaaaagaaataaaggcagccaggagtggtggctcatggctctgattcccagcactttgggaggctgaggtgggcggatcacttgaggtcaagagttggagaccagcctggccaacatgaaaccctgtctctactaaaaaatacaaaaattcaccaggcatggtgatgcatgcctgtaatcccagcttgctcgggaggctgaggcaggagaatcacttgagcccaagaggtggaggtcgcagtgagctgagattgcaccattgcactccagcctgggcaacagagcaagactgtctcaaaaaaaaaaaaaaaaaagtaaaagacacctaaatcataaaggaagaaataaaattatctttatttgcagtggcataatcttgtatgtagaaaaacccaagacttcatcaagaaatctgttcacttagtcaagttctgggatacaaaaatcaacatacaaaaatcagttttctgtctatacactaacaacaaactattcaaaaaagagattaagaggtcaggtatggtggttaacacctgtaatcccagaactttgggaggccgaagcgggcggatcacctgaggtcaggagtttgagaccagcctggccaacatggtgaaaccctgtctctacaaaaaatacaaaaaaaacaactgggtgtggtggtgcaggctgtaatcccagctacttggaaagctgaggcaggagaattgcttgagcctgggagagacggaggctgcagtgagccgagatcacatcactgcactccagcctgggtgacaagagcgaaactccatctcaaaaaaaaaaaaaagggctatctcatttacaatagcatcggaaagaataatataattagaaataatacccaggaggtaaaagatgcctgctcgctgaaaagtataacactgatcaaataaattaaagatgcaaataaatggaaaaatattccatgttcgtagattggaagattattgttaaaatttccatacttcccaaagcaatctatagatccagtacaattcctgtcaaaattccattggcatttttcacagaaataggaaaaaacaatcctaaaattcacgtggaaccacagaaagcacctaatcgccaaaacagtcttgagaacaaagctgaagacattgcagttcctgatttcaaattatattaaaacaatttgcttctggcataaaaacagacacatagaccaatgaaacagaacagaatctcagaaataaacctatgaatatagagtcaactaatctttgacaagggtaccatgactacacaatggagaaagaatcgtctcttcaacagttggtgctgggaaaactggatattgacattccaaagatgaaagtggacccttacttacaccgtacataaaaatttagtcaaaatggattaaagacctaaatgaattccctgaaactataaaacatgtaggaaaaaacactgaattataaaggtatttgatactggtcttggtaatgatttttctagatattacacccaaaaaacacaagcaacaaaagcaaaaataaacaggactacatcaagctgacatgcttctgcacagcaaagaaacaatcaacaaaatgaaaaggcaacctacagaatgggaggaaatatctgcaaaccttatgtctgctaagggtttaatatccaaaagataccagaatattaactcagcccaaaacaacaaaacagcaaaaaaaaaaaaaaaatgacatcacccaatttaaaaataggcaaataacttttagacattttttcaaaagagaatatacagatgtccaaaaggcttatgaaaaggtgcttaacatcacacacacacacacacacgtgcgtgtacatgcgtgggcacagtagctgtgtgggtgatggatgtgttaattagcttgattgtggtaaccatttcacaatgtataccttgtattaaatcatcacaagttaaaccttgttcattatatctcagtaaagctggagggagggattcagtcactaaattgtaagttgtatgcagcacaggaactcatatgtttagaactttctcatataataagcctgactggctgttctcaggataaatcccaaaggacaaagtcagtaataaaaaacaaaacagttactatttccactttctatttcaacatggaagtatgcagctttaaatttttgttttgtttttacatagccttgtaagaaaccttttcttacacagcacttgtacgtaacatgtgccatgtgtgctatgaacaaaaatgtttataaaggcctaatatgtgaaaggttttgccaaatgtttcctgtaatgttaattatattttataaattagctatttctccaggagatgtatttagccacttattaatttataagaattaatatgaaggtaacaaggcataagatgctcgttttccctcaaaacacaagacattttggtatatagtctccaacttacaatggtctgacttaggattgtttacaatggtttgaaaggatatgttttcagtatgcttctcaatttatgatggggccatctccagataaaaccattgcaactgaaaatatcgtaaatttaaaactgcactttcaactcagtggtgggtttattgggacttaactccattgtaagtcaaggatcatctgtattcattctgtatttttaattaaaaacagttgtgctgttaatatatagacacaactcttctctcatcttacatacttgtataagataggcccactagtgttgactagattgggtgtggtccgttttttgtcttctgctaagtgaaagacttcagaaaatatacatatacgcttctgtgaatgtatttgtttcatacccactgtgttgcttatgtgatataatgaaagatttgtaaggcatctaactaaaatcaccctagaaataactagacacaatatttaaaaacattatcaatctgttgtgcaaaaagcaatccaaaaaagtgcattttttaaatgaaaacttggtctctccccttgtccccaaattttaggcacagattacactttatcagttcttagaagtgtttcacttgaggtgcttttcgaaatccattttttattatctattgtgataatgtgttgcaggagtttatctttaattagggaattttaaaaaggttaatggaagggttttctataatgctttacattttgtcattgggaaaaatctatcatttccatttaaaatgaagtgtgagcagcaatgaacagcattagacataattgttgttaaacctcttctttgtaaattaacatgccacttggcagaaatataattggtgagccacagagaaggtctgtatttaaacagtagggttaatgaatatctttttttcccctttttctttcccttcttttggcctttaaagttgcacttagctcaggctgtttgaaatgattttctttgaaagtgttgtgacattgttttaattgaatgaagctgcagaactggcgtgataaatcaacacagaatgaattttaatgcaaggcaaacgatattaacctggaaattggttgcagtagacaaagcagctgctgtgattagttttttttttttcttcattctctacttacaacattttgagaaggatgacctcgacaatttcagctgtactagagtcctgatggcttgcaccttaacatccttgagctgtttgtggcttcttggggcaatagtgattgtttcattttttaaggttagtaaaattttttaaggttagtaagattagtaaggttagtaagatttaatgtgattttaactgctttacaaatatttttaatttagtaaatcataattgtgaggattaattagctgtggtagaagcccttttctatgtaatcttgatgtggttccagccaaggagggttaatacttgaaaagcatggggtatgccaaacaaggctctatatgtggtcacagagtgtagggcagctggtttcacaaagggtacgggtcaaaaccaattacatattagtgatttgtactaaagtatctctgttcataatgcatttttccagacatttttgggccatatgtgaagtatataatgtgtatctagttagtgacaatttgggaaaagatctgtatagagtacaactattattgaagagtagctggcaaggttacttaaagaaatagccaaggtaacaaatatagtagaggcattttcacagacttcttgagagcagagtttcgttattctcaatccttcacatttacttcactccttgccacataataagccttaagtaaatgttttgttgaatttgaaaatgaaaggctgttctctgttttactgaaaaacagtgggtgagtgatagcatgtaaagattattatctctgtccaggttggaggagtggggaagtgaaatttaatgtaatctgtgacttacataaactggttaagtgatgatgttgtagaaaaaagtaggaagagcttattggagatgaacataaggccaggcatgttgatagattttctgttatgtttcttttttattcagatcctcatttcaacattgttacaccatactaccttatgtcaattctagacctctttagattaaaaagcaactgctcaaaaagtgccagtagacttgtgtttgttaaggactgacaagataatgcaggccagctgagcaattccaggaaaataggatacaataggaaaaaaaataactgaacacatcagagattgtgaatcagcaagattgtgaagaattactagttcatggtcttcaggaggtgagatgatcaaggatataaccgtgttttgggggatctctttttctgctgagcacgtttgcagaatctggagaggacagacgaaaggctgagctttgggggatagcgtttggagtctataatttgtctatgtgaacctcccttgccatgcccagcctagtcttcgcttgggggtagggacactgatggatacgtactttaggagtaagaatgaactaggaggttgcaggtcctcacagagacagtatctcagctcaaaattgtctatctcagtaagtgaaattgcattgatcctgggttgctagtaatagtaggtgcctggcagaagctgatatgtatctttcctggaggaagatattattgtctttggccttatattatttttataagctatttagttaagtgtattgtctgacacacactagaaaataagcacacaaggagacaagatgacattaacaaaaatctgaagaaacagtctgaaaaacaatacaaaatccacatactctagattttagaatttcagacccagactttataagatctgtaagttttcaatgtggaaggagacagaagaaaaggaagagaaatggaagctataaaagaaaaactaagccagaaaattatttacctactattaacaggtgggtttaacagaagattagaagcatttgaagagagaattagtaaactggaagatagctcagaagaaaattttcagcatggagatgcagagggatggaaaaaagtagaagggaatgtgaaaagtaaaaggaatatagtgagaaaatttaatatatgtagttgtagtattagaagaagaggagaggtggaatgggacagaagcagagtttgagggaagtgtgtttcagaactataaaaccaaagaaagaaataaagtcacagatttgtgaaattgtggttttctttatatttatcatacttactgataaacatgataaaggagaccacaccttggcaagtgataataaaacaactgaaaagtaatgatgagaaaatcttaaagcagctagaagagagaaaagatcaccttcaaaggagcagcgggaagacttagttcacttccattagtaacagtggaatgaatccaggagatggtgtataaagaatctttaaggtgcccttcaaaaataagacattttcagataaggaaaaatggagagagttcattactagaagacgttcactaaaggaaatgctgaaggttgtttttcaggcagaagaatgatgattatttgaaaagttgaagatgcataaaggaataaagagcaacaaaaatggtaaaattgttgataaacctaaatgcatatttgtacaaagtgaaaataatggcctgttgagtttaaaagatacagaatttgaaaaacaaaggcatataaactggattgaggataaatattacactttctgaggtccttgtatttgtatgggaaaagggtaaaagtacaaatgaacattaatgtttgataagtcaaggaagcatgttgtaaatactagggtgaacaagcatataacttccaaactaatagaagaagagaacgaagaaaatacgttcatattttcttcgtccgcccacatccaggaaaaagaggcaagaaagaaaagaaaataagtagaggacatagaaaacacttagtaaaaagatttagtaaaccatgtagtataaattggctgttcctgttaaaattcaatgaagaaacaactcatttagatgatttttacaggagtcctgtttaaaataaggataacaaaagtttgaaggtaatagattggatcaagaaaagacacataagaaaataaattctatctatattagtatcagtgtaagcttaatggcaaaaggcatctctagagataataaagaaggccacttcataatgacagaaagatatacacttgacccttaaacaacatggggctgaggggcactgactccctttgcggtaaaaaatccacgtataacttttaactacctccaaacgtaactactgatagcctactgtcgactggaagccttaccgataacataaacagttgatcaacacattttgtatgttacatgtattaacactgtattcttacaataaaataagccattgagaagaaaatgttttaaagaaatcataaggaagaaaaaatacgtttattattcattaagtggaagtagatcataaaggtcttcatcctggtggtctttacatggagtaggctgaggaagaggaggaagattcaggtttggtcttactgtctcaagggtggcagaggcagaaggaggtgatggaggtgggaggggtgtcatacttggtgtaatatttgtggaaaaatgttctgtaagtagacccacatagttcagacctattgtttaagggttggttgtaattgcctttacacatatacagcaaaaatagaagtaaaaggcgaaacaggtaaaatttaatcacaccagactttgcattctttttaagcacctatggaatatttacaaaaattgaccatatgctatgctgaaaagcaagtttgaacaaatattaaggatggctattacatataacatgtttgctgattacactgtaggaagctaaaaatcaaaactacaggaaattatatgttgaaatgagcataatatatttaaatatttagtagatgtcaaattaccttgactttgaatgaaaatgaaactgctccctatctgaatttgtgggatacaactaaaaccagcttagaggtatatgtaaagctttagatgtatatgttagaaaagaaggaatacaataagcaaaatattcatttcaaaaaattggaaaaagaacagcagattaaacgaaggaagggaaaaagaacagcagattaaacgaaggaagtagaaggaaggaaataaatgaagagtagaaatcaattaaagacaaacacaaaatgaagtaattcaatatatcagctagctatctctacattataataaactactccaaaatcttaagtaaaccacttgttgagctccttagtccttcttccagtcagctgtaaggtcagtcaggtagttctacttctgggagttgcgtggttgcagatattgatgatgactgggtcactgtctttcattccccaacaagttttagtccaagcttgtttacatgggggaaaggggctctaagagagtagaaccacgtaagactaacactgaggcatatcattctcaccactacgttgtattggccaaagcaagtcacaatgctagcctagattcaggaggtgggaggagatgcagattccctttgcagatggcatggacacaatgaggggcagagaattatggtcatttttgaaatcagtctacctcagtcaacaaaagcaggttcttttaaaacacttttgaagacagatcaagagaaaggaagatacaggtaaccagtcaagaataaaaaaatgagacatcgtttcagatcttattggtattaaaaataaattaggcatctattttaaatcaacaaatttgaaaatttagacaaaatggatacaacttaaaaattgacatacgaaggaatagaaagtctgaatccataatttaaaactttctgtagtgaaatctctaggctcaggtagcttcaccagtgaattttattcattatttaaggaagaaataacagccatatacaagcctatctggagagtggaaaaaggaggaatacattccaacatgttttgctaccagaataaccttgataccaaaacctgagacgtgtattatgagaaagaaaaattgtgggccagttctatttatgaatggacatttcttaaaaaaaaaaatcaccagaatgccaggaaacaatttgctcaagttgattttgcattaaattggtttaacatttgaagttgaaattgaatttgaaattaaatttgaagttgaagttggttaacatttgcagttgaaatccactgcaataacagaattaaggagaaatgttgttttaatagatagtgaaaaatgataaatttagatatccacttgtagttgggaactcttagcaaactaagaaatagaagggaattttaatgtgataaagtgtatcttcaaaaagtttaagattaaactttatggtggactgttgaaacctttattcttgaaatttggaacaagtcaaggatgacacacttttatttggctttgaactcaaggtatgctgaccagtactgtaaggatagcaagaaaaagaaataaaaatatatgataggaaaggaaaatagagaactgtcattatttccaggtgatgtgattgtttagcatccaaaagaatctacaaaagatttatgagagtcagtaatgaagaaatgctggttgtaaggttgatagaaaaaaacccaattatatgttcatataccagaaacagaaaatgaaaacttaaaattatgatatacggtagcagaaaaagccctgtaaatttgtaggaatgaatcttaacaacaacaaaagtaaaaacaagtaaaggcacaatcaaaaaagctctacatttttgaggctagacatggtgtctccagcctgtaatcctagctctttgggaggcttaggcactggggtggagtgggggtgaggatggcttgagtccaggaattttgagatcagcctgggcaacgtagtgagacctgatgtctacaaaaaagttaaaaaaaaaaaaaaaaaaaagccaggcatggtgaggctgaggtgggaggattgcttgaggccaagaattcaaggctacagtgctctatgattgtgccacttgcactttagcctaaacaaaaataaacttgacgttattgagagagatgagagataacttaaccaaatggagaaattttaccatgtttatggtttttgaagagtctatattataatgtgatctcagtgaaaattcttgcaagtattattttagaaattgatggacttattctaaaatatgtggaaatggaaagggccaggaatagctaagccactcttgaggaagagcaagagagaagaacttggtgtaccatataatttaccataattaatacagtatgatgttaaagtaaagataaataaatggaatagactagagagctccaaaacagacctaagtactgtcagctgtttgtatcctcaggttccacatctgtggattcaaccaacctaggatcaaaacttttaggggaaatatttaaaatggttgcatttgtactgaaatatacaaattttttcttgtcattatttcctaagcaatgcagcataacagtgatttacacagtatttacattgtattaggtattttaagtaatctaaagatgatataaattatattggaggttgtgcataggttatgtgcaaatatgacaccgttttatataagggacttgagcatctgtgaattttggtgtttgtgtggggacctgtaaccaatcccccccaccccagataccaagggacgattgtgtagataaatatttatgacagtgatgacactggagcaattggaaaagagtagtcttttcaataacatttctattcagtgacattcgtatatttaataacatttggatattcatgtgagagaagaaaagaaaattataacacagacatcaattcctggtacactgtagatgtaaattcgaaagacttaacaataaagcttctagaagataacaccagaaggtatcttggtgaccttagggtggggagagacttcttttaaacaggacacagaaaacactaaccatgaagataaggttgaaaatttgaactgtgttaaaattaaaaactttcagtcatcataagacaccattaaagagggagaagacgagccacaggatgggaaaaatacttgtcagttgtgtaaccacaaagtagaaatcaataaggaaaaggtaacacaatagaaaaacacaggaaacttcaaatgtccttcaaaaaatgcatatccaaatggccaacaaatgtgaaaaaaatgcttagcttcattagtagtcagaggaatgctaattgaaacatttaatacatacactagaatggttaaaatataaaaacactgataataaatattggagaggaagtggatcaaaataaatttttatactatacggaggataaaaatatgtacaaccattttagaaaataatttaacactatgtactaaagtttttttttttttttttaaatcatcttctttgtcctgtagtcttttttttctcaagggtcagtctcttgtcctgcttctttatcttgtcctgtctctctcatgatagagcccttcttgaacctttggtgaacattgtttaagtgtgaaacaatgaaaatctgaattaggagcttgtgttcatgaacagagctgtccgctgccaaccttaatttggggtgaccccctaaagtgctagtctttgagatggaagtcatataatggaacacagaattcttttcgtgggttatttcaatttttctagagaaaaaactagcccattacctacaagtcagaagcctggcctttgctgttctgtgggcagtgatggtcgcttgttctgtgtattagttccaccttttcattcttgttttctgcaaaactcagtgtgtgagtgtccagtctctctggagttctgtagggcaagtttgctgctgatcacatctccttctagaaatgacattccaagcttaattaagagtaagagcctgaggttttaagcaaaggatttgtattagacttggtaggctattgtgactgtatggcaaactttttactggaccttgtgtatgtattgtgctatacacatacatatctgtacatgccatcatcactatagctctgtcagatagtattatccccattctgtaattgaggcttatttgatgtgattttcagattcctttttttgttttgttttgttttgtttttttttttttgagacagagtctcgctttattgcccaggctgtagtgcagaggcatgatttcagctcacggcaacctccacctcctgggttcaactgattctcatgcctcggccacccaggtagctggaattacaagcatgcaccaccaagcctggctaagttttgtatttttagtagagatggggtttcaccatgttggccaggctcgtcttgaacgcctgtcctcaagtgatccaccccgtcttggcctcccaaagtgttgggattacaggtgtgagccactgcgcctggccaatattcagattcctgattactgttttcatatttgagcttggataccagctagaatctgtgtgctctcagagtttaagcacataaccgtagtatactacttgagtttatttaaattggagatgtaggaaggttaagttgatgagtttctaaattggaagtactttgttaaaatggatcatggtgggggtttaggttgggtaaagaggtaaaattactatatactgaagcactgggcaaagataagggactttatctgaatataaagagctattagcagatttcagaatctaggatcttgaagagctaaattaagtgcttatagatgagtctgtgagtctcaacagagggtgaaataagatgtatgaaaatcccctcggagactttcttcgaatgatacaggcgtactacctaccacctttcatttgagaatcacttttctaaggtgtgacaatgcaaaataagagccagtggagtacagatgataccagaagtacccagcttcttaagccctgatggcagtggattttctagcacgttacttttatgtaatgatatactaaaagttggataaatgcaagaaaaaagtagaagaccatatgcataagaatcagtacctatttgattgaataagtaaatgaaaaccacctgtggaattttgtggaactgatagcaaactcttatattttggcctttatgcagataacatgcaaatgaggaaaatttacttttcacgatttagcccgtttttcctattggctttaaagaaataacttgtatatatgaatattaggaatttctgttgagggatggtaattttttatttttttattgtacagtttttaatttatacaattattgatatcactggcctatttagtgatattcagagtttaggtttcttataagttaaaggtagaaaggaatatgtactcttaagacacatttagtgtagccaacccatagttttcgtaacatatttctgaacagttgctttggagaaagtataatttttcttaggtaaataaaagaggactttccccaaaggattttggtagaaagggcagtgcctaacaatttcttcaacactgtcatgacaaagagacagttggacattttgtggaagggacttgtattcttggctggcatctaatattttgtcaaactctttaatactctctgttttaggttatttgttgatggacagttgtatctgttctagctgtatactgttcccagttcatagtttgtgttaatacttgtgaaaatataatgctaaacgatgagctattgtaattagacctgagacttagcaagaaattcataagggctctagattttatttcaacttagtattaaaaagacatttaacttcagtaagtaaatgtagatatactttatattttaagagctgtaagttccagcaagtgaataatgtatagtgatccttttttttttttctcagtttaattttgttgggagaatccttggacctagaggacttacagccaaacaacttgaagcagaaaccggatgtaaaatcatggtccgaggcaaaggctcaatgagggataaaaaaaaggtaagtccttgaaaatggactaagtctttatgtgagtaacttactatacttttacatcgtaataataaaatttccagatttttttgtgcttaaaacactatattccaggccgggcgcggtggctcacgcctgtaatcccagcactttgggaggccgaggcaggcggatcgtctgaggtcaggagtttgagaccaccctagccaacatggtgaaaccctgtctttactaaaaaaaatacaaaaattagctgggcgtggtggcgggcgcctgtaatcccagctactcaggaggctgaggcaggagaattgcttgaacccgggaggcagagatttcagattgtgccactgcactccagcctgggcaacaggagtaaagctctgtctcaaaaacgaacaaacaaacccactatgttccatgtcaggaacttttaaactttttaaaggtgttgtcatgattttcccttgtttttcttttttctttgtttgttatttttgagacggagtctcgctatgtcacccaggctggagtgcagtggtgcaatcttggctcactgcagcctccgctttctgcattcaagtgtttctcctgcctcaggctcctgagtagctgggattacaggcgcacgccgccatgcccagctaatttttgtatttttagtagagatggggtttcaccatgttggtcagtctggtctcgaactcctgacctcgtgatccacccacctcagcctcccaaaggtctgggattataggcgtgagccaccacgcctggcggtttccccttgttaattatcattttttacttagcaaattaaaatatcagttcatcatgttaccttcctttgtcatttacttttgggaagctgccagtttggaaattagtcagactgtaatgtgtgtaaaagtatataaaagttttgaaaaatctgaaatttttcttttgatgagatttaaataagaatggtcaaataactttggatgtatgtcagatcactgggagagttagaagacagaaatctattagaaggaaggctatcactagccatatgagatacactttgggtttcatcaacattttgtgttgattttacattttaaagtatattgtttctgtttctgtgggtttttttctcctgccatttcttcttttgctggtttaattagtaaagacccaactatctaaatattattgttaggaagattttatttacctagtttgtcttacccagcaatactgtgaggcaggatagttttgaatttctattcttgtaaaaattggcggatttaaaaaaatagattttgatggccaaagattaaaagtgttttaggaaggtttggtctgtctgtctgtctgtctgtctctttctctctctctctctctctttcttccttccttccttcctttctttccattgacttgaaggggctatctaggaatagtgcgtggttagctgtgttagaccagtacgctgaatcgctttgtggaacttacggaatgaataccaaacccacagggtatctgataaacttgttttctgagccctccgcctcatgtcttttaagccctcacataagctgactggatctcattcatttttgctcctcatcaatgatttagtagccaagaaatttgggcaaaaagttctcataatattgattatttaaaaatcttattataatgtattccttgtcaactgtgtttaaagatatgttcatgggagtctgtgagttctccttgagcaattttagatgtattaaatttttttatgagaatcttaaaaaaatgtgcatattttgaatcagttgagtgataatcttttcaaatgaacgttgccatgaagttttggttctcatgtttggacttaggattatgtggtcattaagtaatactagtttaattgcctatggacaatgaatgaagtcttgacaactggcagatatataaatctgtatgaaaagattagtatatattcatcagttaggctattctgtcagccagttaacaaaacccctttttccctgcttctgagtttctgtgaattctgcagtaatggccccccactcaaccttgcttactgtttcaagccctcacagacaatgagcattagagcttataactgtgtgattatactgttgcactcttataagtgtttataataaaagtaaagtcagaatggaatgcttggccatttggtaaagaacatgaaaagagatggaaacaaaaagaagtattgatgtaagtaataaagatagctcattgagatgtatttcaggagttcaagaggaaaagccatttgctttaaacttttctacagagttgtccatgtatatatagacatgtgtgtatatatatatatgtgtgtgcgtatatatataatgtgtatatctatatgtgtgtatatactgattgcctgttttacttttaacacaaaatgctacgatgggaatggtgtgtgctggtctgaatccttccctacactggtaaactcagtgagagcctaccatggctattctatggagtattaatttccttggtgtttggtgagtacctggcacagagtgcaggcacttgaaaaaaatacctgtgaaaggagtgactagttcaggtaataaatgaaaggcttaggtgtaccttccctcatgttccccgaaaaggttggttaaataaagaaaattgtaaatgctatgtacttctaatgctttggggaagttagtactatattattctaagctgctggtttttagcaagtaagttaatatgccactttagttttcattttcttggattgttattatttcagcattattgatgggctcaacagcattgatttagtgcataaatacccagtggaatggtactagttaagatcatggaaccagggcttgaaaactattatgaacttcttgtctgtaccagctattttgtaaatgaagagaccaaagccttggggaggttgagtaatttgccaaaggtcacacgccaaaatgtggcagtcaaatttgaaactaggctgggcacagtggctcacacctgtaatctcagcactttgggaggctgaggcaggaggatcccaggagttgaagaccaacaaggtcaacatagtgagacccccatctctacaataaaacttaaaaaataattagccaggtgtggtggcgtgcacctgtagtcccagctgctaggtaggctgaggagggaggatgcttgagcccaggagttgaggcaacagtgagctatgattgtgtgccactgcactccagcttgggtgacagaacgagactctgtctcgggggaaaaaagaaaaagaaactgaattctgagttctattctgtggtacattattttctaattataaaccatttctaagagtgataacttcagacatactagaacaacttgaatattgagtattagaaactttgactaccatttcacacaccacccagtacacttgtgaccccagatgttgtaagggtttctccccacaaaccaaacaaatctccagaaaacaccatctgaatgtcctctaattcagttcaaaactgggaataatgtcacataccacaggctgagggctcagtcccctaagactgtctccacttcagatgccagttacaagccccagtggtgacctgtgcttttgactgaccagctgtaaatgtgtttttttgcaagtccctcgtcgggttcaattaatttgctagagtagttcacagaactcagggaagcactttacttatttattataaaggatattacaaaggataccgacgaacagcagatggaaaagatgcatagggcaagacatgtgggaaggggctcagagctcccatcccttctccagtgtgccaccctccaggtgcatccatgtgttcagctatttggaatctctccaaaccccgtccttctgtttttttgtggaggtttcattatgtaggcatgactgatgttcatcattggccattggtgatcgactcaacattcagcctttctcccttccttagaggttggggaatgaggctgaaagtctcagccctctgtatacttggttagggtttctctggaatcagccttgatcttgagactatccagggcccccacccatttgtcacttcattatacagaaagacacaaacaggttccagaccaccacaataagggaacattgcgataaagctagtcacacatttttttagtttcctagtgcatataaaagttatgtttataccatactatagtctattaaatttgtaacagcattatgtctaaaaaatatacataccttagtttaaaaatactttattgctgaaaaatgctaatgatcatctgagccttcagagagttttaatcttttgctggtggaaggccttgcctggatgttgatggcatgttgatggctgctgactgatcagggtggtggttgccaatggttggggtgactgtgacgattgcttaaaataagaacaacgaagtttgcctcatggatttgctcttcctttcacaaagatgtctctagcatgcagtgctttttgatagcattttacccacagaacttttgctgttgctttataaactaagtttatgtaatattataaatttttttttgtcatttcaacagtgttcactgtatcttcactacaaatacatttcatctcaagaaatcactttctctgctcatccataagaagcaactcctgatcaagtttgatcatgcagttgcagcaattcagtcacatcttcaggctacacttctaattctagttctcttgctctttccaccacatctgcagtttcttcctccatcatactcctaaacccctcagagtcatccttgagggttggaattaactttttccagactcctgctaatgttgatattttgacctctctccatgaatcatgaatgttcttaatggcattgagaatggtgattcctttccaggaggttttcaatatactttgcccagatttatcagaggaatcactgtctgtggcagctataggcctatgaaagacatttcttaaataataaggcttgaaagtctaaattactccttgatccatgggttacagaatgaatgttgtgttagcaggagtgaaaacaacattcatctccttgtacgtctctaccagagattttgggttaccaggtggattgacaatgagcagtaatatttggaaaggaatctctttttctgagcaatatgtctcaacagtaggcttaaaatattcaataaacctttctctgctgacatccaggctttgttgttccaattgtagagcacaggcagggtacattagcctttttttttttttttctttttaagacagagtctcactctaccgcccaggctggtgtgtagtggtgcaatctcaggtcactgcaacctccgccttctgggttcgagcagtcatgcctcagcctgctgagtagctggggttagaggcatgcaccaccacacccagctaattttttgtatttttagtagagatgcggttttgccgtgttggccaggctagtctcgaactcctggcctcaagtgatctgcccaccttggccttccaaagtgctaggattacaggtgtgagccactgcacctgtcccatttagcataatttttaagggccctagtatttttggaatggtcaatcagcatttgcttcaacataaagtcaccagttgcattagcccctaacaagagaatcagcctgtctttggaaggtttgaagccaggcattgacttctctctagctatgaaagttttagatgtaatcttcttccattagcagtccagacacacacagtacttgtcacttaagttcactgtcttatatgggcatggtttggtgtgccccagaacaattacagtagtaatttcaaagattactatctacaggtcactatggcagacataataataatagtaatgtaaaagtttgaaatattgtgagaattaccaaaatgtggcttggagacacgaattgagcacatggtgttggaaaaatggtgccacaagatttgtttgatgcagggttgccataagccttcaatttggaaaaatgcagtatatgtgaagtacaataaaaggaggtatgcctgtattacttgggagattcaggggttttaggagatttgtgccaggaaacaaagaccaaatatatttatttcatcataaatcacaatatcatactatcattcctttgatttttcaggaattctccctagaggatttaatcccatgtacttaatggaaatctatatacttgagcagattacaaatgctagaggagtaattttagatgagatcgggtgcattcagtatggtatggtgtagactagaggatgaattttaggaaaagaaagtaattatggtgtttcacttgaggttgcagaggccgtatccagggctgtgaaagaaatgtattcatgctaggaggcatggtccacctttagaaacaggtgggagtcagctggttctaatgtagcaaatgagaacatgtaagccattaaaacagaacagtgttttgcacgtaattaggtacttggcaaattgttgtggagtgacaagtagtacattgaagttcaccctttgggtatatatatgcatggtcattgtaggggacaccatttgtggttttgtcttatccgtctgcaatttgtcagctcttctgaggtctcccttctcataaacaagaggagaataagtagccatgcttgtgggttcctccttctcacctcctttttctgtccttccttctctcccattgtgttgatgggtgaaatgtatgcagtttatcaaagccatgtcagtcagatgttgcatcatctggaaatgtgcaaagacttagcaattccagttcaagttctttgctcaaaatgaagggatgtgcattcttcttctagccattccagggctgctaataacagtggctagagcaggggtgtccaatcttttggcttccctggaccacattggaagatgacaaattgtcatgggccacacataaaatacactaacaatagctgatgagctaaaaacaaacaaacaaacaaaaacctcatgttttaagaaagtttatgaatttgtgtggggccacattcaaagccttcctgggccgcatgcggcctatggaccgggggttggacaagcttgggctagatggtatcagccacttttactatctgtgctgcatcatgttcagaccatgggacaaccttggaaaagagttacactcttccaacggaaatggaggcttaaagaagtgtagtaacccacccagttgctcatttctacctgaagagtacacaatatagaaacttccatctctcactggatattcaaattcagttgtacaaatcattcttgtcagaatgtgggctttgaaataaattggtaatagtttttaacttaatatttgtttcccaaatgtatttggccatagtctccccttttttgaagatgtccctataactccttgaagaactacagtgttttgtggaacatgacttacgaaatgttattagagtcatattgtataattgcagacataggtggaattcttgaattttgttttgcaggacttgtttaggtagagcatttaataacttcttttacgtgatgcttttcatatacattacaaattgttaagtatctcttatagcagagatttcttagctctatttgttatgcttaaaatatattttaattggtcaatatgttatatttttagaaaagtgagaaagtcctagagggtctcaagaaaattaaaggctcctgacattccattagtttagacactacccttgtttatagtttggtatgtcttgtagattgttcttcatgcagttatgcatatgtacacgtatggtatattaagtggatcatattatatttgctgctttataatcaacatttttttcctgcaactctgtatcctgtcctatgtcagttcaccgaatagtagagctttatgcctaagactgtgataggtgataggaaagtacacttaaaggacatttcaaggcagcattcatagtgatatgcaaaaataaatttccttgaatttttgggtgggggaacttacaagattttattcttatgaaatatgttactgctgtgtcaacactttgaaagagagaaattaattcagtcttgtgattgatacaccagagtggctttggcaattggtggcatgacatcaagtacaagtagggttccaaacagcctgttttatcgatcttgttgaatacttagctctctgacaagctctagcatggatatagttttttttttttacgttttcattatgcaaaattttaagcaatggaaagacaagtagtgtagtgaactctaatttacccatcacccagggtcagcattgcctctctcctttcaagagggaactctctcattccccattgcctcacttcaccttttgcctcccagattattttagaatcaaattcagacatataatttcaactgtaaatattttattagctatctctaaaatagaactctaatgataacgtgtttgtgttttaatatctagaaaattatttatcctcaaatatgttgtcagtgttaatagtttctctggtgtctcattaatatgcttttaaaataatttgttttaatgagcatacaaatgagggtcatctacattgtaattgatagatctcttaagccacttcccaaacctccatttctctttcttggcttcactgggtgatttgtcttgaattcctgtggattttgctggtggcattttcttggtggtgtttaacatttatctctgttccccatatttcttgtaaattggtaatgacaaaggtttagttaaattcagctctcccaccttctctttctcatttctgttcttttttgaaagaatatttcataactagtgttttgatggtagtacataaagtctcatttcctctctgtgattttagcaatcattgttgattgttttctaggtcctttttttcagtgggagttgcagaatagctatattctgtttcttcattcatattagctagatacagtttttatcagaaaggcggaataaatgtttaattctttatttgctagtttttaaatagtgaattggttcccaagaatcctccagactgaaagaatgagtgtttccctgccccccgcctttgggtatcactacgaatgcatttaaacagttttatatgtttccgttcattgaagatattatttttaactggcgctcaagttgtttggccattggagggacttcttcacattgacttctaaatcttttcaacataatcctagtagatgggaatagtaactttgccgtctagtgtagtgtgatatgacagtctgcactcttgtgagaattcttcctcagacttggaatcagccaattttttagggaagctttgttaacaaaaagtgacaaaaaaaaagagagagagagagaaagaaatagtgcagagattatagtctgggctctagtgattctcattattgtaggcttttcagagctgtgaaatacacacgaatattttcacaagctcattctcattaaatttaggactataatgtttttatttaatcttctatctgtatcttattttctcactgaaaattctggttcctagtgacacaaaataatgattcatttgctttatttcaaaaaaggaatacaaagcagtctcagaataactataaccactactaccagtgatacagttggtgacaattttacactgagctcttactattttgagggtatatccactagatacaagttaccatctcttaaagcagctggaataatttttactatgtaacagttgtgctagtatatagatccatggttaggttcatttgttgcacttgattttctagttgtggaaattgcataaaatattttcctgttttctaccttagaatatttacatggttccaaagcaaaatatgcaaagcagagaatttttataaagtatggctttcatctcaggcccatttatttacctagtcctcccttgctgtttgggtagattttttatttttagtttttggtttatccttccatttctttttttttttttttttaaacataaacagttatgtaacagttttgtgtgtgtatctgtgtataatttctcactcttcatagattattatactctcagcactttttaatacctttattatttattttcacttaatagtatattgtggtcatttaatgggagtgtataaagaatagcctttattcgttttttttttttttgccgttacaaaaaggtatagtgctgtgttgaaaagtgtttcttgaagttgctagtttatgtttgaatagaattgtagaagcaggatcgtgtgtggttttattagttagtgctaaattcctccgtcttagggtgatggtttgcgttaccatcagcagtgtatgagagtagcagtttccccccagcttcacaaaactttgctctgttgtgcaggagattgagcatctgttaaaggcactttcaaagaagttatgctgtgtgtgtgtgtgtgtgtgtgtgtgtgtgtgttgtgtgtaaaagtcattactgttatgaacactgttaccaccttcagtttcccaactattgtttgtagtgcgttttttaatattctgactgaagcagtttataaataatggttagacttacttgaaatggtaccattacaacatgtcttaaggcacactaattgtaataacttctggctgggcttaagaaactacttcttaaatgtttttaagtggaaatatgctttgatttttctgtaagcttaacaagcttttctcagctagggttcctcatctgaaccacagaaaataggaattatttcattttattaaagatgactcatcaagagaaccattatagttacacaggagagaagttaaccacatataatttttatgtcttgggctgggcattgaaatttgattctcatgtggaattcttagttgagaaaggtgtaccctgtgagaaatggtttttttctcgcattttatattactgcctctccatcaaaccatatatatctccccctcttccttcagcctcccttattggtaccatttgacgagatgattagggagtaggaactgttacaggttgtttgttagagggttaatcttacaaattgtattcttatcaactttcatgaatccaattgaatagtagaattcgctatgaaaacagatgagttgctttagaaaagtggcaatgaaatgtatactcttgtataacctggtttagagggaaccttattaagtcatcgagtctaaaccatatttgtgatacttaattccatgatatttcagtaagtagtactataaatgactgttagtcagttttcattaccttctaccatagttaatttaatacaatctgcggcagcagttatgtttttgaatgcctgtgattttttaccctcaaggaactttcacccctggaagactgatatattgacagtgaactgatgcagcggagatgatatatacattgtgcaggaagtcaagaagtaatgtgttctttgaaggttgaaggtttttaaaggaagctggagaagattgaagtaggagtttaccaggcaaataagtaaaatggtattctagacatcagaaatagaatcaaccagggtatgttgctatgtggatgcttggtgtgtctcagtggaaggtatggggcacagtggagtgttggtattggagataaggctgaggtctgaggataaaggaccatgaaccttatatccaaatttatttcattttgaaagcagtgcattgacactcttctcttttaaagatgagaagttatatgatgtattaattgttttggaaaggtaacttgacagtagtggaagagatggacagaagtgggaagtgactaaaggcagaagacaaatgatcaagtctcacacaactctgcctagaaagttctttcttgtgttaggataaaatctttgaaacttcagtcctctgatcttaatctactttgaattcaaaacaaatataatctcttaaactttagagactttgacatatttaatgacagtgcttgtcttaaaggtgttctgccctagctaaaagtttattgcctagtagccttagttctcttttttgttaagatgtcatgtgatatggtgttgaattcaacttcaaaagtggtagattggtttgtttttaaagagaaaagaagacccgctatccttttcagtgcttttcaaactgtctgtggtaagagacccactaaaaaaattcaatccattatatcttttggtgaaatacaataaaattagttaccagagaagtggagttttaaaaagacatatatttaaccccatttaaaactgtcttttatcaagagacataaaattatcctgtcagaatgctcaaagagtttctgaacaggcttgatttctgtacttgtaggctgcacttccggccgcactggtttaaatgccaactgtgacgtggtatgtttgtttcaggattgtaaactagctccctggtgtccctcctcctctacttttgttcttctcactccctcatttttttttattttttatttttttgctctgtgtttataaaataaaattcattttagattattgactagattatttttaagtttgatttctggttacatgttcattaaggactcaaatggcttagctttgttttaagatagaagagtacaaaacagtgcaataaagattaaatgtttatttccgggttgataatatctcagtacatttatagagtggagtcactgctttttgcccattgttaagaaagattgggaagacttgaaataaaaaatttatttgaaaatacatttattaaaatttgtgagacaccctttgagaggtaaaaagatggacccaataagaatagacttttgtttttattagtaaagtaatactttaacctgtaaaacttcatctcattactcagccatagatataactccaggatgagttaattgcccatgtctaatgaaatgagacgtgagtctcagtgaaaagggagagtccattttatcactagatgctcttaatgagccttttaaaattaagtcttgattggattgcagtcaatttcgttaaggaatatttgagcatcttgtagttgtgtcttatgtaacattttctttttagaggattcagctctctgttgacagagtgagtagtggaatgttgctgatgtgtattaatgctgcagcaattgatcttttcaaaacccgttaaccttagttggaaataatttttcagtctaatatattaacaacaaattattaggccgtttcataagcctagggagccatcctattctatcctattagttttagttttctgtttatttcttcagttatgagagaccactattaaattattttgttgttctttagattttaaaacatgtaattgttaactctcgattttaaatacctataatttttagtttggtgtttttaaatcacaggttgttataggacatagtttgtggggctgtgtttaatgtttaacaacaattgtgaatctcagattgttgaaattattcttcattttgtgaattgttacgtttaactatttttaaaaacagtaatagaaattatagcagatagtgaagaatgtttttaaaaaggtctgcaaaaacttaagatttttggtgaattaaaataagtataaggtagtctaaatatagaggactagtcacttcaaatagaaaaaaattttcttaaaaaatctaatgtgaagaaaatttggagtaattttatctttgtaactaataagattaatgtttgaataataatttgtgttatctcagtagtgagattattgaataatagacaagtgagtaaacggatgcctatatggatgcttcagggtgtgtaatattatcaagtgttacaacagcacattgtttagaacgtttaagatttaagacgagtaagtactttaaaaggagtcaaaagaagtgaaagcaaatactgaacctcatcttttataacataaaaatgaattttttcttaattatgaaagcctttcacgtttagtgtagaaaaaatttagataattctggtattttaaaaatctaatcttccccatttagcgccgttacttatgtgttaatacatattttgagatctatgtatttgtgacctgatatattttgtttaagtatattacgtactaaaaagtcatctacaatactttttttttttttttgagatggagttttgctcttgtcactcaggctggagggcattgggcaatcttggcccactgcaaccttcacctcctgggttcaagcgattctcctgcctcagcctcctgaatagctgggattacaggcgcatgccaccactcccagctaattttttgtatttctaatagtgacggggttttaatgttggtcaggctgttcttgaactcctgaactcaggtgatccaccgcctcggcctcccaaagtgctggaattacaggcatgagccaccgtgcccagcctacaatactttttaatggttataagtagcatttaattgtacatatgtgtactgaggtatattgtttgttgaagttttaaaataattgtttttactataaaaatgctgtgaagaatatctgtactttatcattaagataaatgtgactgggctccatggctcatgcttgtaattccagcactataggtgggattccaccactgaggtgggaggatcatttgagcttaggagttcgagaccaccttggacccttctctaacttaaaagtacaacaaaaaaagataaatgtgtagaattggttggttgaaaatgcatttcaaaattgcctttcaacttcactgtttgaaattgtaagctccctcagaaaatgaaaatgcttatgtaacgaatcttctaaaaaatcacttttttaaagggacaaaaatatagacaaaaatacaacaaacatacttctggttaacaaggttaaagcttatctgtttcacttgtttattgcctaacattgctgccatctgtaaacccccactttgtattttcttttcatcctcattttgaaaaaataaaaaattatacatgcagaataattacaaggctaacaaagtgaatttgtgtataccttcaacctaaatttgctgtataccttggccctccatactgcaggccccacatcctccaattcagccaaccacagatcaaaaaatattcgggaggaaaaataataaaaaataataatacaacaatagaaaataatacaaataaaaaatatggtacaatgatttacgtagtctttgcgttgtattcagtattttaagtaatctagtgattaaagtatatggaggatgagcctgagttctgtgcaaatactgtaccttcctatatcagtgacttgagcactggtagggaggcggtcctgggaccaatcccacagggatactaagggacaactgtagtaacgttttgttcatttgtgtgtacacacagtgttgattttttatttggaagttcacctacttgctgaaacttatgtgtaaccctcaaatcattactggcgacactttcacggtcattcagacacgcacacagtggcaataaaaatctgagtcacccaaatgctcttttttgatattgagatagaacaagatgacacaccaccacctttttacttcagttcatacactgttaaaaaaagttattttcatggtctgcttagtgtcacgttttgggttttttttttgtttttgttgttgttgttgttattgttgtttttgtgttttttgactgtttattatgattttgctgtttaaaatgtccctcaagcatagtgctgaggtggtgtctaggatttaggattcctaacagcaagaagactgtgtgtgatgtgccttatggagaaaatacttatgttagagaagcttagttcaggcatgagttaactgctgttggctgttgaatgttaaagaatcataaggtgtctttttttattatttatttatttattttttttttgagacagagtcacactctgtcgtccaggctggagtgcagtggcgcgatcttggctcacattaacctctgcctcctgggttcaggcgattcccttgcctcagcctcccaggtatctgggattataggagcgtgtcaaaacacctggctaatttttctgtttttagtagagacaaggtttcaccagttggccacgctggtctcgaactcctgacctcaagtgatctgcctgcctcggcctcccaaagtgctgggattacaggcatgagccaccgcgcttggcctaaataaggtgtctttgaacagaaacacatataaaactgaattatgtattgatggttgatgaaaatgtgaccagaggtttgtgggaaccaaccctgtatttccttgaggagcagtattggctaattcagtgttcctggtgactttaccacaaataatgagagttgcgtgtgtgtgtgagcatgcgcgtgtgtgtctgtatgtatgtacatatataaaataattattaccgtttgctgaatcatttgagagcaacttgcagacattatgatccttcatccctgtcttagagtgcatttcctataaccaaggacattcttttacatagttactttatcaaattctgaaaatttaatattattacaatattgttatttgatggacattctaaattcaaattttccaatttttcattagttgtaagttctttctgattcagaatctagtccagatcacagattacatttatttgccctgtctcttacagtgtaatttggaacagttcctcagacttttcattatcttttataatttgtgtgtgtctttcttcatgactagattcaggccatgcatttttcacaagaatgttttaaaaagtgatgttgtgtccttctcagccatcacattagcagttagatgatgtcaatttgcctcatttttgatgttgataactgatcatttggttaaagtagtgtgagttatgtattgtcaccgtaaaggtaactttttctccttttaaatcatatctgaggagaaatactttgaaactttgtaaatattctttcctaacaaatttaggattgtaatgtcttttgtccaattttcgtatcattccctttacatttgttagttaacatttaattgtaatgaatatattgtctgacatttctaataggttaagatctctttcagtattacatgtattcttgcatatcccattcgtttgctgattttagagtctctctccttgacttttacactgcttgatatctttacttggttagatggtgatagtagtggttgtggtgacaacaataagttgatgttattgttaagggcttcaacacactgagctttgatttaggcattgttatcagtccatgtgaattaacccatctagtcctctgaaagccctatgaggcagttattgttctaattttatagatgaggaaatgaagcattaaggaggaggtaaagtaccttctgtggctggtgagttgcttggccccaagatatgaacccaggctggcttgagtttaactactacattgtgcctttcctctaagtagtcttctgatttccttatttccattcttccgcccttttctcttttcagcaaccagagtgaatttaaagaataatcttctagcatattttaatgtattcagttagagtcaggtctttactgtggctaaaggccctacgtggccttgtagcttgctcctccctagagttttctactctttgcctcctctttgtgtattcctcgaacatacacatgctctcatcatagggctttggtgttgctactccctctgcctggaatactctgcccttatgtatgtgtaaacttgatttatccctttatttcagatcctgttgaatctcacttttcagagagactttccataaccacttggcccccatagagtaaaccttcttgcccttttgctctcttgtatttttcatcttaccatcaacagccctgacatgttctacatatatattactaccaccctcacttaagggaatatgttcattgagagcagaattgtctcttttattcattgttctgtcctcagtaatagaattgtacatacctgccatgtagttggtactcagtaaatatgtgttgaaggaaggagtagtttgtcttttaattttgcttataattattttgcataaagacattattaattgaatattttaaagtatattttattgtggttttgtggattctgtctttagtgttgtgctttaaggcctttaccttgttcagtagtttatatagatatttgccattttgtgccatcattagttttgttctagcatttgttcagttaaatcttaaattcttttggaaattcattctctaggggaagattggaaatttcgctctttatcaattattttgctgtttaaaacaattttattgacttaaaatttttaaaattgcttttaataattgtatagtttacttttttctgtatgttaaaaatgaaaaattcacatttgaaattcttaattcagtagcaaattttttttaacttattaaagaattttttggtgcagtgttttagtctttggaggaatttgcttttactgtataagtaagaatttactcaggcaattagttctattttatctagatgtgctatatttattacctaatgacctaggaaaaaataagagtagatattgtttgttaaaaaaaaaaaatccatgtttttgttaaaaccctattacctttgtattttgatagcatgcttaatttttcttctcagtatagctttttggaagagtggcattaagttttgatttgcctaggatagttttagctatatttattgtctagttgtaattactaatagcatctccattcacttccaaaacattctggttgagatgaaaacttacatagtcaccttcacatttgggtgttgttttagtgtttgtgacttctacaaaacactagtgtcattcttaaatctacttttgttactagctatcatgggatttgttaaggagtgatactgcggccttatgaagtttttgatataaaattaaaatactactttaaaaatttctgttactgtattgctgttagaataagaactaaattttgtatgatccttgaagaaaataagttatggaaaaatatgtaaatcagtggatgtttttgtcctccagtagtcttatttgcttataggatttaattcatgttcttcattagtttgctcctaccgcagctgtggtaaaaaaaaaaaaaaaaaatgtatatgtggaaggtatcctcaactcctttctgccctcagtcatttaacaaatttgcacaattgtacagcacagataaagaccattctcagtgttaaagaaagttttattggacaatgttggtttaggcagagcaactttatacagaaaatttgaagaaataaaagtttggacaaataattcctgtttgtaattttatgttttgagataattttaaacttgcagaattataaaaacagtacaaagctctcctacacacctttcacctcacttagccatgtgtagcatgttaccatatgtgactcttggtgcattggccaccatgtttgtgtccctcatcccccttcactttcaggcccctcccttcttccttttgttcttatgtctgtctccctctttccctcctctccccttttaagttggagtaattccccagacttagtttgtttttttcctcacattgttttgaagaatattacactagtttttagaaataattttttatgattaaaaactgattctagatatttgacaggtttgtacgtgtgtctgctgttagaatataaaactagactctaagcgtgctcattttatttttccattgcctttgatatcatatgaatttagttatacagatggagcatccgaaatgcttgggatcagtaatgtttcagattttgggtttttttggattttggaatatttgcatatatacaatgagatatcttggggatgagaccgaatactaaacacgaaattgattcatgtgcacatagcctgaaggtagtttcatacagtattttaaattttgtgcacgaaacaaagtttgtgtatattgaactatcaaagggcaagggtgtcgacacaggtatgcagttctacactgcggcatcatgtcagtgctcaaaatgtgtcattttgaagcgttttgtttttttggattaaggatgctcaatctgggctcaattacactggtttcaacgtgttttgctgaaaaactcttgcagtattaaaaataattcaggaatagtgatgattgaacttatttgacttctattcgtgtttacctacatgtatgacatatcttggaccaatttgtaatatttagataggccactgataaaccaaagctaatctagacacagcaagtgagtttttttgacgataaagagatgaagatagtcattgactcatcacttttgaggaattctcctttgatgatatgattaaatgtaaaagaagaacattttatcaagttggcaaggaagactaggacactttgtgacattaagtgctaggcttgattaaatatatgctaaaacgtgaatttgttttgttagcatcactgacagtgtacgtttagtttaacatttaatggaaataattcaaccaagtcctttgtctccctcatacaatgtttccagtatcttctgtgttgtcaatcctgtcctcaccttctaaaaaatcaagctatctttattcctttcacatgttctacccacccgccaccccctagactattctttcttctacttttcaaagtttgttaaatagtttagtaagtattttttatttcctactctggactagacacaaaactgggaggttcttgctctcatggagcataatttaggaagatagaagctaaacaattaaacgaacaaaggtaatttcagatagggataagtgccatgaaaattataataatgccaggcaatgtaagtatgggctgggtggaaggcttttaactaggagggccaatgaaggactctgaggaagttacatctaaacagagactctgatgggtggttgacatggggaggtctgcagactgaatgtttctgggtgaagttagagcaaaagctctcagttgtgaatgtgatgggactgtccaaggaatagcaaagtcagcaagggtggaaagtgggaagagaagacgtttatgtccctgttcatatgcttctctataggcttggtaaaattttattggaagtaccaggaagtcagttgatggttttaagcagagggatgatatgatatttatttttagcaagagttgtctttagcaattagagacatgtatgtgtgtagggagttgggcaagagttgaaggaaggggattcattagttggctgttgcagttgttgaggcaagagatggtggcatggactgggaatcgtcttcagattcattttatttccatgctgttagaattcttcaacatttgctaatcttggctgaatttccaaagcacttgttctctaaatatacaatgtaatgtttatatttattgtttgcttttgtttccttacccaggctgtcttcttcaggagggactaatttatatttccaatttcctcaatgaaatgtggacataattggtgcctactaaaaaaattatgttcttcctacagaattttacaggaaatttcttattttctgacgtagaggagcatcaaataatgcatacacttagaaatttaaataaacaactgaataagacttaaattacagaatagagaaaaaaccaacaaatactttatgaattagttggagtcgaagttaaggaaagttgaatgtaaaacctaaaagtaaaattaattgctaagctagaggcaatatgtgagcatattttgcaaagactttggtggccatattaggttttcaagccacgcccacacacataactgacaatttttgatacttatgaagtacaaattctcccctccaatgtatgcgtgattctccatagtaacacccacatgtagcttctgatagaaacccacaaaataagatgaaaatagtttctcagtcaaatggttggaatatttagtaatacttggcagtattttttattttgttaataacaggtgtcatttaatacaatgacttttaaattatatattaaaagaaatttttaaaagttttatggcaagctgtgaaatactcagatggataatgtgaattaaggcgtgttctttggccactcaaagtgctccccacccaacactggcatggactgctcactaggtggccaaaagagatttcgtttatggatgtgttttgaatgttttaacattaaaaaaaaaaaacccaaagcatttgaggcagagatttttttagacaatagttgtttaatgagctgcttgtacatacaagagaagagaatgggcctgattgctcacaatctgatgtttgaaaagtatttttctggatcttatgcatttgcatatctaattgctatgtaatcaaaactaagtgaactgtagctacttgaaactctaaaatgaaaggcaatcatcttacggggaggagggagaaccccgttccatgcaagctagcctgaatattagcattatagtcttcttgtgtttaattataggtgtctgactgagcagtaaatttatgcttgtgtaataaagaaatgaatgtgaccctgaaacaccagctcagctttgttgtaactgagcattggctttgagagaagttagcatccattgtgtattttggtgataagggattgtgccagaatggctgttaattacgtactttatgtgatacagaaagtagtggttctacactttctttccaccagtgacagcactcaattaaaacttgtattaagcagctataatgcctttatttatctgtttggaagacatgaggtgaagaaaattaactttattttttccagctgtttgttggcagctttgtttttggtaaatcctttgaggataaaaaactgtttgttatgtgtcctgtatgctttcatagactaaatattttattttttgctgcctcacaaaaaggttattaagagatattttatattcttgccgggcatggtgcctcacacctgtaatcccagcactttgggaggttgaggtgggtggatcgcttgagctcaggagttcaagaccagactgggcaacacagcgaaaccccatctctacaaaaaatacaaaaactagccagacatggtggcctgtgcctgtgttcccaactacttgggaggctgaggtgggaggatcacttgagctggggaggtggagatagcaatgagctgagattgcaccactgcactccaacctgggttaaacagtgagaccctgtctcaaaacaaaacaaaacaaacaaacaaacaaaaatatatatatatgtatattcttaatattggttaaatgtcatttagaagccttgtagactagttaaagctttttatatgttaacccatttgaacctaatgttgagtcagtaagcacttctcgatgatttaaactctgagagagcaagttcaacctggaccaaaattaagataaagtgaatgtttatccccctgccccccacctaaaatttttgtgcttcataaaggggctccattgtaggttgaatctaagagaaaaatagtgattgtatttatggcaaacttgaaatttatattatcattttgaacttattttatacatgaactcactaatttccacaactgtttatttggtggtattccatggtatgtatttgctgcattttcttcatccactttactgttgatggtcaccttggtggttccatgtctttgctgttgtcagtagtgctgtgatgaacatgcaagttgcatgtgtctttttggtagagtggttgttttctttcggatgtatacccagtaataggattgctgggtcaagtggtagttctgagttctttgagaaatctccaaattcttttccacagtggctgaactaatttacattcccaccaacagtatataaatgttctcttttcctcttagcctcaacagcatctatgtttttttgcctttttcgtagtagccattctgacttgtgtgagatggtatctaattgtggtttttatttgcatttctctgatgatagtaatgttgagcatcttttcatgtttgttggccccttgtatttttttttttttttttttttttgagaagacacttctcaaaagaaatgagatgaggctggtctcaaactcatgagctcaggcaatccacctgcctctgcctcccaaagtgctaggattacaggcgtgagccactgcgcccagctgctaatttttgtatatagtgaaagatgtgggtctaatttcattcttctgcatgtggctagccagctctcccagcaccattgattgaatatagggggtcttttccccattgctttggcccactttttaatggagtcgtttttttgcttgttgaattgtttaagttccttatagattccagatattagacccttgttggatgcgtagcctgtgaatattttctcccactctgtaggttgtctgtttactgtcctgatagtttcttttgctgtgcggaaactctttagtttaattaagtcccacttgtcaatttttgtttttgttgcatttgcttttgagggtttagttataaattctttcccaaggctgatgtccagaatgatggttcctaggttttcttctaggattcttacagtttgaggtcttacattttaatctttaatctgttttgaggttttttttaaactgttattttttggagacagagtcttgctctgtcactcaggctggagtgcagtggctcgatcttggctcactgcaacttccgcctcctgggttcaagcaagtctcctggctcagcctcctgagtacctgggactacaggcgcccaccaccacaccgggctaatttttttttgtattttagtagagatggggtttttcagtgttgcccaagctggtctcgaactcgtgagctcaggcagtctgcctgcctctgcctcccaaagtgctaggattacaggtgtgagcccccacccccagcggttaatttttgtatattgtgaaatttgtgggtctaattttattcttctgtgtgtggctagccagctctcccagcaccatttattgaatgggggatctgttccccattgcttatttctgtcaaaattcagatggctgtagatgttctggttctctattctgttccattggtctttatgtctgtttttataccagtaccataatgttttggttactgtagccttgtattatagtttgaagttgggtaatgtgatacctctaggtttgttccttttgcttaggattgcttttcctatttgggctcttttttggttacatgtgaattttaaaatagttttttctaattctgtgataagtgatgttggtagtttgatagaaatagcgttcaatctgtagattactttgggcagtattgtcattttaatgatactgatttttccaatccatgagcgtgggatgttttttcatttgtttgtgtcatctctggtttcttttagcagtgttttgttttgtagttctccttacagagatcttttaacttcttggctggatgtatttctaggttttgagggtgtgagtttggctgtcgtaaatgggatgcattggctgtcagcttgaatgttacaggaatgcaactgatttttgtacattggttttgatcctgaaactttactgtgaagtcatttatcagttccaggagccttttggcagtctttagtgttctctaggtatcaaatcatattgttagcaaagagagatagtttgacttcttttgctatttggatgctttttctttctttcttttacctggttcatctgggtgggacttgctcctcttctttttaaaaatgtttattagctctgaatccaagcatactattaaacccattttaaaaacactgcttacacttctttgccttctgaaaaccatgtttcctaatattttgaggaaaaaatgggtgtccggcattccagcagctccagctctagctgtagcttaaagggcctgaggtacagcttgggctgttacttcagaggacacaagccccaagccttgggagcttccatgtggtgttgggcttgtgggtgcacagaacacaagagttgagctttgagaatctttgcctagatttcagagaaagtatggaaacgcctggatgtccaggcagaagtctgctgcaggggcggacccctcatggagaacctctgctacggcagtgcagaagggaaatgtgaggttggaacccccacacagagtccccactgggtcatggcctaatggagctatgagaagagggccaccatcctccagaccccagaaaagtagatccactgacagcttgcactatgtgcctagaaaagctgcaggcactcattgtcagcccatgaaagcagccgaggggactgaaccttgcagagccacggggacagtgctgcccagtgctgtgggagcccatcccttgcaacaacgtgtcctggatgtgaaacatggagtcaaaggagattttggagctttaagatctaatgactgcctacccgggttttggacttgcatagggcttgtggcccctttgttttggccaatttctcccatttggaatgggaacatttacccaatgcctgtacacccattctatcttggaaataactaacttgtttttgattctacaggctcatgggagaagggattttccttttttcagatgagactcagactttgacttttgggttaatgctggaatgagttaagactttggggaagtaactagtgggatcttatggatggttatgaaatgtgaaaaggacatgaaattttggaggggtcaggggcggaattacatggattggctctgtgtctccacccaaatctcttcttgaattgtaattcccatgtgttgagggagggacctggtgggaggtgattggatcataggggtggttttccccatgctgttctcgtgatagtgagtgagttctcatgagatctggtggtttaaaaagtgtttggcagatgtgttcccaccccgcccgcctccatcaccttgtgaagaaggtgctcttttccccttccaccatgattttaaggtttctgaggcctctccagccatgcagaactgtgagtcaattaaactacttttgtttataaattacccagtctcaggtggtacctttgtagcagtgtgagagctaatagaggtatcagagagattttcattattcttgtgagattttcccaattgaattgacatttcagttgttggtctgttgaataatgaatgcactataaaagtggttgttaaaattatgataagggaacaagatcagtcagtaaaatccatgtatgggagttttatgattaagaatatttctgtattttgtacaggacatctgtaaccacagaaaattgcattgtacaaatgaagaagaatacccttttacatcacatgatttgtgatatctggaaaatctcccaagcacctggtttctggattttatttagatttttcaatgtattttctaagtcttaggcaatattttcagaaaagttagttctttcctactcctttgatcctataacttagccacatcattacttaagcacttagtctgctatagtggattacagagttctttaagagttgtaactcttatcttaaccactataatgtgcatagtgcctggcacattgtagacacttaggaatgcttaccatgacccttagtattgtatatttgagttaattgctagaatttttaatatctcctattgaatttattaatactattttcatacataagtacacccctttcagtgttgtgttttaagattatattacccacttcattcctcccatccctcactctcacccaccaatggcaactttgattaacatgtttgtgtatagtaatcctgtggccacaagcacatagctgtaagcaaggactgaactagaagaccaacttgattctgcgatgattaaaggaaaaatggctaggaaattgactttgaacatgagtaatgggcattgaagccagtctcacaagagtaaacgatgctttgggtgtgctgtatcacctgtgaatacagcctaaaagttatgcctgtcaccaaggaaatgtttctggttgcaggaggactggagataatactgtaactagaactagggccaagctgccaaatggcctgaatactggattattgatatttctatggaagaatgggctttgaactgccaaaataagaacttgagggtttctgtttccggtaatgaatgagtaaatttctgtcatacttaatcccctgcaataagcaactctaaaatatgcacgataaaaaccagctactccaaggaactctgtaggatgagtgtaagcagagtctgaaatagagttggtccttgaaggaaggaagttagtacactgaatactagttttcctggcttttagcctgaggcaaagcacattaggtggcactcattccaacccagagaaatctctgctgatggagaaagataaaaagaagggaaagaaaagagaacgggaatccccaaattgtagctgaccccaggattatatgtgcagagtagacataagcatctcatctaaagatgtgaactaagaccatagttggcacctgagaaacaaagtgtgcagccctcaattgaatcaagatattgcctactacaaaacaaaacgaaaacaaaaccagaaccagaaacaaaagaaaaaaattcgtcaaaataatctagaatcaggatacagtttaaaattaagctctgaaagtaaatcatactctcaaaagaaaatcagctgataccaaccattacattttggaactaagagacagattttttgtttttgtttttttaaatagctgttaactagtctcataaggacaaaatacagaaagacagttcttacagagaaatagaagcaaaaaaaggaaccaatggaaaatttaaaactgaaaaatagaatatctgaaataaaaaattcactagatgacataactgttgaggaaagagtatgtgaatttgaagaaagagcaatagaaatgttccattgtaaagaacaaagagaaaaaaaattgagaggtaggaatggcctgtgaagtggtctgacatatgtataattggagtcttagaaaaagagggtaaataagactgaaaaaaaatacttgaagaactaatggcagaaaaatctcacaaatttgatgaaagacataaatatgcaaattcaagatgcttaagaaacagcaagtagaatgaacacaaggaagactacactgagttacatcagagggtaaaactgtgaaaagccgaagattataaagaaccaatcatgaaagcagcaagaaaaaattacacaccatatataggggaacaatgatatgattaattgctcacttcttatcagaacatggtagagagccagaagagagtaaaatatctttttttctttttcttttctttctttctttctttctttctttcttttttttttttttatgatggcgtttcactcttgttgcccaggctggagtgcagtggtgcaatcttggcttactgcaagctccacctcctgagctcaagcaattcccctgcctcagcctcccaagtagctgggattacaggtatgtgctaccacacccagctaatttttgtattttagtagagatggggtttcaccatgttgcccaggctggtctcaaactcctgacctcaggtgatacacccacgtcggcctcccagagtgctaggattacaggcatgagccaccgtgcttgggcacttttacctttttaaagatattttactggccgggcacggtggctcacacttgtaatcccagcactttgggaggctgaggtgggtgagtcacctgaggtcaggagttcgagaccagcctggccaacatggagaaaccccaactctactaaaaattgaaaaattagctggacgtggtggcaggtggctgtaatcctagcttctcaggaggctgagccacgagaatcacttgaatccgggaggcggagttgcagtgagccaagattgtgccactgcactccagcctgggtgacagagtgagactccgtctcagtcagtcagtcaatcagtcagtcaatcaatcaatggtaaaagtaagaaattgccaaccctgaattctataaccagtgaaaatatcttaaaaataaaggtgaaagaaagccattttgaaatgaaagataacagacagaactaattgccagcagataagcacaccaggtaatgctaaagggaattttacaggctaaagagaaatgagagcagatggaaagttgattacatagaaatttttaccctgatactggtaaataccttcataaagaggaaaataaccattttttttgctccttcccccttcttaatttctttatattacatatgaccatttaataaaggggggggggaatagtattgtcctgtggagtttttgattcttgtatgtgtacacgtggacgtatgtggttacaggtttctacatattatgtgaaacagtataatattgattgtaagtaggccttgaaaagttaaagatttgtattgtatcccctatagcaataactgaaaaagcaatgccaaaaggtatatgtaaagagcctttaagtataacttaaaaactgaaatggaattataaaatatattgaaataatgtatatatagatatattgttcaaagtccggaagggaagctaaggaacaaaatacagaggctacaaatagaaaaacagtagtaaaaatggtagataaacacctaatcattcaaaaattccattaaatagaaatggaagaaccacttcaattgaatgaaagagattgtcagattgtcatcatggattaaaaattaaaagcaaggctggctgtggtggctcaggcctgtaatcctagcacttttggaggccaagatgggccaattgccctgagctcaggagtttgaaaccagtctgggcaatatgttgaaaccccatgtctactaaaaatacaaaaaattagctggatgtggtgatgcgcatttgtaatgccagctactcaggaggctgaggcaggagaatcgcttgaacctgggaggtggaggttgcagtgagccaagatcgtgccactgcactccagcctgggcagcagagcaagactctatctcaaaataataataataataataataataataataataataataataagtaaactaaaaagcaagccaagtgcagtggctcatgcccataatcccaacactttgggaggccgaggcaggtggatcacttgaggtcaggagtttgagaccagcctggccaacttagtgaaaccctgcctctacttaaaaaatacaaaaattatccaggcatggtggcgcatccctataaccccagctacttgggaggctgaggcaggagaattgcttcaactcgggaggtgtgggttgcagtgagccaagattgtgccactgcactccagcctgggtgacagagccagacttatctcaaaaaaataaatgtacagtttacagagaaatactagacacaggtaagttgaaaataaatagattgaaaagcatattttaagaaaacagtaaaagattgaagtgactatattaatatcaggtaaaatagattgcaacagaaaaattttgtatcaggtaatgtggattcttcatagtaacaaaagttcatcaggaggacatatcaatcataagtgtatatttatttaatgcagaatgacttaatacatgaaaactgactaaaggcagaaatagttccacaatcagattgagattttaatgtaactctttcagcaactaataaaaaaaaattactcagaagatttgaaatttattattagccattcttgatcatattgatgtttattaaacgttatatcttacagctagagaattctcattcttttcacatgcatatggtacattcaccaaaacagagcatgctgggctttgtacatgctgggccataaggtacaaaggattaagattatatagagtatgaattttgaccgcagtggaattaaatttcaagtatttggaaagtgacacacttttttgtaatcagttggtcaagagatgtcaaagggaaatcagaaaatattttggaccgagtgattgtggaaatgcaagataacaatttatgagatgaagctaaagcagtgtttggaggaaatttatatatttaattatgttggaataaaactacaattatctaatttcctacctaattaaacatttaaagaaaacagcaaggtaaacccaaggtaatgtaaaaaaaggaaataataataaattcttgaaatagaatacggacaactagagaaaaataatgaaactaaaaagagttcttggaaaagttagttaagataaagctctaattagactaatccagaataagagagaagacactaattaccagtatatttaataaaataggagacatcactgtcgatcatatggacattaaaaggccttttaagagaatatcataaacagctttaagccagtaaattagacaacttagttgaaaaaaataaattctttggaaaacacaaattattacccaggagcaatagaaaattttagtatccctatattgattagagaagctgaatttgcaattaaaaattattccaccaagacaatgtgaagcctaaatggccacactaataaattttatcaaacttcaagaaagaaatattgtgaatcttatgtaaagtcttccattaagaaaaaaaaatgagaatacttcttaactcattttattataacaaattggtaactaccattgttactgtgatactaaaattcaacaaagataacagcaaaacaaagaatcagtattcttcatggatatagatgtaaacattttaaacaaaatttgagcaaattaaattaacgataaaaaaagaagatactgcattaccaagagaggtatattttgagaatgcaagtttagttttaacatcttgaaactaatcagtgcagttaatcataacaatagaataaaggtgtaaaactgtataattatatctaggagcaacagaagtatttgacaaaattcaatacacaaaaatgataaagcttcccagcaaacttagaaggaaacttcctcagtttgataaattcgccttatgaaaagacatagtaatatactcagcagtaaaattctcagtgtgttcctcctaaggtcacaaacaagccgaagatgtttgattttaccctgtctattccaatttgtactaaaggtcctagcaagtgcaataaagcagtaaaacaaaataaaaggcatacagaatggaaaagaagaagactttcttattttttaatatagaaagttctaaagtatataaaaaggtcattccagcaagaagagatcatgatcctaagtctctgtgtatcaagtaacatagcttgaaaatatataaagcaaaaactaccagaactaaaagtagaagctcagaagtccacagtcatactgttcacacaactcctaataactgataaagagacaaaaactcagtaagaatagagatggtctgaactacacaagtagcatacttcatttaattatctaattatcaaaaagaacattgcatccaacaatgacactatttacattattttaaagtgtacatggtacatattgtccagttgaccacgtgctggtccataaagcttcaggaaatttcagacatctgaaattattcacagtatattctttaatcatagtggaatcgagctggaaattaaaaactaagagttggccataaaggatgtatgttttaacttaagcatctggaaagaacagtagagtagattaaaagcaaagaggaaaaattaataaatagaaaatgtattgaaaattatatagtagtccttccttatccacagttttgcttttcatggtttcagttacctgtggtacagtacaagatgcttcgagagatcacattcacacaactttcattacagtatattgttatactctattttattgttgttcatctcttactgtgcttcatttataagttaatctttatcataggcatgtgtttataggaaaaaacgtagtgtatatagtgtttggtactgtccatggcttcaagcatccactgcaggtcttggaacatatcgcttgtagataagaggaggacgactatattgatgattaactaaaatgaaggaagtttctgtgaaaatttacaaatgagacaaactttgtaaaactgattaagaaaaaacaagtagaactaaacttgattgtaattattttatgtatcaccaacttggtgatacataaaatacacaaaacaaaaaagcagattaattgacagagaacttgcagttcacagagtttttatttgtatatgaataatatgaataggctttttctaaacttatttcgtcctgtcatttcattgtatcccatggtatacattaaaagaaaaatgtaagtgaaattaattagtgaatgcctaagactttttcgcagagttcagtatcagtttttagtcatgtttttcacaacatacagcaatctatactaccaggaatgttcgatgatgcagcatttttaaaagagtgacttactattctcctctagaatttcttcccaggccatttcaagtttttgtgcacattgttgtcaagaaatgtgaagcgcctaagatttttatcttgtaagataagctagcttgccaccacagtttcatggatgatggcagaagacctaggacttctgaatcagaagccatgctttattatttacagcaaaaatagtagcagagtgttagcatacttgcactgatgccctgagccctggtgtgcacagggtaatgcagaggccagacaacacctgctcttggtgggctggatttcagaagaggagcccagggccaaggtccccacaattcttgagcaagaagcagacaaggcagtcctctacccttgacctcctcagttgcatatgttactgcagaaactgctcagccagaggagatagataacccttgcactctggcacactcagtgggaacattgagggctgctcagagctcacccaacagtggttagacaggaacacctgtggcttactgctgcctagcttatggcattaatgagacacatcgggtttcaaaaaaaccttaaaatactttttaagaaatgtcttagaataggtgacacactgtaagtaagtgaaatcaagggccaaatttgaaatactgtatttaaaaaatcatggaaaatgcaacaaacagctttagagctatacatttgaaaactgataataatttatttatcttaaagatatgtattttcaaatctatatagtaaaatctagttagaaatcctaactttttttctggtggaatttaatgaattgattacagaattcttaagatagtaaaattgcccattggtagttgggacagttctaaaagaaaaatggaggagtgctttcagagcatttaattcttatcagtttcataattgtctgtatgactgagggataagcaggttgaccaatggaattagactaagctattttactcggatatgtatgtgtgtattgtggaaagaaagaggtagatggagagaaatttacatgagaagtgggaagcgtgaatttaataaatgatattaggatagttgaatttgcatatgcaaaaataaatcttttgatggatcacatgcccaaaaatgaagagcaaaaaactcaaccactagaagaaaacataggagatacatttatgacttcattgtaaagcaggattactgactagaaaacttataaaggaatatcagtagctataactacattgaaatgtattttaaaaatcctttttatgcaaaagacaccataccccatttaaaagacaagccacagagtgggagtgtatttgcaacatctgtaacaaaatgtgtatatttagaatttattaaaaattcattcaaaaacaattttaaaagatcacaaattcaattaaaaatgaacaaagtgtgtgaacaggcaattcacagggcagaaaacctgaattaaaaatgaacatttgaaaagatgctttaccttattaatactcaagcgaatgcacctttatatttaatgagaataatgtaccatttgatgtcctcaatgttatcaaagctatgtctaggatatggagaaatatgaactcccacacaagtcagtgtaaatagacacagacatattggaaagcaatttggcactgtcaactaatagtgtgtgtttttttttaagaaggtcctgtttgtggaaatttcattatgtgtcagtttcttaagaaactcttgtcacatgtgttttggaagatgggaatgaggatattcactgcaggattgtttataataataataaattggaatcagtataaatggccataaatggagagatggaaatataaattgtggttctttgtgtagcagaatattttggggcagtcaaaatgatgaaatttgatcaaatacacttgatttaacacatactacatcttgatactataacgttcaaaagaacagtttgaaattattctgtatatttgattagtgataaaaatatattggtgtgttacaacaaaagggaaacttacatataatgatatggcgataggtcattctatatgtgaagtggaatttcaagtgatcttatttctagtttattaaactttcagaaagcactttgaatattgaatatttgactttttctggcataggtaattagtgactgttttctatttaagatacaaaataagtccagcaattgacatttgctatgtaatcaaagcttttcactttttatttttatctcaaatcggtcacctctttgtggtataagctgattatttatctctcaaatctgtcacctcattgtgatgtaatctgattatttatctctcaaatctgtcacctcattgtgatataatctgattatttttaaagagaataccttttgatgggatagtaactaaatgtggtattctaggatgttaaggtaaattaagattaatatttgttttgtgggtccttcattaatgtctttccttaggagtgcaggcaatttttagaaaatctaaagtacatatatgcacacatgacactcaacaacatacatacattgtacccctcagtgtctacctgttgcttgcaactcaattatggggccttttttgggaaaaatgtagagtttctaaagcttattttatttatttatttttttgagacggagtctggctctgtcgcccaggctggagtgcagtggcatgatctgggatcactgcaacttctgcctcccagtttcaagcagttctcctgcctcagcctcccaaatagctgggactataggcgcacaccaccatgccccgctgaattttgtatttttagtagagacagggtttcaccatgttggccaggatggtcttgatctcttgacctcgtgatccacccgccttggcctccgaaagtgctgggattacaggcgtgagccattgcgcccagcctaaaacttcattttaaattaagcttgagggcttttatggtctttcaatatacctttaataccttgatgacttatttcaagaaagctttttatttttctggtcttaactattttccatatttattggattaatctgtttattaaataaaatatgttttcatactgaaatgaatactacttgcgttcttactggtgtagaggtgtaggctagagtatttgacattctggaatatattttaacttacttggctttaaaatactccttatcatactataactgttttgaaacacatatcataatgttagcctcttgggttcatgccatgagctataattatggtgttagtgccataatgggatgttataagttttattttagaatttttatgaaggtgaagctttaaagaaaagcttaacaagtttcaaccagagaagcagtaggggacacacacacacacacacactcactctctctctctctctctctctttctctctctctctctctctctagctctctctagctctctctgtctcccttattatgaggaattggcttgtataagttcatgctgactagtctagagtccacaaagcagatgggtaaggaaggaagatccagagggcactgttgtaaacccagttggtgtttggaatatctgagcttagaaaagaccaaagccctcttttaaagacttctaatggaatatgtcaggttaacccaggataatctccctgttgattaacttaaattcaactgattacttttcttaattacatctgcagaatttctcacagcagtatctagagtggttttttgttttttgttttggttttggtttttgacacagtctctagctcttttggctcaggctggagtgcagtggcttaatcttggttcattgaaacctccgtctcccaggatcaagtgattctcctgcctcagcctccaaagtaactgggattataggcgagcaccaccacgtctggcttagattagttttttcttgagtagctgggagaaggtatgtgtatgctactaaatggccactgttttccatttatagtccacctcttgaaaaaatatactttatagaccatcccaactggaaacatacaagaaatgaaattctggaaactctagttcagcctagttaagctgacacactgcaaaggtatcacaccggataactaacttttttccctaaatagtgtctctctttattttgtcgattgatattaaaggatattttcattgatgagtgagggatataaatttaggtgttattagatgatacacagctatttctagaaggttaaggtaaatctggtaacctggtccttttttggcttgtatttagcttatgtttagtaaatgggtttacctgcccctgccctacatattctttgatttcctccctggcaccaatttcatgtgtttagtgtacaaactccttttaatatttattgtgaaggaaagtggtaattcaggaggtttttgccaaagtaagattctagtttattagaatttgtgtaatctagagagatgtctgattcttatcttaaatggaaaaaagctatagaagctggctagggaaatcttgtttttcaggaaaaactcacatccacggaaggtagttgagttcatttctttatttccttattcaacagacttcaaaaaaatttttcctttgtggtaggctgtgcgctaagtgtttgaatatccagtcaaagataagatacaatcaatgcctgcccttttgtagtcaggatggacagatacaaattttaactagaaggggccgagataagcaaaattggaaatcatgttttacatagatactgttttttgtgggtccatgttcttagttttttccagtaatgggtataattgagaagagccacagactcatttctaaagtagtcattcaggcttcttgcatataattttcaacactcttaaaaaccttatgaacattttaaggaagagagtattttgtacctttttgtgaagaaggctttattttggctgttgatattaaaaaatattatatttcattgatgagcaagagatacaaatttaggtgttactaactgttgtcattaaggtcaagttttatacatgtaaataaattgtagtttatgaactgcagacatagaggttgaggtttcatttattctgtagtagattgtagtaactactctttcagcatgatttacaaagaaaatcaaactaactcaatttatagattgcccttttccatagaatgtcaattttgtattttggcaagaatacctgtgaggtaagcctaggaggcacataacaattctgccatttccaactcttccaaaatgtaaataggtatttagagttacacaccttgtatggctacatgaatttatttatagtcttttctttttagagtagcatagcgacaaaagtagtcactggactttctgttcagtgtgaagatctgttggtatgtgttctgctttattaataaatgtagagagctgaactagagagttgatattcattgaacatgatatatcctctccctttccccaatcagattcatttcactgaataaacacctgagtatgaaggttatttcaagaagtgctgggcaggaatcctaaccttcagtagcacatagtactatgaaatgaaatgatatgtagacatgcagagttccttataaacatatgtcattaataagaacacatgagtaggactgttggagaaattcaggacaaaagagctcttaagacagcttgacaaaaggggacctcagagatcgtgccagatttcctgtgggccttaaaaagagtgggtgtgatgatatttttatgtggcacagtggcgacagagatgaaataaggagtcagatttgagttcaggttctgggccttctatttatggattgaaggacgtaagacaagttacttttgctgttgaaattttcacatgtgaaaaataagttaatatctaccattcaggcttactagatattaaaagtattatttttagtgttctttaatactattttagaatacaggcagttgatagatcctgaggtgtctatggtcgttttcaaaaagatgttttctccaattataatgattactattaacatttaaattgacccttgctctcataattaaccacaataattattaatgatagcttactttttaagcattatagttattttatatagttgttatataaagtttgtttaatatctcagaaaatgtttttcattgcattcacattttagtgttgtgatgaatggcatgttacatctgtttcttaattaaggggaagtgggccaaagatattctgaaagagtagagttactttttgtcttaggaaacatggctttaagcactagtggacaagtaacgtaccctttgaaaatactttcttgtcaccagtgaaacattttagagctgttatcaaagactgaagcaaacaatttttgattctatagttatatgggttgataatagggagaggaattgaagaatttcaatttagcaccatgtctctgaaacttgaacatacttgaaatgatttttaatgaaggaataaaaacatggattctctgaacttctattttgtatttctctgagagattactgttttcttggctgatgtagttgaattcttaggtgtttgcagtaaagtataagataataaaatggttaacgtttaacaaactcatcattataacactggataatgtatagaatatgtaaaaaatggcatttattaaaaaaatcatgagtgtaacagtctaggttttagctagcaagaagttcaggattttttttaaaatagtgtttggggaaattataaatgtcggttgcataattgtaattcatgaaggaagacattcatacagctattttttacctgttttcaaaactatagaaaaaaaaaggtggtacgagattgtcctaagaattatcctattttagcttgactaccacctattagctacaaggctttgcagtggtgctgttagctaggttcatatgacctatgtccaaatcatgctggcaacatcattgaagaatgattttttcaatgctttcaagttattaacatatttgaatgtacatgtcagtcttacccaaaggaagtgaaaataattcagtatcttcaagaaaatggaaaaactgcattatttaatatactctgctgcattaactatcactttttatagttttgctaatcaaattatgatgaagggttatgagaaatatatcactttttatagttctcttgttagccaaattttaatgaaagattggaagaaaagtctaggattattatgggttttggatgacagaggtgatgtaagtgtaggagaaatgtatgcctagaggacctaaagtcacctagtttttctaatgacgtaaaattacccttggtttttttctgctgagaagaatgggtgtgtgaaatacacgttaatactttggaatttaagtgaagaaagaactccatttaatacactaagcatttgtaccatctagtattatatttttaatctgcatcagacattatgtcatccgaatatcagcccatgagtatagaggtatgctcttcttagtagctatacctgctgccacctgactgtgcacaaggagcaccatgctttggatccttccctatgggaatcgctgggatgtgttgtagatacctgtctatatatttttcagttgaaattagaaggataatcttacagatatttaaatgtgttaaaccttaaatgtaacaaatattttagattaacatatatagactattaacattgtgtttgattcgctcttattactctaatcttaattctagaactttcaaaggttgaaagtcattaccaagaaaaaaatcatgacatcacaaataatttggggcaggcagttagatatatattttttgagacagaaactcactctgtcgcccaggctggagtgcagtggcatgatctcggctcactgcaacctctgcctcctgggttcaagcgattctcctgcctcagcctcccgggtagctgggattacaggcgcatgctaccatgcccagctcatttttgtacttttagtagacagggtttcaccatgttggccaggctggtcttgaactcctgacctcaagtgatttgcccacctcagcctcccaaagtgctgggattacaggtgtgagccaccatgccaggcctgtattttttaacccagagggatcattgattaattgctaatttattttttactctggctttgattttgaggcttcttaattgaggaagcgggatgtgcttatgcttgattatgttaatataatgacagaaaggacatcctgcagtggaattaaggggaatccctgttagctgaaaagccattttttaaggaagatacaggaaagaacagagataggagttacatagtaaaagtgttaagtattatgaactagtcactgtcttcaacatactttctttaattcttgcaaccttgtaacctttacagatgagaaaataagatttcaaaaaatagttaacattttgtcatataaccttgatcagtcaaccctcatatgtgcttttatataacacagtgaactttggtgtcactccctcaattgcaaataaatagttactagtctgtaggctccttgagagcttgcatgtctgttcacttgcctgtcataaaccttgttcactcacagggtgtagtagaaactcaattattatttatgtatttattaaataaataaatgaatacgtgagtgagttgctcaagattacaggggcactgaaataagactggcatttgagtctgtgttctttcccccactctgttttctacccttgtctggtttttaggggttatggagtgtggggagaggcctttttgtgcgtttgtttttgagtaaccagttcttttcattttgtctctattctccagtgagcaaatgaaaattcagggtttggagcaaacttttaacgttctgattatcagtcagcaaatatgagtactggataagtgtttagcattgtggtaagccccatggaattataaattatgtgcctgtttccttgccgaggttctcaaagttcatttatgccaacatagctcttccatacacacataaatacgtattcctgaacatcgtcataaactagggtcattattctacacagttgtgtttttgtcactgcttttcagtgatgtgtgcacgattacatgaagacagctgtgaattaatatttggggagtacgaacactgcagctatccacggtgccgttaggttgaataggacgtcgcaacctaccaactacaaatgatggaacctcctgttagtcttgtatcttaacattcccaggatgaaacaccttttataacgtaatgagctatgtgctgttgttctcttacattttggaaatcctaatatataacattaggctgtgatcttttgctcgatgaggaagtaaggcagactgtatgtggatccttcagcataattcttactttgcttgtggttgtagattatgggactctggttcagttctttagttttgtgtttttcattttaatttcacagaaggcaggtaaaatactctctttcagtgttgcctgactgagtttttaaaataaatcttgaatttttcaggtatttattttccctaactaatgtagaaaaatacacatgtggaatgcacgagaaagcaccatatggcttaggagcttagtaatcatgtaatcagtatattttatgttacttgctattttcagtttataatctaggaatattaagagacaataataaatgtcttttaagtgcttactatatgcaaaacattttggtagatattttagggaagtggtttcttatatagaaggacagtttagcatttgtttaataaatgcataattacagaaaccatggattccatagttccttttaaatgagtttgtagtatgttaattatgatagtatgcatatatgttgagaaactagtatatttgtatttgacatgatttcatttgtagaattcagagctactttgtttatacatatgtgagacaaatacagaaattaatattttaaaaactgatgaaaatatcataaaagatgtgcaagttcaaatctgggcagggcagaagagaaatagaaagctactggttgtgtaccaaacttttctttttagatgccttgtgaataatacaaaaaattaaaaggagcagtctttgctttctagagcttaccattatgtacactggaatattaaagtatgatcttaagtatcattttagagccataagattcgatggaagcaaagtggggcttagggagaatgaatttggatagagtagggtagtggaaaacagttaaggagtaggtagtatttgaactcattcttcaatttcattaagtttttataaattccctttactattcgttgtccattttgtgtactgcttgtgacatactaatagtcacggtttatcccactttccaggaaactgtaccggtcgtcctttacgtatcatttctaatccaaatgcttcatggagggcagggttagtgttttgatcagctgagctgtcttccagtctgaaacccaaagattacagttgtgtctttattaagctttttcttaaaaaatgtatttaaaaataattttattttcatgtaatggttaatatattttattaactaagttggttaaaaaataaattatcttaagctactctttttattctgaaaatacatttccaattttatttttctaaattataaccgctagtcatgtcattttatttgttgttcagtcatcccttcccagtatttctgtttaattttcttttttttattttttgagatggagtttcgtttcgctcttgttgcccaggctggagtgcagtggcgtgatcttggctcaccgcaacctccacctccgaggttcaagcgattctcctgcctcagcctccctagtagctaggattacagacatgtgccaccatgcccggctaattttgtattttagtagagacggggtttctccatgttggtcaggctggtcttgaactcccgacctcaggtgatctacctgccccggcctcccaaagtgctggaattacaggcatgaaccaccacgcccggccttctgtttattttctacgatcatctattctagattctagcctgtccataccatatttacattatggtaatattaatttcagagctcttaaatgacgggtactcgtccatgtgctatccgaacactaacatttaaaactcattgtagctctgagatgtaagtactatctttatgtctcttttgtagatgaggaaactgaagcatagataaattcaagcattttgcctaacactaaacagctattaagtggttagtaagtgtggtatcataaattccttagagtatcagaaaatgaatattaaacattcgcattttaaaaatcttattaacaattgttttgttttgaatctctgggatttatacctaaataaaattccctctgaagaatggagcatttgcacagtgaaccatgtgctgccactctcaacatcagtacatgaacctgttactgacctttgcttgagacaggctatatctggatttttcaaaacatcttgtttccttttttttttttttcctgttctctctctctctctcccctctctcctcttttctctctctcctctctctctcttctttcttctttccctcccttcctttcctttctttcttgacggagtcttgttcttgtcacccaggctggagtgcagtggcacagtcttgcctcactgcagcctctgcctcccgaattcaagcagttctcctgcctcagcctcctgagtacctgggattacaggcgcctgccaccacacctggctaatttttgtatttttagtagagatggggttttgccgtgtcggccaggctggtctcaaacttctgacctcgtgatcctcccccctccccgcctcggccacccaaagtgctgagattacaggcgtgagccaccacgcccggccatcttgtttctttttattgttttcaatgaaataattgtaatcttgattatgttgacttttaagtattgtatttagcctttcttcttaataggtagttgtcaggtcgtcaaaggcagacgagtgtcatctgaagtcggtttaactgagtacattgagtcaggtttctgcccattagtttatctcatttttaaatgagaaataggtgattataataatgcctacctctaagcttattaaaaggattaagttattttatgaatatcaagtactgagtgtaaagccctatatgttttttgtttgtttgttttagttgatattgaccacactaactttatttttgtatctcatgctgtgctctaaggagcacttattagaaagtctgtgctgaaagatatggagatgagtctgattccatacttcttgaaactttttaggaatcatacataccagactgattatgtcaaaagaaaaaatagtagaatgggagtattcagggaacatcctgtctggtaggacctggaaggggtatatttgattgaaagatgattattgaaactgggattttgatgatttaggaaggaaaatgggtataaggtagaaatgtcttttatttatgtattttcccctggtatactatcagtaaatattcaattgagtattttaaaacatcacattctaaaacaattatttcatttttctgtgccttgaattgtgtatcttagatgatggtgagaataacacctaatgtacaaaatctcacatatgaattagtgaaaggattcttttaatgctgtgaggaaatattttgtgcttcagtgggcagtaacgaattttatgtgggtgcttgttagcaacaggagctcagcccaaagtttccagtttaaaaggatttcccaacaatcatttatttctatcaccactatttttgttaaatatttgccagctttaaagttgttgggaaataacaaatcacttgatgatttttgtctttttatgggaaactttttgtacatatttaattcactcttctcagccttaactatgtttcatcaagatcatattcagcttttcaactgatcttgttcagaattgtttctgcatgctaaaagcagctcatatctagagaaatctcactcattttggtcatcgtggggccagaatagtattttggctagtattggggttatataaatttagtttagacataaagtaattaacaattaaattagtggaattaatgtatgaaaattggaagacatacctggcagtcaataagtactgtgtctgtaggagatagatgttacagtcctgttgtttttaacttggaaattatttggcaaaaaacaataaaagttatttgaatttaggaacttgttcctttgtgcctaatttaataagtatttgttgaatgcctgctgaatgacttcctagatgatgatttcaacggactgaatataaaattatcctagtcttcatactttattagaaaataatttgctttgaaagtgtagtttttgttggactcacattcaagtttttaaaagttttttgagggctgcttttataactgagtcagtgaggaattaaattgtgtgaaatctactttatgcccagtttctcaatgaacatttcttaaactaatctaagattatatgcacaactggagatggtaatatgtctaagctttaatatttgtcttaaaatgtatatattcacaatacgcctgttattggaaccttggtttttgtcagcatgtaacacttttgaattctgtttggaggggttttggcgtatgaactttcacattaagagcacagtaggagcagaagtcttgtgctcatttgcttgactacaaatgcataattaagagagtctgtcagcaggggtgggacaatcagtatactctaatgcttcatgttatagaatccctttgttgttcaaacagatattgtaattagagcatacttttaaacttcatagggcagcgattcataaagtttttcaaaaatccaaaacaaatactttgtttctcaaatgttgagattatttttttactttgtagttttacatcactgtatacagtggtttcaaaaatgaaatggaagttgttttattgaaaagtggtaaaatttagttataaacaatggtgttaattagtcctagcttagtaataatgttggaaatacgcattgtatattaagatctaattttagttttaacaacctcacctattcgaaactttaaagactagaggaattttatgtaagtgaaatagctacagatttatcctttttgtatatatgtatcattgtaatttgataggtgagtctctcatttagggagaaagcatttttctccctaggtgcaagagatctgttataccagatgtttttctcccagttcttgacattattctggatgtgacttactcagaaattcatttatggagaaagaatacttgacatgaagttaggagataggacaaatatagaaagacaaaaccaaatctactaaataatctcatctacccctttatctgccttcaaagtccccgccagctctaaattctgtgaggacaagatgtgcttagaattctacacaaaaatatttaaaggtaaaaaggtaaagtaaaaaattctagagattccactattctgtgttccgtttctggtttttcactattttatgatgctgttttaggtcaatttctcatgtaggtgatatgttgtgctaggaattagttgttgagggtgcatgctgtaaagttgctctggctaggtatggtagcttggtggtacaacacttactgcctgacttgactaaatcatgttaaatgttggtaagactgatagtcccaggttcatgtaacttatgaggattgaatgaggtgatgtatttatattgcttagcattgtccttggcaagcagtgtttgataaaagcttagttgccatcatcattttcatttttaagtttgcttatcatcacctaagtctaatttatctcaccatagtatacttgtcaagaatacagaccatgataatcattaataattgtgactatctgctctgccatcaagtgtgaatgaattataaagttgcttagcaagctactatatttgaatttgctgtataaatagagctattttccatttccatggaacctctgttgttattgcaccctaccttaaaagatggaaggttagtggttttgagctctgaacatatttatacatatagtatttatatgtaatttgcctgtgctgtcactttgctgttatattgagatagaactttagtcttgcagttttaaaaaatacttttgacccatcttgtctcctgttcttctgatacatcaaaacaaggtagcattgaggttttagagtaaaagataagcagactgatgggttgtgaaaaccagacctcataagagttattgtagaaaatctgggagaattgactagaagtaagagaacaagaaatggagtaaaaattgtcatgtacacctggtaactaaggagaaagaaagaagccgtcccagaaaccaagatacaggagcaaaggtgaatatatgagggattaactaaacaggcacagtaatcattagtacagcagagcactgtgggaaagctgcagcatattcatgcacagtttgatatttgatttgaatgatgctcttcaatcataaaagttcaattattcaattcagtaaatacttgtttagagccagatatgtgtaaaatcaatgtgtacatactgtcaagagtatgaaaatcagtgagatgattctgatattcaacttggagcaaactttctaatagctagaaaaagacaatgcacagataactaagttactatataatacagaaaaaaaagaaataaaagtaaatattaaagaaagggaaaggaggcattctagacataggcgatagatagcttccataaacttatgggtgtcaaaatccatgtttggggaatggccagtaatacagcatggagtaaatctgagcctatcgtgggaagtgaatgaacacggcacttcagattagtgtggatagatgtgaatgggtgcatgtgcagcagccagtctctgtcaaagcacaagtaagccatgactgtgtttcagagatttcctacaaagcctcaaattctaaaaatccacgctgattcagaaaattcattcaggatgagtaatagctgagacattaattgtggacagatttgcagcagggaggtatgaaccccagtttggacatgtggagttcagactgctttagagattgaagtatacatttctagttggcagttgaatttggaggtctagaaatcagggggcagtactgtgctggaaaatgtaaatttgggattatattgatgcaaaactgtgttcttctatgttgctgaagatttagtagcacatatattatgaaatgatattgcaaatggtaactgtaaaacagacattatagtagtcatgagctatttttttccacatcagtatctcagctattaacccctgaaagattgtttgaaacataattgtaaaaagaaacgaatgcctcgtagaacacagtttctaataaatacagcatttttggtgtgcgtccatatcatcatccctcctgtctgcttatttcaaccttttctttgtcctgatgtatttaagtgagaaaatgggttttgtgagtggattcttactatggatattttaagttcagaagtaaccatcctataattgcatctatccttaatgactaaaagaggaccttttggggtgtgttttattgatttctctataaaatttgccaatcccagattgcctttataatctgcttaagaaacagaatgagatcacagggagaaatagtatttgtattaaataattactaagctgcaaactcactgaaggaatttaactcttcttgagctggaatggactacagtagaatgaatatggcttcatggcttctgcgtagcatgacctcaaagcctttttttgtgtgacatttgatatgcacatggatgaaagattttctgaggcctgatgtggaaaacgggttggtcggtctctctcttcacactgcaccccctccaaaacactaaaagaggtgcttttagtgtttactttcaattgctacattgtttgattttgaagtgtccgcttcatttattccttgtggtgaaaactcagtgggtccttttcatctggaacctcagatccctcagttttgcgaaaaatatataacattgatgatttttctccctctgctttctttttctttatattggtagttggacatcgtcatttcagtttctgaacctcttggactagtcctctaatattatacagttctgcaccatttttttttatctctgtcttttgctacttggagagttcctcagtttgtcttccaaccttctatcaaatttttcatctttgctctcatgtttttagttgcaggagtttctatttgtattcaaaatttttaaatagagcattcttgtttcagcattgtagcgtcttttattaactctctgaagggtcagttgttcaagtctcctgtccccaagatgtgggcctgggttcccttgctctgcatttggagcaggggagatggccggccagcccagctgtcagagtggaagttaccctgtcaactgttctcctaatgcagtttgagcaagtgttttacttcctttcagaatcagtttctggtctactgctgggaattagggagtgcttgatagctagccatttgtggacaggggatatgggaattgaactgctttttaatgagagttcatctagtcctgcaatctcagccccaccctcacccctgttttcactgctgcccatttcctcagtatttttggggtggattgcaaggaaataagtttctttcttaactagagtcaggtaaattttaagttaaccatctgctttctagattcttaatttttaaatggtttcctttcccattttcttacttcttacgagtttggacattttatttatttatttataatagacacagggtctgactgttccccaggctggccttgaactcctgggctcaagcagtcttcctgcctcggcccccccaaagtgctgggattacaggcatgagccactgtgcccggctgagtttgtacattttaaactctgcagtgttgtagaagaatttttggtgggagtgtaagtacaccattccattaatttctccatctttgaaaatttttgctccttcaattgtttttaaaagtgtaagcaaaaatgttgattttgaagcaaacagggtagatgtagctttctcctatttttatatttttaactatcatttaagttgttgaattagtaagagtacgcattggccaagtgttttacaccttaaaaatgtattcttgcaataaagcaaaatggcaaataatactgcaactttgtttctatcatgaaaataattattccttttaaattctctggcttttctccatgtatagaggtttctgcggcaagtcaaattttagtttttattcttaagcttttctcaaaaatatttgattataaatgttcctcagttttcattgtattagtatctatatgcattagtgtttgacacacacacatagagcttttaatgtctttgtctatttataataacatctgtatcacttctagattggtttgagtgaattgattgggggggaggtggtctcatgtattgcatttttcttgctgctttgcatgcccgttaatttctaattgtatgccagacttttaccttgttgggtggatatttttatattcctataaatatctttgagctttgtcctgggacacagttaagttagttggaaataatttaaggcatttggtcttacttttaaaatgtatttagttgtactggaacaatgcttagattaggtttagttttttcctaccactgaggcaagacctttctgtgtcccttacccgtggggaatccttcaagtctttcagtcttgttcttttctagagccaggcactttgacctggaatccttttgggtggttccttccctggcctctggaattttctctcaagtatgcaacgatcagggttcagctgaatatttgagtggcagctctcccaggactctctgagctttggctgcctcggtttccctggactcttgcttagtctcctcatggagttgtctggagactctcaagccagtgagtgggggcagttataggctcacctcagttgttcccaactctgtgtcatttgtccttagttgtctgatgtccagtggcttgaaacttgttgatacgttttgccagttttttggttgtttcagcaggaaggtgaatctggttcctgttactatatcttgcctggaaacagaagtccctcatatatatgcgtttagtcaaataggtgcacttttaggcccaccaaataataccagactagtagcttttgtaggtctctctccaaaaaaaaaaagataaataaaaaatacaaattttggtgagaaaatactgttagcattcttgaacataacaattgaaatctggataacaaaactaatttttcttgccacagttagagatattaatacatattttatttcttgccatacagataaccctgaggtttccagtttcttgattcattgactccttggtttcccacttagtttgcatggtgtaccctagtcaaaaagaaatatttcagagttctgtttattaaatatttactctacaaacaactttgaaacagactatacattttatttgtaaataaccacatttttactaatagcatttgtattttcttgggtgctgttcaaattctcaaactttggaatcagatttttgtactgccaccctcttttcttgttccacattgatttttgtatgttcttgatttttattacagcaaccgctaacaacctagctttgtaaagatatgacatcatcaaaaagaatgtaatgtgaccttatgttgaaacagtcagatattctgagctggtggttcacacagtgtcggacagatggtacccctgtaaaatgctgtgctgactcagagttcactctttggaaatacatagatggcccagcataggattgtgtctgattttttttttccctgtatccccttacaatatgtggatgcattttcatcctaaagatgtgttgattaaaaataaagcagaagcatttactgttaaaatttatttttagccttattggctaagctcaatcatgtgaattgttgcagaaagcagttttattatgcctgctttattttcttttactggaatctaacaattaggtaaatagtaacttctaaaataaaggtccaaaaacagcagatatttattgtttgtctgtggtaccttgctgtgtgccaggtgcatttatatttaccatttatatactacttatatattcttattttatttaaaaacactttctctaaaacttaaaacagcgattctgaaattatgccaactctacttaacctccctgtagcttaaccaggttttcaacatggggatatttcttactagaagacattaaggaaatgaaatatagatattctctatgaaactagtatttatgacttgatataattaaagagtagccttacagttaattttaaggtatggtctgatgggaatgtagataaaactattttcataattaccgcatgaaatacttaaaacaagtggtagtcatcttacacaataacatctatatgcaaaataccttttctaatgctgaatacaaccaatgtcattaaaagcagttgtaatgaagtggaaagatcactgaattagagaatgaaaactttgattcagttcctaaattgacagcctgttagctgtgtgacattgagaggtgacccccttaaccttgatgaccttctgtttacttataagtaaaatgcagaaataatgacatgtttctcaaagtgtttttcatggtaatgtaaggacagtatcagatgtatgttgtatgatactacatacatgatgatgatgtatgcttctagtgcataggagcacgcttggggctgatctttagcatctggttctgtgcctggtagatagtaagtgctcagtaaatacttgttggattaattatcatttgacccagtagtctcacattcaatcaaactttgaggacccagaaaaataattcaaagacttgtgccatcatagcaaaataatgcaaacatagctgaaataattgacaacaggttagtattatgaaagatgtgctctctctttaaagagaataattttaatcatgtactctgtaaattttttaggaggagcaaaatagaggcaagcccaattgggagcatctaaatgaagatttacatgtactaatcactgtggaagatgctcagaacagagcagaaatcaaattgaagagagcagttgaagaagtgaagaaattattggtacctgcagtaagtaataatttccagaccttagatttgggcctcgtccttttttgtcagtttattttaaattatcgtaatgtttataaggaataggttattggttgttagaaattttatttaagcataaaattaaaaacacctgactgatacttctaacataataaaaacataaagttcgtatttaggggtccttagatagacattaaaaggggttttggagggtgacgtcgcgtaagaaattatccagatgcttttgtgttatcagttctccttccttcccggtttctctttccccatctcttcccccatctcttccctctgccccccttccttgggttggctccttattgtttcttaaccctttaatatttttagaaataataattgtactactcccagagtataatttttttttttttaacgggaaatgaaaaataaattactggttttctgggccattaatttttttttcttatattagtaaaactatatggaaatgtggaccctaatttgctattggaaaaactagggctgggtgcggtggctcatgcctgtaatcccagcactttgggaggctgaggcggttggatcacctgaggtcaggagtttgagcccagcctggccaacatggtgaaaccccatctctactaaaaatacaaaaaataagtcgggcgtgatggcaggcacctgtaatcccagctacttgggaggctgaggcatgagaatcgcctgagcctgggaggcagaggttgcagtgagctgagattgcgccactatactccagcctgggtgacagagtgagaccctgtctcaaaaaagaaaagaaaaaactaggaatactgttagtattgtgtccaacaaaaagcattggcatttacataaatgagttatggttcctgctgctttttatttcccagatagcctattctttattaccctgatagtttattcaacaagtaaactcacaaatcataaacatttcccattaacttgatgaaattaattccttggaataaaagagttttatgtaaattctgttggtagattttattttttttaatgatcaggttaaatataaactaaaatctatatttgtgtactattaaaccatggaatctttctcattttccttaattgtgtggtattgaaaaactgtatagaaaaggagattttcgaaagtatactgggcaattagtgtcatgatcagaaaaagttatttaacatcgacttttactgctttaataacattttcttttctgtagcttactttattgtaagaatacggtatataacatataaaacatacaaaatatatgttgatcaactgtcaggcttccagacgacagtgggctattcgtcacattttgggagagttgaaagttacacaaggatctttgatgcagtctgtgtccctgacaaccgagccctcccttgttgtttgaggatcaactggatattgttagcgtcttttaaattttctatttacccagtcacctgtaatctcttgtcttgcttaccatttttatttctagtgtttagcatagagcccagcacatagcaataacatagtaaattgaaaaataaatgattgaatcttgtcataaggtaaaatcagttccttcatcttcactatagaaatgcataaaataactgaattttcctgaagcatcaaatgatttgggtttttacttaaataaatgtgggagacagaatacatcatatttttgagaactcacttcctggtttttgaatcccagctttgtgacttttaactgtcttcattttctcatctatgagattggaacatggccaagcatggtgactcatgcctataatcccaacactttgggatgctgtggtgggaggatcatttgaggtcaggagttcgagaccagcctgggcaacatagcaagacccctttatacaaaaaatttaaaaagtagctgggcctggtggcacatgcctatagtcccagctacttgggaggctaatgtggaaggatcgctgaggccaagaagttgagctatatgatagcaccactgtacgccagcctgggtgacagaataagaccctgtcccttaggggggaaaagaaagtttggagcattataaacccctatacttcttgtgaagattaaatgagatgatacatggagaatacttagcataatacctagcacatacatgctctgtaaaagtttgttgtttctatacaaatgctgatgataaaatgaatgcattaattcaagttggaaaaaatgtacagaaatagttccttgccaactcactgttactgttttcagagaagttgctttgacttctatgaaaatgcaacataaggctcattagtttatcattgcctaaatattttcaaaggcagatattttatcttgaaatgtacctttaatagtgaggttcataattttgtattgttgacagtttgaatagcagatgcccagttaccctgtagattaacagaaattgccagtcttttggtttggaattctcagttacgcttttcttgttcgttcagtattctgttgggtagtttagactttttaacttgttaccatggtcctgaaaaacaccaagttcagtcaggttacttcctgtctctctcagatttgcttaccttccttcatccccattgcctttacctacttcaaaccatgcttgtatcagaaccccacaactggtattccttcttctggggcctctcaaacccatctttactttgcagtctattgttacctttccctgcttaaagattttttagtggtgccttcttactctgagaaaagaatacaaaactccttagcatggcctacaaatttcttcatgatttgccatagcttacctttcaggtttcatttttttactttttgctatacctaaagcacacagtcatatgtacacatcttcctcatcctgccacagattgagttgttctgtatttatttgaatttcttaaagcttcagtcttctcacctctgggccttgtagttcattcaacaaatgttaattgaatgcctgctgtaggttagacactgtgacagctgtggaggatacaccaataataaataaaagctgaccagcaaagtgcctgccatcctgaagtttaccttctgattggggttggattactgacaatgaggaaaacaaatataggcaatattctcttttaacaaagtgctgcgaagaaaaataaagcagaataagggaatagagaactgggattagggaactgctattttatatgggctggtaagagaaggtgacatctaagaaaaaaccttagcgaaaagtgcggtggccctgaggcatgttcagagagtatccaggggtccagtgaggagcttaatgagccggctgaagagcagtaggaggtgaggtcggtggggatgggaggcactggcagatccaaggcctgtaggcatgggaaggagattgggttttagtccgagatgagaagctatttattggaggaatttgaggaaaggagggacaaagtgtaatttgtatttcaaaggatcattccagctgctggcctgagaatagactttgagggcagtggggaaagcagagattccaataaggaagtctttgcagttcaagttagagattaaagtgatttggagtagtgttgaaggtgctgagaagtagtaagatcatggatttttttgaaggtaaaactgctaggaattttgacagacttggtgtgatatgatatgtgataacttcaagcaattggagttgtcatttatttacacttttacaagagcaggtttgggaggaaagtcaaaaaattggatttgaaagtataaaagttgatagcaaatgttgaatgggcatttggttataacaggaattaggtgagaaagtggaacctagaattatgtagaattaagaattgtcattagatcaaaagctttttgagccatgagactggatgtatcatcaagaaattggatataagtatagaagaaaaattgaggactaaacctggggtgcttcaccatgtaacaggaagagagggaggatccagcaagggagagcaaagcggggagcccctggatttggaggagagctgtgagccagtcctggaagtcatgcttagaccttttaggtaggaagaggaaatcatcagccattggatttggcaagtagtggtcattggtgaccttgccagtcaatttgttttttttggaacagtggggttaaaagtgtcactggcatgggttcaagaaagaatggcacaagaggaaaaagaaccgggattttttccctcttcaagattttattatgtttataaataatccttcctgtactttggcctctcggttcttttatattgcttcacctgcagtgatcgtgtcttccactccacctcagtcatccagcctcatggtagttccttagacctttccattacccctaaaggtgccttcctaaataggcagcggcagaatgtccagtagtgaacatttcccttctcttcagtggaagagatgctgcatgaatttggcagagttaattaacttgtgcatatatacttgaagggtaagtctcttgaagtagaattactaggttaaagggtaagtgcattttaaattttattattttattcatgttaaaatttctaagttgccttccagaaaatttgtaccaatttactgtcctgccagtattgcaggaaaatgcctatttctcaacacacactcaaaccctggatattatcaaactttgatctttgctttttctaataggcttttttttaaaagccctttaatttcaaaaatgtgagtaaaatctcttggttcttataactatttgttatttaggtcagtttttatattaaggagctttgtgatacttacgtaacatgagatacttttgtaatgtaagaactttaatgttcacatcttgaactgttttttttttaaatgggaaatattccatttacattatcatataaagaatgaataagttctttttttttggaaagatagagtaagttcttttttagaaagacaaagagttttgtgtattttaatcattttgtgcccattttgttaggtgttgtatagcaaaaaacatctagtccaggcttgagggtcctagcagggtggggagtagggaggggcagactttttgcttaatataaactttcataaggaaatgtaataagtattttgtgtttttccatctgcagaaaagactaataaatctgattaaagattgtacttccttctctttgtgatgcagttctaatttttaaaataattttgaagattgtaatatttcatcagtttgcataactttacatgtaactaaatgtttagtaaatcagttcaaatcttttcaattcgtatttgggtttcaaaacaatctggtagacttctttgttttttaccaacaagcgtgcaaactcagagaagcttgagcattataagggcaattatacttgtttcttttcagagaacagttcagtattgtaattatactgcaaaagtaataagggttttgagataatcacacaccatttattagtagaatcaaataatattttacctggagaacatgtttactctgaaaatgacaccagtttagtttcagatgtttctttgattttgatgatcagtttactttattcctttttgtttcctggtgacaaaacatgaaataactaggcaggagttgtttctcaagttatatgtaggtttttatctagtctaaattggttgacttcaaatatctactttttttttcttcctgatccaatctagttctagagttttatcaattttatcaaccgttttctaagtatcagcttttggttttgttgatttttctcaattttttttctgttttattgatatctgcttttacatctttattctactaaggtttagttttacttggggacattttaaaggtagatcagtgatttgagacctgtcttttgtacttctgagacttttcttttaaggacatacattcctctctgagtaatgttttagctgtatccaacaaataatattatgctgcgtttttattatcattcagtgcaaaagttttttccaattttctttgtaatttcttctttgagccgtgttttatttccaaatgtttccaaatatttgagacttttctgtttatcatactgtagatttctaatttgttatgctcagtatgtatttgataagatggtagtcttttttaatctaataactttttgctagcccagcatatggtctgttttgatgaatgtttcacatacacttaaaagaatgtgaattctgcagtagttgcgtgtatattctgtattgtttatagatgaagatagttgaaagcactcagatcttctcctatgtctaccctttttttttttttccttttctctagttttgtctgttactgaaagaggtgtgtaaaaatctccattcattcattgtaaagatatacacaccagtgtgttcttgatggcactgaaagggcaaaagaatttggtgtacacatgtacatgcatgcatatctatgtatgtaatccgtctattatagcttatacaacctgtatgaacatggtttggcatcattaaaaagttttactctatttcatttcatatgatagtaactttttccttcttttaattttgaggtctttatttttttctccccaaataccagttatttcatgcagcatcttttctataaagatagtaccaagcagtatggcctacttgccttagagagactttgtaaaggtagtaattattttaattatgtggacatgaaaggcgcatacatgttcctccttcctctttttgcattttatgtgtttaactatgtatgggctggttacttgcaactgcatgtcatttgcaagttcaatttgtgatcccaaaaggcaccataccaggagagagtacttctgttccttccctacaaaatgtaccctgtttgctagtcatgattttgtcaatataggtttttatttacaaggaatagtagcccccccaaaaaatgaaattatataacattactaaaaaataatatatataaaactgtctctccacacaggagcacacattatgggaatgcttctcaggtatcagataccatccttcctccttccgttaatcctcattcatctagttggaaagtaatacatgatatttcttagtcaagactttatatggatacatattctatgatcttttgtgatcttctcagaagagacctaaaacacacaagaatggtgaaattggaggaatattatgctggattatgctgatattaagattgagaaagtagagatgagggatcctgtatcttgttttcactataattttcccacagttttaataggaagtggggaggtaaaaggtttccattggggagatagagtactaagcttgaaagggctagactgtaatacagaaattaagtgatttcaggcaactgaagttcattgatgttgtctcataagttaaaacagactttggcacacaatatgcctgcagacctagacatttcataaagtgttgaaataacaggtctttttctttatggtgagtaaaattgtgtgtttgttttctctgtatgtttactttataggtatttgagaacttgaatacagatcagtttttaacatatagctgatctctttatatacatggaggtcagtactataaaagtaaatgatctaatacatgacgacaaatttccacctgcgggttttgaaatttaaagttgtcatcaggattattatgacatttgaaaatattcatcaagacaaaagtaattttaattcattttcaaaaaactacatgttatagaaatttaggataataggcatctgttacaaaatgcactttttaaaagtttgcattttgtgttccccaaaggttcttttctgtctgccatatgcatttgactcataattaactgagtattaattataacctgaaaatgttaagagttaatgaatgagaaggtaacagacatcatgaccatagagatgtggtaattttatctttgatttagttctttaaccaaattgttaatgagttgatggtactgcatttatttagtccttgatcaaatattgatcattgaaatttgtggtattgttggcctccttttcattttattgggagagaaggagaggtattaaaacatatattaaaagacatctagaggtaatgtactggctccttgatagagctgtaatgggaaagaacatctctcatgtaaagaaaggtgttgttactgatttctaccttgcagagccattttaatctgaatgggaaatctgtctggggacccagagtgtggtaattagcagtctcttcagagtgtcagagcagaattaccattgtaatggtttagctgcaggataaaaggattgaatttgggactgcctgtctgccaacctaaatgagttgccatgggaattttcaaatgtgagaggcgtttacttaaagctttttggcgggtttgaataaactcataaattgtaggtttgaaagaaggcagtccttgctttctctcatataggcaaaactgtatagaaggctgatagcttcttaaattcttaatgagaatgagttgatccatgttcccatgcaggccatcagcttattcaggcttgttttttcactaagtatggtacaatgaagcacatttgaggaaagacattttgttccaagtctctagaggaaaaaagtgacaactctagatataaatttcctatcttaatttaaatatattttcagataatgctattatactgattctttgttttgctttaacaaacgttaaccagatactgactttaattttactcagtgcatgtaataaatacatatttttatctttgtcatagtttttctcacaaagtaagaacatttaaagcccttttggaaaataaataagatacttaggtttataaactggattattcaggttgcacgtaactacaacctgcatatacatgggttccgcacccatggattcaaccaatgaggaaattccacagagtttcaaagagagcaaaatttaaatttgcatccttcaagcactacattgaatccacatgaatgaagtgatatgtgggcgttgcattaggtattataaataatctagagatgatttaaagtagactggagcatgtgcgtaggttatatgcaaatactatgccattttatatcatggacttgagcattcatgggttttggtatctgtagattttggtgttctggaaccagttccccgcagataatgagggacaactgtactttcagagagagcttcagctctcccctgtctctcaaaagaattaaattttggtgtaatgactttttagtgagaaaatgttcctgcaaatgtctaaggtgatgaacatcacttacactttgcgtgccctcactcatcctcaagatatcagtaatagtagttcttacattggtggtttgtatctgcccttcagaaaaacacccgaagtacaatttaagccaaaaattttgagactgtaaaatacttttaacaaaataaacatggtctttttggaggtcttaacattgtcaagccattagggtacagtttcattaagcaaataataatttggttgttactaaccctagtggcctgaaaatgtggatgcgtctttcaaaaatgtttcaccagggaaatgtgggtattttgaggagagaacagttgttcaattcaggtgagaaagagtaaaattttttaaggtacaatacattttttgtttatatattaatctttactggtcagtatgtaatcacacatcagtataatctgattaaaatcctatagctctaagataactgttgaaccattaataaaacttgagtcatggacagataatgtcctaaaatctcatttaagtgtagaatacaaagacccatgtagtccttaccagagatctctgtaatttctgcagagctccttggaatcacagccatcacctttgtgtgctgtgtgtaaggtactctcatgggtatgtgctgctttgggaaaaatgatggcggagaaaagcactgcagaaatttaaatgtgctgattttctttcctgtgaagagggacaattctaagtaaaatccagttctatatcagggtcataatttttatattctatctccacattttaggtgtgtggcttattctgcattaggacaacaggcacattgtcctaactagaattttcacaaataattaaaagtaccatttgattcgggtgacattccatacagacagattaagaagtttgccaaggtataggttttaagacttccctcaacatcataccataattatctaaatcattgagcagctatataaatctttgcttaccgtctgtccagccgtaatgttgcctggcagtgactgtggcttcttaaacacaacagagggcttttccttaaacttggatgtttagtttgatcaggctccttggtgataaaaaaatatgtcttgtttctgggaactgaacaagctttcccaaacagagactctgtatgaataacatcctcctcttgattcttgtaaggattcgttgaggaaccgcttctttcaacacaaggttttgttcaggttgacaaaaggtttagacatcttgtaattctaaattttaggcgttgcaaatgtaaaaaaaagggaccctgggcatagaataatagggcctaggatatatttattaaattaaatatttataccctaggaaatatttttgcatttgtaaaggaagtccttttactttaactttgtattccttgcacccctcagaatttgcatatttagtttgtaacactttataattgcttgtacctattaatttaaatattaatgtaaactattttataattggtataaatatataattatatattttatataattatataaaatattaatatttatgtcccataaacttctcattggttgggataatgcattttagtgtttggggttttttaaaaatttctttattaaatgtatgatattctacacagatactgtaaaattaatttcaatacatatttttaaatatttattagaaaaataggcgagtgatttctcagtgtattttctttagttcaattattttcatacatcactttaatttgtttgcatgcccttctgatggtgggtaagtagcatcatgttccttatatataggcaaagtatttgtaatcattcactttatagtagggtgcattgaacttgaattagtaaaaacattaagaaaatcacatgtcccttactatatttaatgtgcctgtgttttaactcatatttaatttttctttaccacatttcagatgtttcagtgttttgatatttttaagtaaaattattaggagtaaccattgtgacattttaatagtatattaaatgattcagaaatcacatgtcccttactatatttactttgcttgtattttaactcatatttaatttttctttactacatttcaaatgttttagtgttttgatatttataagtaaaattaagatagtaaccattgtgacattttaatagtatattaaaagattcaaataagcattttagtattaagattatttcaggtaagcttttggtagctcattatagctttatttttggttgtatttttaataactattttggaaaatttgaaagtgactattaaattatcctgcttattcatcacaaggctcctttatgggaattaactctgaaaccatattgccaaattatataaataagtagatgtgttttgttttttaattctcttaacgtttaaaacctttgtggagaaaaacttaatctttaatgtataaataaaatataagagtatttatcaaagtatctttagttatgccttcaaactgacatggcctcagcttatttttagtaaccttctttgtagaatataatatttctgagaaagttagctaatttaggaattagcttactttgtgacagtaatttatgaattctcctgtcaatttaaataaaaatactttgattttttaatttagcatttgtggaattatacttgggaatatttctttcaagcgttgaacgactgacaaaagaaattcatctcacaaacttctattgaaaaactgcagtttacaaggctagaaaacaagacttcattatatacagagacttagatttcataatgtaaatgagaatagaacaaaattactgtgttcttaattgcttttttgatcacttgatgtctgctcttcagaactgtcatgctctcactagaattaaagtatttcatttccaatttgttcttcctggacagtctgttctgtaaccatgcctccagttaccactgcagtgctgattgtttttcagcgtgcttctaatctggaattccaaatcaaccagccattcaacacctccatgcgggtgccacataggcatctcatacataacattttgaaaactaaaatcacattcttccttgccctccccgtatcacaattcttcccttgttttctctgtagaatgatacttgttgcctaggactcatccaccttccttccacctttttctctcctgccctggctccttcccatctatatcatcaccaagttctgtctgattctacctccaaatagcacatatcttttctccatctccacagatggagtaggtctgttacccatggtgctccctcctatccattttctacttgaggccaaaactatctttttaatatatagaaatgtcatcacactccttgttgtaggttttcagtgactttctatcaccccatggtaaaaatgtatggcattcatagcttatcttcatctaacatgtgcctacttttccaactttgtttcatgtcattcctcctttacacttacacccacacatattaaataactcacagttttcagaacactgtttgtttacgtagtgtattttggcgatctccagaaagaactccgtgatttgtattctgtactaatattatagtagaatgcattgtctatgatagtggtgtagaattattagaactgaatgtttgtttgtttgtttatccccttgcccctgactgggcatttcagtgacatgcatgaagtctgtttcatcttagtatctctccctcactgccttgcacatacatagaaagtgatcagtgtttgttgaattaatgattgaatattagtcctttaacaaagcacttgttgtttgcttttcatagtaaaaatggaacctaaaaacacattcatgtaaaatcttccacaagtcacttacccctttttgcctcattttttaaatcacctcattaggttgctctgttctaaatcaattaatatctatatatgtgtgatggtcatacacatacgtaagcctacccaaaactattaaattttacactttaaacaggtaaatattgtggtatataaattatggcagtaaaactgttttttaaaagattgtaatagaacatggtcagtttggtaaggaactgaaagaaggtatgtgtgaccagagtgcagagtgagagagattgaggtgaggtacccagtgagtagacctagcatgtttgaggatattgatcttatctgaagaggactgggcactactgaagagttttaagaagtgagccaggtttattttcattttgtcaatatcactctggctacgaggtgagagaacatcttaatgggccagagtggatgtgtgaaaggagacccggaaggtgacagttgagttagaactgggcaaaataagcagttatttaagtggtaatatagactggatgtggtgaatggtgattgactagaaggtatcgtttgtaactccagaattctgacttgtataactggatggatagtgctgtattcactgtaattatagaatatggttgctgactgggtaatttataaagaaaagaggtttaattgactcatagttccacatggctggggaggccttaggcaacttacaatcatggcggaaggcatctcttcacagggcggcaggaaagagaatgagtgccgagcgaagaagatttgaccagagaaatgtgggaggagaggccagagaggttgaggggggggacatgaggttggtgatgatgaaatttgagcagagccaatgtctcattttccttagcagtcctggaggggatatgcaggaagtatggacaggaaagggtaggaaaaggcttgtgaacatcccagagaaagtgttctcctgcactccagtgctgtgaataacttaagactcagtaaaagaccaacagctagaaaacttacttcctgaaaacactgcgctgggcatggtggctcatgcctgtaatcctagcactttgggaggctgaggcgggcggatctcttgaggccaggagtttgataccagcctgggcaacatagtgaatccccatctttacgaaaaatacaaaaaattagttgggcatggtgacgcacacatgtaatctcagctactccgaaagctgaggcacaagaatcatttgaacccaggaggtggaggttgcagtgagccgagatcgtgccactgcactccagcctgggtgacacagtgagaacttgtttcaaaaaaacaaacaaaacaaaaacatactacatggtgttctttacttgtctgtggtttttaaatgaacatatattagtatgcaattgtagtaatgtttcactttgttattctctacctcttaaagaaaaaattaccttcttttacaatctaacacactacttttttaacaggtgttttaagcatctgtttgtatttgttgaacaaatatttcttaacattgtatataccaggaacctaattgtatacttttcagagttaaactatgcaatctgttcttcagtaaatgaagctttgaccgtgcagtcaaatccattaaaagttcttccatgggaaattaggagagtttcaaactcacagtgtcttagtttgatttctgttccttataacaatacctgaaactgggtaattaataaagaaaaggaatttatttctttgttatggaggctgggaagtctaaggtcaaagggtggcatctgtgagagccttcttgctggtggggactctgcagtgtcctggggcagcgcaggcactacatggtaagggggtaagtgagctagtgtgttcgcccaggtctgtcttcctcatcttatgaagccaccagttctcttcctgtaataatccattaatccactaatccattgagggactgatggcagagctctcatgatccacgttgaggattaagtttcaacatgagttttggaggggacatttaaaccatagcacatagtaacaaagctactttttcttacaggaatttcagtaatcagagatttggggggaagctatgtggctattattataaaactgagttctaacagggcaaatccagagttgaattacttctaactctggtaccacagcagttgcctttttttcaccactaacaaactttcttaaatatacgtacgaaatatttaaagcaccagccaccccaacaaagagctgcttatggctgggcgtggtggctgacgcctgtaatcccaacagtttgggaggctgaggcaggtggatcacctggtcaggagatcgagaccatcctggctaacacagtgaaaccccatctctactaaaaatacaaaaaattagccgggcatggtggtgggcgcctgtagtcccagctactcagagaggctgaggcaggagaatggcgtgaacccaggaggcggagcttgcagtgagccaagatcgtgtcactgcacttctgcctgggcgacagagcaagactctgtctcacaaaaaaaaaaaaaagctgcttctgttggaggacaggggcatcgatcaaattttaaccaataatggccagccaatgtgaatgttatctaattcattgattcctgattccatttcttatgaaaactagtatgttagaaaaataccagtgcttgcatatctgtgtgcataaggatataggaaaacacaaaatcagtttctcctactatactctcacaacaccgagtgcttctgtgatctatggtcatcaaaatttgtagggattcctcctcaccaccagcagccagtcagttctgacaccagattcctcagcagacaccagctgggtgtcctccgatttaatcctgttctgacgctgtccacctggagatagccttaggtcccacaggttgagggctccagttccacaagatgtctcccacttcaggcaccagtcacaagtagtaggtggtagcccatacttcttccttaccttactttattctagcttagcctaactctgaggttcaagattttagtcagtaatttgtgttgaagatatcattagacatctgatgaaacaacaaagaaaaagaagatgggcagcaaccaaaattaattacctcctctggtgttaccaccatgactacatctgaaaatagtttgggtataagtgaaagtagcagtcatgacataaaattgttttaaatgccaggtagttttctaccgctactcttctttgaggttgtcaaggttgtgcttgatagcttagaaatctcatgaatatctcctggtgaaaagcatttactttgaggagtcagagtcactttgaaaagtacttcattactgtgatgaatacattacacagcattaccttggaagtcttgtaagagcagttggagtgaattaacaatttaatcacatatcttcatgggtaagtttaatactcacagttaactccctaaaattgtatgcaacattttttgcttacacagttgacttttgaccatagtagggattaggggaaccacccccacgtgcagttgaaaatccacgtataattttgactccccaaaaatgcagctcctaataacctgctgttgacaggaagcattactggtaacagttgatcaacacatattttgtatgttatgtatattatatactgtattcttacaataaaataagctacagaaaagaaaatgttattaagaaaatcagaaggaagataaaatatatttgctattgaagcagaagtggatcatcataaagttcgttcgttcttttttttttttttttttttttgacagagactagctctgttgcctaggccggagtgcagtgacaagatttcagctcactgcaagctccacctcccgggttcatgccattctcctgcctcagcctcccaagtagctgggactacaggtgcccgccaccacgcctggctaattttttgtattttttgactagagacagggtttcaccgtgttagccaggatggtctcgatctcctgacctcatgatctacctacctcggcctcccgaagtgctgggattacaggcgtgagccaccgtgcccagcctcataaagttctttatcctaatcatcttcatactgagtaagcggaggaggaggaggggttggtcttgctgtttcagggctggcaggggcagaaaaatgtctgtataaaagtggacctgcatggttcaagcccatccatattgttggaaggttcagcggcgtactcatatatgttttctagagaaggaatctatagttaccgtaagattcttaaagctgttgtgattatttccctaggccttttcatttggtcttccaccgcagcctgtgcaaactcccatcataatcccagttgcacatcatttatcttgctttacctttctatctcagtgctcattctgaatatgaatgagtttaatatattttcactttcagaactttttgtcaggcaatcattacaacatcttttaggttctgcccacctctaagcctcctcaccaccactcccgccacagtgggtatacttttcttagtttttgcctctctgtcccacctctcaaaactaagagatttttagatagtcataattttcaggttcttttttttttttaatttttatttatttatttatttatttatttatttatttatttatttatttatttattttttgagatggagtttcactcttgttgcctaggctggagtgcaatggcacgatctcggctcactgcaacctgcacctcctgggttcaagcgattctcctgcctcagcctcccaagtagctaggattacaggcgtgcaccaccattgccagctaattttgtatttttagtagacatagggtttcaccatgttggtcaggcaagcctcaaactcctgacctcaagtgatccacccacctggcctcccaaagtgctgagattacaggcatgagccaccatgcccagctcaggttcctattttttatcttcttttgtagaacaaggctctattaaactcccaattggtatttatatgtcccaccttccagcccaaccatatcatctaataatatccgtctaaaataacaatcaactttggaggtgttttttttttaatctataaattgatgtggctctactaatgctagtatgtagccggagttgtgaatcatataaatccttcagaagtttatcatgcctgttaagattactattactattaataaaatcacagattgtatcgtatttagcattagaagaagtaagtacttattattgagacttggcttttgtttttctgagattgttacttttcagtggatctgtttataccttgttgaagagaggcagaacatattgtcctaaggtacaaatctttgttagttttatatagttttaatggaggaggagcatcatatttgtgtgtgtttgagtgttttaagcttttctggttaaataaagtgcctgacactttccctctaccttgctttatgtgtggaaagttgttgattggttacacaggtattgagaactgaaaaaggagaaagacaggctgtaatacagatttagagactgcaaaaaaagcatcttttaccccagggctggaataaaagagaagaaagcctgtgtaatgatgaaaccaagaccctggcttggtgtctctcagctacttcaagaacctcaggagctcagaggggagaaccctagctggctgcttctggtgtgcatggagggaaggggctgcacgatgagactggttctagaggtgctgggaaagaagcatggagccaggagacagctgcctctggcaagtgcatagctgaaaggagcgcacatgggaaagagaacatctctttccctcatccagctcttcagggtcggctgctttacacccacactatgcctcagttctgagaacctcacagaaaaccggctggcaaagaagtagtagttgtagagccctacacttaagcattggaaagcatatggcagttcagtttggaacttagtaactgccacatcatcttggagaaaaactcaagaaatatgaaagcatatttttctaccagctgtataggataagttggaataagttgatcaaccagatgaatattcagctgtgtacctgacatctccacttaggtgtttaagataagtatttcagatgtaacctttcaagagagaacttctgattccatattcctcaatttttatctcattacatggagccaccgtctactttattgcttaaatcaagaacctaaacaacattgttcaatttctccttttcccttaccctctgtgtccaactgatcatcatgccttgtcaggttaacataactcttttcatctctgttacaacctctaccccagtccagttcatcctctctgttgcctggacaatgttaaagctcatgacctatctcagtaatcacgctggcgcccccagcagtccatccacaaaatagcacagtgaggctttaaagacaaatccttttttgtcacttccctctcataaacccctttgtttaagtcaatcaaattatctgtcatcttctgtctgccaaagctgtcggagcagatcaaggtggtaattggcatcaaaattcttcataaaatgtggtcactcatcattttttcttttctctgatatttgttattcttcaattaaattagtcatgtatttttttgtttggaaaaccttttctacccattatttcaaatttatagaaaactaattcattttttaaaatatgttgagcacattcatttttgcaaggctttgggggttacaaggaacatgggtttattggttattggttagtttatggatacaattcatgataaatttctttaaaaatcaaaaaatagaaaaactgagatagcacatggctatatatgattgatcaccccaaaaataagcagcatgaatttataaaagcaagaccagtatgcgtgagagttcttagaaaagcctgcactcaggagaatcgcttgaaccagggagttggaggttgcagtgagccaagatcacgccacagcactgaagcctagcgacagagcgaaactccagctcaaaaaaaaaaaaaaaaaaaaagcctgcactctgagagaaaacgatgatctgtgttattgaggaacacataggatgcttccaataggagcctaacttggagaggaaagtaccaggtctactcggggattgtggacagatgattttgactaatgcaaaacatttgcaagcaaggttgctcatgtaaactgttcaatgccctgaatgctggagaatttgggcctttgtgataggcagtggttcgaattatttttttagttgacatagttttaatgatttggaaatacacatttgtttcagctctctggtgattactttgttgtagtttttttttaaaagtatatagatctaaaaatactaaataaacatgattttcttactttctaacatgtggtctgatgaattcaaaagatacttccacttaaaggcatcataagactggagaaggtataatgtaatggttaaacatgaggactttgaatcaggtattctgggttggaagccaggtttgcaatgtgaacttttgtggctttggataagctgcctaccctgcctgagcctcagggataatgcactgccaataccacacatttgttaggaagcgtaaatgattggataatgaagatctagtgtctaagtctagaaaagttgaacaatgtataaattacattaaacgtgacctgtttgtatccttttaacatagctgttaaacatagttgatgatggctgggcacagtggctcacgcctgtagtcccagcactttgggaggccgaggcgggtagatcacctgaggtcaggagttcgagaccagcctgaccaacatggtgaaaccgcatctctactaaatacaaaaaattagctgggcgtagtgacatatgcctgtaatcccagctacttgggaggctgaggcaggagaatcccaggaggcgaagttgcagtgagctgagattgcaccattgcactccagcctgggcaacaaaagcaaaattccacctcaaaaaaaaaaaaaaaaaaaaacaacaaacaacagaaaaggcatagttgatgacatatctatatcttaatatgcataaattaaaatatatatatttatacatacccataattggtcatccttaattgcaagcctccagaagttcagaaaactgtgtttatagttgcttcaccccacttagtgtgagtatttttctgtttgttacggactggattatgggcgtatcccccagactctgctgggtgttatagaatctgtggtgtattatctttctataaatctgaaaaactctgaaattctcaaggcacttccccgataccacaggttttagctattagcaggagtgtgttctatttttgagcttttacatttcaaaagtaaagacattctttgttgtgctttaataatctttccataatattttccatacaaatacaagggaaatattcatctgccctatgacccagcaactctactcataggtatttatccaagataaatgaaaacttatatttgcaaggaagccttatatgagaattttcagagtggctctgttcacatatccaaaaagtggaaacaatcaaatgtctgtagcagaagaatagataaataacatactactcagccattaaaacagatgaactacagcgacatgcagcaatatggatgagtctgacagctgtcatactgagggaaagaaattgaacacaaaatattatatatgccttcattatccgaaaatctagtctaggcaagttaaatcagatggatggttagggcttgaggtacaaggagactttctgggtttgtggaaatgtcctgtgtcttcaatggagtactatttagccataaaaaagaataagatcctgtcatttgcaacaacatggatgaaactggaggtcattatgttaagtgaaataagtcaggcacagaaagaatcttcacatgttctcacttttttgtgggagctaaaaaaattaagtcaattgaacttatggagatagagtgtggagcgatggttccagaggctgggaagagtggttgggagggcacagtgggaatggttgacgggtacgataatatagttagaatgagtaagatctagtgttcaatagcacaacagggtgactacagtcaacaataatttatcgtacatttaagaataattggattgtttataatacaaagaaaggataaatgcttgagttgatgggtactccatttaccctgatgtgattaatatgcattatatgcctgtatcaaaatatcacatgtactttatatatacatacacctactgtgtgcccacaaaaacttaaaaaaaaaaaaagaaatatcgtgactggatattgcacttaagatcttgggagtccactgcatgtgaattttgcttccattttttaaatgattcttgaatatagggcaaatacttaggaaaccagaagctatgagattgcggataatcacttgagttcagtattacaaaatatgattttaatgacccattttacaaatgtaaaagttagtgttctgtctctgtagagttgctcattcccgatcaaggttctgcaggatactttgtgaactatctggagttgacatgtacaacttcaggatgccctgtttgatcagttgtaatttttgagaaaaaaagaggtcagaaaaactggtctgaagactggctcatagaagctctagagcctctagactgtagaagaaacttagttttctcatctgaacttatatttgaaaaagaggtgatatataccttcaacagtgtttaggcaggtacgtactaccaaatctcattgtcaaaaccaggacagtaacattgaaataaccacttcgatgtgaaagtaattctataattaaccctgttgtctcttctttttttttctttttctttttttttttttttgagacagtctcgccctgttgcctaggctggagtgcaacggcgcgatgtcgactcactgcaacctccgccttccaagttcaagcgattctctttcctcagcctcctaagtagctgggattacaggcacctgccaccacgcccagctaatttttgtatttttagtagagacagagtttcaccatgttggccaggctggtctcgaactcctaatcttcatgatctgcccacctcagcctcccaaagtgttgggattacaggcgtgagccaccacacccggccagtgaaccctgttgtctcttaagacacacaagatcatgccctgtgaatacagagtggaggggagtggtcaggacatgagggtgaagcccatcctggactgagaccacctagaatcaaattccagctctgccaactaaatcctgtgcatttctgggaggcattagtgtaccctgccaaaatgtggctgtgatctggtgtaacataccatcacctccagcaacaccatatggcagactacaccaggatctcctcctatgccactcttatttttattctcttagggtgatctctcctgttttcattatcctgcaccctgcacattacacaagtgtgcacacacagtggtaatcacacacatttcttccatcttttttctctgtggttttctcccttgtttgacactccattgccaaataatgaatagctattttgctatccaaaaggagcagttcagtgccaacatgttttcaagcacctgcccttctgcaggaaactcgtctttcgtctttatttcctctttgttggaagtaaatttgccctcttatttgctgacattttcaaacttgtaccacttctttctcgctatggtagatgactggaggtaaacctactgctcttctttttctttttttatgtagagtataatttataatacttttggtattccatttttatatataaatttaaatttgaaacttttgataagttttaattttcaaatgactagtttttagcaaataagttgaagttttattataatggatttgattcttcttttccaatagtagcttacttatttcgtttttaacaaataatttctgtgtctttctaagatcatacttactttttgttcatctttgtacaagttgataggagtcaaattctacaggcatgccagtatccactccctgtaatcttctgcaaagtaggtttttaaaaagttggcagtagcacatcaaaacattgtctttatgtttcactcatatttttctttaattcttgtgatttactaaaagctcactgaacagttctgtttactatatattatagtataccattcaccattgtaatgccaaaaacagaattttgtgtgtcattacagctataatagaaaggagcactgagaagcctgtgtcctggtcgtccttcccgttttatgaatcagagaggaacaagactatgtcagaaaattgttgcattgtgttactttcattgtaaatatcacaaagtcatgtaaagttactgggattcgatgatacataaaagtggggggcagttagttctatctctacacagactttgatagatggaccctcatctactgcttgctttgactgtggctaaagtttatgttggactgagacatttgggatttggaaataaagttaaagacttctcataccagtcacttataagttctagtaagtaacttgggggtggccgtgggaacatggggttaggcagtggtaacaaagaaataagccctattctcatggagcttactttgggagtggtgatactcaacagactgtaatttgaagtagaagcaattaactttgtcgataggttcttggagatacaactttaaacaaaatgagtatattgcagcctcttttttcatcaatgttataatgaaaccatgttgaaagcagtggcattatttgaggacctgctgtctgtcgtttcacttaaagttagttttaaagaatgtattgaccacattaagtgaggatctactgtaatctagggatggcccctggagaaggaaggtagcatttaggttgaagtttgtggggatggggataggtaggatttggaaagagagaggggcagagagtgcgcaggggagcacttaggatggaggaagtgggaatatgcacagagatgaatggtggagagcaatctggtttgacttaagatgttacttacatacgtgtgtaaagtagtaggttaatgggcttaacttgagaggatattttgggaccagattataggcaactaaagttttttatatttttagatagtaagaaaccatgaaagtgttttgagacagaatgttgagaaacatgatgaaaattgtgatttgggcaattaatctggcctttgtgtagagattgattggagaacagacaccattagcaacatgttttagtagtttgggtgctatgccaaaaacaactggaacagtaaatggaaagagtgggaccaattcaaggtaaatggggtggccaaacattttgatgattagagactatcctgatttaggtttgaagcatggttattattagtgcccatttttgcttttaaaagttttctgctttggacaataaatcatatggtgaccctaaagatagattgctgtagcattgatagcacttgctaattagattgaaaggagacagggatggtggtgtaagagagcagaatatacggttttttagaagttattatttcactgagttttcatattttgaagaattaagcataaaatttccttacctaaattctgtaatcaaatcttgtatgtttaaaattatttgtgtgcacatgtgcacgcgcgtgtgtgtatgtgtgtgtttaacgaacacatttgagttttggaatattcagcaaaatgatagttgctggctgggtgcagtggctcacacttgtaaacttagcactttgggaggctgaagcaggaggatcacttgaagccaggagtttgagaccagcctgggctacaaaacgagaccttgtctctacaaaaaataaaaaaaattagccaggcatggtggcgcgtgcctctagtcgcagctacttcggaggctgaggtaggaggatggctcgaacctaggaattcaaggtggctgtgagctaggatcatgccacagcactccagcctgggtaacagagcaagaccctgtatctaaaaacaataaaattaattaaagtaattttaaaatgatagatgctgaagagaatatgttatgtttagtcttatctcacattttcattcatttctatttaaaagtaaagctttttaggggaactttaataaatcggtgtaactgacaaaaaatttacaaatacaatatgctgtatatcaacatgcctttactgagaaaacagagaaagcaggctccattatgttagcaccttttggttcttgatctttttttccccttattaaaacaggtgactgtagtcacatgatacttactttaaggcttgtgtgtagatatagctattatatttgtggacagtctcaaatgctttcatctcatgtgttttccatgtataggcagaaggagaagacagcctgaagaagatgcagctgatggagcttgcgattctgaatggcacctacagagatgccaacattaaatcacgtaagaatgagctctgaggcccagggttactgctgtctgcgttgttttgtctacagacttttttccttttggcaacacaataatagttcatacaaagtgaacagttgaaataactgttctgtcacttgatatggggctgacagtcctatttttaatgctttcatgaattgaggacactcttgattttattttatgtcagcatgttgaagttttaggttgataaacttacattattttaacaaaatcacaatctctaaattcaagtgtttaagaaattttctgtttggttaactacagatgtagggcttgcagcttatcttgttaaatttatgcaaaagacaacatattgaaagcttattttgaaaaagtgaatttgaagtgtatctgactgaaatgaccctctgttgagttctgtcctttcactttgcttgcatgttttccctctagtatttttccctgttgatttcatatgtgagttcacagagatcagtgggtgataactctttttccgtcctatttttgtttttatgcgtatctatgcatgtttttataagccgtaagaggatagcttttttatgtttaaaatgaaataatgtgaaatttatattttgaaacaatttaagattcatacatgtcatttatatgaacataccatgtttaaattactgattatttaaaacacatagaatttttaagttctagccagatttagaagatgtaggcaacctctagtgggtttagaagtagttactttacaaattgttgacattaatttactcatgaaaatgtgagtatatcattaataggaatttagaagtagggctttttaagattttctgtgacgttggttcctaaagatgcaggaaatgtacatatatacctagtggttacttgactaaatgcaataaaaattattctttctttactttgaaactaaagctgtactttagaaatataccaaaactgagtttctgtggactccggttacaaattattttttaacatctgttaatgcaagtgtttcattgtgcttctgcatgtactttagtcttatgaaatcttagatatatcaaatgtatgaactaaatataacctatttaatggtgtcattcatttttttacaatggctttttaatttttgcacattcttaatcacattgggaaagggaaagctgtttgcatttgattggtgtgcttttgcatttttgtgtgatcagcgcatgtactaatgatttccttttatttttcttctttcctgcttttcctttctttttcctactcccttctcatttttccgtattttatactgctgtctctatacttctttctaaatttctttgcttactgtagcagcccttgccttttctcttgcagcaacagcccaggctgctccaaggatcattactgggcctgcgccggttctcccaccagctgccctgcgtactcctacgccagctggccctaccataatgcctttgatcagacaaatacagaccgctgtcatgccaaacggaactcctcacccaactgctgcaatagttcctccagggcccgaagctggtttaatctatacaccctatgagtacccctacacattggcaccagctacatcaatccttgagtatcctattgaacctagtggtgtattaggtaagttcttctccccatggggttaacaacattctctttataaatatttttgctccctcagattttgaaatgattttatcagttttagagaggcaagacaagtttgcattagggaagaccgaaaactaaattttgttttatttcagcctctcataagggttcccctttgaaataattgcaccttaaatttattcagatccactttggtaactgaggttcagtttggttgggtaaatctgttgtgtacatttcaacaaatacttttacatttgacattactgaggtcattctgagttttaggtcccaccccattggcccgtcacctccatcagctccacttctcaaactttatttgagatttattttatctcacaggtttgtgtaattgttggaaaatttttaaacttctaagtgaaagtagtctgagcgctttataaaattcatattattgtgtgtgttttgtaatatttgcgatcacttaaaatatcagataaatacagtcatgcatcgcttaacaacggggatacagtctaagaattgccttgtctggcagtttcatcatttggtgtacatcatagagtgtacttacacagacatagatgatatcgtttactacacacctaggctgtgtggtatagccattataatcttatgggaccacatagtataagtggtccgttgttgaccaaaatgttgttatgcaggcatgactgtagttgtattcttaagatttttcagttcttgtgttttggagtcctagtcatattaaacatgagagatactcttaatttcagtagtacagtatggaatagttaaataaatagcaaatcactgaattcagaatctcacttaaggcagaaattaaaatcataatataaacgcttggttttacatgaacaaaaagtggtgtgctcgaaaaagacattactgatgccttttttttatagagtggattgaaatgccagtcatgcctgatatttcagcccattgacttgctggatgaaggactagaatacagcagctgttataacacgaccagtcaatgtggaacaaactgtttctgtgcaacccctttgttttaccagacaaaatttgaatactttttttcctgaattgtatatgaccttggtgctgcatgcatgctgttgacttttaggactttgatcttttaaggttttttttccccagcattaatattgatttataaagatttgaaaatctttaatgaactggagaacactaagatttaaactcgaaaattcgttgttcaagtaaagaaagccatgatgctctgtatgttatctgtgtgtgtgcatgcactcaggtgccctttgtttcatgaacaaatacatttcattgtacatgttttctgtttaaatcattgtataaagtaattgcaggtcagaattataccacagaactgtttatgagaggcttgtgtctgttgcacatttcttgaagcatttttaaaataacatgtaacctgtaaccttgttgtttaagttttcttttctattaatactctgtcctgtggtcccgtgcatgctccttttcccagaactcctctctgctgcacccacagcatctgttcccgaggagttatgactcttgacttcctgcagggctggggctcttagccaccagctgctgttccagcactttcagcgcaagatctccctgattttgccacgtggaattgtacttgtatatgattaccttatctaaaatgaataagaggtgatggaccagtttactgcttagaaatagcaagaggcactgcagtaaaacttgtttctcattgtaaagcttcatgtcttttgtttgttggaaaatttttacttatagaaacttaattattagactggtaaaataaagaccaaaatatgcagatttctaattggcattcataaggtgaataataataagtgcccaatgaaaaaatctattatggttaatttcatttcttgctttgccacctaagcagtaaaacatgatattgaccacttggagaactcagaaaattattttaaatttctaagttataataaatttgcacacagataacatgcatgctatttatgtcacatctcacattaaattattttaaaataagcagtgcccttcaaaacagatgcagacatgtgtgttggtagtagtgaggagattggtattagcatcaagtcttcattgatgactaatttttaattcccttccttttatctttaggtatggctttcccaacgaaaggctaagaattcaagaacggtcttaactgaaccctcatcagatctgaatttaacaaatgcttagtctcagcagcctccgggggaaaaaagcttagcctagcagtcagtgacttacttgcactttttgcacatagatataaagtaaaattatgttattaatttggtttagtctgtaatattacacagtaatggtaatttataaaggagtgtatagtagtatactgactgctaagtgtactatactgtttgctttactaatcacctaattcattgcagttcatacttattgactcagagtttaaaaccacaatctgtaggctttaagaattgcttttaaacctttttaagtgataaactcttgaagtggtcttaaggttagcaaataattgtctgtattaaacatggcgttatttaagtagtcctgctgcaagaaaggcacttcagtgaatatgtgactagtaaagcttaaatatgtttggatctaatagagctcatgaaatactgtaacttggggattgtaaatgaatggatacaataggttaaggcccaagatgtttgaaatgaatgcaatattaatagatgcatatatacatgacatattgtggttaattttaaaactactgtgccttaacgtgtttcttaaacttttgtagtaaatgaacatttgaaatccattttgataaacctgctgttaatgttttttccccccttgtgaatgttttctaactttgtcttggtaattgcaatttaactaggtgcggtggctactaaagttcgaaggcacgatatgcgtgtccatccttaccaaaggattgtgaccgcagaccgaggttagtttagttctgcagttcttgtctataagaaatgcgttgggtgtccataaaatttgcaacaccacacctgatggaaaaatgtaattgcttacctgcacacagttttttttccttttctaaatttgtttgtgatcattgacagatttaagggttgggttttttgttttggtttggttttttctcccttcatattttaacttattaaatcttctattattatgtactatgatttattcctactaaaacataaattagtcaatttggggagctattaaattttgatgttgcctcagttattttcattaataacttttgctaaaatttaccttttaaaagttctgaaatttggggttggtattgcaaaatgaattgaacggatggtgctttaatgagagaagtaaaggaatatatgcctcctgtgtacttttggagaaaactgaagatttagtttccatttttctttataataaaatatttgagccactgtataaacatgagggtgcacatgtcaagtacttagctaaaatcaacaaagtgaggactgtgaagataactttcatgtagttggatttttttatataaattagagcgtcaattcccttttattcttacattctcttgccaaaatggatcctttgatttttgtcatttgctgctaaaatatattatctgaaggtaaactgtctcttgaagttctgtggtggtatgagatccagctgtgtgaatataatttttgactaacctgtgactggaaaataagtcttcatttttctcctttacaacttttataatttgtgtcctgtatatgcccttggacccttttataaattatttttgcttattgaggaatggtattttcggtttattgacattgaattgttttctaagtttttccatcacaagataggaatatcaagcatggtagtaaaaaaggattttttacaagtttctattatggcacatttgtttcctacaaattaaattgatcaaagtctcaaatattagtggaggaaaaagtgctggatttgtacttcaattgtacatcttctatgaatttatgaaggacctacctgctttatgtgattaccttgagtactgatgataaatactagacacagctgttgtccacatcagaaataacatgcctttcccaaagacattgttaatatttgtttagcataatccataaagaacgttgaagacttttaaactaatgtattcatcactaacagtgattgttcagtgaacaaaatagaccccagctccgtggtcgtctatgaggatggtattccttgtaagtgttctgtatgagcacgtctcatagggatggggaagttttttggaaagtcacttgagttttaaagaaactggacaattccatgaataatttgaggttttattcatattgctaatgtctacaattgttctaaagagtattttaaagtagtttttttcccccagctaatctttccctatgtatgcccccttttaggagatttctcaccattttgctttaacacaataattattggcctacttaatactccagtgccttgagatagttggacatattttaggaagattttattgtatttggaaaattacgacacattccatgcatggatcttttgtagttacgtgttttgattagtcctgtcactgtttttatttaaaatgcagtgtcagcaaaccgaaatgctcatttcttttttagtaacagagtactcgtatacttatcactgtatgcactcattgggtctaagggagatgtctgaagtccttcacttaaattattttatgatactactgttttctctatttttgaggttaaaattattttaaaaaaatattttgaagagaagtttattagtataagcattcatttaatgcaaaaaaactcagcccttggacatataaccatgtaattctatatgaacgtttagagtactacatggttgaccaatatctagttctttactaaaagtaaactcacctctttatatatatttcaaaactgaatttgaagcctattagagaccaactgaccttttagttgtaatgaactgtatggtcttgcatggtgacacacctgtgtaggaggccaagacttggcagaggatgtagtctaggtgtcctggtatatataaatggtcactcacttcgtttatatattccacactcagaaaagaagtcagagttttttctgagtaatacaaaaaccatcacttacataatttgtataagaaactttaaacagttttaatcaaaactatgagtaacatcaaaaatgctttaagattctatattttggaatacctgtgaagtagtatgactacatcatgttaaatatttattttcttctatgattttggagcagcaatttttattttaaataaaattccacttttaagaaattcagggaagatttggtcacattgaagatacagtatttttgtagtatttataaactgttctaaatgatagactatagaaaacatttttgtcatatgaaggtaaatcagtccattattttggatcatttaaactgaacattacaccttctgggctttgatttatgaagtggcacagaatctaactttgcactgaatatcttttcattcacatctcctctgccttagtcacaatggcaacagtacagaaaattctatcaaacctcagaatatagtagaaataattaagctgttgaatgagtcttaaaaattatactactgttaagtggaccaagtttggtgaagcagaatgtgacaaaggttgattaaggaaggaacaactcaaggacattgggaatgataacttttccacttgagaactactttatgttttactgtaatttttaaagtttttttgtcctttttgttattttgcaaaagaaaatagtatttacaggtggcttcttttaaaatataaaaatataaagcaggaatgtatatgaaatgtcagattttattgtatttgcagagtattagctttgaaattgaataaagaaagctgtttgtagttttaaaatgccttattggtatcaattagaaatttcttctattttttgatgtacttagagctttttgagtgtagaattttaaatggcaggattttacagtgtttacatgcaagtgcattttataagtgttctatatgtgtaaaatagtattttcaactggaaagtgttggctagtgcaaaaggcctggccattttctggttcccatgatgttgcctacactgctagactacagtttagtatcgctttgtatcatgaggccaagaaattccatgttgtttgtaaatagaataattgaaaaagcaataaacatttattgaacaaaagaaaccttgtttggcccagagtttatgttgaaccaaatggttcatagaattttgaatttctttagattcatttctgttaatcatcagttattaataatcacatccatcattaatagttgctttaatgcttgcatcttagagaatactaatgcttaatagcagtcagatactgataactgcactgactgtttaaaaattctgtataccaaacctattcattgttgtttttcaatttccttcattgactgctactaactattagttgttagttgtattttatttctattttttcttgttaaccagttttttttttttttttgtgggagtgggctcttcttcatttgttcaaatcatgatacgaaattgtcatgattttaattgtattctgtagatggtgtcaatcagtttgtcagaattaaacatgcttttttttttattagttgtgattagtttttcatgttgtcattaagccgtcactagctgaaactaatctcttctctatcttttcttttcttttttttgttttgtttttttttgtttctaaccacccagccgccaccggcaactaacctatgaccttctgacctctgaactcttcacccaatgatgacctgaccatgcctgcctgctgatcagttaactggtaatcgcctttgcttgcctgtcgtcagtgcagcgagctgaggcacttgtccgttcgtcttaccatctaaccaaacaaaagacaaagaaattgttgtcctccaactcagctttttttttttttttttcctgtttgggtgaaagtggttctagaaactgcactgaatagtagtaaagcaataaggcccaattcatcccacagcactgatcatcttttaatatcccaccctaagcgaacggtaagaaggcctctcttaagaaggggagacagatggtccttaactactcaatgacagaggcagttactgtgagagacttctaggaatctttttcttctcatagcgaagtcaaagctctctctgaatgtactgtgtgatgatgcatcatgcatgaaccttcggtcagggatatcattggtgaagtgatttcaaaaagtattcaaaatttgatatgctgtttagtcactacagtgccctcaaagggcagaagttgcagccttttttatattgcctgccaaaatttgaagtattagaagaaagtgtgccatgagagaaaaacttaaggagttttgaaaagtaatgcaaataacaaaactgcaacactatttttaaaaagataaatatctgagttaaaattactgaatctttattttacacctaaaaaaatatgagaacaaggtacatgcattatgtgtcacattactgggcaaactgttcaagtatttttttttaaacctccctgtatagaaaaaaatcattaaggatgtaaaagccatgcttgcctatttgctgtatacatgtaatgaaattgtagataaagtgtagtgcattgaaacaaatgaacaaaaagtagatacttttactatacaagggtgctggtgcagaaaaaaatatatatatttttggaaatgtagcattttatactttcaagtgttataaaaaaaaagaaaaagaacaaagaaaccctttatttcattaaatgattttttctggtggttgtcaagaaacaaaagaccaaaagagcctttttgtctttcttttttattttttaagtattggaataagtctaaagaaaagaaagtgccttattttccttcatggtcattcagtcaagtgtctcataaagatcagctcctccctcagaatacaagttaatgcatgttactcagtcgcccagtatgtgaaagaagatatgagggagacaaatgagttgatagtttgaattataggatttttgccacgttaccttgatgcaaatttgccaaatctgatactgtgaaaagatttgataacttttctaatgcctgactagtaagttttaaaacatcattctttaaaagaatagtttaatacttttggttattccaatcttcaatgttaacccaaaggattctgagataaaaattatgtgtcatctaaacttcagtttacagacaaaattagagctttgttttatttggagaagggggatgcattcatttgattttgttttttgtttttaattaattgaatataataggatacctagcaaatgttttcacaaaagtgaatctttgtgattttcattctagaaaacaatgttttaaatcacccaaatttaatcacatgtctttatatcataccagataactggtgtagtgtatccttttactgtttcaataacctctgcctcccccaccccttaactttaaaagaatgtgaaaaactagcatcatagtgcatataaatactcaaccacatttcagcttaagcttctaatttctcaagaagttgaaacagttagttatgtagttgaagcaatttgtgtgcaaagcctacatctaaacacttgagtgaataatcattaactgctcagtaccagacgtggcaaatctcaagtgacagtggacccccccccccgcccagcttatcaactcgtcccctcttccacaccttcccagcatcacagtcagaggtcagcaggaagctgagtcattaccttcttggtgacctcccccaccatcttgtctcacttggaaaccgttgtcagtttatttactatgcaatagacattcattgttttgtatccagctagctttggtttaatatgccactgctttgtctttctgttacacatacagttttgatttatttacaatatatgctgtgccattttgatttttatattgtttggttggttgaataaatttgcgacactcaaacacttgaggtgatagttgttaaaaacaataccagtatttgatccagtttcatctcaactatgttatacctggacattttagatgtttatcaaaggtcttttatggggaagaaagtaaatgtatgaaataataaatgtgtgacatttatagggaatgttggctctgaacaaacttttgaaaacttggttgacaaaattttggatcaactgaaaaaagcatgacttttgaaatctctgaatgccttggttctcagtattatcattctttattgaatttatttcttattaaaatatgtagtttttaagactttttttctgacagtattatgtaattttttagcgtgggtagatgggagtgtcgcttgtatgttatcgtacagctgacatgtatttttgtctattctttattatcttagtttcatgctatgtatgtaccataaccaacctattgcctatgagaaacatgtaagataatgtatttacagccattgttacaagtttataatgtatttttctatcttgttttatatgtatgttatataacattcaaaaagaatttttttcttgattgagaaaaggatacaaaatgcaaaatccacaattttgataactgaaaattgccaattgttttgcagtactttattatattgggtgtcttgtcttttttgggcttttagttagctaatgaatgaatacattagacacttttgggttttagttgggattttacatagcttgcattttaattctttggttctttgctgtttctattaacccacagcattattttaataaatgttataataccaacctactaactctggactttgtctgtttattaacctttatatgtttaattaaaataaacaaataaagacaaaagaatgtaaagtcttgagttactggactaaccctgaagaacgtgaccacagataacatagtgtgacttgtttttatgttgtttgtcagctgacagttttgcacactactgttaaaatggctttggttattctggtcttttagcttgggggccggggcgggtgtgggtgtaggtgcagtagagatgggaacaaagtagcctttgtttgaggcattcttctcttgccacttgcctttcagcatctgcattactaattccacaccttatttttccttctccaaaataatcactgtctgctcacagcatcgagtaaaaagatacattgtcatggtatatacctggagaaaccagtttgattctttgttgaaccatttcttttaatttctgtaaataagtaattttatggtcacattgtaaagcagagaaactgttgatattttacctttatttagatttttgttttaacttgacctttttcctttggaaagcgttgaacttgtttagctaactactcctattaggggagcttaacttgggtaaagtcaaaggcccactggctgtttctcatggtggcctgaagccccgctgctttccagtgaggaaaagaaccctcagcacgttactgagctttcctcaggtatttttttctctgtcacctactttacaaatacagtcagactaaaacattaaacaagaagtttaagggggaataacattgggatttgaaaagttgtatgtgattaatttcttgcatgcaaatcaatttaagatttcttaaatatcttcatgcttttcttggctggaacttgggtaaattaacctgtaattatgaattatttagtttaactcaaggtgcatttttatttcacagattgatacttgcttttttctgtgaaacagttctcctagataccaaaatcctcaggatgaatttttgcatttgtaaataacaatttatctttgtaaattacattttatttatttagagttctaaaattgagggctgaaatgaatgacatgtaatagaagacaaaactgtaccaatacgctgttaaccaatcagactgtccaggtattctcatttcataaatatgtatacacacatataaatacacagagtagtgttagctgatgtgagtgataacagcgaatcaccattgagatggtcatttggatcatgtaaacaaaccgtgtcatgtctctgatgggagcggtgggtagtgtactgttaggtggacaggtcttttaacatgctactatttgtaaaagtttccgttgccataacagttcatgtagtagtctgtgaaaattcgaagctgtctgtcaagctgctaatatacaagttgtcatggtaccatgtaacttaaattgatgaatttctatgcaatttactattctctgaagcctatgaatgcagcctcatctcattttcagttaagggtactttatgtataattatgtcattgctgccttttatgtaggaaatgtaaaaaagttttaaaaggcgaaaataacattacagatttcatttttatacaggttacaatattaaacatgaaactaccaaattccaagaatagtaaaattctagagtcagttgattgattgaaactatttgacaaaacagttgaaagctgagctttcagtcaagaagttggtgaaattaatgtccttaaacttttcctttaggcaaaatgtcagggaaaacttaacatgaaagtagttactcccatacgcaccagtgcagtagctccaggtgtaagaggtcacgaaggcaccctgcccataaaaccaggatgtgcatacgtacacacaatcggtgtctggttatggttttctaaacactacataaggatctgaattttttagtgtgccaaaagcaaacgataggtttaaatgaaattgctcactaccaaatcaagactcttcatatatatacgtgtgtgtatatatatatgtcataacactttcacgtgtgacagcctaaggtttctttagttttaggatggggtggggtgggagtggttttgcatgtttgaccaggatgtgatgcctgtctttattccttgataagaagtagctgttacagcatttcaagcaaattattttaggtagaatagaacttactgccaaatgattatttatttagcaaaaattattctccaacttttcgaattcacaatttttctaagtgcatagagtagttttttacattgtcacgaattccttaatacctttatttaaaatggaaaaaatatggacaatattttagaaatatgtgctacacatctaattttatctgtagtttaagtgatctaaattgagatgcctttgatatgaagagatttaccaacattatatgcactcgtgccttcaatgtggaatcaaactggttaacctcagcacagtcctctcgtttctgtgtttctgctgcactaattgccagggggtgggggagaaggcgaatgaattaattcatagtagaaggaggcgataggtgcagcaaagggcagccacagtgttgattccacattataatgttgttgcctcttcttggcaaaagacaccacattgtgggaagatctcagcttccagggtaaaagttaatttataacttaaaagtgctattaagtttttattaccaaatatatcttttatggtttatattgtagtggtatgtatgaaacatttaaaattttactgtggaaattgtgtatatatatatatatagtcgaaataggtgttcacaggtcacatgtgaacggagaactgcatgaccgtacatgaaatgcaataaaccaactggaaaaagtgcatgtgcttcatcctctcaagccaactgcagctggaaagtgctgcttatcctccacccccagaaaatgcatgtatcaatatgagaataaagaacgcacactttcaattttattgaggctttcaacactatttaaaagaaaatgtaagaatttgacattctggagttattataacattagaaaatgagcataacattcactctgattttagccattaagggagattagtaaacagactgctacagtgttccatagttggactgtgcatccaaaacatttttttatctttaataaatggtacagtttttatgtagttttcgaatgtaagaagaaaggaatgctgaccaaaacttgatttcatcagcttcatgaaaaggactagtgtcattaacctgttgaacagaattggtttattaaaaaaatcatttccagtagtgtgaaacctttacgagtctttaacatctaaatgttatgactccttgtaccttaagttttccagtctttcttatttatatcatctccaagtacctctggctcctttcctcttgctcaccggaaccttagttttcctcaacagaatgctttgttaaagtagcccacagttgcaggatccatagcaccgtcgtgcagactagcagcccaaaggtgtgtttggtttggcttatacggtgttttgctttttaaactacttgccataatttaaaagtggcaacactagacttaaaaaaaaaaaagtctgattgcccatattagattttttttttaattcttcacaaaatctgctcttcctgaaagatcaaagtgtctagaaagcccaaacatgtattcttaacatagtaggcaccagctgaaactgagtaattaaacggtcccctgaagccaagtattccctggttagtcgccaccccaccactccttcctgtctctagtgtcacacttgggctgttgattttcttactcttctcttgctttaatccttccccccggctctctggctcctgtggatatctgtgcttgtttcctggtccaggatggtgatctgactttcaaaccagcttctcaaaaggggtgacataaaatcagttttgatgtttttcctcctgaaaaatcagatgaatattttagtcactgttactcatgcacattgtgttcttatgtttacagaagtgcttaagtgaatggaagcactagacatttggatttccttcctaacgtaatttttaatgattaccccttttatacagtaatttgtggtctttagaaagcagttaaactaattgaccatctaatagttgtactatacatatgtctaaaataatagtcatggtaagtttggcatcatatcttccccaaaaaatgtttattaaaattagattattccagtttaatgctattttgtgaactgtataccctctgaaagcgcttatttttacatgctacacaacagttccaattttaaggagtgtctcctaaaattgggatgaaaatctactgtagtctgttttaaagtatgctatactatgttcattggttacttaactggatattaatataaaggttattacaagaaaaatgatgaagagcaaaaggagaagaaaatatttgcaagtgaatccacaattcttgcagaactatttgagttgatactaaagattttatgttcactcctttaccttagaactgtcaagcttgggaatgaggaaagtgccttggctgtgcttgaaacctgaattttagtgcttcccttattacattcattgttttcaatgattgatttataaaattaagacatactggtagtacaagttgaaagttggtttgaatacattttaattaaatgataatatgttaatatgcttttgttcattgctttctcactgaggtaaaacagcattaaaaagttgtccagaatttaaactga

SEQ ID NO: 2 - DNA, 37859 bp, Homo sapiens
aatatcaacctgtttcctcctcctccttctcctcctcctccgtgacctcctcctcctctttctcctgagaaacttcgccccagcggtgcggagcgccgctgcgcagccggggagggacgcaggcaggcggcgggcagcgggaggcggcagcccggtgcggtccccgcggctctcggcggagccccgcgcccgccgcgccatggcccgaagaccccggcacaggtaacggggagccgggcgggcggccgagggcgggggcgcgcgggggcgcgcggggcgccaggctcccgggagcaggtgggaattcgttccgggatcatctgaggggctgtcagaccctccgaggacctggagcccctgcctcggcagcagaagccgctccagagactgatgaatggaagagtctttgcgggaaatgtatccggacgttgggaagcacgccctgcggctcattttgcaagttgcatggggatacatttttctttagggggaaaaagaaagcacgttccagccgagggcagagcggtgcccaccagccccggggagcccaccaggtgcctggcttgatgccgcgggtgtcggcgagggatccgcagcgtaattgctggatgcattgagatatgtttggacttggaagtgcagagcatatggcagatacagggaaaatgctggattgtttagtagctgcaggtagttcatttaaatttcattcattctttatggaggcgaggaccagtgtcagctgacaggcggctgaaaagcagattttagggggataaaggggtctctacttttgcaaattgtcttaagacctggttttggaggttcgaatcatttttctctgaaaggcttttacttccactgcaaacattgcacgcgcacccacacgcgccgccgtcgcacatgtcactgcacttagggaggctcgccgggaagatgggtgcagtcccttcacatttttggccggacagatgggaagagggagaggaggaggaggagggaaatcctcgtccgaactgtcagttgctcgttccccagtctccaccggcctctggactttcccagcagcacacgccggggctccctgcgagcggcgggtgccagggctagctcggcagccgtcgcagtcgccactcggctcaaggggacagaggccggcagcacccaaggccgctgccgcgcaaccgggacgcgatccccgcgggctctgcccccaggcgaaaggtccctgcgcggcgcgcacacgtggatgcggctgaggtcgccgcgccttctcgcaccctccttcagccgccttaggtcgccccggccgaggcgcggtgaccggacagaccctcgcgaaggagttctaaggcgaagtccatggaggcaaagcaaatccattcgcctttccttagattgtttgattttctcctactggtttgttgttgagcctgcgggcgctggtccccgcgcggcgctgggaagttgcaaagagcgtgggtggagaccctgcagcagcacccgcgggctgggctgggcggggagcagcgccggggcttggttgggctctggggacgagagggcgacttgggggagctccgggctccctatgcctactccggagcctggccgcgcaggacggtgtgctgcccctcgagggctccacttttcgcctttaggacttcacaggacagctaccggggtcatctgttttttttttttttttttttttcgagagctgaaactggcagcaggaggagggagtcgggcaggggtgctgggaggaggcgcggcagcggctccgcgccccgaggtctcggccgcccccgcggggcggggccgcgcaggttccctgaggctccaggtagcccagaggggaggcggctgggaggggcgggagccttgaacctgcctcaggggaagagaaacccgactgcgggctcggagccgtttcttgcaagggcgagcgtcaactcggcagggattatattaaccaggtcagcgaaatgagtttgaacccctccgaatcacagtagcggccacaaggcacttcctcccgctcccctccctcttcctaacccccagcacctcttttctttttcgtttctttgcacggtttgtttccttgacagaatgtaacattcatagttgtactcttgtaaccacaccgggtctagcaggtggggagtcgagtgcatcaaccctgaaatcagcctttgaaaatcctctgactgttgggtctgagatcttgtcagagcagataaaggaaaagttatttcaggttccttagtagaaattaatcatgcttctaggacgacttagagcaagcaaatcgcatgcaaagttcaaaggcgagcctgggtggccagcctggcagggaaactcaggagctgggcgcgtgagcagccccggactcgccctgctgcgtcctggccccttcaggtcctgcagaagtcactctccaccttgcccccaagaccttagttttgcacatctttgcctctgtgcttagcttttaagaggcacggaaggacatggcatggtcatgtattagcgctggaaagtaccttagaaataggatccaagcaagcccttattgtaaaggcgaggaactgaagtcttgagagagaggaagtgatttgtccaagtcatacagctaggtggaggctagactagaacctttgcctccagaaccctagtcctgttgattcccgtgctctcacctgacgttggctgccccctgcgggcggctgaataggttgcttgttcctgggttgttaaaccgctgcggagagagaggtgctcaatgactactctgaaaaatgtgctcagttctgaaaatgtgacctctagatccagccaaacctgaaaagttaattcgacttgaatctatgactgcagtttcagattcctctggttttcttggtgtctctcacatttcacaagggactgcatttggggtctttccaaggagttccctatgaatctttcaaaagatgcagacttacacctctgggctttatagctttttttttttttttttttttttgctttatcatatcgtgttttacatttttgtagttcttgttcagaagttgtttagtgcctcctgttgacagtggaaacagaactaggctattgtttcaaaacacagacactaaatgaaaggaattattacagattataaatatatatttcctgggattttatttggtggtggtggtattttaaatttggattacatcatgtcgccattcaacctggactatcagattgttgcttaaaataatagctttaaattatgagaacccccaattcaggatttttggtaagcacttcagcttcacttgtagggggctggtaatatgcataccagtgagttagtttgggaagacaaaacctgtaatgaatataacttttctttttaaaaacaatgatttacattatgatatggctttatatcattgtttttattggtcttagtttaatgggtttcccaacattgcaatgacaaattttagtgaagtttctaagatagtatttttcctattttaagttttataagaaaaactataaaataaaccgtgtctgtaaaattattgtcatctgatcagttgattagattaacaaacattatgattatgattaacaaacagtaagcatatagttacttcttcaaaagtcataaaatttcctaggttaggttaactaaataaaagaaaaagctttcgaaaatagtaatgggttcttttaaatccatggcttttttctggggaaatcccttctggattaggttacattttataatatttcaaacaccattccatcagagacagaaaaaacaagagtaagtaagactctgactaacaagtggcctaattattcacttagttactctagaaactaagtattgtaaacatgggcacaagttggatcaaccaggcctggagttgtgagcaatttggtattaattttatttacaaaacattaaagcttgatcactcaatgttcttatctttgctttggttttaaaatcctttcctcttagattctcctaatcctctagactttatgggatcactataattctgttttgcgctgtactacttcttgatttttttcttcttttaataaaacaaaaaccccattggaatagcatagttgaattgtttattatgtttgagaaatattatttaaacgatgtgacagatgccaaagattttgagtgtgcacttatataaaggacatgggttcttgttccttttcttatccttaaccttaagttttcaacttaaaccttcactggttggaaggtggccaaatgtgtaacttgtccctggtctaatagtaacagcaggttcagacatgcaggggaataggaaggtgccaggtccttggccgtgtctgtggatacccataacagcagaaccagtttacaatactagagcaacagaatgcagcaaacaatcttgttgtgcaagttttcaaagttttgtcttcataacctttgaaaagattgttgaggagttttgtgtaagttttgtaatccagtagtagtctaaatcctcttgtttcagcccacgtctacccattcttatttctgcagcatatatagcagtgacgaggatgatgaggactttgagatgtgtgaccatgactatgatgggctgcttcccaagtctggaaagcgtcacttggggaaaacaaggtggacccgggaagaggtaactaatcattttatcaagacgcagaaaatagatagagtgtataatttataaaaaacaaaatttcaactaaactacaggtgctcaagctcattagcaggctccttgagatcttatcctagcccgcatgtgcagcgtgccctatgccccccacttcattatattatccatttccacactatttgcagttctttgaatgtcctcttctcattttaggcttcatctgtgtttccttttgcctgttgagttctcatccttttgtcccacctcttcttcctacttttacattacccatgaaggcatcacctcttctgtgaagccatccctggcttcgccggacaggattggatgcatcctcctgtgatcccatgcatgggtgcatgttctactttattgttgctgggacatatatacctcctccagctgactgcaagtctgttgagggcagggctcttgtctattcatctttgtgtttcctgaattcacaaatgcttggacccaaaataggatattaataaatgttctctcaataagtgaatgacttgctactcccaaaaacaaaacaaacaaaagaataaaacaaaacactcagagaactcatctttttacagtgtattgatttaatatccttgatatacttaacagagccatctatatgaaggtatgttttaaaatcatccgataatggatggagacacatctgcttctcctgtttctttttcggctggcttatcaaaagtttgattttgtaggaatggaaaaatattttagaagcaaaattattaatatgtggagaaaagtttctctaatgttcttaaaatttttgcaactaattttagatgattcatgttacaataaatggtattggctcttcttcagtttcactgttctctgcttcatttaaaccagagctccttttaacaggcaggtaggattcccagagaggcccataggaaattcatattttacctattaagattatccacttggataatccttatgatacggaaattctgttgtcaacattggtttcctaattcttgtgtctgtattgtaccagggacctccactgtgtgtgaatgttacctttgcagaaacataagaaggaatgcatttatatacttatgaactggtaggattgtgaacagcatataaactaaaagaactgattagctggaaatgtttaatgtcttttatgttctaacacatctggcctatctaaatctaactgattttcttatatagctttcaaatttaattttaaaaatggtctttatttctttataaaaagttaaactaactggaaatattctatttcttgctcaagatcctatttattaccataggtaaatgtgtactgaatctagcactggaatgcattttttcctaacagagtaaagcagttatttataataaacactcaatatatgccaggcattatgataaatgttttgcaaaattgcctcatgtaatccccaaataaccctgttaatatcagcctcatgttaataaattgccccatgtaatccccaaataaccctgttaatttccattttatagatggcaatactaaggcccagaaaggtaaaatggtatacccgctttacacaattaatagttgagtcaagatgccaactctggtctgattctaaataaatctatactcccaagtccgtatattacattaatatctcctgtattcggatactttagagagactttttattctagattcgttccctcattcactttaacttgaacagagttatatagtaaataactgatatcctaggattacctttaaatgtcttaggatgaaaaactgaagaagctggtggaacagaatggaacagatgactggaaagttattgccaattatctcccggtaagttagtaagtttttcttataacgaaaggaaagcttctcccaaagatttctataaaatattctaggaggagttattaagatagtgtataatttactcacatttaagaagtgggagaaatgccctacccataacttatattttatattataatgcatataattaaattattagaatttagattattagaatcgattattataatttagattatcaaattttaaaaacagttgcttcttgttttatcttttcatttataggataattagtattttgatgatttgttacatggtagttgattaaacttgatatttcctccaagtcagtttttcctgaattccctatttttgtcaatagcaatacttttggcaccaaggtttaaaattctgaagacctttcttgacttatcacattcccccaacccctacatctaagcagttactaaggaccctggaattcctttatagtcagtcccgtccttttcattctcatggctgtgctgaaggttgggctcctgccctctcatttggactatcatataacttcctaactggcttccccacttgtagttttccatttttctttcttttttttttttttgttttttgttttttgttttttgagatggaatctagctttgttgcccaggctggagtgcagtggcacgatctcagctcactgcaacctccgcctcccaggttcaagcgattctcgtgcctcagcccccttagtagatgggatcacaggtgcccgccaccatgcctggctaatttttgtatttttagtagagacaaggtttcaccatattgatcaggctggtcccaaactcctgacctcaagtgatctgcccacctcagcctcccgaagtgctaggattgcaggcatgagccaccgtgcccagccagtcttctacttttccattcatcccaaacaccattggcaggtggaacttcctaaaatgcagctgtaccttatcttttcaaaaccaaccacagtgacagaatctcacaaattaagtaaactgccttgacttggcacacagaggccctcctctattgccccaacttacagctccagttctgtttcctgcttcctctctctaagcaggtttgcttgaatttatttctgctttacctcctctgggcttttgctcctggtcttcccacctcctagaatattctccctttatgtcaccaattaaagttctgtccatccttcaaagtccttatgaagagctcttcaagactcctcccaaggaaaccatttctcctcctaagaagatgcttctctcctcacttcagtatcattttgctttggtcttttcttccaagtgaatcaaattcagcatctgtgatagtcagtcatctacttgtcttttccttcttataagtttacaaactcctgtaacataggacctatcttttatgcatcttttttctttctgcaatataaatactttggtcctggattgaagcacatctcttgtctgaatgggaagcaaactttagtcttttagggtggtttccatgaaagcctgacaacatagaatctctatcaagcctcaggctatgtagatactctgcaaaagtatctgaggttaaggttaacttggcaggcacatgtgtagtatgatggtcctttagatcatgtggcaggaatgtttccctcaggaggcagagtgtctaattttagatgcaactttgatatattacttgcatgttctgtagcctgtaagtaatgagggatagatgggctctaaacatgctctaaaatgttcttaaatattttcactgcatatggtctatgtgtgattcctattacaacaaaaaattcacgtgcttatcacatcatatctttatggtggtgcatactttatttgtctagaatcgaacagatgtgcagtgccagcaccgatggcagaaagtactaaaccctgagctcatcaagggtccttggaccaaagaagaagatcagagagtaagttctttcttcattggtgtgtgactcataattaagaatattcccaaaatgctaatattttcctatttgaggaagcaggtaaaatgggcaaacaggaagtagaggactctattcccatatttccagtgaatgaaagcaaattttggaaattttctaaagatcttgtaacactgaagaatgattatactgactcattacataactttaaaacataggttatttttgtgtgtttatctgaaggtgatagagcttgtacagaaatacggtccgaaacgttggtctgttattgccaagcacttaaaggggagaattggaaaacaatgtagggagaggtggcataaccacttgaatccagaagttaagaaaacctcctggacagaagaggaagacagaattatttaccaggcacacaagagactggggaacagatgggcagaaatcgcaaagctactgcctggacggtaataatatgtcaaaaaatatattgatcgggaagaatgagggagtgggtattgaacattctgctttaaatgtatggtgagtaaattatatttcatcagtcgtaagtgttttattagataaaccagctacatagcttttagagattgaagacatcttagagtttatttggttgagaacttccaggatccacctgacagtgagccctgggtgaagacatttagctttccccagcctcaatttccccatttccaaaatgaatcatacagggccagtgttaggatcaagtaaaatgaatgtcaaaggacttggtaaactataaaactctaaatgcatagaaagaaactgaagacagtttgtctctggcagttcacgtagctggttaagttactcaaaggaactcagctaccattttcctgataaccatcattcatttcttttattttttttaattattattttttagagacagaggcttgttctgtcacacatgagtgacagagtgcagtggcatgatcatagctcaatacatccttgaactcctgggctcaagggatcctcccacctcagcttcttgagtagctaggactacaggtgcaccccactacacccagctgattttaaaatattttttgtagagatggaatcctgctatgttacccaggctggtatcaaactcctggcctcaaacaatcctttcacctcccaaagtgctgggattataggcatgggccaccatacctggccaccagtattcatttcattataccactttgcttaattttttacttcattgattaaagaaataatgtatttcttgaattctcatactacttccaggtttttaaataatgtcagggtaataatgctttctttttaatgaacaaggcaagaaaatgacctcattatgagggataactatggaagctcattttgtcccatttttcccacttaagtagtatataacagattttgttcctttggaaagcaatacttttaatgataaatcttgcattaaaatgatgtagatcaaaaatattaaaaatagttttattcttgagcttcataatccaggagaaagtttatatagtattctctagtgatgctccaaagcatttgcacccttgtggttcagtataaacttagaaattttaagaggttggtaaaaaaaggcctttcctcccatatggcctgagaggatacactgctgattcttctcaaaattaacagggctattgaacttcctctttctttagcatacattcccccacccccattgaaatagaaaaagggagacttaaacggtttttttgaattgagaaactgttttcctgagaatggaaagactttagtaaaaatgcgttgccttttgaaaatatactgggggttaggatctttccctttgggatcccattttccactctcgtctctgtctgattcagaaaacaattggggtggctttcaaagagtaggaggttttttggtctcagaagttctcactagtgctttgagagataaagagtgaagtattcttcatcagcagcatcttatcatccaccagacttcaccatgtccctctcgtggtgtgtaggccctgctgtgccacattcaaaggagtaatttccccaaggcagctggaactttacattgtgttggagtcttgtaccgcgaccggtcagaataaacatgaagcgttatcgtattgaaaatactcctttaccaaggaaatggaatggttaaaagatactgatatttctagaaaacgacatgcattccagattgtgtaactaagagaaatctaactaggcaatagagaaagcctgatctcagagggtggagaagaaggaaacaattaaaaagagaaaacaactaataggcccaagggaaaggtctctgttagaaacagttttctgtcaattcactttaatctgaattgaaatttttgtttgtgaaatttgtgtgatatatttctgtgcagaactgataatgctatcaagaaccactggaattctacaatgcgtcggaaggtcgaacaggaaggttatctgcaggagtcttcaaaagccagccagccagcagtggccacaagcttccagaagaacagtcatttgatgggttttgctcaggctccgcctacagctcaactccctgccactggccagcccactgttaacaacgactattcctattaccacatttctgaagcacaaaatgtaagccattcctgtgaatctagtttaatgagagagacggttagtttagctccaggtaataatcatactttgcagttgtatacttttcagagaactttcctgagcatgatcatggccagtcttcacctatgccctttgaagtagatagggtgtaggtatggtgtgttacccctggtttatgtctccagaaacgaggatggtaagcgctttgcccagggtgacacagctcatagagatagtgctgactagaaaacgagctgtccgattctaaattgctgcttcaaatctaatttgaaggcaacctttgaatgtaaatttacttaaggtactattgaagaataggggaatggagtcgcatttgtagatgtaatgtttttctcttccaaaacgaaaagggaaggtagtattatgtttaaagaggagagagtttgacactgaaattcagtggttatggtatgtttcattctggcagtgatcaggcagacatgtcttaatttattcactcagtaagtgtttaccttcttactctgagtcaggagcagaactttgtgacattcattttctgatgtctccttttagtctccgttggatcagtgtgatcccttccttatttctagatatttatatttagggaaagtgataatccataataggtggaaaaagaagaagaaagaggaaaaaaatcgagaaactttaatcttaagaataacaatattaaatgataaaacttacccaagaatagagagctcatatgcaaattatatgcaaaacatgttcataaatagtttttattcttctctgtccatggatttgactacaaactttacagaaaggtctcagatgaataaattatcataagaattcctgtgctttttattaaaactcaaaattattccaagaaaaatatttttatttacaaacagctggtgccatcaatttttcccatagaaacaattttgaaacattcattaatgtattttataatatattttttattgtttttttcattgtgtttttctctaattaaggaacacttcccaaaatttaaagtgctcatcaacacctcccaaggatttttcaccatgttcctttagctgtttagccattttaaaggcctggctaaatttcacattaaaaggttttaaaaatttgtttagtgtattttatcagtaaagtgttttttttctctcagtcccagaacgaaacctcaggaagccaagtcccctgaagcatatgtagccctgaatgacgtggcctttgttttgtcttttgcatctaggtctccagtcatgttccataccctgtagcgttacatgtaaatatagtcaatgtccctcagccagctgccgcagccattcaggtaagatcattgatcattcactgttcaaagcaccacttttaagaaatgcttatttctttcatctaaacacatatttttacctgagcaactaaagtaaaaaggaaagggaacatggagaggagagcagagtcttggccagcaatctattaatcttgtcttctagaatgagtttgctctggaattttctgattctttgatgttgcaaggtgaaataactcatcagtcagcatccactctagcctgagatttcacatcactcccttaagtctgaaataacattcacaaagatttctgcaggagccaaatttggttggtggtgttgtcacagtgaggctctttgggctgtgcggcacctcatgaccatattggctacacccattgtatttgcagcttccaatcagacctttgtctttatttcccttactttgtaatttcagagacactataatgatgaagaccctgagaaggaaaagcgaataaaggaattagaattgctcctaatgtcaaccgagaatgagctaaaaggacagcaggtgctaccagtaagactgtcatcatgtgcttgaatgaggggatagcagctttgcctcagtttacctaagcgctcttctcttctaaatattacacttagcaaggctccatatatccattcagaatgtctcaacacaagaagttgcttgtagtaaaatgtagttggtatcagattatatgctgattaaattggaagcagtctttttgtaattgcaataaaaatgcaatatccacttatgattgttatcatcaatttttttcttcagaaggactataatcagtttaaagactagcaaagataaaatgaatgaaaatctgcattttctacagagctatgtatctatatggtaccattttggtttttttctattattttgttaacctatattttcatattttataatttcagacatgatttgatgcatgttcagtatatacatgtaaaaggttatatataaaacctcttcataaataacattagctgctctcttttatttgcatgtgtgaaagttatgtgggccattactgaattctgacatctttagcgactgggcagccaactgggatggctccttgtgctttgcaacatacatagttaaccaacaaagacaataaaagagaagtgatgcccagtaggtattcctaggggtaaatttattttttgttaaataataagatgtttatgtgcacatgctagttcgaccactttttaactttagttaatgttttgcaatttctcctgttgtgtggtcatactgtaaggagacttctgttaatttgctcttctgctttcctaaccacgttgatttggcagtgcttcactgcatcagtgcaatctgtagcattagaaagggatagaagcaacaagaatatgtgtagatcaaagcaactctaactcctgagtcctctagcttttttgtggttagaatgcaaataggacagttcttcagttcttcactcacagcaagcatttatggacctttggcaaattatattaaagtggaattttaagacaatatgataattatacttcgaaatatagacctatgatttgacatttttacaaaatgataacaaggtaatcatataggtacaacctctagcactattaaaaacagggtcttgcattgataaaaggaaacatcttgacaactgtttcataggcgtaagtttgggatgatgcaactactcttatctttcctccaacagcatctgataccttgtgcaacttcattgctaagttccttctccctttcttctgtcctctctttatttctacacccttcccccttccttagacacagaaccacacatgcagctaccccgggtggcacagcaccaccattgccgaccacaccagacctcatggagacagtgcacctgtttcctgtttgggagaacaccactccactccatctctgccagcggatcctggctccctacctgaagaaagcgcctcgccagcaaggtgcatgatcgtccaccagggcaccattctggataatgttaagaacctcttagaatttgcagaaacactccaatttatagattctgtaagtagaattgtgaagggaagttaacgattctggaagataaattagaaaacccatttcttaaatcattgccattttctatagccaagctgttattagcatatattaatgtaaaggtagaagtatgattttcatcttcatgaatatgttttttcaaagatctgatttcatagcctaccgagagagacaaaagtatttataaatacttacaaaataggacttttgaaggaagagttttttttcacattttcacacttttccttcaaaagtcatatttttaaaaatagtcagattgatgacttataaagcagagcttggattatgggaacggaatggtgtttgagttacttttaacttttttttttctttgcttacttcatggtgcttctctagaattttgtcttgcttttgaacagcacattaatgatttggcttactcagagctgaaagcaatcttttagtgtatttgaagtaataatcagctccttggtcatacctggctctttccacaaaatggtgattattgattactggtcccattgcttcactgagatgaatcatctttcgtgaaacaaatttgtattcagcacctcctcagcgaacacttggttcctttcccagttgattaaaaatcatttattgcacactcattggttaggagatgaccattgccgtaatatgccatctagaaaaggtcttcattttgctttccattgcatgcagatgtagagtgtcgggaggtccctgcagcacatcagagggccagccttgaggcagcccactagaccctgctctactgcagtataatagagcaactgctcatcttgaagctgttgctcaagaagccaaaccaacgggcctagtgtttgctttgcgttgagcatctctctctcagtgctgtttcaggtgatgatggcacacactatctcaaagttctgtgcctcccacattgtttcaggattcttcatcatggtgtgatctcagcagttttgaattctttgaagaagcagatttttcacctagccaacatcacacaggcaaagccctacagcttcagcaaagagagggcaatgggactaaacctgcaggagaacctagcccaagggtgaacaaacgtatgttgagtgagagttcacttgacccacccaaggtcttacctcctgcaaggcacagcacaattccactggtcatccttcgaaaaaaacggggccaggccagccccttagccactggagactgtagctccttcatatttgctgacgtcagcagttcaactcccaagcgttcccctgtcaaaagcctacccttctctccctcgcaggtagaacacaatttctgcacagctgttactcattttcaacagacacctgagccttaataatctagaaactaatacttcggaacaaatgactaattactgctgcctttgctgatttcattctgtcgttgaatctagctgttgctaagttatactgcctcccaaagtttaggctgtctgcaggctaactgttgattttggcatcattctctctcttttacttttgggtggttttgaaagatagttgcatgaaacgtacttttatttccttcaacactagagtttaacatttctgtccctttctttttctcccatctgatccctccctttaaaattgttaacatcatttcttcctcgttgttcatttattcttatgcgattcactgacccaggggaggagggaaccaaattgtgtttatataacatttgggttgcaactagaaaatttaaagctagaaaatttaaaggttccaattcatctttaaatggaagagtttgggaagaggatgagaaacaaatgcttaaggaaaagaacatatttttaatttcagtttctcaacattggctcttttgagttgtgccatactattttactagtgcagtttttgccaaggaatatttttatttcatacagcttttttaaggcatgaaaatcacaattcttatcagagatttgtgttcctaaattatttaaataattaaattaatagataatttggcactttaaaacatttttccaattgtatctgttgaatttgttttaagaacagatttaggtatgatataggtatatacatttgggaaacactgaatcaaacaaagttacaagtaggtttcttagctgggcatagtggcatgggcctgtagtcctagctactggggaagctaaggcaagaggattgcttaagcacaggagttcaagttcagcctggtgaacatagcgagatcccatttctttaaaaactttcttttttttaactgcaggccttctcagagttattaatatgccgttgtgccttgtgaatttccaggaggagattatgatatgttgtgtttcccaaatttattttaccttggaattgtttaaggaacacctagttttttcccagaactttagtgttctgagagacacagtttattattcaagaattttccatataatgtaaaccttctgtaatactaattttggttcatagattattaaagagaaaaaacaaagtatgaaaggaaactattaattattaactattattttccccaaaatcactaaagccttttaaatgttaataaagaacacatctcattctctttaatgacaagtgtcatttattgagagtattttctcacagttttgcaagtttttcagcaatgaccctaaattttatttcttggaaaccacacccaaatcctgggtgctcaattgaaataaaacaataccctagtcaatataacatgcttgttagcatctttattatttaatatagtgggtcaggaaaacattcttgttatctaggtgtgatttttaaattggggagaataaagaattaccatctaatctaagtattttttctttctctccatatttagttcttaaacacttccagtaaccatgaaaactcagacttggaaatgccttctttaacttccacccccctcattggtcacaaattgactgttacaacaccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataattttactgaccatttatatataatcatgtctattcccacattgaatatatctgtcctgggcatattacaactacctacaggtagaagatgggaagataaactagattccttttgtaacttactccaaaatttcctatatatgcacaagtagttttcataaaacttttccattcggtttgtttgttccctccataaggccttcttatgaaagccagattttcaacctcctttcctctctaattgtaagcttcagaataagaacgagtatggtttcttacctagtttgtatttttatttaattttatttgtttattatttatttatttttagtttgtattttttaatcttattttttctttttgaatttatgtgtagataggacctcttctgacatccccaggaatattatatgattagaagccaagggatgagtaagcagtcatttttattcaataaagccttaatcaattcagagaaatggtagagaaagtgtgaattatcagaaaccttgaaaagaaatgcactgttatttcgaagtacaaaaagtaaattttaaaagtttaacatgttttgtagtatgggctctgttttcatgaatagggaaacctgatttgtaaatgacatcaggtcttcttgatgctcatagtcttatacagatcataggcactacatgtgtcttcagagccattaaggaaagatgttaaatcatttaatgcttttagtcttcgaggtataatattttgaaatatttgaaaatatgtgatatgcatattctctttttcttaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccagctatcaaaaggtcaatcttagaaagctctccaagaactcctacaccattcaaacatgcacttgcagctcaagaaattaaatacggtcccctgaagatgctagtaagttctagaaaagtttttggatttcattggtttattagtcccattaggagttctctctgatgcaaaaatacccactcttccgtttagcctcagacaccctctcatctagtagaagatctgcaggatgtgatcaaacaggaatctgatgaatctggaattgttgctgagtttcaagaaaatggaccacccttactgaagaaaatcaaacaagaggtaaacacgcaaccctttggaagcaacaagctgagaatcctgccccctttattccttgtgtgcagcttgatgtgtctgccattggcactgtgcaacaccacgacttttcgtgcaagattttcatacaaggtgcagcgaaaaggtactgccagactgtaggcatcatcaacctttcctttgggaagccaagccatctctatctacttcatgtttggacagaaaacatactagagagtatcaagaaaagggagggaaagaggtttttaatgtacttttaaacattactgattatataaagttgatatttcatttttagatgtgaagaattggaatttaaattaaaataatagtatctttaattaattgtattaaaaaaagggaggctgggcgcggtggctcatacctgtaatcccagcactttgggaggctgaggtgggcagatcacaaggtcaggagttcgagatcatcctggctaacatggtgaaaccccgtatccactaaaaatacaaaaaaattagctgggtttggtggcgggcgcctgtagtcccagctactcaggagtctgaagcaggattatggtgtgaacccgggaggcagatcttgcagtgagccaagatcgtaccactgcactccagcctgggagacagcaagactccgtctcaaaaaaaataaaaattaaaattaaaaaagagaatctaacctgcaaggatcaaagttttagcttgttggcaacagctgttttccaccaggttagatacagtgcttcttgaatttattttcatgtaaagataagtatttttttttatagctaaaaatctgatttgattatttagtaatggagaattttaccagaatgtgattaaaaaccagtcttttgtcaccaaaagatcttttatgtcaccaaaataaaatccttgacggatacaacactactaggtgtatcattaaaatatattcttaatagaataaacacctagaatcagaacattggttgaaaacgtaggagctttacaggggcagtaaatttggagctagtcacatttcaaatttgccatcagtgtttctttgttgaatccagaagatagagcaaattgagctgatagtgtaagttagcaacatagttttataaaagtatttgagggactggccagaaatatactcctgactgtacatgtttcataggaagttctagaaacaacaaagcactttctatatgcttttaggtggaatctccaactgataaatcaggaaacttcttctgctcacaccactgggaaggggacagtctgaatacccaactgttcacgcagacctcgcctgtggcagatgcaccggtaagtacgtgtgcaccagcccccaagttgttatgtgacactggtgcctcatgaaatcctgtgtgtggcatagggctgctgcggtttcagcactccctctttaacacatcttatttctaaatgttctcctgcacatgatttttaaaaaatgattattaatgtagagtataaagttgagattttttcatgtattagaaacattgcagactttttcattatttgttttctcattatttgagtgccttggccatttaaaaaaatctacatggatataaaaactatgttcatttatatattttcctttatttgtcttctaaataatattgtcttcttgttaagttgtctcttgtttggtttgtgtagcatctcatatggtatcaaccacgttgtctatagtagatgcccagtattgtacccaattgctttgggttactaccaccatttttaaagtttataatttgtgaagggttgtccttatgtcttaaggaaaatcagagctaataacagctgctaagatttatttagtaggtactaagtggtaagtactgctctaggggctttcgatgaattgattcagttaattctcacaattatcctgtaaggttggtattatttttattaatttttattttttttgagacggagtttcgctcttgttgcccagactggagtgcaatggcacgatttcggctcactgcaacctctgcctcccgggttcaagcgattctcctgcctcagtctcccgagaagctgggattacaggcacccgccaccacgcccagctaattttgaatttttagtagagacggggtttctccatgttggtcaggctggtcttgaactcctgacctcaggtgatctacccgcctcggcctcccaaagtgctaggattacaggcgtgagccaccgcgcccggccaggtgggtattattatcatttctattatgtcaactggaaaacagaggcacaaagaggcttagtcatttgctcaagattgtatacctaataagggatggtgctgggttggaactcagaaggtcttgttctaaattccctacttttcaccattttttgccaataatttatagctctaaaatattgatagttctgtaccaatatatgaaataaagtgttctctccccagaacttagtaaatcttctattttgaaaggcatgtatttattaattcatttagcagatgttttttgagtgcctaacacatgcctagaactctgggatcccaaaggagctttgtgttgtggtgatgtttgccacaatgggattaaggttcagacatagttgatttctccttttagctcatagtggggagaatttctggcagatgactgcatttttttatgctaatgtgtatcatctcatagtaatctactctccacagtgttagaaaaatgtggctctcatattatccaacctcatttaaatttcaaattattctcttccctttagaatattcttacaagctccgttttaatggcaccagcatcagaagatgaagacaatgttctcaaagcatttacagtacctaaaaacaggtccctggcgagccccttgcaggtaattactattccaacattggtattttaaaattcattcactgaaaaactgtcatctttggtggtggtggtggtggtggtgatggtagtgctggtggtctgtttttcgttttcaacattttagacataggggagtaatgcttttatacaattcttgatgtctagaaaaatccaataattcctttttgctactcatatgagacatgctagaaatttttatcagtgcagattccccaaagcatgatgaaatgaaacatttcttactgtgactgagactaagatgtattacacgtgtgtcactatcttcttctgccctcaaatgttttataaaaatatatcaatatagtttactttatgttccaaattttttattttttatgctgtatccctaggcaaccaaagctcagagactgttccaattttaaatgaagtctgaaattagtcagacagaaaataattgcaatgtatttgactgtcagaataagaaagtcaactgtcaatgttgtgaggccgatctgaagttgaccatctgctgtcaggttttaatatcaaaagagatttcccttgtaaccctaaagtgggtgtgtgttgatgaaatataaccaattccaaattaatcctgagccaaatgtaaataagaatgcaaattttgcttccctaaattatttaaagcagtttctgacattaaatagcccaatacccagccacctgcagagccctctcctccatccccctgtgccatgcagagggaaacttcataaccctagaggaagtgaagagcctggattcagaagctgaaggtcatatttcttgctgtaagttaaatggcacgatcataggacagcagcctttgagtctgcccattaccttaaagggatttcccattcttgtgatagtttctccttccaaattgtgagaatctgaaaacttgcccggtaagtatttgtgatgcttagtttttcagggttttttgttgatgttgctttgttttttgaaacagggtcttacccggtggcccaggctggagtgtagtggggtgatctcggctcactgcaacctccgcctcccagattcaagtgattcttatgcctcagcctcccaattagctggaattacaggtgtgagccaccggcaacccagccgtttttttgtatttttggatagagacaaggttttgccatgttggccaggcaggtctcgaattcctggtttcaagtgatctgcccacctcggcctcctacagtgctgggattacaggtgtgagctaccgcacctagccagtgcgtagtattattgtaaaataaggattgcaagcacattttgtttaattaaatcagattgcttccttcctggcctctctctgacagcaattgttttggtaaaatggctgtggactggaatgtgagacagggagtcagtatttatggggaatctggacaagttcctgcgtcagtgaggctggagttggccccgtgtggagaagactcagaataccacaaagaggccagacgcagtggctcatgcctgtaatcccagcactttgggaggccaaggcgggtggataacttgaggtcaggagtttgagaccagcctggccaacacggtgaaaccccatctctactaatcccagctactcgggaggctgagacaggaggatcgcttgaacccaggaggcagaggctgcagtgagccgagatcatgacactgcactccagcctgggtgacagaatgagactccatctcaaaaaaaagaatatcacaaataattgggagatgaggaagagaatgaactccacccagacataagaggaacccagaaagggagttacaaagatttcaaggcaaagggcagccatttcaaaattttcctgaaagctgttcttaggtccagaagaaaagcttttgcttttttttttttttttaattattcccacctaagttacttggctcttggtttctagagaacagaattcaactactgctattcgtgttttaattaagttagattaagttttgcatatattttctaacctagtctggggccaacttgtatttgtagtagtacagaaaaagtctgttctaaatttcaaaaagccaacacatcaatatacttggggacaattaccattacataagttgaaacccatagttcagtattgagctttatcagtatatttgtattttatttaatggaaatattggttatctttgaaactcaaaattaagttatagctctattttattaatctgtattctcctgtctatcagtgagtcatattggcactttatgaattctaacatctgtacactgagattaagttgtgtatggtgaaggtaatatttgtcaattctatcaaactgaataattgagacttgaaagttaattttagagtcaatactcctgtctaaaccatatagtttactggtttacactgaccagatatattttaataatctgctataaagccaggctcagtggctcatgcctgtaatcacaatactttgggaagccaagactggaggatagcttgagaccaggaatttgagaccagcctgggcaacatagtgagactcagtctctattaaaaaataattagctgggctgggcgtggtggctcacgcctgtaatcccagcacttttggaggccgaggtgggcggatcacgaggtcaggagatcgagaccatcctggctaacacggtgaaaccccgtctctactaaaaatacaaaaaaattagccgggcgtagtggcgggcacctgtagtcccagctacttgggaggctgaggcaggagaatggcatgaacccaggaggcggagcttgcagtgagctgagatagcgccactacactccggcctgggtgactgagcgagactccatctcaaaaaaaaaaaaaaaaaaaaaataataataataataataataattagctgggtgtagtggtacatacctgtaatcctagctacttgggaggctagggcaggaggatcccttaagcccaggagttcaaggttacagtgagctatgattatacccctgcactctagcctgggagacaagaccaagaccttgtctctaaaaaaaattttttaaggccagccgtggtggctcacacctgtaatcccagcactttgggaggcagaggcgggtggatcacaaggtcaggagtttgagaccaacctggccaacatggtgaaactctgtctttactaaaaatacaaaaaatagccaggcgtggtggtgcgcacatgtaatcccagctacttgggaggctgcggcaggagaattgcttgaacccaggaggtgaaagttgcagtgagcccagatcgtgccattgaactccagcctaggcaatagagtgagactctatctcaaacaaacaaacaaaaatttaatctgctataatagatactaatttattttaagaaatacttagtgaatgttgttctacatacttgccaattgggaggaaaaaagaaatattcactaaatgaatgaatgttatacaccaagtatctccttcattttatgtcatggagttctagcttctgatatcacagaaatatgtgatttctaaaatgtttcagactattgtgataagtattggaaaatgcatcaagtgactctaaatatttaaacatacatcacctatatttcatataatcctgattcaataatatttctatagattttttatgcaatttggtaattttgtttgtttgaaacacagtgctatttcatatatttctattgataatccaatttgacctaattaggcatatttctcacacatggtattattgaacatattgcacatttattaaatgatatgtattttacttaagagtactttatataatctataatttttgtaatttgtacttataataaatcaaatattaagtgggattttagccaaaccatctagacatttttgtcataatgtttttattaaaacagcaaaataggagtttcaaaatattttaaaatttgaatacttctaaaatgtaaaatgtttgcaactggtgtaatgcagttgattgtctatccctctaaactgtctaaataacttaggttcaagaacagtttattgaagaacataggaactgcgcaattctgtgttttatcatttcagttttaatcaccccggcttgattatggcaacaacattttagacaagcaattattactatttttgaggcccatatactgggccattatggttttgcttctaacgtacatataaatgcacatgcaatatatataaattatgtttgctgttttaaaattagaaatgccgagaagctatgtgtttgacattttattataaattttgtacacacatagatgtatggaaaatatactatgttctagagtcaacaattgttatcattttgccatatttacctcatctctttttttttttttttgagatgaaatctcattctgttgcccaggctggggtgcagtgacgcaatctcagctcactgtaacctctgcctcccaggttcaagcaattctcctgcctcagtcccctgagtagctgggactacagatatctgcctggctaatttttgtatttttagtagagatagggtttctccatgttggctaggctggtctcgaactgctgacctcaagtgatccacctgcctcggcctcccaaagtgctgggattatagatgtgagccaccacgcctgggctacctcatctcttttttatgaagtttttatttttattttttaattttttttttaatttttttttttttttgagacagcgtcttgttctgtcacccaggctggagtgcagtggcatgatctcggctcactgcaacctccacctcttgggttcaagtgattcttctgccttagcctctcgagtagctgggactacaggcgcttgtcaccatgctagctaatttttggtatttttagtagggacggggtttcaccgtgttagccaggatggtctcaatctcctgacctcatgatcggcccgccttggcctaccaaagtgctgggattacagacatgagccactgcacccggccagttttttattttttatttttttgagacaaggccttgctctgtcacccaggctggagtgcagtggcaagatcatggctcactgcagcctacctcctggactcaagcaatcctcctccctcagcctcctgagtagctgggactataggcacatgccaccatgcccagctagtttttttttttttttttagagatggggtctccctgtgttgcccagcctggcctcaaactcctgggctcaagcaatccttctgccttgacctcccaaagtgctgggattacaggcaggagccaccctgcccagcctgaagtattttaaagtaaattccaactaatataacattttattctcatgtacttccagatgtgtctctaaaaatagacatttttatatataacctaataccatcattacatatttaacaataattctttaatcttctcattctcaggtatatttaaattttcccattaatccaaaaaagaatcaaaattgaaacaatttcacacattgcatttggttaagcctcttaaatctcttttactctctagtccagtgctgtctaatagaacttctacagtgatgtgaaggaaatatctaatatgctgctatccactatgatagccagtggcttttgagcacttgaaatatggcaagtgctgctgaggagcagaattttcaatttgaattaatttaagttgctttaatttaatagccacatttccttactctcttactggaatcagccatttccccatggtgctctgaatcctcttagtgagaaatggtacatagacaccaaaatcagggtgctaagtctgtccattgttatgtggtttgtcattgagatacaaataattttttttgttttgttttgagacagagtctcactctgtcacccaggctgcagtgcagtggtacaatcttggcttactgcaacctccacctccttggtccaagctattctcctgcctcagtttcccaagtagttgggattacaggcacccaccaccacactcagctaattttcgtatttttagtagagacatggtttcaccatgttggccaggccaggctcaaactcccaacctcaggtgatctgcctgtctcagcctcccaaagtgctaggattacaggcgtgagctgccacacctagccaagactcagtgaattttaataatccagagctctccatgtatcccctaatctataatctacagtgcttaaatagaggcttttataagccattatgtacactcatgttatgtttgtttttttaattacttagtctttttccaaaaggaatttgcaacagctacataatcttataattgttatgtttcttttatattttcacagttgttttaattttaaattgtaaaagcaaacaggatattcttataataaaaatgttctgagatgtttttcatatttgtgtctttcatgtggctcagaatatatgagattttcatgtcatatcattgtcagtattcagccttttctccacattgtgtggggtagaggatagagtctagaagcttataaaatggtcacaaatgaggaagtagagaaaacattcagcctagaaagcgtgcatttgtggtatgaaaggcactgtggaatgctgctggattctgcccttcagtagttacccgagagagaccgaggcagacgcatagctccctcctcagcaaggagaaaagagttgcctgctttcctcagcgtgctttccatttcctttgttttgaaaaaagcacattatgctaatcaactttccttaaggtagacatttttattggtagatcaaaaaccgtgctttaagttataagccttctgaacttctgtatttttctcttaaattataatttccttttattcctatttaaaacccaagtgtaaaagatgtgttcataggctttctactcactcacttgcctctttttcgtaaccacagaatgacagccttaaattttaatttcctgacttggcttgcccatggcctgacttttttaaaacaaggagtttaagttaaagaaatttctccagccattattatttaaacacatttacacagtatcaaagaatttaaaaatgttgctagcagttgatactcccctcagctttcggttcagcgtgttttagattgctcctattttgttatttgtctatgaacttttatatttttcaaaaaaataaggcaagccctttttgcctgataggcactgctgagcagtataataaacaagcatgggggcaacagaacctgggttccaggctagactccactgttttcaaacggcatgactttaattcatcaagtgaacacaattaagcaaagtataagtttgggaccatggaatatccttcactcatcacacaggttactgtgaagatcaaatggatataatattgctttcaagatggcggcacacttgccactatacacaggtgatattattaggcagagtcgccacatagtccctgtccttgaaatgcaggcaaggagccctcaccaaaggccagggtggttttttgtttttcgggtttttgtttgtttttttcattgtggtatattatttcctctggcatgctattccctgtcccactcttacagaactgtgtcagtgggtcaccaaaagagagagctgattcaactgattccttgaaataaaggtagtcagacctcgcggtatgatggagatgaagcaactgatttactggttttctctatttttctgctgttcgtgttgtcatttaaacagacatggaaaccgtctgcacacgacagagatctttccgagcctttttcatctagtgtgctgtgctgacccgctcccctggatgcctgcacactcatttggttctcccttttggtcacttttttccacttaccctggcagaaattcacgtggctgtcagtagtaattcagtgacctgctttcaaaaaatgttataaattaagcagcttttctgtgtatagcttttgggttgaactgagaagggctaagtgaagggagaagctatgttgagctcagagtcagaatgccctgccccagacattttgagatgtgaatttttcatttgttttgtttggtgacacttagagagaataaacattctgagataaaaatcttccttgggcaggccagcaaagatagaattgctttagagcaatttggcacttggtcttaatcttaatagtttggaactcatatgtgactaaggaaaaggatattgcaaaaagttgtgccaccctaggctatatgatataaacttagaaagtctatcaacgtgaaaggtgaagagacctctagagacatcggggacaaactccaaatttgggaaaggtccatccacatttgcaattgttagttaggcttgggaagtggagtcattggtggatgaggcaacttccaatcaccatctcgatttttatttccctaacttttaagattattttctatagaaaatcagtagtgctgaagttggagtcatttaattgatttgtgccaacctagggaagcatttactctcgagaaaagtttggtaaccattaatcttaccttagcaacatttcagtttaagcttatctctgactttccaaagacaactaaagatttctacagtctctcttataactgatccattttttcaaaagatgtccctttgggaaggaattctcatttttagaattttagtccagaggaagtatttttttttcagataggtgaaatagaataagcagaaaattcttattttttactgttttatataccattgatgcctgcttattttaaaacaaaacacaataaaaacatgataccatctgatgagttgctttggttttactttttttttttttttttttttttttttttttttagtttatacaagtttcttattaggtcagcaaattgtaaaacacataatttgccaaggcttttgaagccagcagatactgggtgaagttgctgtatgaatataataataagaaccgtttttgttcagcttgatgaaaaaactattcatgttctaaatgaaaatgtgaagatcatctaattctgacagaaaaccacctgtggacatggtcttgtttctttttatttcttctgttttctacaaaagatggcaatatttatttaaagtgctttaacaggatatcccagttaaagctttgacagactgtttatagttcacttctgcccaggggcaattaaaggcacattacaggataagcgagatgactccccggccacaggaggaggatgtgtgggtaggattattaatgttgaaatgaattatgatagagtgacatgtctgcaagagcaccatgggttagtaacttaacaagaattcatatgcagaaacccataattctcagcagttctttagggtaaatgaaactaaagtttagggagagagaacaaggagaatttgcaaagatatgtatgacagtatttaaaaatgttcttccagcacaaagacattaatcttcctttgtcaaaataattctttctgcatgaaaagttggcatggggctcccaggcttcaggtaagatgggagaaagggagcgtttctttttttttgtaccaaagtactcaatcttctgatcttccttgactttcacgacataggcatatgtctactctgaccagtttgtttcatttattgtggtaagatgtgcttgtcttgtgctttcctggtgcagaaaaggagctgcctcgctcatgcttggtggcctttttgttcctacacacagtggttctcgtacatggtggctaccacatcctggtgtttgtctatactgggagaatcaggcgttccaagtcatcttggctgccctagctctgagcttcaggggatatatgactcagaaatgctatgctttctggaatttggatatttcatttttatttgtttcttggtaaattctcttttgacttaggagaagctaactatttggaaaggtctctcagaactctaattacaaatatatggtaccttctttatattgatctcatttttaaatacctagtcctggaggaagagctaaatatgaatacagtgatgagctgtgtaggggatctcacaggaacacagtcagatttcaagttgaaaatagattcagttgataatacttagtactagattctcctgaatcgactttgggaattctgagtcaacctaaggactatattacaaaggatcagaccttgacttttaggccaggtgtagtggctcatgcctgtaatcccagcactttaggaagccaaggcaagtggatcacttcagtccaggaggttgagactagcctgtgcaacacagcgagaccctgtctctaccaaaaatacaaaaattagccaggtgtagtgtagcacctgcctgtagtctgagctactgaggaggctgaggttggagaatcaactttaaccccggatatggaggctgcagtgagctgtgatcatgccagcctgggcaaggagcaagaccctctctcaaaaacgaaaacaaaaaagacacagaacttgacttttaacaggaggcatagtaatagcaagcataagattgttttaacatagcatttaagataatttacgcatttagaaaattaattctggctggttgtagtagcttatccttaatcctaaccctatgagaggccaaggcaggaagatcacctgaggccaggagtttgagaccagcctaggcaacatagcgagacccccgctctctacaaaaaattttttttaaaaaacatagctgggcatggtgacacactcctgtagtcccagctactcaggaggctgaggcgggaggcttgcttgaggccaggagtttgagggagctgtgatcacgcccctgcactccagcctgggcaacagaatgaggccctatatctaaaaaaaagaaaaatggacaagaaaattctatattgattgaagcccagctaaatagaaaaaaaaaaattctaataggccattttaaagactttggaaaaattcttagctagagtaaaacagaagcaatcctttctttcctggttctcacatctccattctcaccttcattccccttcccctccatttgtttcaagtatttttcttttctatgtgtgtctttatgtaaatttaaatttacctacaagatattatgatattaagatatttcattctgcttcctgttttgtcacttgttctttaagatccaagtccttggctccatgtgcatctaatccttgccataagccctgcatcgtacttcatattcaccatattgaactattcaccctcaagtagtcactattaaatgtcacactgttgtagcgactacaaatgcaagctcagtaactgaaaagataacattaaattttgttttttaattggtttcctgtgtcaatttcatgtttcgggaatttgtatggacagatagacaccagagagatatgcgtatttcacctgcatgcttgaggctagagaaacatgatttattcagaactatttcaaatactgtaactaacctggtaattttgtttaggaaaacattaaattcggaatagaaggcaattcagaatacctgtctgttatctttgtcttgtcatttttgagatactcagttcctggaaaatattatgataatccatcttgctatgtagtatttcaagttatatgttgagcctctgagcttgcagagtgaacatttctattcatagacagtgctgttgacatcagtggggagatggcacaagctttgtgccctaggaaagtttaaaagctagaacattacactgcaaaccctatgtcaggaaactttgttgcagcaactaggcaaggaaaccctaccactgtacccataccttgttttagtagaattatgtttctttgctaagaagtctggtgactctcacaccccatctcagcactttcctggattccaaggccctggtgccctgagcacacaacttcccaggttccttttattgtttctgataagaactcgaattagaggcagcgtctggtactcaccaggggtgtggtcccatgggtgacattcttgctccatcatttcttacccaggtaaccttgagtatggcgcctaaatcctctcttcctttcaaattgcctggtctgtaaaatggatgcagtgaatggtagctatgccacaggtgttttgtgtgagaattaaatgagaggagttatatcaataacacttagaggcatgcggtgcctatagtgagggcttgttttatgttgtcgtttaaccctttctttctcagccctcccccactccacatcccctcccactggcaatgtacaaattgcagcttcctgcctctttcctgctatctcgctccctggatgacatccctgcgtttccacctctctctcccctaatcttagctgttttcaccctttccccacagcattgccttgacagttcctcagtgtcccatcccctcttcccatggacaacaccacaaacctgcgcacacggcctgttattattcctcctgtttcacctgtgtccattctgtctcataaaggacactgtaggctctgaacagctaaggcctttcttattcttttgtcccatggaaaataatccctccagatagcaataccaggaatagtgccttttctaacccactggtttccttttgagcttacagccaccaaatcttatatctgtttgtttctgttcccctctctgcattgactgctagagagatcagagtcaagcaggaggttacatctagctggggcactgtgctttatgctggtgtttcgtgtgcaaatctcataccatcttcatgttacagttgttccttagtgtccatgggggatttgtttcagaacccgcaaggacaccaaagtccagggatgctcaagtccctgatataaaatagcattgtgtttgcatataatctacacacatcttaccatataattttttttattatttgtatacattcgtggggtacaagtgtaattttgttacatggatgtattgcatagtggtcaaagtcgaggctgtgagtgtatttatcgttggaataatgtacattgtacccgttcattcctgtatactttaagtcatctctagattacttataatagctaatacaatgtaaatgctatggaaatagttgttatattgttaggggataatgacaaggaaaacacctatacatgttcaatacacagcaattatttttttctgaatatttttgatctgccattgattgaatctatggatgtggaacccacagatatgggacccacagatgcagagggccaactgtgtttattttttccaaatttgtttccctgttatgactcttctttatacacatttttaaactctgttgtcttgtatttcttttctaatcatcttacgtccttttggaaaccaagtggggcataaatagtacatgtttgcatttgaggcaacacagaggcatcgcaggagctggagacagcaaagttcaaagccagtccaccacttacctacagagtggccttgggcaagttacctccattttctgtctccattctcgagcctgtctagagagagaatgaacctgcctcccgaggtgatggtgctgttttaatgaaataatgcatgtgaaatgtgaagtgccactgtccctggcatggagatgctgtccttagatattaggtccatcttgtatcttctagtgtatctttaaaaattaaactatactggcttcatgcctgtaatctcagcactttgggaggccaagaccggcagatcacttgagtccaggagttcaagaccaacctgggcaacatggtgaaaccctgtctctacagaaaatacaaaaattagtggggcttggtggcacgtgcctgtagtcccagctgcttgggtagctgaggtaggaggatcacttgagcccaggaggctgaggctacagtgagctgtgattgcaccaataccgatcagcctggtatgacagagcatgactctgtctcaaaaaaaaaaggaaaaaaattaaactatactgataaatatttctaaagtaattgatgaaaacatatatatgacacttagcaggtgttgatcaattccagtgcgtgcatgagctgctttgcactgtggctacagcactcagcagtttgcttagtgagtagatggccatagctcactttaggtatctggagaataatgctcattcctgtctccagctttgtaaaattgcctaatagacggaaggcattaaaatatgaagatgggattatatcttatgaagctgcttggaatgggttttccctcttaacaaataatttctgatcatccatatcccttaagaacaccggatttctgggggccagggaggtaagattctatctgacaaagccttcctggtgtcaaccacttgccatctgttggtcagtgctggccctgctgggtctatagaatgagcttctttgtctgacgctcctgttgccatccctttctccatcagccttgtagcagtacctgggaacctgcatcctgtggaaagatggaggagcagatgacatcttccagtcaagctcgtaaatacgtgaatgcattctcagcccggacgctggtcatgtgagacatttccagaaaagcattatggttttcagaacacttcaagttgacttgggatatatcattcctcaacatgaaacttttcatgaatgggagaagaacctatttttgttgtggtacaacagttgagagcagcaccaagtgcatttagttgaatgaagtcttcttggatttcacccaactaaaaggatttttaaaaataaataacagtcttacctaaattattaggtaatgaattgtagccagttgttaatatcttaatgcagatttttttaaaaaaaacataaaatgatttatctgtattttaaaggatccaacagatcagtattttttcctgtgatgggttttttgaaatttgacacattaaaaggtactccagtatttcacttttctcgatcactaaacatatgcatatatttttaaaaatcagtaaaagcattactctaagtgtagacttaataccatgtgacatttaatccagattgtaaatgctcatttatggttaatgacattgaaggtacatttattgtaccaaaccattttatgagttttctgttagcttgctttaaaaattattactgtaagaaatagttttataaaaaattatatttttattcagtaatttaattttgtaaatgccaaatgaaaaacgttttttgctgctatggtcttagcctgtagacatgctgctagtatcagaggggcagtagagcttggacagaaagaaaagaaacttggtgttaggtaattgactatgcactagtatttcagactttttaattttatatatatatacattttttttccttctgcaatacatttgaaaacttgtttgggagactctgcattttttattgtggtttttttgttattgttggtttatacaagcatgcgttgcacttcttttttgggagatgtgtgttgttgatgttctatgttttgttttgagtgtagcctgactgttttataatttgggagttctgcatttgatccgcatcccctgtggtttctaagtgtatggtctcagaactgttgcatggatcctgtgtttgcaactggggagacagaaactgtggttgatagccagtcactgccttaagaacatttgatgcaagatggccagcactgaacttttgagatatgacggtgtacttactgccttgtagcaaaataaagatgtgcccttattttacctac

SEQ ID NO: 3 - DNA, 26198 bp, Homo sapiens
gtttgtaacttttttcattcctggaagaagcagtggtgagggcacagagatagcgtagcacattgtctttgaaccttaatgggaattacttcttttcagggaaatatagcaatggccaattttgtctatacattttaagattgtttcttatagttgttagagattttatgagaagcttgaatttctttcagtaatcctaaagaactgttataaagaacttatttatcagaagaaatattccttattgggaaaaactaaaatatatcaattaaaaagttaaaagtactcatcatgcactgatagctgctcttaatgtttccttgtagtccccccccaccagtgtttatggatgtagcaaaattggaattacggtttttaaaaaaaattatatcatgagcaaaatttttccttgccatttcatattctttgcaaatataatgtttatagtaatatatttcatgctgtcgaggtatattataatttacttaactaatctgttggacttgccactttgctttcaggaaatgtgtgtaatgatagcattaagtggctttagaagttacttttttaaaagccttaagttacaaaatactgtaactgggaattatgaataccaaactcttatttttgcaatgcagttaatctgaaataagcattttatgttgcattgcagtagaaactggttgttaggatgcaacgtgattttcgaataatgtcagagttgcatatttcatgtattagtttttagtgacttaacatacttaattgaaacttttcattctattctaggctgtttggttgtatgctattaatatgtgtgcgatgttatgagggacacactaaagcttcaaattcattccttcaagttatctcagtagttggaaagaatatgaaaataagagtaacaaaggaaatagaaacataccaagaagactaaagactggtgctaaagagggtgtttttggctgaggaggctatggaatgtttcaggagaggaggtaaaatttcatctggctcttaaaggacaggtaggattgaagtggagaacaggtaagggagtggcgttctagacacagggaacggtgttcttgaatgtgggaacattctgagtaggaaggtgtaaataattctctattagagagagctttttatacctttggtcttagctttttcctttatctgcctccatatttctgtttttagtccagattttcttatctctatcctgtacaggcattacattatagagtgatatattaatgtggtagattgctgtgattgctgctgtccttctcttcccttcattttcagatgttcttttttcttgactttgactctctttaggtttttggagaagaggggtatcttactgcctaacttttaattttaactattgtttatctgtatgcttaatttggctttttattttttttgttttcctagtgcatttggattttacttagcttttattatgaagccatgtgtttcagtttttttctgtatgtgtaggtgattgggcttatgaattgatcaggagcatggaggaataggtacatctggattagcgagtgtgtgaattcttgagctgatgggcctaagctctaaaggatagattagttgtcagcatattctggtctagatagtatggcatagtttttaccagtatgctttcttttccccatcttcccagtttgctttttactctaatggcttgaaaaagtataggttaggttggaagtgagagaacaaaaatctgctaatttaactctgaacaaggaaggctttaggctgatgtttgaaaagtgtgttccatggaatactggtctcctgaggtagtctgagaaaagaagggtcctgtagtccaggaagttctgcatactccttttgtgaattcaggatgtgttgccatattaagtctccaggatttgcagtcaagaaagttaacttggtttaactgagcattttctgaaattacttggttaaggaaatacggtttttctttcttttattcttggtactagttctgaggatatgttaagggaaattttaggggattgttaaagatctgtggatccttgggtggaggcaggtttaaacaataattgtttgggaatcttaattttgaatgagagtgctggctcaagtcaaatcaaagtaccccttggaaaaaaaaaagttatattattccacttaggttgggcttcaggtttcaattgaaaggaagcaagaaaactgtccttgtctggctttcattaaataacaattcagtcaaatgatttcagtaatgaattttgtctaactcaaatgcatttgttatttaaataaaaattaagatgtgctgttttttaaaaatgtaattctctaaataaaaaaaattaacatagaatcctgccagcttaaacatcaaactgaaacttactttaaaataggtatatacagagaaatatgctcatgttgtaactcatgcctggtttacatctacattttttaaatttgggaatgactgaattgtgaggtgaagaggaattagtaaattttggaaagaaaaatacttatctttgttaggtcatttaaacaaacattaagatgcaattttgtgagatctgtcagtaacaccggttgttttctgtgaacttagattttaatctattaatagaatatattatctcccctcattcagttatatcataaagttttggtgacctttgtagcctttataaaagggaagtctaagggtggatagtagaatagcattttatttggaaggccctaaagatttttgtaaaaaaaaaaaaacaaaaaactgttcttattaaaaaaaaaaaaaaaccctgtaattctgcaattttaaagcgggtgtggatttgagagatcgtagacccattcttcattatatatgtaaatgctgttcctttacaaaggatatgaatacattttatattttggaaactgctggtttcaggcatgttagatcatttggctcttgggacagaaatatatttaagttacattattaacaagtaagagtacctttgggcctgatcccctctaagcagaataatgtcatgactgataattgtgtaatgtggctaccttctgttgtgaatagtcttaattaatttaattctgttttgttgctggaagtatctacccagggtattttaaattcttcctccaaataagaatacaatcaagcatctaatgcacagtcatattatcattaaattgatgttaaaccaaagacaaaggctctcctaggtagtgtccattagcattttccagcagggtgaatctaggtctaggaacaattcactgaatatttgcctaagggttttcactttccatgttttacacaatataaataatttaaaatcccccccctttttttttttttaaagagatggggtttcaccctgtctaggctagagggcagtggtgctatcatagctcactgcatcctctaactcctgggctcaagcgatcctcctgcctcagcctccagagaggtaggactgcaggggtgagccaccatgcctggataattaaaaaaaattttttttttggtaagagacagggtctcacgatattgcccagcctggtcttaaacttaaggcctcaagtgatcctcctgtgtcaggtgtcagccagtaatttaaaatgttttgttaagagtagtctctctctttccccactttgttatagatctgagtcatttgctttggaataatgtgataaaagtctaagataaaggattgctagactaatatttagacatgtttgtgttgatgttctgtacaggttgtagttttataggctagaatagactaaattttgaagggtgattagttaagaatagactaaattttgaagggtgatctgaaaagactttatattgttcttttgtaaatcttgagtaaaagaaaataaccaaacagggtagtgagtagagagataaatgaatcccttaagagaatgatagtaataaaggcaggtggaagtaagatcagagttagaaaatcttagatttgttttaggttataggaaaattgatttattttacatgttggtaaaaggcacaagaggcttctttttacattttactttatcaaagtgaaactttttcatttgatttttaaattggaacacattaactgaatggcataggatatatcatagtgatgttgtttaggtacatggtaactgcagataggtctcaagttgaccacatttgaatgttacattattactttgtggatatttaaagacagatggcttgaagcctgagaagggggaaggagtgaaagtataggagcaagtggtggtaatttaagaagatcctcaagtagaagtctgattttgtgatttcagagtctctttaattgttaatgttactattattaaataacaggtacatatatcctaacagtataaatatgaaccagaaaaatagtataagaatgaaccacaggccgggcacggtggctcatgcctgtaatcccagcattttgggagaccgaggtgggcagatcacgaggtcaggagtttgaggccagcctggccaacatagtgaaatcccatctctactaaaaatatacaaaaattagccgggtgtggtggcacgtgcctgtagtcccagctactggggaggctgaggtgggagaatcgcttgaacctgggaggcagaggttgcagtgagctgagaccacgccattgcactccagcctgggcaacagagtgagactcctctcataaaaaaaaaagaatgaaccacgaaaggtagcatgccttgcacttttaaaatgctacctataaggttaaatattttgttatgaacttaaaatgtgtacatacacatttatataactcaacaattgaaatgaaagtttcatgaaataatacttgtctgtactgcctgtgatgcagtatactatttttctattctgtttcatttttaaaaaggttggttgtgattgataatattgatttagtggcttgtgtgctgtgcagtcttagcaaaaccgatgtcgattcctgctgtaatttagttttcaacaaacttttttttttaatccagctctttgctgggtgctggggggatacaaagatggataagacatagtctagtgctcaaggagccacacaatggcatggagagtaatgaaaatagaagctgcaatgctgtaactgccctaacagaggcatgcccagcctgagatggaaggggccacatcatttcctagcaggaagatggcagtttcttctaggttcacagattactactgcgtataatctgataatccctcttatgacgggcttaccttgatgggaaagttagttttttacctagattttataaaaactaaacaataatcaagtttacttgaaaataagatatttttgaacttttagcttactggccaagtttgttaaaaaataaatattaatcttggcactttgggaggctgaggcgggcggatcatgaggtcaggagatcgagaccatcctggctaacacagtgaaaccccctctctactaaaaatacaaaaaattagccgggcgtggtggtgggcgcctgtaatcccagctacttgggatgttgaggcaggaaaatggtgtgaacccaggaggcggagcttgcagtgagccaggatcgtgccactgcactccagcctgggcaacagtgtgagactctcaaaaaaaaaattaataaataagataaatgttaattatatagataatctttaaaaaaattaaattttacaaatgagtaatattttcctaaatatgtcataggaactagctttaatattattattggagaaatacttctttcctgactttttcccccctcagtattgtgacttattaagaaaggtaaagtcaggtagaagtttaatataaaaatatatttgattatttgtacagatggtaaattaaactgtaaaataaaccattaacttttaaataatggaataaaagatgggtacttaattatccaacacttaaaaatgatttttttaaaaaattgcaacttgtgtatattctgtatattgactttttatctcacagtgaattcacaataatactgaatctagacgttaatagattagttgcatattatccaaaaaacataaagcatgacatttctatagaatttagtttctgtaagttacgcaattgttacaggtttttcctgtgaaggataggctgctacctcaggttgaagaagctaatgttggagtgaatgtggtggttgccttagaatgattttgccggcagcagtgaaaaatgttttccttatagttcttgtgcagcaggccatggagtaaagcacatttttataataaaggtgatgaatttaatcgcaaactggcagcctaatggaaattgcagaccttgatactgggagaaaattgcctataaagcaactcttgagagaacaggctttaaatttatccacccattttgacattgaggagaacattggaatcatagggtattttgcttggcctgcttagttcagatgaatgacacttgagtattctatttaagtgttagagatggtttaaagttaagttttatacattaaagcctttagcacatgcatttttgtattttataaagtatttaaaattttgctggtttttaacattttagtagatgatatgcctaatgaaatccatttgccacgtttctagtcatatgccgtgtatttcatgtttctgctgtatatatgaggaactgcaatcttaaaggatatagaatattgtgggcttataattaacactactgacagaatgtttgtcatataaatgatcagtctttactattttctattgtatttaagccatttatattataatatttaaacacagaatggtagaaaatttctattacaggaaaatctgggtttttgtcattttttactctaagtgactaatgtttgtaatttcttaggaataaccaaacccttgattcagtacacactatttaaagtgtttaatccagaatttaggtgcattgtagtgtgtattttgagatgcctgttctgtcccgcagtatacatgcttgttaacagatagctaatgtccaagttcagagaattatttgtggctattggaaacatttacccatccaagcaaggtaaatgtttaaaatgtagaaatgctgatgtaaaatataaaaataacatttctcattatacttattgaaactactggggtttaaaggtaaagaagtgttaacttctagaaggttattcgattttgcttccatacacaaccctgattcttttgactgtagttttgggcagcatggtacattacataataagggtatagctgtggtagatctttgttctctttaggtatagaattggagaagtttaagtttaaagcaagtatgagagcttaatattttgatggagaaagcaagaaaaacgttttacaaattctctattcagggaacaacttactgaggtggtcatgagactgaaggtatgaaaaggaagaaatggcaaggtgcggtggctcacacctgtaatcccagcactttgggaggccgaggtgggctgatcacttgaggccaggagttcaagaccagccaggccaacatggtgaaactccatctctactaaaactacaaaaattagctgggcgtggtggtgtgtgcctgtagcaggagaatcttttgaacctgggaggccgaggttgcagtgagccaagatcactccattgctttcaagcctgggcgacagagcgagactccgtctcaaaaaaaaaaaaaaaaaaaaaaaagggaagaagaaatagaagtattccttttcttgctaaagcaagaaccagtaaattgtatatttttcattgaaagtagttccatctaaattaagagcctgactttttggagaatgattacattcataggagaagagcatgtaatgttggagatacatttgaatgtttagccatctctcaatttccatggagaggagagttcaaaataaatcaactctttaattcccttatcggtgacatagatacttggtcatttgttttatttggctgaaggaagctgtggcaagttgagaattcagaggtaaagcctttgtgagagagatgatgtagtttaattattttaactctagttctcctgatccaggaaagctttatttttactttcagatgaaaattaaaatttgttgcaatttaacgtgtaacacgaagctagaataaggtatatacataagctatatatgtatataatttaaatgatacaagtgatgtatgtatacatatagtacttatgttaaaattttactgtttttctgtaacaggctataaatttagtgtatcgattatcaaagtataattagctgaatataattatttgtttatgagatgatactaaattcagttcccatagatactggtggtgatttactttttaaaaagagtttttcttgttatgtgcttaaatccacacccgaaatgtagccataatttagttcaagattgtttatgccacagtctggaaaacacctgaagtaattgagatttttgagtggttgtgaatggattttagaatcatgtgttgaacagtgacatcactaaattagccatagtaatgttactgtttcacattgagggtttcaggtgtgatgcttttcagttttttgtttttgtttagataactattttagggtttgagttatagttatgcaaatttacattacggtaatgcaaatataccttccaaacttgtgtcaatatgtattagacgctaaaatgccctcccctcccatctctgtttccctcaaattattcatttgtggttaaactaattttaaactccggaaaagtggttaagccaaaaaaggtcagatcctggaagaaaaagctctgaccttttaaaaagagtgttggaaaagtatttgacggtcattttcataagctaaatgtactgtgaggtttgtgtttgagtagcttttaaggttttctggattatgtggttgaattttaaggaggaacttagtaaaaatgagttgggaaaattaacctgttacattctaccttccttttgctatgtatagtagatctacatgtgagaagtcatttgcaaacagcatttgttaggtgtcttttctaagtattttttatgcctacaaagtctgggaaaagaccattttcagatttcagataatatttcagataaataatgatcagatcagtagatactgtctttcagatctatcagaacacagtatttccagaatataattttacatcacagtcatcatttataatgacattgatgaccagtactattattattttaaagagaaagatatatatacacatacacataaagtcatgcattgcttaaagacagtgatagattctgataaatgcatcattatgctatttccttttgcgaacatcatagggtgtacttgcataaatctagatggtatagcctactacacacctcagctgtatggtatagccttttttgttcctaggctgcaaacctgtacagcatgctactgtactgaatactgtaggcagttataacacaatggtatttgtgtaaacgtagaaacattctaaacatagaaaaggtacagtaaaaatgtggtataaaagataaaaaatggtatacctgtataaagcacttaccataaatggggcttgcaggactggaagttgctctcggtgagtcagtgagtgagtggtgagtgaatgtgtgaaagtcctaggatactactgtggatttaataaacattgtattcttaggctgccctgaatttataaaagatatttttcttaagtaataaattaaccttaacatactataactttataaacttctaaattttttaaacctttgactacaattacagtttaaaatacaaatacattatacagctgtacagaaatattttctttatatccttattctataagctcttttctgttttcgaacttttctatttaatttttacttctgagtcctttttgttgaagacaaacacacacattaacctaggcctgcacagggtccagatcatcagtttcatcttctttcacctccacagcttggcccactgccaggtcttcatgggcaagggctgtatggcttaccagtgtgtctccagcacagtgtctgataccacagaaagataggaactactgcattaataatagcagattcttgagcatgtattacatgacagacacctacaaatatttgttgttcaagcgaaagaatttattctatcaaactcagaattttcgctctagaataatccttcgtggtttttttttgttttttttttttttcgggggggtgggggacggagtctcactctgttgcccaggctggattgcagtggcacgatctcggctcaatgcaccctccgcctcctggtttcaagtgattctcctgcctcagtctcccgagtagctgggattacaggtgcccgccaccacacatggctaatttttttttttgtattcttagtagagatggggtttcaccgtgttggccaggctgttctcaaactgctgacctcaggtgatccacttatctcggcctcccaaagtgttgagattacaggcatgagccaccgcgtcctgaatcctttttttttttttttttgaaacagagtctcactctgttgcccaggctggagtacagtggcttggctcactgcaacctccatctccagggtgtgagcaattctcatgtctcagcctcccgagtagcaggtaatacaagtgtgtgccaccacgcccagctaatttgtgtatttttagtaaagattgagttttgccatgttgaccaggctgatctcgaactcctgatctcaagtgatctgcctgccttggcctcccaaagtgctggaattacagacgtgagctactgtgcctggcctagaataatcttagaagtcctcatggatgagtaaatatggatggtgaaaagcttaaatgagaatgtcttgtctgattttcattccacttttaatttgaatataccatttcctttcccccctcattgtatatgtctctacttttttgttctttactttattcatagttctaatttctactcactgccccatcttttccttcttctctatctagctcttaccatctgtcatcactgcttttcccactatcatgatcactcactctagcagcaaccaaatttaactttggggatggagcagtagtgctggagaggccaaatggtatgagtaaaagaatacagcttttggagtcagctagattttcatctttatgtggtctttggtgaaagtacttaatccccaaatctctctttcctaactatgaaatgaaggtgaaaatattacttacctgtaagggaatctacctgataataatggaataaaatgctgacttccaccacagagttcttggggaggctttttatgtggaaagacattagacttcattactctgaattctggatttgtatctcctagccatatagactgaatgactgtagaatccaacataatgcttttaatattggcataacttccttagtttaagttatatgatgagatgtagaaactcaataaaaggtaaaattattctttgtgttatgaagtatgtttagaggtttcttttaagaggtcttattttgctactctgcttcaactatagtttttgttcatttagtttgttatagaactgggaccatgtctgtatttgcacatgactatattcctagggccttggccagtgcctggtacagagtaggtgtaaaatacatagttgttgaatgcatatacgaaaaaaatcattatattgtatcattggtaagacagttttttttttttaaaacaggttatactttattcaggtcagttcaattttgtaaagtttttacttttaggaaattaaaaagcttttattttattatgttctgaatatatttttactaaatatactttgagtacagggttaattaggactgtgcaggctatgaatatagggtttatgataaagctcagtaggagtggatgattgcagtccttttgtgcaatcctggcaggagcagccttttccttattggctcggtttttttcttaaagccaagactaagcacatatttagatgcctgtatattttattttctcatctgatgtaagatacactggaagaatttagcattgatgtaatgaacatatgttattagcaaagtgttctaaacttctcaagtccagaataaagagaccaagccatttgcagaagagcactgtgcatttatattactttaaagggaagaggagtaagctagctagaattgtcaagcagatgccatattgatgcttgagtgaagttacaagaaatatttttggagctttgcgtgtttttgtgctaatagtcctaatatttgttcaaaaggtgttttttctccaaggatgtagatgttacaaatatagcaaaaggcaaaaacaaagctatctgccctctcagctaacattctaatgtcaacaggcaataatcaataaacaagtacagtaatgtattaggaaatatgaattatagaaataaaattgaagggtaaaggggatctggagtgttcctatggattgaggcaagttgctgtttaaaaataggaagatcaaggttggactccttgaaaaactggagcagaaatctgaaggaggtgttagtgtgagccatactgctctttggggaaaagctttccaaggcagggaaagagttactgcaaaaccttgagggcagggctagcaattccttgtgtggacttgtagccagggacggaggttaggttggttaaggacatttgatcagaagggcaaaaaaggtgagggcaaggggagcctgtgtggatgctgtaggctattgtttatctttgtaaggacctgggcttttataccaactgagatgggaaatcattagagcagtggttctgggcatccttttagggaatctgtgaaatcagactatctttgtgataataagatgttatttgccattttcaccaccatttacatacgaatgtacagtggagttttccagaggctgcgtgctatgtgatattgcaacagattgaattcaagagaagagagaatctagctgtctttattaaaacagacatttaaaagattgcaaaagtataaaactgaccttttcagtaatttgtttagttggaaaataggttattttctgtaaaatgtgcatttttggttaatatataataagtttactagtattttaaaatgaaaaaataatttaaacatttttagttttaatttctagcgtggtaagttttattagacataactttcatgaacagggatctctttgggctcctccaattaatttttagagagtggagtcctgaaaccaaaaagtttgaacagaggagtgacatctcacttggatttcagcaggctaactccaaagaataaagaatagactgaccaaggtagtggtggaagcaggtagaccggttaggttaagattaggtaggaaatgattgtggttagagctgtaatgttagggcttgtgatggtgaagtgaaattggggatatagtctgaaagttgagtacacagtatttgctagtggatggaatatgaagtgtgagaatggacttgacattgtgtcattgcccgagatcaggaggaacaaattttggggaaggcttaggagtttagtcttgaatgtatcagataggttgagaagggagttggtgataggatttcattgtggagggttggtctggaatgtccattaccagggtttttgttggattgtattagtaatatatattttttaaatatagtatttagcttctaatcagttatacttttacaatctcaacacagagggctgtattctgcattgaaaaactgattaaatttgctttccaagtatgttattatatgatagaatgtacacttgaagaggcagtaaaaaagtaataaatgtagatttgccagtagcatgacatgacttagattttctgaatctttgtgaagtaaaccaactgtggtttaattaaaggagatcatattgcttaccatattaaaataatgttttcaataccaattactttgacatttgaaggcactgtgtaatatgttggtcttgttacagaccaagatatttttgctgaattttatttttcgaattcaatgtcagtgcttgttgtagttccttttggaatattgtgtatagtagattgtttcttaagcctttaaaaaactcttcaccgggtacagtggctcacgcctgtaatcccagcactttgggaggccaaggcgggtggattacgaggtcaggagttcaaaaccagcctggccaagatggtgaaatgctgtctctgctaaaaatacaaaaattagtcggtgtggtggcgggtgcctgtattcccagctacttgggagactgaggcagataatggcttgaatccgggaggcagaggttgcagtgagccaagattgcgccactacattccagcctgggcgagactccgtctcaaaacaataacaacaacaacaacaacaactcctcttggctttttcataatatgagagaattctcttgcaacacctgtgaatgtcttttgacatgtattttctttcctaaaaatgtcatgaagtgacagacatgtatttgctttgtttatggtattaaggtgatactgaggtaaacctatgctacatgattataaaccttaggttctttctgtattgtacttaggtgaaattttgaatacaatcgaaaaggttaattaaaaatcaaataaattattacctaggaaaaagcaatttacccctacctcctacacagacgtaactccttcagaatttttggggcatcggtttcattttcagtctgatttaccaaattccttttaaagccctaagggatttggagcttcaagagggcgaaatacataggtacagacctttccccacttcttcaagagcaagtgtagaggaatctaattacatgaggatttggtgttttccgaggatgtaatttaaagtaaattctggtggccagtagtagtttaagacattttagaatatatagttctggcaggagtaggatagtctggcctggagttctcaaccagtcattgagtatatacagcccttgcctgtagggggtcagtacagaaggagcacaaaatggaattctctgtaagaagacaaagagatgcattttagggcagatggaagccataaatgtgatgtggtctaatgtgctcatttcgcagatgaagaaaatgagacccagaaggttcagtttgaaggcacacagctcagctaaatcatgctggcgaaacaggagtaaaatccaggttgtgtaaactcaaggccagtgctttttgccttaccacatgtattaggaagataggctttttatagaaaggtcattgttttggattgtaatctttctccaccttctttaaggaaagttcaaagtgtgtgtgttttcttttaaagcagtatattaaagaaaatctaaaacgtatagataaaatttacaaagcttttgcagggaaagtcatctgtagttgaggttaacattagatcttttatctgtggatatttattttggagaagagtaacgaatatattaagcacttatatttaacatatattttggatgattagtaatgattgcttatttgaagaaagcaaatatgtataattaaaattttaacaacattatttaaactgtagaagataccattttgagtgatttttttccctcagttttctttatcttttttcacagctttcatactttgtcacattatttccccccttccctcattagaataaataaccccaaagtccaaagcaaacactttttaattattattattattatttttgagacagggtcctgctctgtcacccaggcgagtgcagcggagtgatcatggaccactgcagcctctacttcctgggctcaagcgatctttccacctcagtcttgagtagctgggactacaggcacaccaccacacctggctaatttttgtggagatgggatttgccatgttgcccaggctcactttttaaattgtaattctttattttgaaataaatttagatttacataacagtggaaaaaaataatgtggatgttaactcatataatcatagtatagttatgggaagcaggaaatgagtattgatacagtgctatcactaatctgcaaatcttactcaaattttacctgttttcctcctcactctgttggtttttctggtccaggttttcctactctgttggtttttctggtccaggttttcctactctgttggtttttctggtccaggattccacattgtatttagtagtgatgtctgtttcgtttcctccaatctgtggccatttcacattctttcatgaccttgacacttttgaagaatgctagttattttataaaaatgttcttgaatttgggtttgtttgatgtgttctcatgattataatgaggaccatttttggcaaaacaatcacacacaagcgctgttgtgcccatctcagtggatcatgctgtggagtatatgtcttttttttttttttttttagttggagtcttgctccgttgcccaggctggagtgcaggggcaccatctcggctcactccaggctccgcctcccgggttcatgccattctcctacctcagcctcttgagtagctgggactacaggcgcccgccaccacgcccagctaattttttgtatttttagtagagaagggatttcactgtgttagccaggatggtctcgatctcctgacctcctcatccgtccacctcggcctcccaaagtgctgggattacaggcgtgagccatcgagcccgaccgttgtggggtatatgtcttaatactggtaatattaactttgattagttggttaaggcgatgtctgtcagatttctccactgtagtttctccttttttccttagtaattaataaatgccttacaggggagatactctgaaaccgtacagctatcctttcatatttttgctaactaatttagcattcattgaatgatttttgtttgcattgatttttactaattaaaattctttaaggaagagctgtcccctttcccccttgtatttatttattcagttatttatatcaatcttttcttatgggttgtaattcattactatcactatataagtaaaaacttttaagaaaagtttctgaaaagttaatacattgagataattcaaaattcagagactcgtaagagtacattttcttctatccctgttatcttgggtacccgtttccattgctaagagtttatttttattctttcagaaatattaaaggattgggaaaagagttgttaataataattgctggattctcaggtatttctttttacctggattctgatgatatcaaactaacttgctgttgcatcttcttcggatattcattaaaatgttacttaactagttcgccattagatttaaatttacatttcagtacagtgactattatatatagatggggactggcttcccctgagtatcctaagaaacgacatgaccagaagaccacattttctattaaataaataatagttaaatattaacgtgtaaaagtaaacaggtttaaaagtaaactcttttttggtaagttgaaacaaaagtcagtttaaatgagaaaggctgcctatccaattaagtaactggtttttcaaaagtgaatgaaggccctccccaccaagtcccccgctctgttctgttcttttcagtgacctattttactttctgtgtctactggcagtatccgtttattcattcaggatgtcactttaggaacctagacctggagttcatctacagaactgtgttctgttaaaatggagtgaaagaaaagggctaaaaagactttctttcacacgctattaaaacagtgaacatactggtctacacttattatagtacagccagcaatacatcaaaaggtggcttattccattttatttccattctctaattaccgtttcttaaaatgaattttaagagtttcttatgccaggaaatcctctatgaaactcttaagtaaagctggttagactagctgtgacttggtccagtttcttttatttttttgatttaccttttctttatttttattgatagatattttacatatttatgaggtgcacgtgattttttttttttttttttttgctacatacatagaatgtgtaatgattaagtcagagtatttgaggtctccatcaccttgagtatttattatttctgtatgttggggataattcaagttctctcttctagctactttgaaatatacaatgtattagttaactgtagtcactctaatctgctgttgaacagtaaaattcatacctttatctaattatatgtttgtacctattaacatacttctcttcatctcttctcctactcatccatccttcccagcctctggtatctataattctactctgcctttatgagctaacttttgtagcttccctgtatgtgtgattgaggacataacgatatttgttaattctttaatcactgagcattagttgttttttctttttattttgaggggacagtaaaaagggtattgtaatatatcctccttcagattctggtgaagaccagttgagtcatgtaaaaaataactagatcgctttatttttagagttgggaggcaatgaattataacatctttattatgctactgtttttatataggcagtcccatgcttgcagtgttttgatgtaagcttttctgctttctactctcctaataggtgattattgtaagaaaaatttatataataacatgaaagcgaaatagtcctcgtaaattcatctcccagagatgattactgttaatagttgtatcttttttgactttgttgtgtctaccaaaaaaatgtatacttaaatataaatttgatcatacagtagaggttcttttctataaccccaggaacgtgtgtgcaacttactgttaggtacttctgtaatacagaactgctgctgagaacaatcaggaaaatcgaattaacattgttacaaagtttttgctgttttgtatttttgcatgtgaaatttatcgccagttgtggacaattgttagttttggtagtacaaagaggagttatgattaatttaattttgtagtttttgtttttggacttaggattgtgaagtacagagtacaatttaacccttggaataaggttccatctagggaattactaatagcttgggttttttttttttttttcttgccagtttgaaagcagttactttgaaattaaatcagatttatttggttgttagagtaagcaaacatatttcacttaatagtctgtactctaggctaggcgcagtggctcacgcctgtaatcccagcactttgtgaggccaaggcgggtggatcacctgaggtcaggagttcgagaccagcctggccgacatggtgaaaccccgtgtctactaaaaaaaaaaataataataaattagctgaccatggtggcgggcacctgtaatcccagctactcggggaggccgaggcaggagaatcgcttgaacccaggaggcggaggttgcagtgagctaagattgtgccattgcactccagccttggtgaaaagagtgaaactccatctcaaaaataaataagtaaataaataatactctagtaacttgctttttaatctagttgctctctgtaccttgtgaacttggcctcttaaaaagagcacagtgaagttaaattccatgggtggttctcagctcaaaaataactttaaaactttctctgtatttagtcaagccttaattaaagtttatttctttttacagaagagattgttgttaaccctaaattcataaggttacacaccagccacctctttatgaagtgacagttcttcagttttaaatagattactaatattattgttttaagatttaatgagtattatctaaataaaaacgagtaatttgttattaagacctaaaataactaaatcttactttgctgtttgtgttagtgagctactttaatgtctagcaagaggtagggattgcagacagtgtggaaatactggagcctatttaagcttcagatgaaagcagtcattttaacttacctcataaaatgtattttatgtatatcaaattatgtatgtgtttatatatacttgtcgttttactgacaactaagattttccataagttaatggaaaatgttaaaaagcacagttatggtcaaatgacacagtgaccttttcattcctttattagctcctgctctagtacaatgataaggcagattttgttgatggattcaaatgtacaaagaaacagcagatagatacccagtgaaaaaatatatatgtctataacatcagcattgcaagttgagcaattgaatcagctaccacaacactaagtataagatgcattttatgtgagtttttcttcagtgttttaaaactgttttgtttttttacaaatcctgaaagtaattctttggtgcattttggctttgaattttacttttcattaatattttgttgcttatgattgagttagtaaatagtttttgtgtttcatttcctaattttgattttcttagtttaaatttgctgaaaaatttctaaaccagaaagtaatatagagctgtatttgattctttggctttccaacattgcagtattgattttatacacagccttagctatgtatttatatattttatgaaatgactgctcaatgctgtaatttagatttctcaccccatatttttttattctcttaacagtttttcctgttcttaattccagtgttctagttttccagtgattgatctgttagttaaatccaataattcatatatatgtaattggatttaacatatgtgtgtataacatatatgtataacatatatatacacacacatatatatgtatatatgtgtatatatatatgtatttcttttttgagatggagtctcaccctgtcacccagactgtagtgcagtggcatgatctcagcttactgcagcctctgtctcccgagtttaagcaattctcctgcctcagcctcccgagtagctgggatgacaggtgcctgccaccacacctggccaatttttgtatttttagtagagatggggttttgccatgttggccaggctggtcttgaactcctaacttcaagtgatctgcccacctcggcctcccaaattgttgggattacaggcgtgatccaccacaaccggcccagtaattcagatattaaaaggatatgtacaaaagtagcagagtgcacgtacaataatttggatacagtaaaagagtagtggtaatggaaaagccaaagacaaacatgagctgaggctgttgaaacatgaaacctaagaattactttaaaaccttttagtgaaatccatagttgggtgaattaaggggaaagaaaaaagaataaaggtcacataaaacagaactgtgcagtgacatcggagcagaattaactgacagttgagccttagtttggaaaagaaaaaaaatcaagataattaaagcagagaaatgtgacgaggcagtaagtagtaacaggttttcaggcctaaacaaaaaatagttttcagcgtagtgaaattcgaaaaaaaaaaagtaaattcaaaaaattgtaggaaatgggaggagcctaaatgctgtaaaggcaaatactccccaaagctgaaaaatgtatatgctaaatttgatggcataacctgtagattcctaagcagattatttcctatttaaactggtggctccagaaggaggttctggtcccattgtagcttgggatttcatcaagtagtagataaagatgtgcctgtattttgggatctgggccaaagaaatctgtgctctataaaattacggttggattcttgactcggagactattcatccttaaagaataaagaatatcagctgatagattaatatcagtctgctgaagggatatgtgtagcagaaaataaccaggaaaaaaattgaaatcttctgttggtgtctaaacaaacagttgtggaattatggaatggtagagacttgtcttaacaccattagcaaaatgtcagcagtgtgctgtgtttgccaaggaagtaaagatattcttaaacatcatcacctcttaaacagtgattatggatggggcagagtttgggactagaatttgatagcttcagttggcttatccattgatcattatggaatgttgtgtacagtatagggttttgcatattaaaagggacctagacagatcagagtgcatctaaacaagtgggagggtataaaaccatgtcattggtttagggaagaggacacttagatattttagaatttttccaatgtttatggggctgctgagttgtggccaatggtagaaattttaggagagcagattttggtttattaaaacaaatttataatatttaagatctttagagatgtctaaaaatttctcttgatactggggatatttagatagaaaatacatgcctggggttcatgttgggagattttttgtactatgcgtgattaagcaaacggttctgaacttcagttctctgaacaactgctgcgggttgttgaaggtagtgcagttggtggtggggcatttgagtgttgtaatgtttgatacagcaaaatagtgtgtacagcatattagttatgaagcattcctatgtttcatgagaacacctgtgaagctattacttacaaaaggactaaagcatttcccaaacccgtactcgttgcatgtttccatagtagtttggctataattgtatcctcctgctaggataacattcccagaagctctgattgccacgacttacaaatggaaagttttaaagacaaaacttttaaaaacaggattatggagaaaaagaataaatggggctcttgcatagtaagattggaaaccagtggatttagttcattcattgaataagtatatgaagcacagacacagagaggttgtaagaaagagacttgtctaaaatcacagttactgttagaactggaatcagaacttgctctgactcgagtgttctgaataacaccagaatgctgatgattttctacttttttcccctgtttataaaggtagtacatagttggccaaactcaacagtgagtaagatttttaagtccttgaggactggaactatgtcttagttaccttttcattcccatcattactatcttatacattaatttgtgttctgttatgaaattgagtgatatatcttagagactgaaaaaaatcgtggtagaaagacttcttatctgtcttccttattagaataagttatttgagagctggaaccatgacttgtttaactttaatgctccagaaacctagctctttcttgcctttgtttacccccaccaagtatttattgagggccagctgtgttccaggcactgttcaaggacctgcggacacactgggtaacagaatctcatggaattttgtgttattaatcaaggaatcacataggtatttataaaaatacaacttaagtggtaaatggcttgtatcatctaaccagatagatgcttatatatgaggactgtttgttttatacattttctttttatttatttttttagacagttttttgctcttgttgcccgggctggagtgcaatggcaccatctcggctcactgaaatctccgcctcctgagtacaagcgattctcctgcctcagcctcctgagtagctgggattacaggcgtctgccaccatgcccagctaatttttgtatttttagtagagacggggtttcactatgttggctaggctgctcttgaactcctgacctcaagtggtctgcctgccttggcctcccaaagtgctgggattacaggcatgagccactgcgcccggcacgtttatttaattttttaatctagtcattttatctgaattttctgataacttgcaatagattcttttgttttttttttttctaagtttgataaatttgtttatctggaaaatcagtattttttcttccctagatttattatattactatggttacttataatagtagatttttcaatattgaactattcttgcactcagggtataaattctactatatcttagtgcatttttctttttctatttatttcagatgtttgcatctatatttttaagcaaattttatctatagttttctttcatgctgctattctcaggctttgatattagtgtggtgctaactgcagtaagaataggtagtttccgaagaagatatttaaataataattatatggaaagtgcttttttagtccagagacctggctaaaggatgtatgtgtgattttaattagattaaatttttctaatgtcccttggttaattacttgctgtaattatgtaaatgatgtaaaggagtttaaattgttgcttggttgttgaaaacttgatttgagaattgctgtcatatgctaatttaagataaacaccaagttgatactggacattattaggaaagatttaattatgaagctagaagtataaaaagaaccttaggaaataataaaattctgcccttatcatgtagtcaaatccaaggtaatctctagatttttgttgattttatttgctattcagagaaaagcatttcgtaagctttgacaaacaaattgttatttaagagataaaatttgtaatatgaatattggtatcactgaagcagaaaatgtattacagtgccttttttaattgattaagattttataactgagtaagagtacctttgatcctgcctttttgctgtgttggattttatttaaccgaaactgatttaaataactagttagaaaaattaaagagtgccttcatatttgatagttcctaaggaactatcaataaattgaacttccccatgtttggaaagcatacaaaatacatttaaaaaatacattttatttttaaatactaatggatgcaagcctttctcccaaggaggacacagtctgtaggatagagagacattacatctgcatctggctaaccttaataatgacaacccttattgggcagtatgttccaaaggacaggagcctgtcttctggtgcatactagaagcttaataatttttgaaaagatccattttctttatgggactcggctcattaaaatagaattctcctacttttaaaaaaaatgtctatgtatatccaagaacagtaatacaggtctttaaaaacaggtcattgattttaggtattgctacttccttttttatatttaatatttcaatcattaacccagtgtagcttgatctgttaccctctttcccttgaaacttctctgatcaccagtggcttagtggtcaaatccaaggtaccatcttttgctgtttatcttacattcttttgtagtattcgatactgttggtcaccctgtccttgaatctgtcatttcttagcttctataacacctctcttctctagttctcctgcctacctccatttcttcttggtttggatcttcttttccttaccccttcctttttctcaagtggattccgtccttctttctcccattacttgctctaagacatctcttctgatagctttaaccattaaatttgtatatgaaactcagacttctgctgaacttgcagtctgttgtcttttgaacatctctgtttggatattccaaagatatctaaaaataatggaagtggtcccgtttaaatatccttcggacaaattagttctgaagtacaaaagaggctttggtattagttttgtctgacaggcattcttatagttgtttactgaagctatgatttggaatgactggcctcgtttagacttggaaaaattagatgaactaacaagtccatattataccggttaggaaggagctcgggctttgaagttaatttaataagatatgttatttgattcgtataaaaattggtttgtagtagaaattaattaaggctatagtcttgtttaaacatgggcttaatccagagaatgatagaataggccaggagctccttatttgagttttaatgctttatcaatgaaaccctcccctgccctctctttt

SEQ ID NO: 4 - DNA, 23 bp, Homo sapiens
agttcatctttggcagtagcatg

SEQ ID NO: 5 - DNA, 666 bp, Homo sapiens
gtaggtattcctaggggtaaatttattttttgttaaataataagatgtttatgtgcacatgctagttcgaccactttttaactttagttaatgttttgcaatttctcctgttgtgtggtcatactgtaaggagacttctgttaatttgctcttctgctttcctaaccacgttgatttggcagtgcttcactgcatcagtgcaatctgtagcattagaaagggatagaagcaacaagaatatgtgtagatcaaagcaactctaactcctgagtcctctagcttttttgtggttagaatgcaaataggacagttcttcagttcttcactcacagcaagcatttatggacctttggcaaattatattaaagtggaattttaagacaatatgataattatacttcgaaatatagacctatgatttgacatttttacaaaatgataacaaggtaatcatataggtacaacctctagcactattaaaaacagggtcttgcattgataaaaggaaacatcttgacaactgtttcataggcgtaagtttgggatgatgcaactactcttatctttcctccaacagcatctgataccttgtgcaacttcattgctaagttccttctccctttcttctgtcctctctttatttctacacccttcccccttccttag

SEQ ID NO: 6 - DNA, 1584 bp, Homo sapiens
gtagaacacaatttctgcacagctgttactcattttcaacagacacctgagccttaataatctagaaactaatacttcggaacaaatgactaattactgctgcctttgctgatttcattctgtcgttgaatctagctgttgctaagttatactgcctcccaaagtttaggctgtctgcaggctaactgttgattttggcatcattctctctcttttacttttgggtggttttgaaagatagttgcatgaaacgtacttttatttccttcaacactagagtttaacatttctgtccctttctttttctcccatctgatccctccctttaaaattgttaacatcatttcttcctcgttgttcatttattcttatgcgattcactgacccaggggaggagggaaccaaattgtgtttatataacatttgggttgcaactagaaaatttaaagctagaaaatttaaaggttccaattcatctttaaatggaagagtttgggaagaggatgagaaacaaatgcttaaggaaaagaacatatttttaatttcagtttctcaacattggctcttttgagttgtgccatactattttactagtgcagtttttgccaaggaatatttttatttcatacagcttttttaaggcatgaaaatcacaattcttatcagagatttgtgttcctaaattatttaaataattaaattaatagataatttggcactttaaaacatttttccaattgtatctgttgaatttgttttaagaacagatttaggtatgatataggtatatacatttgggaaacactgaatcaaacaaagttacaagtaggtttcttagctgggcatagtggcatgggcctgtagtcctagctactggggaagctaaggcaagaggattgcttaagcacaggagttcaagttcagcctggtgaacatagcgagatcccatttctttaaaaactttcttttttttaactgcaggccttctcagagttattaatatgccgttgtgccttgtgaatttccaggaggagattatgatatgttgtgtttcccaaatttattttaccttggaattgtttaaggaacacctagttttttcccagaactttagtgttctgagagacacagtttattattcaagaattttccatataatgtaaaccttctgtaatactaattttggttcatagattattaaagagaaaaaacaaagtatgaaaggaaactattaattattaactattattttccccaaaatcactaaagccttttaaatgttaataaagaacacatctcattctctttaatgacaagtgtcatttattgagagtattttctcacagttttgcaagtttttcagcaatgaccctaaattttatttcttggaaaccacacccaaatcctgggtgctcaattgaaataaaacaataccctagtcaatataacatgcttgttagcatctttattatttaatatagtgggtcaggaaaacattcttgttatctaggtgtgatttttaaattggggagaataaagaattaccatctaatctaagtattttttctttctctccatatttag

SEQ ID NO: 7 - DNA, 964 bp, Homo sapiens
gtttttaatgtacttttaaacattactgattatataaagttgatatttcatttttagatgtgaagaattggaatttaaattaaaataatagtatctttaattaattgtattaaaaaaagggaggctgggcgcggtggctcatacctgtaatcccagcactttgggaggctgaggtgggcagatcacaaggtcaggagttcgagatcatcctggctaacatggtgaaaccccgtatccactaaaaatacaaaaaaattagctgggtttggtggcgggcgcctgtagtcccagctactcaggagtctgaagcaggattatggtgtgaacccgggaggcagatcttgcagtgagccaagatcgtaccactgcactccagcctgggagacagcaagactccgtctcaaaaaaaataaaaattaaaattaaaaaagagaatctaacctgcaaggatcaaagttttagcttgttggcaacagctgttttccaccaggttagatacagtgcttcttgaatttattttcatgtaaagataagtatttttttttatagctaaaaatctgatttgattatttagtaatggagaattttaccagaatgtgattaaaaaccagtcttttgtcaccaaaagatcttttatgtcaccaaaataaaatccttgacggatacaacactactaggtgtatcattaaaatatattcttaatagaataaacacctagaatcagaacattggttgaaaacgtaggagctttacaggggcagtaaatttggagctagtcacatttcaaatttgccatcagtgtttctttgttgaatccagaagatagagcaaattgagctgatagtgtaagttagcaacatagttttataaaagtatttgagggactggccagaaatatactcctgactgtacatgtttcataggaagttctagaaacaacaaagcactttctatatgcttttag

SEQ ID NO: 8 - DNA, 111 bp, Homo sapiens
gtggaatctccaactgataaatcaggaaacttcttctgctcacaccactgggaaggggacagtctgaatacccaactgttcacgcagacctcgcctgtggcagatgcaccg

(19) United States
(12) Patent Application Publication
(10) Pub. No.: US 2019/0062846 A1
(43) Pub. Date: Feb. 28, 2019

(54) COMPOSITIONS AND METHODS FOR SCREENING PEDIATRIC GLIOMAS AND METHODS OF TREATMENT THEREOF

(71) Applicants: DANA-FARBER CANCER INSTITUTE, INC., BOSTON, MA (US); CHILDREN'S HOSPITAL OF PHILADELPHIA, PHILADELPHIA, PA (US)

(72) Inventors: RAMEEN BEROUKHIM, BROOKLINE, MA (US); KEITH LIGON, BROOKLINE, MA (US); ADAM RESNICK, GLENSIDE, PA (US); PRATITI BANDOPADHAYAY, BOSTON, MA (US); LORI RAMKISSOON, WEYMOUTH, MA (US); PAYAL JAIN, PHILADELPHIA, PA (US); GUILLAUME BERGTHOLD, STRASBOURG (FR); JEREMIAH WALA, CAMBRIDGE, MA (US); MARK KIERAN, BOSTON, MA (US)

(21) Appl. No.: 16/073,564

(22) PCT Filed: Jan. 27, 2017
(86) PCT No.: PCT/US2017/015448

§ 371 Date: Jul. 27, 2018

Related U.S. Application Data
(60) Provisional application No. 62/288,979, filed on Jan. 29, 2016.

(51) Int. Cl. C12Q 1/6886; G01N 33/574

(52) U.S. Cl. CPC C12Q 1/6886; G01N 33/57407; C12Q 2600/112; C12Q 2600/158; G01N 2333/82

(57) ABSTRACT

The present invention provides methods for screening and diagnosing pediatric low-grade glioma (PLGG) based on a correlation between angiocentric glioma and MYB-QKI rearrangement. Specific methods for detecting the rearrangement encompass cytogenetic methods, DNA sequencing, RNA sequencing and antibody-based methods to detect the fusion protein. The disclosure also provides methods for treating PLGGs, especially angiocentric gliomas, by suppressing the expression or activity of MYB-QKI fusion gene

### RELATED APPLICATIONS

This application claims the benefit of U.S. Provisional Application No. 62/288,979, filed Jan. 29, 2016, which is incorporated by reference herein in its entirety.

### STATEMENT REGARDING FEDERALLY SPONSORED RESEARCH OR DEVELOPMENT

This invention was made with government support under grant numbers PO1 CA142536 and R01 NS085336 awarded by the National Institutes of Health. The government has certain rights in the invention.

### BACKGROUND

Pediatric low-grade gliomas (PLGGs) encompass a heterogeneous group of World Health Organization (WHO) grade I and II tumors that collectively represent the most common pediatric brain tumor. They encompass tumors of a variety of histology, such as pilocytic astrocytoma, diffuse astrocytoma, oligodendroglioma and angiocentric glioma. Angiocentric glioma is a WHO grade I tumor that has an indolent clinical course. It arises in the cerebral cortex and shares histological features with astrocytomas and ependymomas. Angiocentric glioma causes medically refractory epileptic seizure in children. Once correctly diagnosed, it is usually cured by gross total resection of the tumor without a need for radiation or chemotherapy.

PLGGs undergo frequent alterations in the mitogen-activated protein kinase (MAPK) pathway and in MYB family genes, including MYBL1 and MYB. Alterations in BRAF, a component in a MAPK pathway, are commonly observed in a large number of PLGGs and are widely considered as a molecular marker for pilocytic astrocytoma. BRAF undergoes duplications or point mutations that render the kinase constitutively active, which are signatures for individual subtypes of PLGGs. Alterations in MYB are also heterogeneous. Several fusion partners have been reported as rare events in PLGGs, but the nature and incidence of MYB alterations in PLGGs has not been determined. Furthermore, the oncogenicity of MYB family transcription factors in the central nervous system and the mechanisms by which they contribute to gliomagenesis are yet to be defined. In light of the importance of MYB genetic alterations in PLGGs, the present invention identified an association between angiocentric gliomas and MYB-QKI rearrangement by performing a combined analysis of newly generated and published PLGG genomic data sets. This work sheds light on molecular diagnosis of angiocentric glioma and may obviate unnecessary aggressive treatments that lead to serious side effects.

### SUMMARY

In one aspect, the present disclosure provides a method of determining the likelihood that a pediatric glioma is an angiocentric glioma, the method comprising obtaining a sample of the pediatric glioma, isolating genomic DNA, RNA or protein from the pediatric glioma, and screening the genomic DNA, RNA or protein for the presence of an MYB-QKI rearrangement in the pediatric glioma, wherein the likelihood that a pediatric glioma is an angiocentric glioma is increased if the MYB-QKI rearrangement is identified in the pediatric glioma. In some embodiments, the pediatric glioma is a pediatric low-grade glioma. In another aspect, the present disclosure provides a method of identifying incidence of MYB-QKI rearrangement in a pediatric glioma cell, the method comprising screening a genomic DNA, RNA or protein from the pediatric glioma cell for the presence of an MYB-QKI rearrangement.

In some embodiments, the MYB-QKI rearrangement comprises a fusion of a MYB gene and a QKI gene. In one embodiment, the MYB-QKI rearrangement comprises a rearrangement breakpoint, wherein the rearrangement breakpoint is located in intron 4 of the QKI gene. In another embodiment, the MYB-QKI rearrangement comprises a rearrangement breakpoint, wherein the rearrangement breakpoint is located in intron 9, intron 11 or intron 15 of the MYB gene. In a particular embodiment, the fusion of the MYB gene and the QKI gene is an in-frame fusion.

In some embodiments, the screening of the genomic DNA comprises use of a cytogenetic technique. In some embodiments, the cytogenetic technique is fluorescence in situ hybridization (FISH). In one embodiment, the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 4 of a QKI gene, wherein the second region is located within 100 kb from exon 4 of the QKI gene.

In some embodiments, the screening of the genomic DNA comprises use of a molecular inversion probe. In some embodiments, the screening of the genomic DNA comprises use of genomic DNA sequencing. In one embodiment, the genomic DNA sequencing comprises whole-genome sequencing. In another embodiment, the genomic DNA sequencing comprises whole-exome sequencing.

In some embodiments, the screening of the RNA comprises screening for the presence of a MYB-QKI fusion RNA. In one embodiment, the screening of the RNA comprises use of RNA sequencing. In a particular embodiment, the mRNA sequencing comprises whole-transcriptome sequencing.

In some embodiments, the screening of the protein comprises screening for the presence of a MYB-QKI fusion protein. In one embodiment, the screening of the protein comprises use of immunohistochemistry. In some embodiments, the screening of the proteins comprises use of an anti-MYB antibody and an anti-QKI antibody. In one embodiment, the anti-MYB antibody and the anti-QKI antibody are each conjugated to a fluorescent moiety and fluorescence resonance energy transfer (FRET) between the two fluorescent moieties occurs if the two moieties are in proximity. In another embodiment, the screening of the protein comprises use of a proximity ligation assay. In yet another embodiment, the screening of the protein comprises use of an antibody that binds specifically to a joint region of the fusion protein wherein the joint region comprises at least one amino acid of the MYB protein sequence and at least one amino acid of the QKI protein sequence. In another embodiment, the screening of the protein comprises use of mass spectrometry.

In another aspect, the present disclosure provides a kit for detecting MYB-QKI rearrangement, comprising at least a first FISH probe that hybridizes to a first region 5′ to exon 16 of a MYB gene, wherein the first region is located within 100 kb from exon 16 of the MYB gene. In some embodiments, the kit further comprises a second FISH probe that hybridizes to a second region 3′ to exon 4 of a QKI gene, wherein the second region is located within 100 kb from exon 4 of the QKI gene.

In another aspect, the present disclosure provides a method of treating a pediatric glioma in a subject in need thereof comprising obtaining a sample of the pediatric glioma, isolating genomic DNA, RNA or protein from the pediatric glioma, screening the genomic DNA, RNA or protein for the presence of an MYB-QKI rearrangement in the pediatric gliomas, wherein the likelihood that a pediatric glioma is an angiocentric glioma is increased if MYB-QKI rearrangement is identified in the pediatric glioma, and performing surgical resection on the subject if the MYB-QKI rearrangement is present, thereby treating the pediatric glioma in the subject. In some embodiments, the pediatric glioma is a pediatric low-grade glioma. In another aspect, the present disclosure provides a method of treating a pediatric glioma in a subject in need thereof, the method comprising screening a genomic DNA, RNA or protein from the pediatric glioma for the presence of an MYB-QKI rearrangement; and performing surgical resection on the subject if the MYB-QKI rearrangement is present, thereby treating the pediatric glioma in the subject.

In some embodiments, the MYB-QKI rearrangement comprises a fusion of a MYB gene and a QKI gene. In one embodiment, the MYB-QKI rearrangement comprises a rearrangement breakpoint, wherein the rearrangement breakpoint is located in intron 4 of the QKI gene. In another embodiment, the MYB-QKI rearrangement comprises a rearrangement breakpoint, wherein the rearrangement breakpoint is located in intron 9, intron 11 or intron 15 of the MYB gene. In some embodiments, the fusion of the MYB gene and the QKI gene is an in-frame fusion.

In some embodiments, the screening of the genomic DNAs comprises use of a cytogenetic technique. In one embodiment, the cytogenetic technique is fluorescence in situ hybridization (FISH). In a particular embodiment, the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 4 of a QKI gene, wherein the second region is located within 100 kb from exon 4 of the QKI gene.

In some embodiments, the screening of the genomic DNA comprises use of a molecular inversion probe. In some embodiments, the screening of the genomic DNA comprises use of genomic DNA sequencing. In one embodiment, the genomic DNA sequencing comprises whole-genome sequencing. In another embodiment, the genomic DNA sequencing comprises whole-exome sequencing.

In some embodiments, the screening of the RNA comprises screening for the presence of a MYB-QKI fusion RNA. In one embodiment, the screening of the RNA comprises use of mRNA sequencing. In a particular embodiment, the mRNA sequencing comprises whole-transcriptome sequencing.

In some embodiments, the screening of the protein comprises screening for the presence of a MYB-QKI fusion protein. In one embodiment, the screening of the protein comprises use of immunohistochemistry. In some embodiments, the screening of the protein comprises use of an anti-MYB antibody and an anti-QKI antibody. In one embodiment, the anti-MYB antibody and the anti-QKI antibody are each conjugated to a fluorescent moiety and fluorescence resonance energy transfer (FRET) between the two fluorescent moieties occurs if the two moieties are in proximity. In another embodiment, the screening of the protein comprises use of a proximity ligation assay. In yet another embodiment, the screening of the protein comprises use of an antibody that binds specifically to a joint region of the fusion protein wherein the joint region comprises at least one amino acid of the MYB protein sequence and at least one amino acid of the QKI protein sequence. In one embodiment, the screening of the protein comprises use of mass spectrometry.

In some embodiments, radiation therapy is not provided to the subject if an MYB-QKI rearrangement is identified. In some embodiments, chemotherapy is not provided to the subject if the MYB-QKI rearrangement is identified.

In another aspect, the present disclosure provides a method for treating a pediatric glioma having an MYB-QKI rearrangement in a subject in need thereof, wherein the MYB-QKI rearrangement comprises an MYB-QKI fusion DNA, the method comprising administering to the subject an agent that deletes at least a 100 bp portion of the MYB-QKI fusion DNA. In another aspect, the present disclosure also provides a method for treating a pediatric glioma having an MYB-QKI rearrangement in a subject in need thereof, wherein the MYB-QKI rearrangement comprises an MYB-QKI fusion DNA, the method comprising administering to the subject an agent that generates a frame-shifting mutation of the MYB-QKI fusion DNA. In some embodiments, the agent comprises a Cas9 protein or a polynucleotide encoding a Cas9 protein; and a CRISPR-Cas system guide RNA polynucleotide targeting the MYB-QKI genomic locus.

In another aspect, the present disclosure provides a method for treating a pediatric glioma having a MYB-QKI rearrangement in a subject in need thereof, wherein the MYB-QKI rearrangement comprises an MYB-QKI fusion DNA which is transcribed to an MYB-QKI fusion RNA, the method comprising administering to the subject an agent that reduces the amount of the MYB-QKI fusion RNA in a cell in the subject.

In some embodiments, the agent comprises an interfering RNA that targets an MYB-QKI mRNA. In some embodiments, the agent inhibits the activity of at least one enhancer in the region within or 3′ to the genomic location of the MYB-QKI fusion gene that is operably linked to the genomic sequence of the MYB-QKI. In one embodiment, the enhancer is located within 15 kb from the genomic location of the 5′ end of the MYB portion of the MYB-QKI fusion DNA. In another embodiment, the enhancer is located between 100 kb and 500 kb from the genomic location of the 3′ end of the MYB-QKI fusion DNA. In some embodiments, the agent comprises an antagonist of BET. In some embodiments, the antagonist of BET is selected from the group consisting of JQ1, GSK1210151A, GSK525762, OTX-015, TEN-010, CPI-203, CPI-0610, RVX-208, and LY294002. In some embodiments, the antagonist of BET is JQ1. In some embodiments, the agent comprises an antagonist of CDK7. In some embodiments, the antagonist of CDK7 is selected from the group consisting of THZ1, BS-181, flavopiridol, P276-00, R-roscovitine, R547, SNS-032, and ZK 304709. In some embodiments, the antagonist of CDK7 is THZ1. In some embodiments, the agent reduces the amount of MYB-QKI fusion RNA comprises inhibiting H3K27 acetylation at the MYB-QKI locus.

In another aspect, the present disclosure provides a method for treating a pediatric glioma having an MYB-QKI rearrangement in a subject, wherein the MYB-QKI rearrangement comprises an MYB-QKI fusion DNA which is transcribed to an MYB-QKI fusion RNA, which RNA is translated to an MYB-QKI fusion protein, the method comprising administering to the subject an agent that reduces the amount or activity of the MYB-QKI fusion protein.

In some embodiments, the agent increases the rate of MYB-QKI protein degradation. In one embodiment, the agent inhibits the activity of at least one deubiquitinating enzyme. In some embodiments, the antagonist of DUB is selected from the group consisting of PR619, VLX1570, b-AP15, PX-478, and WPI 130. In some embodiments, the antagonist of DUB is PR619.

In another aspect, the present disclosure provides a method for treating a pediatric glioma having a MYB-QKI rearrangement in a subject in need thereof, the method comprising administering to the subject an antagonist of c-Kit. In some embodiments, the antagonist of c-Kit is selected from the group consisting of axitinib, dovitinib, dasatinib, imatinib, motesanib, pazopanib, masitinib, vatalanib, cabozantinib, tivozanib, OSI-930, Ki8751, telatinib, pazopanib, and tyrphostin AG 1296. In some embodiments, the antagonist of c-Kit is dasatinib.

In some embodiments, the pediatric glioma is a pediatric low-grade glioma. In one embodiment, the pediatric low-grade glioma is an angiocentric glioma.

In another aspect, the present disclosure provides a method of determining an increased likelihood that a pediatric glioma is an angiocentric glioma, comprising obtaining a sample of the pediatric glioma, isolating genomic DNA from the pediatric glioma, and screening the genomic DNA for the presence of an MYB alteration in the pediatric glioma, wherein the likelihood that a pediatric glioma is an angiocentric glioma is increased if an MYB alteration is identified in the pediatric glioma. In some embodiments, the pediatric glioma is a pediatric low-grade glioma. In another aspect, the present disclosure provides a method of classifying a pediatric glioma, the method comprising screening a genomic DNA from the pediatric glioma for the presence of an MYB alteration.

In some embodiments, the MYB alteration comprises one or more of: a copy number alteration, a truncation, a fusion, a rearrangement, a 5′ deletion, a 3′ deletion, a breakpoint, a translocation, an inversion, and an insertion. In one embodiment, the MYB alteration comprises a 3′ deletion of MYB. In a particular embodiment, the 3′ deletion of MYB comprises a deletion of a portion of the MYB gene comprising all exons and introns 3′ to MYB intron 15. In another particular embodiment, the 3′ deletion of MYB comprises a deletion of a portion of the MYB gene comprising all exons and introns 3′ to MYB intron 11. In yet another embodiment, the 3′ deletion of MYB comprises a deletion of a portion of the MYB gene comprising all exons and introns 3′ to MYB intron 9.

In some embodiments, the expression level of MYB exons 1-9 is higher in the pediatric glioma with the MYB alteration than in a control sample. In one embodiment, the control sample comprises a sample of pediatric glioma wherein MYB alteration does not occur. In another embodiment, the control sample comprises a sample of normal brain or spine tissue.

In some embodiments, the screening of the genomic DNA comprises use of a cytogenetic technique. In one embodiment, the cytogenetic technique is fluorescence in situ hybridization (FISH). In a particular embodiment, the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 15 of a MYB gene, wherein the second region is located within 100 kb from exon 15 of the MYB gene.

In some embodiments, the screening of the genomic DNA comprises use of genomic DNA sequencing. In one embodiment, the genomic DNA sequencing comprises whole-genome sequencing. In another embodiment, the genomic DNA sequencing comprises whole-exome sequencing.

In another aspect, the present disclosure provides a kit for detecting MYB alteration, comprising at least a first FISH probe that hybridizes to a first region 5′ to exon 16 of a MYB gene, wherein the first region is located within 100 kb from exon 16 of the MYB gene. In one embodiment, the kit further comprises a second FISH probe that hybridizes to a second region 3′ to exon 15 of a MYB gene, wherein the second region is located within 100 kb from exon 15 of the MYB gene.

In another aspect, the present disclosure provides a method of treating a pediatric glioma in a subject in need thereof, the method comprising obtaining a sample of the pediatric glioma, isolating genomic DNA from the pediatric glioma, screening the genomic DNA for the presence of an MYB alteration in the pediatric gliomas, wherein the likelihood that a pediatric glioma is an angiocentric glioma is increased if an MYB alteration is identified in the pediatric glioma, and performing surgical resection on the subject if the MYB alteration is present, thereby treating the pediatric glioma in the subject. In another aspect, the present disclosure provides a method of treating a pediatric glioma in a subject in need thereof, the method comprising screening a genomic DNA from the pediatric glioma for the presence of an MYB alteration; and performing surgical resection on the subject if the MYB alteration is present, thereby treating the pediatric glioma in the subject.

In some embodiments, the pediatric glioma is a pediatric low-grade glioma.

In some embodiments, the MYB alteration comprises one or more of: a copy number alteration, a truncation, a fusion, a rearrangement, a 5′ deletion, a 3′ deletion, a breakpoint, a translocation, an inversion, and an insertion. In one embodiment, the MYB alteration comprises a 3′ deletion of MYB. In a particular embodiment, the 3′ deletion of MYB comprises a deletion of a portion of the MYB gene comprising all exons and introns 3′ to MYB intron 15. In another particular embodiment, the 3′ deletion of MYB comprises a deletion of a portion of the MYB gene comprising all exons and introns 3′ to MYB intron 11. In yet another embodiment, the 3′ deletion of MYB comprises a deletion of a portion of the MYB gene comprising all exons and introns 3′ to MYB intron 9.

In some embodiments, the expression level of MYB exons 1-9 is higher in the pediatric glioma with the MYB alteration than in a control sample. In one embodiment, the control sample comprises a sample of pediatric glioma wherein MYB alteration does not occur. In another embodiment, the control sample comprises a sample of normal brain or spine tissue.

In some embodiments, the screening of the genomic DNAs comprises use of a cytogenetic technique. In one embodiment, the cytogenetic technique is fluorescence in situ hybridization (FISH). In a particular embodiment, the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 15 of a MYB gene, wherein the second region is located within 100 kb from exon 15 of the MYB gene.

In some embodiments, the screening of the genomic DNA comprises use of genomic DNA sequencing. In one embodiment, the genomic DNA sequencing comprises whole-genome sequencing. In another embodiment, the genomic DNA sequencing comprises whole-exome sequencing.

In some embodiments, radiation therapy is not provided to the subject if an MYB rearrangement is identified. In some embodiments, chemotherapy is not provided to the subject if the MYB rearrangement is identified.

In another aspect, the present disclosure provides a method for treating a pediatric glioma having an MYB alteration in a subject in need thereof, the method comprising administering to the subject an agent that deletes at least a 100 bp portion of the MYB DNA from the genomic location of MYB alteration. In another aspect, the present disclosure also provides a method for treating a pediatric glioma having an MYB alteration in a subject in need thereof, the method comprising administering to the subject an agent that generates a frame-shifting mutation of the MYB DNA from the genomic location of MYB alteration.

In some embodiments, the MYB alteration comprises one or more of: a copy number alteration, a truncation, a fusion, a rearrangement, a 5′ deletion, a 3′deletion, a breakpoint, a translocation, an inversion, and an insertion. In one embodiment, the MYB alteration comprises a 3′ deletion of MYB. In a particular embodiment, the 3′ deletion of MYB comprises a deletion of a portion of the MYB gene comprising all exons and introns 3′ to MYB intron 15. In another particular embodiment, the 3′ deletion of MYB comprises a deletion of a portion of the MYB gene comprising all exons and introns 3′ to MYB intron 11. In yet another particular embodiment, the 3′ deletion of MYB comprises a deletion of a portion of the MYB gene comprising all exons and introns 3′ to MYB intron 9. In some embodiments, the MYB DNA is deleted or mutated by CRISPR technology. In some embodiments, the agent comprises a Cas9 protein or a polynucleotide encoding a Cas9 protein; and a CRISPR-Cas system guide RNA polynucleotide targeting the MYB genomic locus.

In another aspect, the present disclosure provides a method for treating a pediatric glioma having a MYB alteration in a subject in need thereof, the method comprising administering to the subject an agent that reduces the amount of the MYB RNA in a cell in the subject.

In some embodiments, the MYB alteration comprises one or more of: a copy number alteration, a truncation, a fusion, a rearrangement, a 5′ deletion, a 3′deletion, a breakpoint, a translocation, an inversion, and an insertion. In one embodiment, the MYB alteration comprises a 3′ deletion of MYB. In a particular embodiment, the 3′ deletion of MYB comprises a deletion of a portion of the MYB gene comprising all exons and introns 3′ to MYB intron 15. In another particular embodiment, the 3′ deletion of MYB comprises a deletion of a portion of the MYB gene comprising all exons and introns 3′ to MYB intron 11. In yet another embodiment, the 3′ deletion of MYB comprises a deletion of a portion of the MYB gene comprising all exons and introns 3′ to MYB intron 9.

In some embodiments, the agent comprises an interfering RNA that targets a MYB mRNA. In some embodiments, the agent inhibits the activity of at least one enhancer that is operably linked to the genomic sequence of MYB. In some embodiments, the agent comprises an antagonist of BET. In some embodiments, the antagonist of BET is selected from the group consisting of JQ1, GSK1210151A, GSK525762, OTX-015, TEN-010, CPI-203, CPI-0610, RVX-208, and LY294002. In some embodiments, the antagonist of BET is JQ1. In some embodiments, the agent comprises an antagonist of CDK7. In some embodiments, the antagonist of CDK7 is selected from the group consisting of THZ1, BS-181, flavopiridol, P276-00, R-roscovitine, R547, SNS-032, and ZK 304709. In some embodiments, the antagonist of CDK7 is THZ1. In some embodiments, the agent inhibits H3K27 acetylation at the MYB locus where MYB alteration occurs.

In another aspect, the present disclosure also provides a method for treating a pediatric glioma having an MYB alteration in a subject, the method comprising administering to a subject an agent that reduces the amount or activity of the MYB protein.

In some embodiments, the MYB alteration comprises one or more of: a copy number alteration, a truncation, a fusion, a rearrangement, a 5′ deletion, a 3′ deletion, a breakpoint, a translocation, an inversion, and an insertion. In one embodiment, the MYB alteration comprises a 3′ deletion of MYB. In a particular embodiment, the 3′ deletion of MYB comprises a deletion of a portion of the MYB gene comprising all exons and introns 3′ to MYB intron 15. In another particular embodiment, the 3′ deletion of MYB comprises a deletion of a portion of the MYB gene comprising all exons and introns 3′ to MYB intron 11. In yet another embodiment, the 3′ deletion of MYB comprises a deletion of a portion of the MYB gene comprising all exons and introns 3′ to MYB intron 9.

In some embodiments, the agent increases the rate of MYB protein degradation. In one embodiment, the agent inhibits the activity of at least one DUB. In some embodiments, the antagonist of DUB is selected from the group consisting of PR619, VLX1570, b-AP15, PX-478, and WPI 130. In some embodiments, the antagonist of DUB is PR619.

In another aspect, the present disclosure provides a method for treating a pediatric glioma having a MYB alteration in a subject in need thereof, the method comprising administering to the subject an antagonist of c-Kit. In some embodiments, the antagonist of c-Kit is selected from the group consisting of axitinib, dovitinib, dasatinib, imatinib, motesanib, pazopanib, masitinib, vatalanib, cabozantinib, tivozanib, OSI-930, Ki8751, telatinib, pazopanib, and tyrphostin AG 1296. In some embodiments, the antagonist of c-Kit is dasatinib.

In some embodiments, the pediatric glioma is a pediatric low-grade glioma. In one embodiment, the pediatric low-grade glioma is an angiocentric glioma.

### BRIEF DESCRIPTION OF DRAWINGS

FIG. 1 is a diagram showing at least three mechanisms through which MYB-QKI rearrangement contributes to oncogenesis, as suggested by our data. The MYB-QKI rearrangement disrupts both MYB and QKI, resulting in hemizygous deletion of the 3′ portion of MYB and the 5′ portion of QKI. (1) This results in proximal translocation of H3K27ac-bound enhancers in the 3′ region of QKI to the MYB promoter, resulting in MYB promoter activation. (2) The MYB-QKI fusion protein that is expressed is oncogenic, functions as a transcription factor and exhibits the ability to bind to and activate the MYB promoter, resulting in an autoregulatory feedback loop. (3) Hemizygous loss of QKI results in suppression of the expression of QKI, which functions as a tumor-suppressor gene.

FIG. 2 is a graph showing analyses of significantly recurrent somatic copy-number alterations across all samples with whole-genome sequencing data

FIG. 3 is a graph showing recurrent rearrangement involving MYB and QKI in angiocentric gliomas. Driver alterations were identified in 154 of 172 PLGGs profiled with whole-genome sequencing and/or RNA-seq. Histological subtypes included pilocytic astrocytoma (PA), pilomyxoid astrocytoma (PMA), angiocentric glioma (AG), oligodendroglioma (OD), diffuse astrocytoma (DA), dysembryoplastic neuroepithelial tumor (DNT), ganglioglioma (GG), pleomorphic xanthoastrocytoma (PXA) and PLGG not otherwise specified (NOS). Tumors for which histology is unavailable are designated NA. The dashed box highlights angiocentric gliomas.

FIG. 4 is a series of photographs showing three patterns in PLGG: MYB disomy, MYB rearrangement, and 3′ MYB deletion, by FISH analysis using probes flanking MYB. Formalin-fixed, paraffin-embedded tissue sections were examined using homebrew probes RP11-63K22 (5′ to MYB; directly labeled in SpectrumOrange) and RP11-170P19 (3′ to MYB; directly labeled in SpectrumGreen) that map to 6q23.3. MYB status was assessed in 50 tumor nuclei per sample. A CEP6 (centromere of chromosome 6) aqua probe (Invitrogen) mapping to the centromeric region of chromosome 6 was co-hybridized as a control. Scale bars are 5 μm.

FIG. 5 is a graph showing recurrent genetic events across 54 pediatric gliomas profiled by whole exome sequencing or array comparative genomic hybridization (aCGH). Histological subtypes included pilocytic astrocytoma (PA), pilomyxoid astrocytoma (PMA), angiocentric glioma (AG), oligodendroglioma (OD), diffuse astrocytoma (DA), dysembryoplastic neuroepithelial tumor (DNT), ganglioglioma (GG), pleomorphic xanthoastrocytoma (PXA) and PLGG not otherwise specified (NOS). Tumors for which histology is unavailable are designated NA. The dashed box highlights angiocentric gliomas.

FIG. 6 is a schematic showing breakpoints observed in MYB and QKI in angiocentric gliomas. Sequence across the breakpoints, as determined by RNA-seq, is shown for each rearrangement.

FIG. 7 is a graph showing copy number profiles from whole-genome sequencing data for MYB and QKI in three angiocentric gliomas (BCH_3084, BCH_DF005 and BCH_DF003). Arrows highlight breakpoints in MYB and QKI.

FIG. 8 is a schematic showing structures of the MYB-QKI fusion proteins. The N terminus of QKI exons 5-8 includes QUA2 domains. MYB-QKI5 retains an NLS. Two variants (short and long) of MYB-QKI are depicted corresponding to different breakpoints in MYB. The long variants comprise a negative regulatory domain.

FIG. 9 is a graph showing MYB expression (by Reads Per Kilobase of transcript per Million (RPKM), mean values represented by the horizontal bar) in brain cortex (n=47), normal human colon (n=12), breast (n=27), whole blood (n=51), esophagus (n=38), and skin (n=25).

FIG. 10 is a series of photographs showing (left) MYB immunohistochemistry on human adult frontal cortex and (right) human adult white matter (scale bars, 100 μm).

FIG. 11 is a series of photographs showing Hematoxylin and eosin staining (left) and MYB immunohistochemistry (right) of human fetal neural stem cells generated from the ganglionic eminence at 22 weeks of gestation (scale bars, 100 μm).

FIG. 12 is a series of photographs showing (upper left) Sagittal section from an E14.5 mouse brain (scale bar, 500 μm); (upper right) hematoxylin and eosin staining of an E14.5 mouse ganglionic eminence (GE) including ventricular (GE-VZ) and subventricular (GE-SVZ) zones (scale bar, 50 μm); (lower left) MYB immunohistochemistry on the E14.5 mouse ganglionic eminence (scale bar, 50 μm); and (lower right) MYB immunohistochemistry of the region indicated by * in the lower left panel, which demonstrates positive staining in the subventricular zone (scale bar, 50 μm).

FIG. 13 is a series of photographs showing (left) hematoxylin and eosin staining of the periventricular region of adult mouse brain (scale bar, 100 μm) and (right) immunohistochemistry for MYB in the ependymal/subventricular zone layer of the adult mouse brain wherein arrows highlight MYB positive cells (scale bar, 100 μm).

FIG. 14 is a series of graphs showing (left) significance of deletions (x axis) along the 6q chromosome (y axis) in adult human glioblastomas (G score refers to GISTIC score (Beroukhim et al.)) and (middle and right) heat maps showing copy number profiles at 6q for individual adult human glioblastomas.

FIG. 15 is a graph showing the expression signature in mouse neural stem cells (mNSCs) expressing MYB-QKI5 or MYB-QKI6 relative to cells expressing eGFP control. The genes on the left panel are expressed at a higher level in mNSCs expressing MYB-QKI than in cells expressing eGFP control. In the cells expressing eGFP, a higher density of color indicates a lower expression level of a gene. In the cells expressing MYB-QKI, a higher density of color indicates a higher expression level of a gene. The genes on the right panel are expressed at a lower level in mNSCs expressing MYB-QKI than in cells expressing eGFP control. In the cells expressing eGFP, a higher density of color indicates a higher expression level of a gene. In the cells expressing MYB-QKI, a higher density of color indicates a lower expression level of a gene.

FIG. 16 is a series of graphs showing heat maps of (left) MYB-QKI5 and (right) H3K27ac signals in MYB-QKI regions, with data generated from chromatin immunoprecipitation with parallel sequencing. Each row is centered on a MYB-QKI peak. The regions are rank-ordered by MYB-QKI signal. Scaled intensities are in units of reads per bin.

FIG. 17 is a graph showing MYB-QKI binding to the endogenous Myb promoter.

FIG. 18 is a graph showing percentage of MYB-QKI signature genes with evidence of MYB-QKI ChIP-seq binding for upregulated (n=25) and downregulated (n=25) genes. **P<0.001, paired t test.

FIG. 19A is a graph showing the induction of mim-1 reporter following transfection to express truncated MYB encoded by exons 1-9 (MYBtr), MYB-QKI5, MYBQKI6 or full-length MYB in HEK293T cells. The values shown represent the means of three independent measurements ±s.e.m.

FIG. 19B is a Western blot showing the expression levels of MYBtr, MYB-QKI5, MYBQKI6 and full-length MYB in HEK293T cells used for the mim-promoter assays in FIG. 18A.

FIG. 19C is a graph and a Western blot showing the correlation between the expression levels of MYB-QKI and its activity in inducing mim-1 reporter in HEK293T cells.

FIG. 20 is a graph showing expression of the MYB-QKI signature in normal pediatric brain samples (n=8), PLGGs without MYB-QKI rearrangement (n=8) and angiocentric gliomas with MYB-QKI rearrangement (n=4). Bars represent mean expression of the signature in tumors ±s.e.m. Expression of the signature within each tumor was calculated as the sum of the RPKM values for each gene in the signature.

FIG. 21 is a graph showing expression of genes associated with MYB pathway activation as compared to normal brain.

FIG. 22 is a graph showing MYB expression levels (in RPKM) of tumors with MYB-QKI rearrangement (“MYB-QKI”, n=5) relative to normal brain (“normal”, n=10) or BRAF- or FGFR-driven PLGGs (“Non-MYB-QKI”, n=10). Values shown represent means±s.e.m.

FIG. 23 is a graph showing exon-specific expression of MYB in angiocentric gliomas that harbor MYB-QKI rearrangement (n=3) relative to PLGGs that harbor BRAF alterations without MYB-QKI rearrangement (n=4). Values shown represent means±s.e.m.

FIG. 24 is a graph showing ChIP-seq binding peaks of (top track) H3K27ac binding within the Qk (a mouse homologue of human QKI) loci in mouse neural stem cells and MYB-QKI binding within the Qk loci in mouse neural stem cells (bottom track).

FIG. 25 is a graph showing H3K27ac signal within the MYB and QKI loci in human frontal and temporal lobes (original data from Encyclopedia of DNA Elements, ENCODE). Values shown depict the mean number of nucleotides that are associated with H3K27ac (per kb) in MYB and QKI across both locations ±s.e.m. (n=1 ChIP-seq map for each location).

FIG. 26 is a graph showing predicted H3K27ac-associated enhancer elements in MYB-QKI, with translocation of genomic enhancers from the 3′ region of QKI to within 15 kb of the 5′ end of MYB. The enhancer maps shown are derived from ENCODE data for normal human brain (frontal and temporal lobes). Q3E1 represents an H3K27ac-associated enhancer present in the ENCODE data from normal brain.

FIG. 27 is a graph showing H3K27ac enhancer peaks in proximity to MYB and QKI in a BRAF-duplicated pilocytic astrocytoma (top) and an angiocentric glioma harboring MYB-QKI rearrangement (bottom). Q3E1 is an enhancer associated with the 3′ UTR of QKI. Two super-enhancer clusters (Q3SE1 and Q3SE2) are located within 500 kb of QKI. Angiocentric gliomas were associated with aberrant enhancer formation at the MYB promoter (M5E1), which was not detected in the BRAF-driven pilocytic astrocytoma. The breakpoint for the MYB-QKI rearrangement between exons 1-9 of MYB and exons 5-8 of QKI, as determined by RNA-seq, is depicted for the angiocentric glioma harboring MYB-QKI rearrangement.

FIG. 28 is a graph showing the presence of active enhancer elements that are translocated proximal to the MYB promoter.

FIG. 29 is a graph showing super-enhancers associated with the 3′ end of QKI (Q3SE1 and Q3SE2), presented for two angiocentric gliomas (AG1 and AG2). SE means super-enhancer.

FIG. 30 is a graph showing the lack of formation of H3K27ac enhancer peaks at MYB in the pilocytic astrocytoma.

FIG. 31 is a graph showing MYB promoter activation following transfection of the MYB-luc construct into U87 cells and U87 cells overexpressing MYB-QKI with and without the Q3E1 enhancer cloned into the MYB-luc construct. Changes in luciferase activity from the MYB-luc reporter are shown as the means±s.e.m. of three individual replicate experiments with n=5 each.

FIG. 32A is a series of graphs showing that MYB-QKI induces the MYB promoter in HEK293T and NIH3T3 cells.

FIG. 32B is a Western blot showing expression of MYB-QKI in NIH3T3 cells expressing full-length MYB, MYBQKI5, MYBQKI6 or vector control.

FIG. 33A is a graph showing in vitro cell proliferation (number of cells relative to baseline) of mNSCs overexpressing eGFP or MYBTr^{exons 1-9}. The mean values for five independent pools are depicted. Error bars, s.e.m.

FIG. 33B is a graph showing the expression levels of full-length and truncated MYB in mNSCs used for the cell proliferation assay in FIG. 33A.

FIG. 34A is a graph showing tumor growth following flank injections of NIH3T3 cells overexpressing MYB, MYBTr^{exons1-15} or a vector control. The means of five measurements are depicted. Error bars, s.e.m.

FIG. 34B is a Western blot showing the expression levels of full-length and truncated MYB in mNSCs used for the tumor growth assay in FIG. 34A.

FIG. 34C is a representative image for intracranial mNSC-MYB-QKI6 tumors.

FIG. 35A is a series of photographs showing hematoxylin and eosin analysis of severe combined immunodeficient (SCID) mouse brains after striatal injections with mNSCs expressing eGFP, truncated MYB, MYB-QKI5 or MYB-QKI6. Scale bars, 2 mm (top) and 50 μm (bottom).

FIG. 35B is a graph showing Kaplan-Meier survival analysis of orthotopic SCID mice injected with mNSCs overexpressing truncated MYB, MYB-QKI5 or MYB-QKI6 that develop tumors with short latency in comparison to mice injected with mNSCs expressing eGFP, which never develop tumors (P<0.01).

FIG. 35C is a series of photographs showing that the tumors described in FIGS. 34A and 34B expressed OLIG2 and GFAP in a subset of tumor cells, a pattern similar to that observed in human diffuse gliomas.

FIG. 36A is a graph showing in vitro cell proliferation of mNSCs that overexpress MYB-QKI5 (short), MYB-QKI6 (short) or eGFP control. The means of five independent pools are depicted. Error bars, s.e.m.

FIG. 36B is a Western blot showing the expression levels of QKI5 (short) and MYB-QKI6 (short) in mNSCs used for the proliferation assay in FIG. 36A.

FIG. 37A is a graph showing tumor growth following flank injections of NIH3T3 cells overexpressing MYB, MYB-QKI5 (long), MYB-QKI6 (long) or vector control. The mean of five measurements is depicted. Error bars, s.e.m.

FIG. 37B is a series of photographs showing representative images of intracranial mNSC-MYB-QKI tumors.

FIG. 38 is a graph showing exon-specific expression of QKI in angiocentric gliomas (n=5) relative to BRAF-driven PLGGs (n=5). Values represent means±s.e.m. RNA-seq data for exon 8 of QKI showed a high number of duplicate reads and thus are not shown.

FIG. 39A is a graph showing cell proliferation of mNSCs expressing human truncated MYB, MYB-QKI5, MYB-QKI6 or eGFP control with suppression of wild-type mouse Qk. Values represent the means of four independent experiments ±s.e.m. shLacZ, control shRNA targeting lacZ; shQKI, shRNA targeting human QKI and mouse Qk.

FIG. 39B is a graph showing expression of wild type Qk in mNSCs administered a short hairpin RNAs (shRNAs) that targeted the first four exons of Qk.

FIG. 39C is a graph showing proliferation of mNSCs expressing human truncated MYB, MYB-QKI5, MYB-QKI6 or eGFP control with and without suppression of wild-type mouse Qk.

FIG. 40 is a graph showing a defined signature consisting of the 50 genes whose expression was most correlated with Qk suppression by shQKT versus control LacZ. Red indicates higher expression—the more intensity the color, the higher the expression level; blue indicates lower expression—the more intensity the color, the lower the expression level.

FIG. 41 is a graph showing expression of genes from the Qk suppression signature in normal human brain (n=8) and angiocentric gliomas (n=5). Expression of the signature in each sample was calculated as the sum of the RPKM values for each gene in the signature.

FIGS. 42A-D are a set of graphs showing percentage viability of mouse neural stem cells expressing GFP (FIG. 42A), truncated MYB (FIG. 42B), MYB-QKI5 (short variant) (FIG. 42C), and MYB-QKI6 (short variant) (FIG. 42D) after the treatment of increased doses (0, 0.05, 0.1, 0.3, 0.5, and 1 μM) of JQ1, THZ1 (“CDK7i”), and DMSO as negative control.

FIGS. 43A-D are a set of graphs showing cell growth of mouse neural stem cells expressing GFP (FIG. 43A), truncated MYB (FIG. 43B), MYB-QKI5 (short variant) (FIG. 43C), and MYB-QKI6 (short variant) (FIG. 43D) over a time course (0, 1, 3, and 5 days) in the presence of 0.5 μM or 1 μM of LY2835219 (“CDK4/6 inhibitor”).

FIG. 43E is a Western blot probing CDK6 and β-actin (loading control) in samples of mouse neural stem cells expressing GFP (“GFP”), truncated MYB (“MYB Tr”), MYB-QKI5 (short variant) (“MYB-QKI5”), and MYB-QKI6 (short variant) (“MYB-QKI6”).

FIG. 44 is a graph showing the percentage viability of mouse neural stem cells expressing GFP (“GFP”), truncated MYB (“MYBtr”), MYB-QKI5 (short variant) (“MYBQKI5”), and MYB-QKI6 (short variant) (“MYBQKI6”) after the treatment of increased doses (0, 0.05, 0.1, 0.3, 0.5, and 1 μM) of dasatinib or DMSO as negative control.

FIG. 45 is a graph showing the quantity of truncated MYB or MYB-QKI fusion protein after the treatment with PR619.

### DETAILED DESCRIPTION

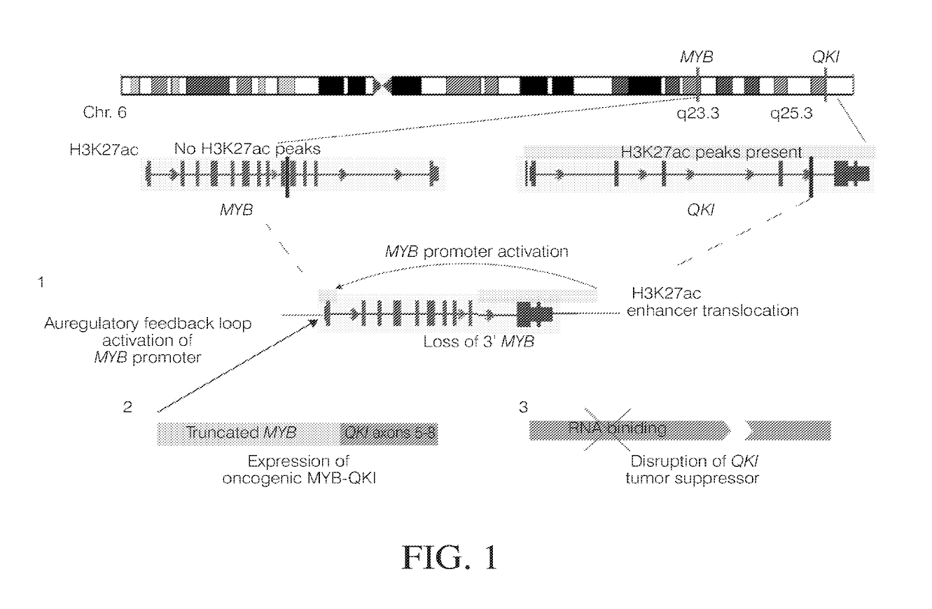

The present disclosure identifies novel factors associated with angiocentric gliomas. Among subtypes of pediatric low-grade gliomas (PLGGs), angiocentric gliomas are specifically associated with MYB-QKI rearrangement on chromosome 6. Almost all angiocentric gliomas undergo MYB-QKI rearrangement, and none of the other subtypes of PLGGs carry this rearrangement. MYB-QKI rearrangement may contribute to oncogenicity through three mechanisms (FIG. 1). First, the alteration results in proximal translocation of H3K27ac-bound enhancers in the 3′ region of QKI to the MYB promoter, resulting in MYB promoter activation. Second, the MYB-QKI fusion protein that is expressed is oncogenic, functions as a transcription factor and exhibits the ability to bind to and activate the MYB promoter, resulting in an autoregulatory feedback loop. Third, hemizygous loss of QKI results in suppression of QKI, which functions as a tumor-suppressor gene. Taken together, disruptions of both MYB and QKI appear to contribute to tumor formation in a cooperative manner. Each of the three mechanisms may be associated with angiocentric glioma, and disruption of any of them may provide a treatment for PLGGs.

In one aspect, the present disclosure provides a method of classifying a pediatric glioma, the method comprising screening a genomic DNA, RNA or protein from the pediatric glioma cell for the presence of an MYB-QKI rearrangement. In some embodiments some embodiments, MYB-QKI rearrangement is examined using a sample of PLGG that has been removed from a subject by surgery or biopsy. The presence of a MYB-QKI fusion DNA, RNA or protein is examined. If a fusion DNA, RNA or protein is detected, MYB-QKI rearrangement has occurred and the likelihood that the PLGG is angiocentric glioma is increased. If a fusion DNA, RNA or protein is not detected while control assays confirm that the absence of detection is not due to low quality of the sample or other technical issues, MYB-QKI rearrangement has not occurred and the likelihood that the PLGG is angiocentric glioma is decreased.

In some embodimentssome embodiments, a subject that is diagnosed by the method above to have an increased likelihood to carry an angiocentric glioma is treated with surgical resection, wherein radiation or chemotherapy is not provided. In one embodiment, if surgical resection has been performed by the time the sample is analyzed, no additional treatment is provided. In a specific embodiment, the subject is monitored for possible relapse.

In another aspect, the present disclosure provides a method of treating a pediatric glioma (e.g., an angiocentric glioma, a pediatric glioma comprising an MYB-QKI rearrangement, or an angiocentric glioma comprising an MYB-QKI rearrangement). In some embodimentssome embodiments, a subject having a PLGG is provided a treatment that antagonizes one or more effects of MYB-QKI rearrangement. In one embodiment, the amount of MYB-QKI mRNA is reduced by suppressing the activity of at least one enhancer that promotes MYB-QKI expression. In another embodiment, the amount of MYB-QKI protein is reduced by accelerating the degradation of the MYB-QKI fusion protein. In yet another embodiment, the amount or activity of the wild type tumor suppressor protein QKI is increased, thereby treating the PLGG.

In some embodiments, a pediatric glioma is examined using a method of classifying a pediatric glioma described herein, and the subject having the pediatric glioma is treated using a method of treatment described herein.

As used herein, “a glioma” encompasses, but is not limited to, a tumor that originates from glial cells in the brain or spine. Glial cells encompass, but are not limited to, oligodendrocytes, astrocytes, ependymal cells, radial glial cells, and microglia. Types of glioma encompass, but are not limited to, pilocytic astrocytoma, pilomyxoid astrocytoma, fibrillary astrocytoma, angiocentric glioma, oligodendroglioma, diffuse astrocytoma, dysembryoplastic neuroepithelial tumor, ganglioglioma, pleomorphic xanthoastrocytoma, anaplastic astrocytoma, glioblastoma multiforme, diffuse intrinsic pontine glioma, optic pathway glioma, tectal glioma, ganglioglioma. Whether a tumor is a glioma, and the type of glioma, can be determined by histology and other methods such as molecular diagnostic methods.

“A pediatric glioma” encompasses, but is not limited to, a glioma in a subject that started to form in childhood. In humans, childhood refers to the period from fetus formation to 21 years from birth.

“A pediatric low-grade glioma (PLGG)” is a pediatric glioma of grade 1 or grade 2 by World Health Organization (WHO) grading. It is the most common type of brain tumor in children. Types of PLGG encompass, but are not limited to, pilocytic astrocytoma, pilomyxoid astrocytoma, fibrillary astrocytoma, angiocentric glioma, oligodendroglioma, diffuse astrocytoma, dysembryoplastic neuroepithelial tumor, ganglioglioma, pleomorphic xanthoastrocytoma, optic pathway glioma, tectal glioma, ganglioglioma.

“An angiocentric glioma” is a WHO grade 1 brain tumor. It is diagnosed by the histological feature of elongated cells with a perivascular orientation. The clinical manifestation often includes epilepsy, wherein >95% of patients have intractable seizures.

“A sample of the pediatric glioma” encompasses, but is not limited to, a sample of pediatric glioma obtained from surgical resection or a biopsy. A biopsy includes, but is not limited to, an open biopsy, a needle biopsy and a stereotactic biopsy.

“Isolating genomic DNA” encompasses, but is not limited to, isolating a substantially pure genomic DNA preparation, isolating a mixture containing genomic DNA, and isolating a population of cells containing genomic DNA.

“Isolating RNA” encompasses, but is not limited to, isolating a substantially pure RNA preparation, isolating a substantially pure messenger RNA preparation, isolating a mixture containing cellular RNA, isolating a population of cells containing cellular RNA.

“Isolating protein” encompasses, but is not limited to, isolating a substantially pure protein preparation, isolating a substantially pure nuclear protein preparation, isolating a mixture containing cellular proteins, isolating a mixture containing nuclear proteins, isolating a population of cells containing proteins, isolating a tissue comprising cells containing proteins, wherein the protein may be intact or modified during the isolation procedure.

“An MYB-QKI rearrangement” encompasses, but is not limited to, a change in the physical structure of the genome that leads to a fusion of at least a portion of a MYB gene and at least a portion of a QKI gene, which are not adjacent to each other on a chromosome in at least 90%, 80%, 70%, 60% or 50% of cells of all individuals in a species.

“The presence of an MYB-QKI rearrangement” encompasses, but is not limited to, the presence of cells that carry at least one copy of MYB-QKI rearrangement, wherein these cells may constitute at least 90%, 80%, 70%, 60%, 50%, 40%, 30%, 20%, 10% or 5% of all the cells in the sample.

“A fusion of a MYB gene and a QKI gene” encompasses, but is not limited to, a joining of at least a portion of a MYB gene and at least a portion of a QKI gene on a chromosome or microchromosome, wherein at least a portion of a MYB gene and at least a portion of a QKI gene are adjacent to each other.

“Screening the presence of an MYB-QKI rearrangement” encompasses, but is not limited to, examining the presence of an MYB-QKI rearrangement by a technical method, and obtaining data from an agency that examines the presence of an MYB-QKI rearrangement. The method of screening encompasses, but is not limited to, screening for the presence of a fusion of a MYB gene and a QKI gene by a cytogenetic technique or by DNA sequencing, screening for the presence of a MYB-QKI fusion RNA, and screening for the presence of a MYB-QKI fusion protein by antibodies or mass spectrometry.

“A rearrangement breakpoint” encompasses, but is not limited to, a location on a chromosome or microchromosome where at least a portion of a first gene and at least a portion of a second gene join.

“An in-frame fusion” encompasses, but is not limited to, a fusion of at least a first gene and a second gene that leads to the formation of a third fusion gene, wherein the third fusion gene can be transcribed to a messenger RNA that maintain at least a portion of the first gene in its original reading frame and at least a portion of the second gene in its original reading frame.

“A cytogenetic technique” encompasses, but is not limited to, a genetic method applied in the context of a cell. Common cytogenetic techniques include, but are not limited to, G-banding karyotype analysis, fluorescence in situ hybridization (FISH), and chromosome microarray analysis.

“Fluorescence in situ hybridization (FISH)” encompasses, but is not limited to, a laboratory technique for detecting and locating a DNA sequence of interest on a chromosome, wherein at least one probe conjugated to a fluorescent moiety is hybridized to the DNA sequence of interest.

“A FISH probe” encompasses, but is not limited to, a nucleic acid conjugated to a fluorescent moiety capable of hybridizing to a DNA sequence of interest. Two DNA sequences of particular interest are a first region 5′ to exon 16 of a MYB gene (SEQ ID NO: 8), wherein the first region is located within 100 kb from exon 16 of the MYB gene, and a second region 3′ to exon 4 of a QKI gene (SEQ ID NO: 4), wherein the second region is located within 100 kb from exon 4 of the QKI gene.

“A molecular inversion probe” is used to capture a target DNA with a region of interest. It encompasses, but is not limited to, a single-stranded DNA molecule containing a 5′ terminal region and a 3′ terminal region, wherein the 5′ terminal region can hybridize to the target DNA at a location 3′ to the region of interest, and the 3′ terminal region can hybridize to the target DNA at a location 5′ to the region of interest, thereby forming a circle between the molecular inversion probe and the target DNA. Different versions of molecular inversion probes include, but are not limited to, padlock probes and connector inversion probes.

“Whole-exome sequencing” encompasses, but is not limited to, sequencing of all protein coding genes in a genome.

“RNA sequencing” encompasses, but is not limited to, sequencing of at least one RNA molecule, and sequencing of at least one nucleic acid molecule that is synthesized to be complementary to at least one RNA molecule, wherein the at least one nucleic acid molecule includes, but is not limited to, at least one DNA molecule.

“Whole-transcriptome sequencing” encompasses, but is not limited to, RNA sequencing of all detectable RNA molecules, all detectable messenger RNA molecules, all detectable pre-messenger RNA molecules, all detectable small RNA molecules, and a combination thereof.

“An anti-MYB antibody” encompasses, but is not limited to, an anti-MYB antiserum, an anti-MYB polyclonal antibody, an anti-MYB monoclonal antibody, an antigen-binding fragment of an anti-MYB antibody, a variable fragment of an anti-MYB antibody, and a protein that binds to MYB specifically.

“An anti-QKI antibody” encompasses, but is not limited to, an anti-QKI antiserum, an anti-QKI polyclonal antibody, an anti-QKI monoclonal antibody, an antigen-binding fragment of an anti-QKI antibody, a variable fragment of an anti-QKI antibody, and a protein that binds to QKI specifically.

“Fluorescence resonance energy transfer (FRET)” encompasses, but is not limited to, a distance-dependent interaction between the electronic excited states of a donor fluorescent molecule and an acceptor fluorescent molecule, wherein excitation is transferred from the donor molecule to the acceptor molecule through a non-radiative process referred to as resonance. The presence of FRET is detected by observing the wavelength of the emission, wherein the wavelength of the emission from the acceptor molecule when FRET occurs is different from the wavelength of the emission from the donor molecule when FRET does not occur.

“A proximity ligation assay” encompasses, but is not limited to, an assay capable of detecting the proximity of a first epitope and a second epitope, wherein the first epitope is recognized by a first antibody that is associated with a first DNA strand and the second epitope is recognized by a second antibody that is associated with a second DNA strand, wherein the first DNA strand and the second DNA strand can both hybridize to at least a third DNA strand when they are in proximity, wherein ligation occurs in at least one of the first, second and third DNA strand following hybridization.

“A subject” encompasses, but is not limited to, a mammal, e.g. a human, a domestic animal or a livestock including a cat, a dog, a cattle and a horse.

“Surgical resection” encompasses, but is not limited to, a surgical procedure to remove an abnormal tissue, wherein a normal surrounding tissue may be removed at the same time. An abnormal tissue includes but is not limited to a tumor.

“Radiation therapy” encompasses, but is not limited to, localized therapy of a glioma by a certain level of radiation from an external beam and/or internal radioactive seeds placed into the tumor lesion.

“Chemotherapy” means one or more anti-tumor chemical substances. Chemotherapy for a glioma encompasses, but is not limited to, temozolomide, carmustine, vinscristine, procarbazine, lomustine, cisplatin, methotrexate, cytosin-arabinoside, MX2, topocetan, paclitaxel, and a combination thereof.

An “agent” refers to a substance which may be used in connection with an application that is therapeutic or diagnostic, such as, for example, in methods for diagnosing the presence of a disease in a subject and/or methods for the treatment of a disease in a subject. Exemplary agents include without limitation small molecule compounds, nucleic acids, proteins (e.g., peptides, polypeptides, multi-subunit proteins), particles (e.g., nanoparticles, viral particles, liposomes), and a combination thereof.

“Deleting at least a 100 bp portion of the MYB-QKI fusion DNA” and “generating a frame-shifting mutation of the MYB-QKI fusion DNA” encompasses, but are not limited to, genome editing procedures using a zinc finger nuclease, a transcription activator-like effector nuclease (TALEN), or CRISPR technology.

“CRISPR technology” encompasses, but is not limited to, a method of changing genomic DNA using one or more CRISPR RNA molecules to guide an endonuclease, such as Cas9, to a genomic location, wherein a change in the DNA, such as a deletion or a frame-shifting mutation, is introduced when the cleaved of genomic DNA is rejoined. In some embodiments, the Cas9 protein is introduced to a pediatric glioma (e.g., an angiocentric glioma) of the subject as a protein or a polynucleotide (e.g., a DNA, RNA, modified DNA, or modified RNA) encoding a Cas9 protein, wherein the Cas9 coding sequence is optionally operably linked to a transcriptional regulatory element. The coding sequence may be codon-optimized to increase the expression in a cell of the subject. The Cas9 protein may be either a wild-type Cas9 protein (e.g., having a wild-type sequence as in Streptococcus pyogenes or Streptococcus thermophilus) or a mutant, variant, derivation, or fusion protein thereof. In some embodiments, a CRISPR-Cas system guide RNA polynucleotide targeting the MYB-QKI genomic locus is introduced to a pediatric glioma (e.g., an angiocentric glioma) of the subject as a polynucleotide (e.g., a DNA, RNA, modified DNA, or modified RNA). In some embodiments, the guide RNA targets the MYB-QKI genomic locus or the MYB genomic locus. In one embodiment, the guide RNA hybridizes with a sequence in the MYB-QKI genomic locus (e.g., a sequence in the MYB portion, or a sequence at the junction comprising a MYB portion and a QKI portion). Cas9 proteins and CRISPR-Cas system guide RNAs useful in this invention are disclosed in WO2014093701A1 and WO2013188638A2, which are incorporated by reference herein in their entirety.

“RNA interference” encompasses, but is not limited to, reducing the amount or activity of a first messenger RNA (mRNA) molecule by introducing a second RNA molecule that hybridizes to the first mRNA, or by introducing a DNA molecule that is transcribed and/or processed into the second RNA. The term “interfering RNA” refers to the second RNA molecule or the DNA molecule that is transcribed and/or processed into the second RNA. The activity of a messenger RNA hereby refers to the efficiency that the messenger RNA is translated into a polypeptide. Commonly used RNA interference technologies include, but are not limited to, microRNA and small interfering RNA (siRNA).

“An agent that inhibits the activity of at least one enhancer” encompasses, but is not limited to, a DNA-binding molecule or a DNA-binding complex that binds to the enhancer sequence and reduces its activity, and an agent that inhibits the activity of an endogenous enhancer-binding complex. An enhancer hereby includes, but is not limited to, a regular enhancer and a super-enhancer, that enhances in cis gene transcription. Exemplary endogenous enhancer-binding complexes include without limitation a complex comprising a protein having a bromodomain and extraterminal domain (BET) domain, and a complex comprising CDK7. In some embodiments, the agent is an antagonist of BET. Exemplary antagonists of BET are disclosed in Andrieu et al., Drug Discovery Today: Technologies 19:45-50; Schaper, Nature Biotechnology 34:361-62; Chaidos et al., Ther. Adv. Hematol. 6(3):128-41; and Wadhwa et al., Cureus 8(5): e620, each of which is incorporated by reference herein in its entirety. In one embodiment, the antagonist of BET is selected from the group consisting of JQ1, GSK1210151A, GSK525762, OTX-015, TEN-010, CPI-203, CPI-0610, RVX-208, and LY294002. In one specific embodiment, the antagonist of BET is JQ1. In some embodiments, the agent is an antagonist of CDK7. In some embodiments, the antagonist of CDK7 inhibits the activity of CDK7 with an IC₅₀ lower than (e.g., lower by 10%, 20%, 30%, 40%, 50%, 60%, 70%, 80%, 90%, 95%, or 99%) the IC₅₀ of its inhibition of one or more other CDK (e.g., CDK4, CDK6). Exemplary antagonists of CDK7 are disclosed in Lapenna et al., Nature Reviews Drug Discovery 8:547-66; Kwiatkowski et al., Nature 511:616-20; and Wang et al., Drug Des Devel. Ther. 10:1181-89, each of which is incorporated by reference herein in its entirety. In one embodiment, the antagonist of CDK7 is selected from the group consisting of THZ1, BS-181, flavopiridol, P276-00, R-roscovitine, R547, SNS-032, and ZK 304709. In one specific embodiment, the antagonist of CDK7 is THZ1.

“Inhibiting H3K27 acetylation” encompasses, but is not limited to, inhibiting the amount and/or activity of a histone acetyltransferase, increasing the amount and/or activity of a histone deacetylase, modulating one or more other histone modifications that leads to the reduction of H3K27 acetylation, introducing or modulating one or more non-coding RNA molecules that leads to the reduction of H3K27 acetylation, and a combination thereof.

“The MYB-QKI locus” refers to a genomic location of a fused MYB gene and QKI gene resides. It encompasses, but is not limited to, the region of MYB and QKI promoters, enhancers, exons, introns, and intergenic region between the fusion gene and their nearby genes.

“Inhibiting the activity of at least one deubiquitinating enzyme (DUB)” encompasses, but is not limited to, reducing the amount and/or activity of at least one deubiquitinating enzymes, thereby increasing ubiquitination of an MYB-QKI fusion protein and increased degradation of the protein. Agents that inhibit the activity of at least one DUB are disclosed in Huang et al., Cell Research 26:484-98; Huang et al., Oncotarget 7(3):2796-2808; and Farshi et al., Expert Opin. Ther. Pat. 25(10): 1191-1208, each of which is incorporated by reference herein in its entirety. In some embodiments, the agent inhibits the activity of more than one DUBs. In one embodiment, the antagonist of DUB is selected from the group consisting of PR619, VLX1570, b-AP15, PX-478, and WPI 130. In one specific embodiment, the antagonist of DUB is PR619.

“An antagonist of c-Kit” encompasses, but is not limited to, an agent that reduces the expression (e.g., mRNA, protein) of c-Kit, an agent that increases degradation of c-Kit mRNA or protein, and an agent that inhibits c-Kit activity (e.g., kinase activity). Agents that inhibit c-Kit activity are disclosed in Galanis et al., Haematologica 100(3):e77-e79; and Babaei et al, Drug Design, Development, and Therapy 2016:10:2443-59, each of which is incorporated by reference herein in its entirety. In one embodiment, the antagonist of c-Kit is selected from the group consisting of axitinib, dovitinib, dasatinib, imatinib, motesanib, pazopanib, masitinib, vatalanib, cabozantinib, tivozanib, OSI-930, Ki8751, telatinib, pazopanib, and tyrphostin AG 1296. In one specific embodiment, the antagonist of c-Kit is dasatinib.

“An MYB alteration” encompasses, but is not limited to, a change in the physical structure of the genomic location of an MYB gene.

“A copy number alteration” encompasses, but is not limited to, an alteration of the DNA of a genome that results in more or fewer copies of at least a portion of an MYB gene.

“A truncation” encompasses, but is not limited to, an alteration of the DNA of a genome that results in loss of a portion of an MYB gene sequence on at least one chromosome.

“A fusion” encompasses, but is not limited to, an alteration of the DNA of a genome that results in adjacency or joining of the MYB gene and a second gene, wherein the MYB gene and the second gene are not adjacent to each other on a chromosome in at least 90%, 80%, 70%, 60% or 50% of cells of all individuals in a species.

“A rearrangement” encompasses, but is not limited to, a change in the physical structure of the genome that leads to adjacency or joining of at least a portion of a MYB gene and at least a portion of a second gene, wherein the MYB gene and the second gene are not adjacent to each other on a chromosome in at least 90%, 80%, 70%, 60% or 50% of cells of all individuals in a species.

“A 5′ deletion” encompasses, but is not limited to, an alteration of the DNA of a genome that results in loss of a 5′ portion of an MYB gene sequence on at least one chromosome.

“A 3′deletion” encompasses, but is not limited to, an alteration of the DNA of a genome that results in loss of a 3′ portion of an MYB gene sequence on at least one chromosome.

“A breakpoint” encompasses, but is not limited to, an alteration of the DNA of a genome that results in separation of an MYB gene to two or more portions by DNA not in an MYB gene in at least 90%, 80%, 70%, 60% or 50% of cells of all individuals in a species.

“A translocation” encompasses, but is not limited to, an alteration of the DNA of a genome that results in movement of an MYB gene to a location where the MYB gene does not locate in at least 90%, 80%, 70%, 60% or 50% of cells of all individuals in a species.

“An inversion” encompasses, but is not limited to, an alteration of the DNA of a genome wherein a segment of a chromosome comprising an MYB gene is reversed.

“An insertion” encompasses, but is not limited to, an alteration of the DNA of a genome that results in the insertion of more or more DNA sequences in an MYB gene, wherein the DNA sequences are not in an MYB gene in at least 90%, 80%, 70%, 60% or 50% of cells of all individuals in a species.

“The expression level of MYB exons 1-9 is higher in the pediatric glioma with the MYB rearrangement than in a control sample” refers to that the expression level of MYB exons 1-9 is at least 10%, 20%, 30%, 40%, 50%, 60%, 70%, 80%, 90% or 100% higher in the pediatric glioma with the MYB rearrangement than in a control sample.

Furthermore, in accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The following examples are provided to further elucidate the advantages and features of the present application, but are not intended to limit the scope of the application. The examples are for illustrative purposes only.

### EXAMPLES

### Example 1

### Angiocentric Gliomas Exhibited Recurrent MYB-QKI Rearrangements

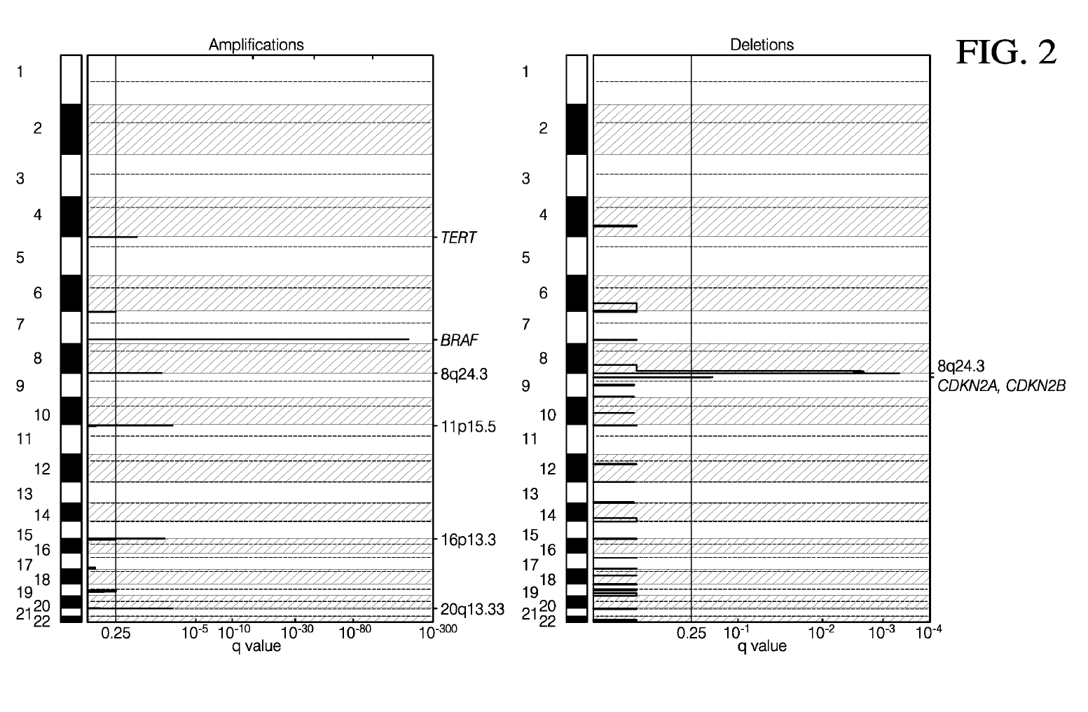

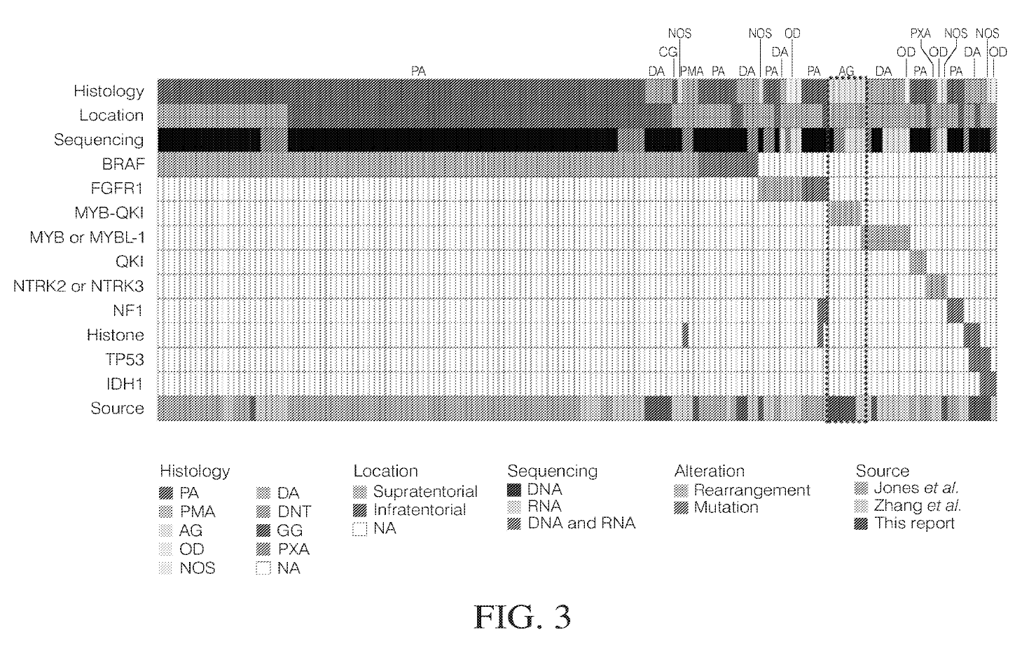

Previously published genomic analyses of PLGGs did not individually contain sufficient numbers of rare histological subtypes to achieve statistical power to detect recurrent aberrations. To address this, a combined genomic analysis of whole-genome sequencing and/or RNA sequencing (RNA-seq) data from 172 PLGGs spanning ten histological subtypes, including 145 published samples and 27 rare PLGGs that were new to this study was performed. Analyses of significantly recurrent somatic genetic events across all samples with whole-genome sequencing or RNAseq data were performed (FIG. 2). Recurrent somatic alterations in 154 tumors (90%), including 140 tumors subjected to whole-genome sequencing were observed. Rearrangements or structural alterations were observed in 129 tumors (FIG. 3).

Six recurrently amplified and two recurrently deleted genomic regions were identified (FIG. 2). The most significant focal amplification peak encompassing 16 genes was observed on chromosome 7q34, immediately adjacent to BRAF corresponding to well described truncating duplications of BRAF in PLGGs3,28. The second most significant focal amplification peak encompassed 34 genes on chromosome 5p15 including TERT. A deletion peak on 9p21.3, which was previously observed, includes CDKN2A and CDKN2B. Five genes were significantly mutated (q<0.1) across the cohort: BRAF, FGFR1, H3F3A, NF1 and TP53 (FIG. 2). Recurrent mutations in the known cancer driver IDH1 were also observed. Recurrent rearrangements involved BRAF, FGFR1, MYB family members including MYB and MYBL1, QKI, and NTRK2 or NTRK3. Taking mutations and rearrangements together, the most frequently altered gene across the entire cohort was BRAF with focal duplications/rearrangements in 100 PLGGs (including 90 Pilocytic Astrocytomas (PAs)), and mutations in 12 tumors. FGFR1 was altered in 12 PLGGs, with mutations in five PAs and rearrangements in three PAs, three Oligodendrogliomas (OGs) and one Diffuse Astrocytoma (DA). Genes encoding histones were mutated in five PLGGs, including two PAs/Pilomyxoid Astrocytomas (PMAs) and three DAs. Four of these mutations were in H3F3A; the fifth was in HIST1H3B. Mutations in H3F3A have been observed in pediatric high-grade gliomas, most frequently K27M or G34R/V2,3,29. Consistent with mutations affecting H3F3A in pediatric high-grade gliomas, the presence of at least one other driver alteration in four samples with histone mutations was observed.

Rearrangements involving MYB family members (MYB and MYBL1) were the second most recurrent alteration, affecting 16 tumors (10%), predominantly diffuse astrocytomas and angiocentric gliomas. Six of the seven angiocentric gliomas, including all tumors subjected to central pathology review, exhibited intrachromosomal deletions resulting in MYB-QKI rearrangements. The other angiocentric glioma, which was not centrally reviewed, contained a MYB-ESR1 rearrangement.

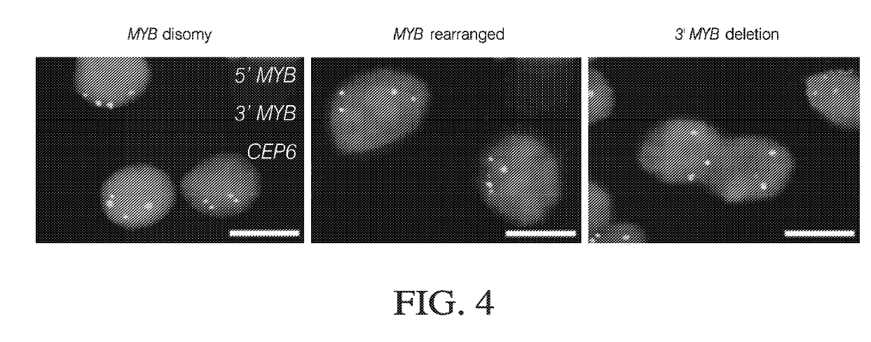

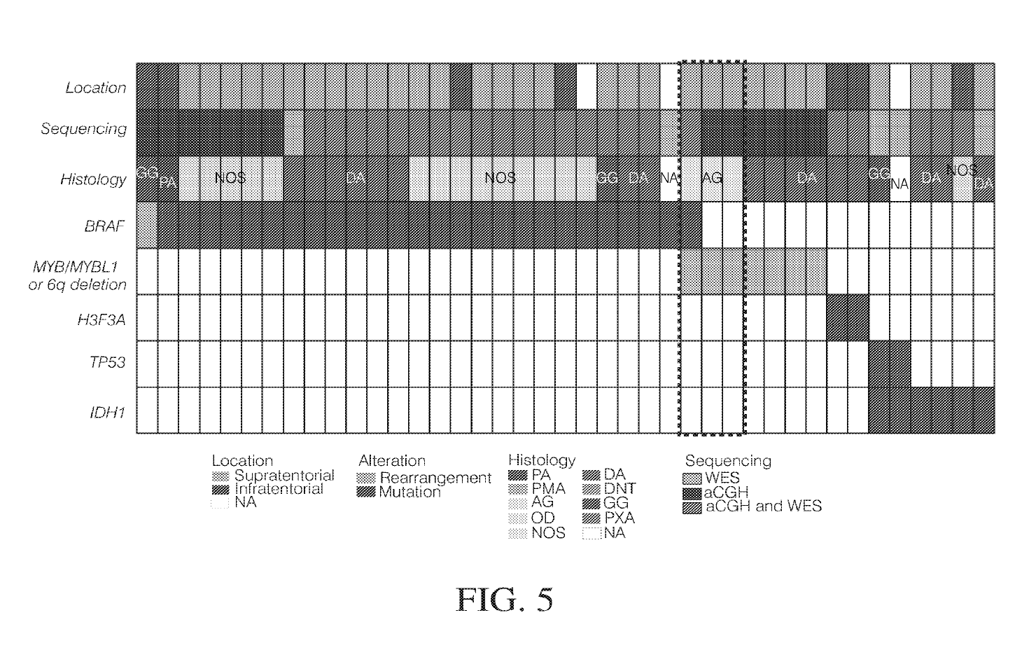

While MYB rearrangements had been described in PLGGs by Zhang et al. as a sporadic event, our results revealed that QKI was the most frequent fusion partner, and MYB-QKI fusions were nearly universal in angiocentric glioma. For validation, we studied 12 additional angiocentric gliomas with only formalin-fixed, paraffin-embedded tissue available using targeted assays. Nine angiocentric gliomas were analyzed by fluorescent in situ hybridization (FISH) to detect MYB rearrangement or deletion (FIG. 4), and three angiocentric gliomas were analyzed by whole-exome sequencing and/or array comparative genomic hybridization (array CGH). All 12 harbored MYB aberrations.

In total, all 19 angiocentric gliomas profiled by whole-genome sequencing, RNA-seq, whole-exome sequencing, FISH or array CGH displayed MYB alterations, and, in six of the seven cases in which the fusion partner of MYB could be detected, MYB was fused to QKI. In tumors confirmed to harbor MYB-QKI rearrangement, the genetic event appeared to be present in the majority of cells, although evidence of heterogeneity (aberration in ˜50% of tumor cells) was observed with FISH analysis in two of five tumors with sufficient numbers of cells for quantitative scoring.

### Example 2

### MYB-QKI Rearrangement was Specific to Angiocentric Glioma

MYB-QKI rearrangements appeared to be specific to angiocentric glioma. None of the 147 non-angiocentric gliomas profiled with whole-genome sequencing or RNA-seq exhibited MYB-QKI fusions (P<0.0001; Table 1). MYB alterations were also evaluated in an additional 65 PLGGs from two separate cohorts: ten non-angiocentric gliomas analyzed by FISH and 55 non-angiocentric gliomas evaluated by whole-exome sequencing and/or array CGH. Only one of these tumors exhibited alterations in MYB (as compared to 19/19 angiocentric gliomas; P<0.0001; FIG. 5). This tumor was designated not otherwise specified upon research review but had been diagnosed as angiocentric glioma at the referring institution. Five tumors evaluated by whole-exome sequencing or array CGH exhibited alterations of MYBL1; these tumors were all diffuse astrocytomas. The FISH assays, array CGH and whole exome sequencing, although able to detect MYB alterations, were unable to characterize the fusion partners of MYB.

TABLE 1
Frequency of MYB alterations and MYB-QKI rearrangements in diffuse
astrocytoma and angiocentric glioma.

| | | | Other | No |
|---|---|---|---|---|
| Tumors | n | MYB-QKI | MYB | MYB |
| Angiocentric | 7 | 6 | 1 | 0 |
| glioma | | | | |
| Non-angiocentric | 147 | 0 | 9 | 138 |
| glioma | | | | |

P < 0.0001
The P value represents enrichment of MYB-QKI rearrangements in angiocentric glioma.
MYB-QKI alterations were identified by whole-genome sequencing alone (n = 1), whole-genome sequencing and RNA-seq (n = 2), or RNA-seq alone (n = 3).

In the cohort analyzed by whole-genome sequencing and/or RNA-seq, rearrangements involving QKI but not MYB in three supratentorial pilocytic astrocytomas and rearrangements involving MYB or MYBL1 but not QKI in nine tumors, seven of which were diffuse astrocytomas were also observed. Across the entire cohort of 172 tumors profiled with whole-genome sequencing and/or RNA-seq, 10% of tumors harbored alterations of either MYB family members or QKI.

### Example 3

### Breakpoints in MYB-QKI Rearrangement

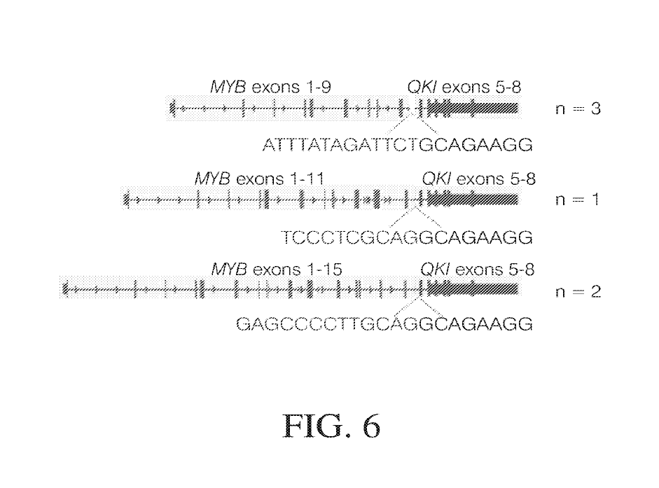

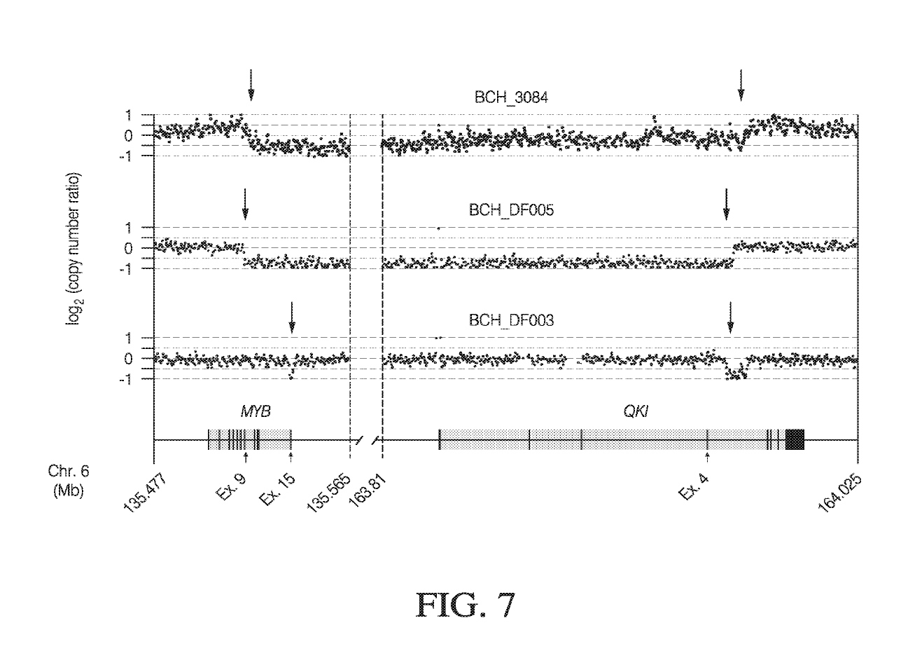

We identified fusion mRNA transcripts by RNA-seq (FIG. 6) and observed copy number breakpoints in the MYB and QKI genes in whole-genome sequencing data (FIG. 7). All MYB-QKI rearrangements had breakpoints within intron 4 of QKI, whereas the MYB breakpoint varied in its position from intron 9 to intron 15. All the rearrangements were predicted to result in the expression of an in-frame fusion protein, MYB-QKI.

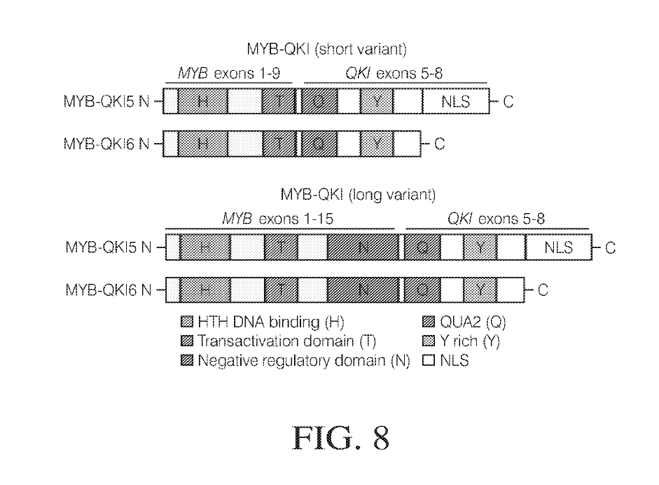

MYB-QKI breakpoints mapping between introns 9 and 15 of MYB were predicted to result in C-terminal truncation and gain of oncogenic potential of the MYB protein. MYB proteins are transcription factors characterized by highly conserved DNA-binding motifs. First identified as v-Myb, the cellular proto-oncoprotein counterpart c-MYB comprises an N terminus that contains helix-turn-helix (HTH) DNA-binding motifs followed by a transcriptional activation domain and a C-terminal negative regulatory domain. It was expected that the MYB-QKI fusion protein retained the MYB Nterminal HTH DNA-binding motifs fused to the QKI C terminus (FIG. 8). The QKI N-terminal KH RNA-binding motif was lost, while C-terminal alternative splice sites were preserved. The MYB-QKI5 splice variant retained a nuclear localization sequence (NLS), which was not present in the MYB-QKI6 splice variant. Fusions that contain only exons 1-9 of MYB also lost the MYB negative regulatory domain (designated as the short variant). Therefore, both MYB and QKI were disrupted by the rearrangement.

### Example 4

### Expression of MYB in Cortical Brain

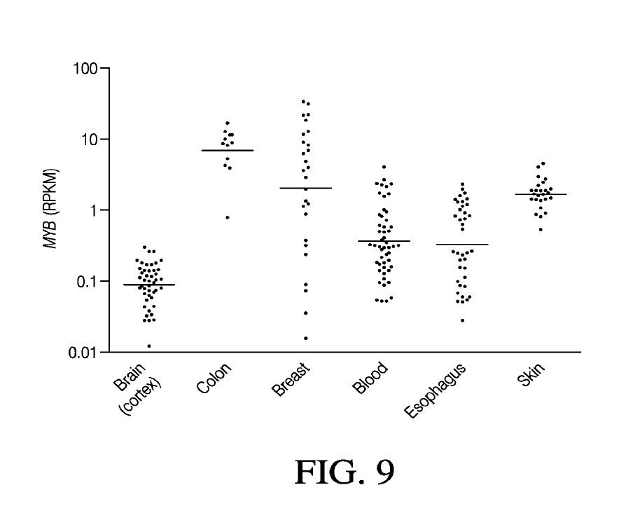

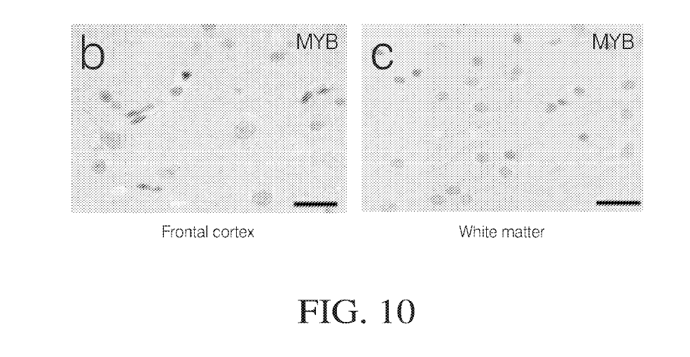

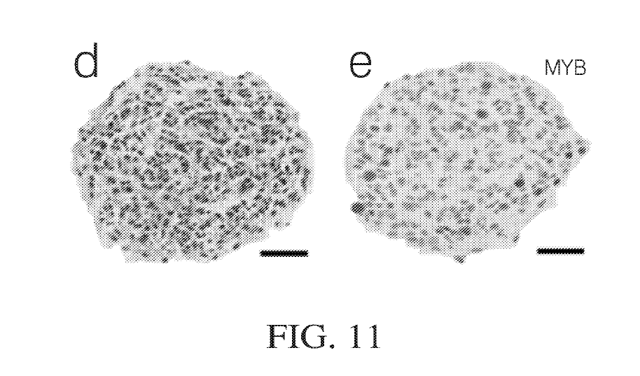

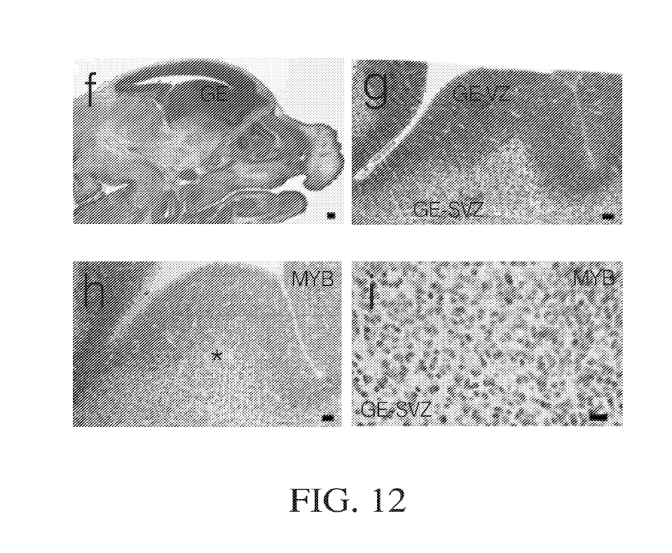

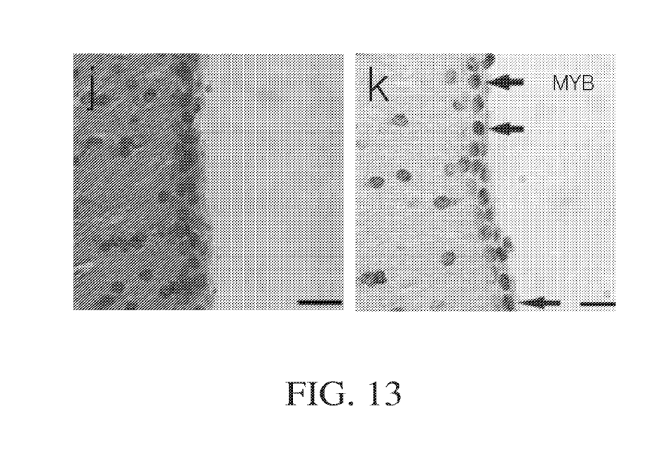

MYB is not expressed in the postnatal brain cortex, where angiocentric gliomas occur. RNA-seq data for normal tissues were examined and it was found that MYB expression was negligible in human brain cortex and substantially lower than MYB expression in colon, breast, blood, esophagus or skin (FIG. 9). Likewise, immunohistochemistry of adult human frontal cortex and white matter was negative for MYB (FIG. 10). In contrast, high MYB expression in human fetal neural progenitor cells generated from the ganglionic eminence at 22 weeks of gestation (FIG. 11) was detected. In mice, MYB was expressed in embryonic day (E) 14.5 neural progenitor cells of the ganglionic eminence subventricular region (FIG. 12). In adult mice, expression of MYB in the ependyma/subventricular zone (FIG. 13) was detected, consistent with previous reports of MYB expression in mouse progenitor cells but not in cortical brain.

### Example 5

### Deletion of QKI in Glioblastoma

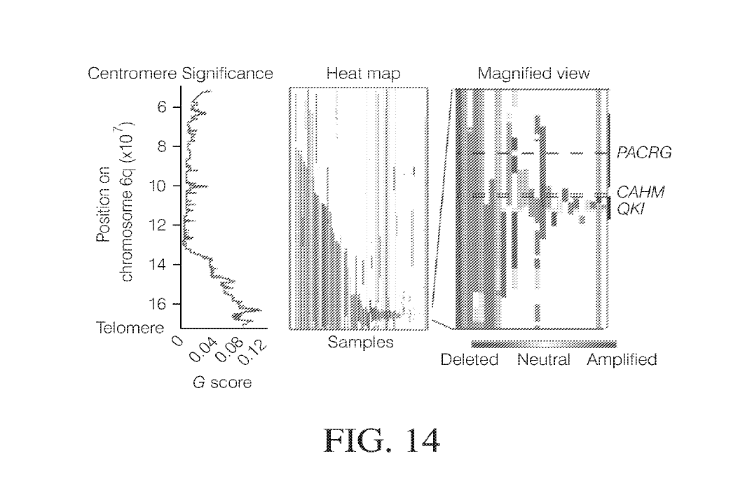

QKI encodes the STAR (signal transduction and activation of RNA) RNA-binding protein Quaking, which has an essential role in oligodendroglial differentiation and is widely expressed in the nervous system. Deletions of QKI have been suggested to be oncogenic in a number of human cancers, including glioblastoma, prostate cancer and gastric cancer. Copy number analyses of 10,570 cancers from The Cancer Genome Atlas (TCGA) identified QKI as one of two genes in a deletion peak in adult glioblastoma (FIG. 14), renal clear cell carcinoma and cervical squamous cell carcinoma. It was also in larger peak regions of significant deletion in low-grade glioma and bladder and adrenocortical carcinomas. Focal QKI deletions were observed in over 10% of glioblastomas.

### Example 6

### MYB-QKI Functions as a Transcription Factor

The findings that both MYB and QKI were disrupted suggested that MYB-QKI rearrangements may be oncogenic through the additive effects of alterations in both MYB and QKI. The lack of expression of MYB in normal postnatal human cortical brain regions also suggested that rearrangement drove aberrant expression of the fusion allele. Therefore, mechanisms were characterized through which MYB-QKI rearrangements might contribute to aberrant MYB-QKI expression and evaluated the oncogenic potential of both genes.

Genome-wide gene expression analyses of three independently generated pools of mouse neural stem cells (mNSCs) engineered to stably overexpress MYB-QKI5, MYB-QKI6, truncated MYB encoded by exons 1-9 (MYBtr^{exons 1 9}) or enhanced GFP (eGFP) were performed. Relative to eGFP-expressing cells, those expressing MYB-QKI5 and MYB-QKI6 exhibited significantly different expression for 1,621 and 1,947 genes, respectively, with 1,029 genes overlapping (P<0.0001). Gene set enrichment analysis (GSEA) showed that expression of either MYBtr^{exons1-9} or the MYB-QKI isoforms was associated with enrichment of signatures of MYB pathway activation (P<0.0001).

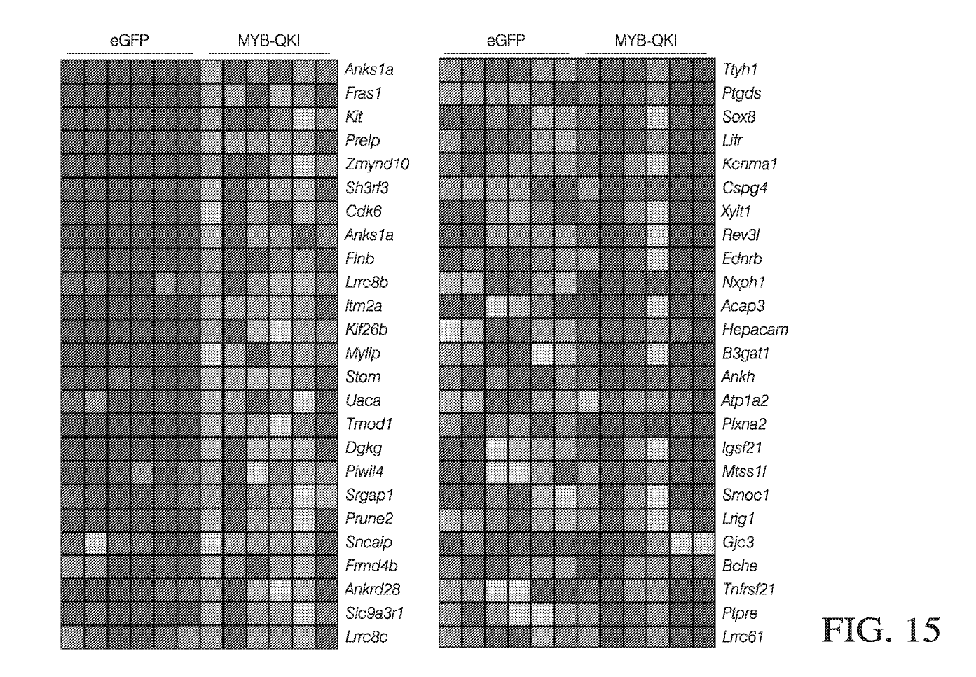

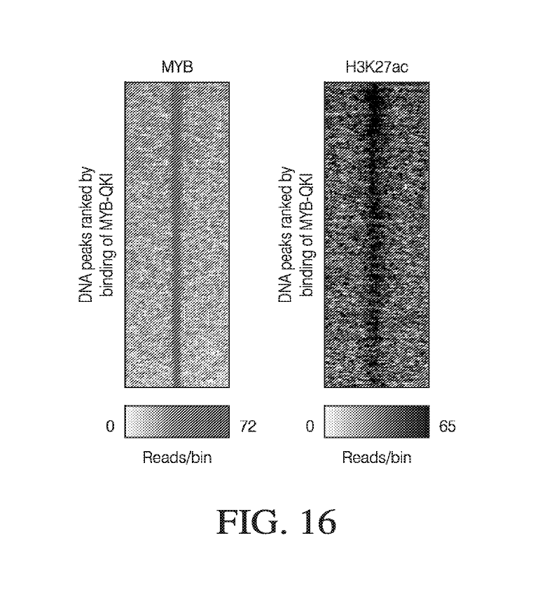

A MYB-QKI gene expression signature was defined comprising the 50 genes whose differential expression correlated most with the expression of the fusion protein (FIG. 15). These genes included KIT and CDK6, previously reported to be associated with MYB activation.

Chromatin immunoprecipitation was performed with parallel sequencing (ChIP-seq) in mNSCs expressing MYB-QKI fusion protein, using one antibody that recognizes the N terminus of MYB and another antibody against acetylation of histone H3 at lysine 27 (H3K27ac), which defines the location of enhancer regions. It was found that MYB-QKI5 bound 3,672 sites (P-value threshold=1×10-7) across the genome (92% of these sites contained a MYB binding motif) and H3K27ac was present at 9,122 sites, with an overlap of 1,907 sites (52% MYB binding sites, P<0.0001) (FIG. 16). These findings were consistent with reports in T cell acute lymphoblastic leukemia (T-ALL), where MYB binding was highly correlated with H3K27ac-defined enhancers.

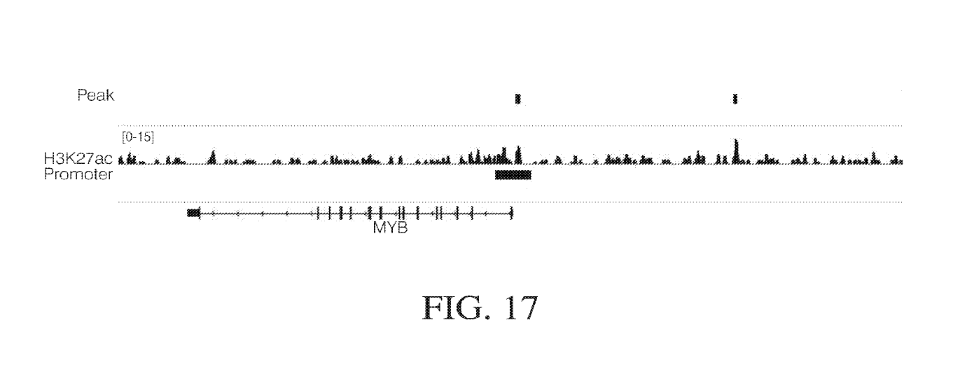

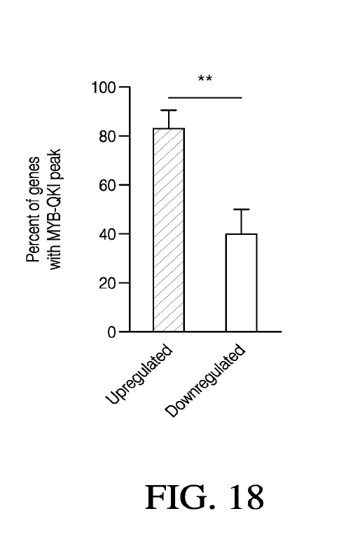

MYB-QKI binding to the endogenous Myb promoter was also identified (FIG. 17). MYB-QKI5 binding sites (P-value threshold=1×10⁻⁵) were located within 100 kb of 88% (22/25) of the upregulated genes in the MYBQKI signature but only 40% (10/25) of the downregulated genes in the signature (P<0.001; FIG. 18). Each of the MYB-QKI binding sites associated with an upregulated gene was associated with an H3K27ac marked enhancer peak, whereas only 70% of the MYB-QKI binding sites at downregulated genes overlapped enhancers (P=0.003).

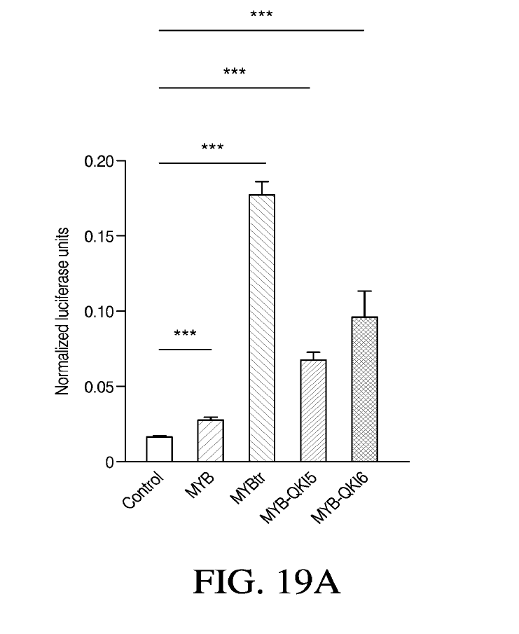

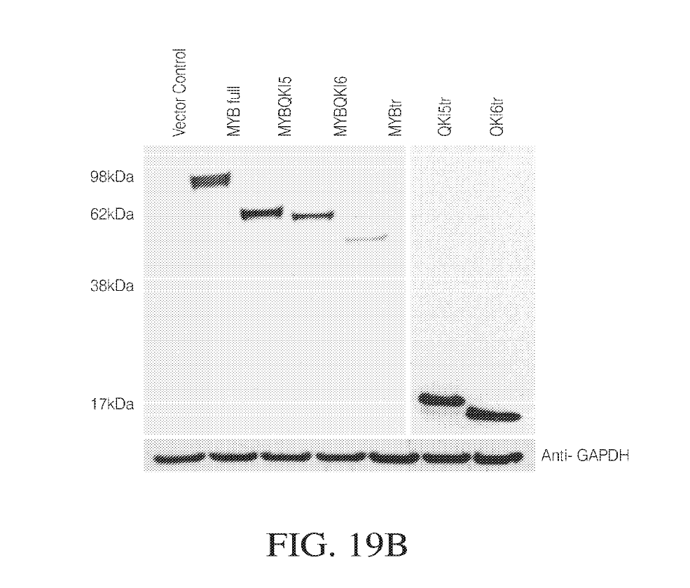

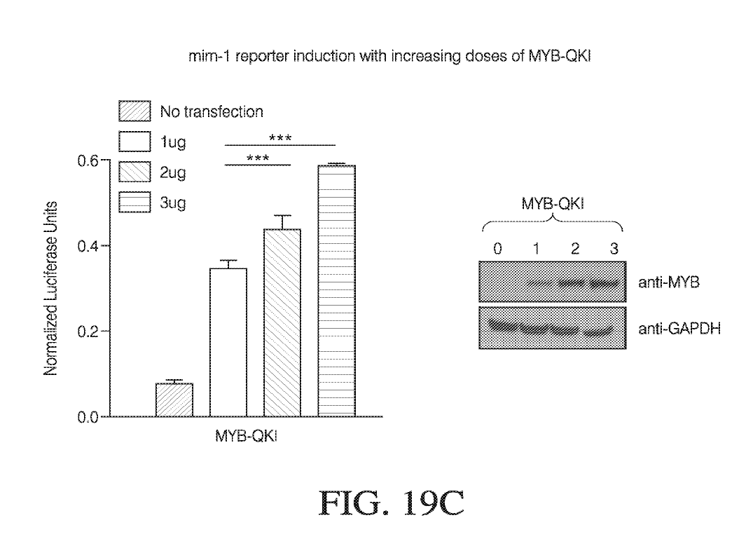

The MYB-QKI fusion protein can activate transcription through binding of MYB consensus binding motifs. A luciferase reporter construct was generated using known MYB binding sites from the mim-1 target promoter and cotransfected this reporter construct together with a construct encoding MYBtr^{exons1-9}, MYB-QKI or full-length MYB into HEK293T cells. A slight induction in mim-1 promoter activity with transfection to express full-length MYB as compared to the control vector was observed. The greatest induction in mim-1 promoter activity was observed upon cotransfection of cells with the construct for MYBtr^{exons1-9} or MYB-QKI, with MYBtr^{exons1-9} resulting in the highest level of activity (FIGS. 19A, 19B and 19C).

### Example 7

### Angiocentric Gliomas Exhibited MYB-QKI Expression Signature

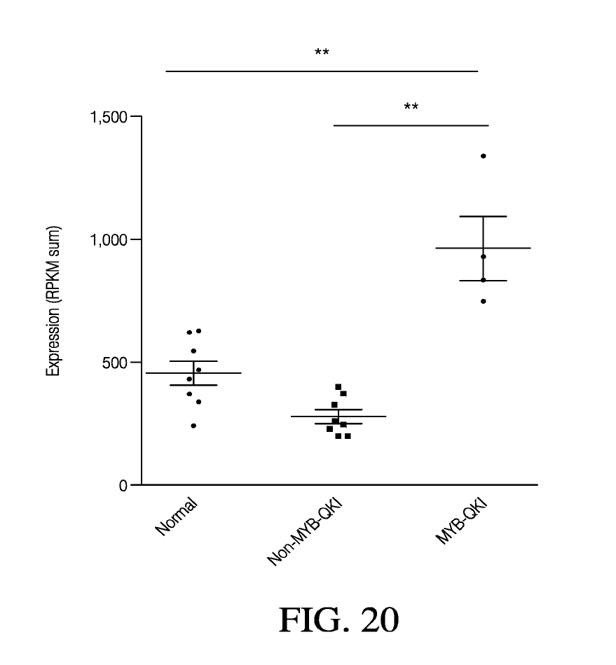

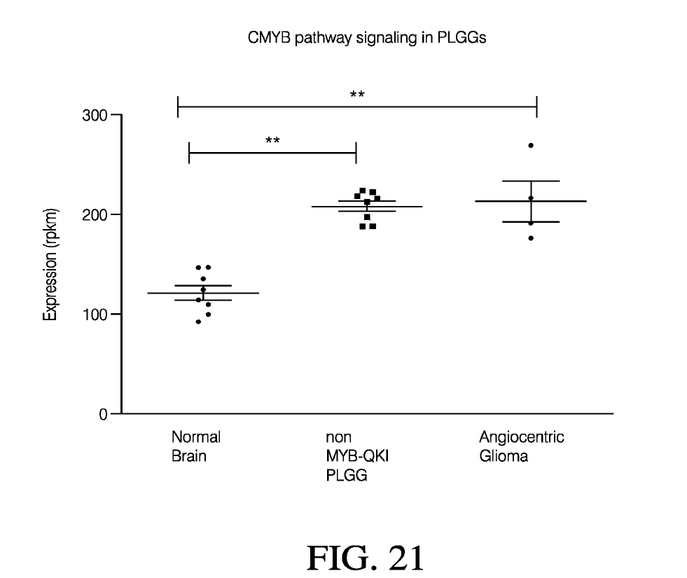

Angiocentric gliomas exhibited significantly higher expression of the MYB-QKI signature than normal pediatric brain (P=0.001) and PLGGs without MYB-QKI alterations (P=0.0011) (FIG. 20). PLGGs exhibited increased expression of genes associated with MYB pathway activation as compared to normal brain (P=0.0003), but this increased expression was not specific to tumors with MYB-QKI rearrangement and was of lower magnitude than the difference observed for the MYB-QKI signature genes (FIG. 21).

### Example 8

### MYB-QKI Rearrangements Drive Aberrant Expression of Truncated MYB

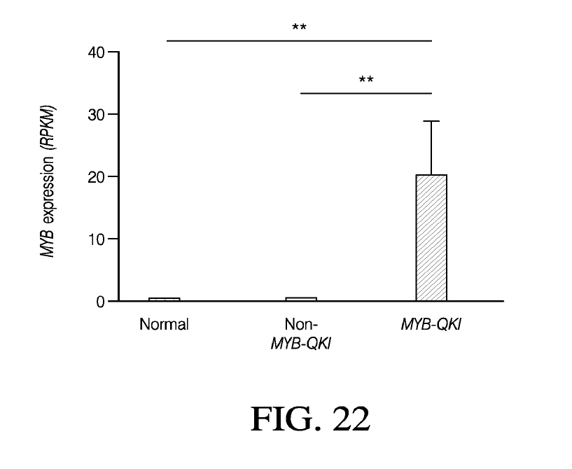

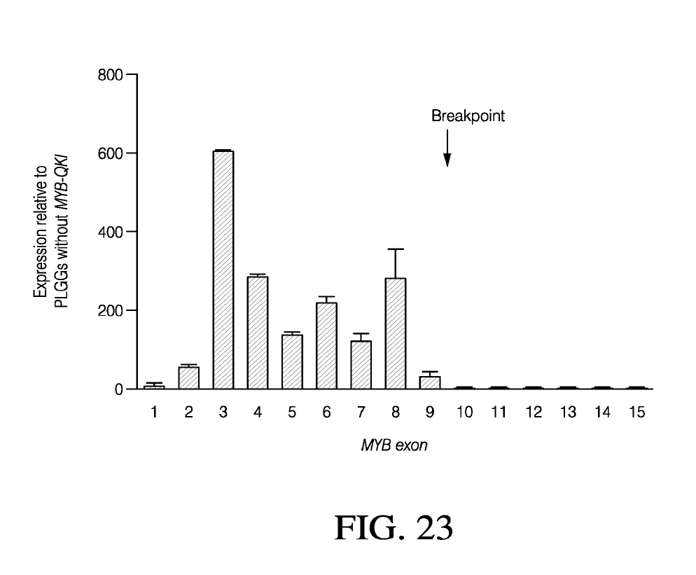

Angiocentric gliomas with MYB-QKI rearrangement exhibit significantly higher MYB expression than normal pediatric cortical brain (P=0.0062) or PLGGs with BRAF or fibroblast growth factor receptor (FGFR) gene alterations (P=0.03) (FIG. 22). The MYB transcript that is expressed in angiocentric gliomas is truncated and corresponds to the exons retained in the rearranged MYB-QKI allele. Three angiocentric gliomas harbored MYB-QKI rearrangement breakpoints between exons 9 and 10 of MYB. These tumors exhibited increased expression of MYB exons 1-9 relative to PLGGs that did not harbor MYB-QKI rearrangement (P<0.05) but had minimal expression of the remaining MYB exons (FIG. 23). These data support selective, aberrant regulation of the expression of truncated MYB via MYB-QKI.

### Example 9

### MYB-QKI Rearrangement Results in Enhancer Translocation

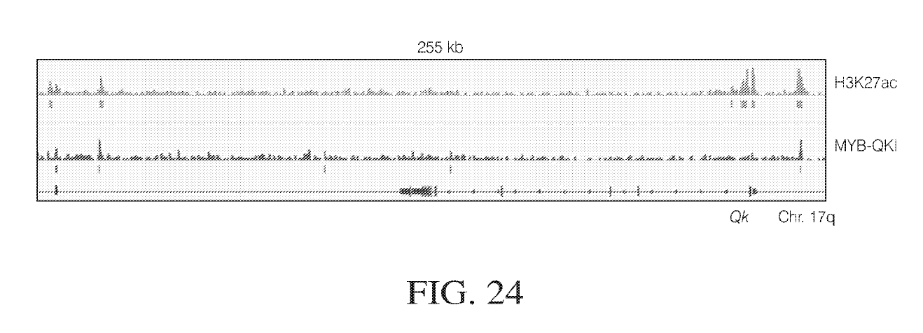

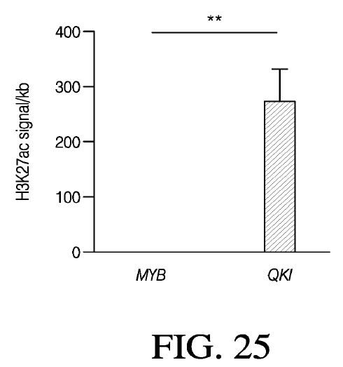

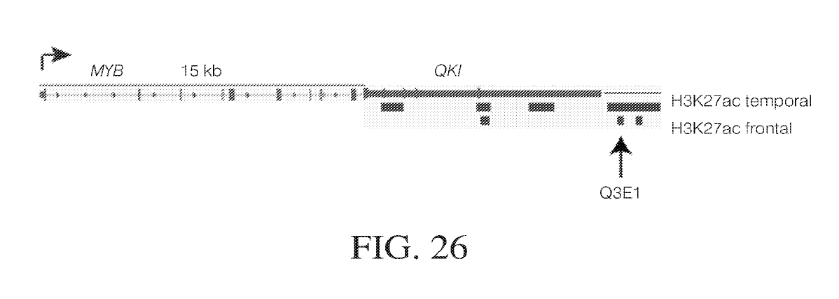

Aberrant oncogene expression can result from enhancer translocation. In published H3K27ac enhancer profiles from normal human cortical brain samples, the MYB locus is not associated with H3K27ac enhancer peaks, consistent with the finding that MYB is not expressed. In contrast, QKI, which is expressed, was associated with several H3K27ac peaks, including sequences at the 3′ end of QKI (FIGS. 24, 25, 26). MYB-QKI rearrangement is predicted to bring these H3K27ac-associated enhancer elements from the 3′ end of QKI to within only 15 kb of the MYB promoter (FIG. 26).

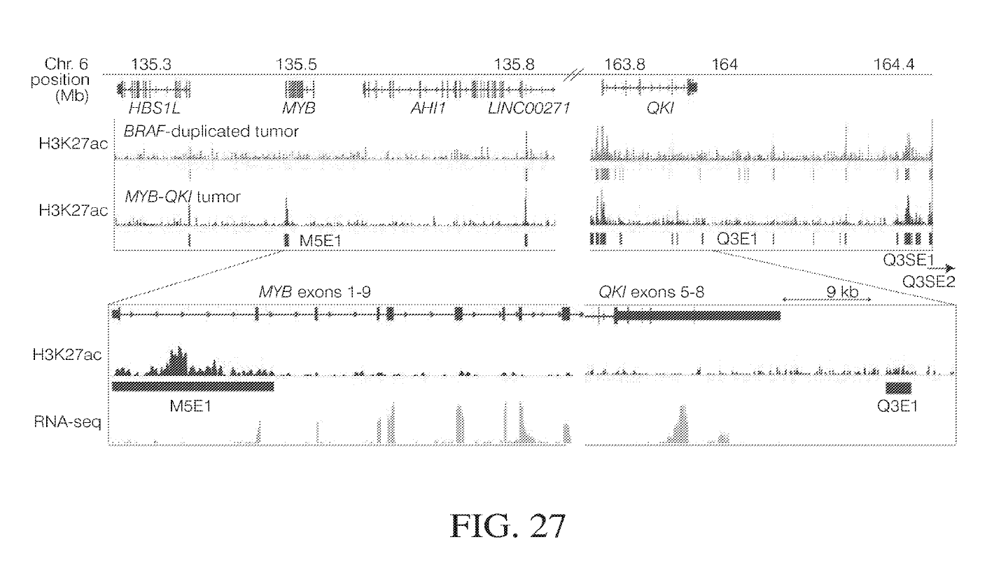

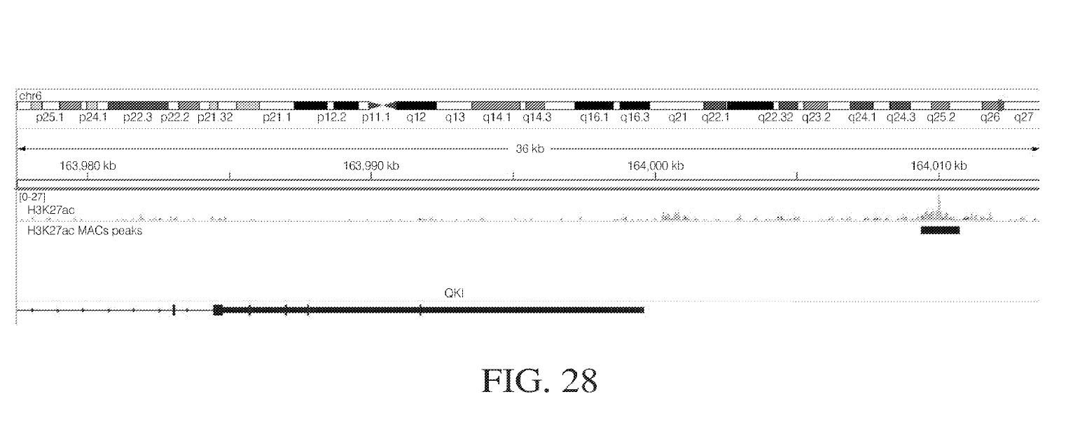

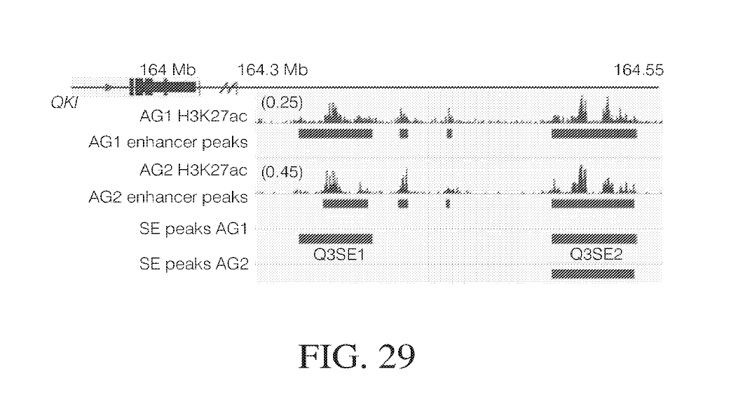

Profiling of H3K27ac-associated enhancers for two human angiocentric gliomas expressing MYB-QKI confirmed the presence of active enhancer elements that are translocated proximal to the MYB promoter (FIGS. 27, 28). ChIP-seq analysis identified multiple H3K27ac peaks associated with the 3′ end of QKI, similar to the peaks observed in normal human brain and in a BRAF-duplicated supratentorial pilocytic astrocytoma. Enhancers within 10 kb of the region 3′ to QKI and a larger cluster of super-enhancers 100-500 kb 3′ to QKI (Q3SE1 and Q3SE2) were also observed. In angiocentric gliomas with MYB-QKI rearrangement, these enhancers are translocated proximal to the MYB promoter (FIG. 29).

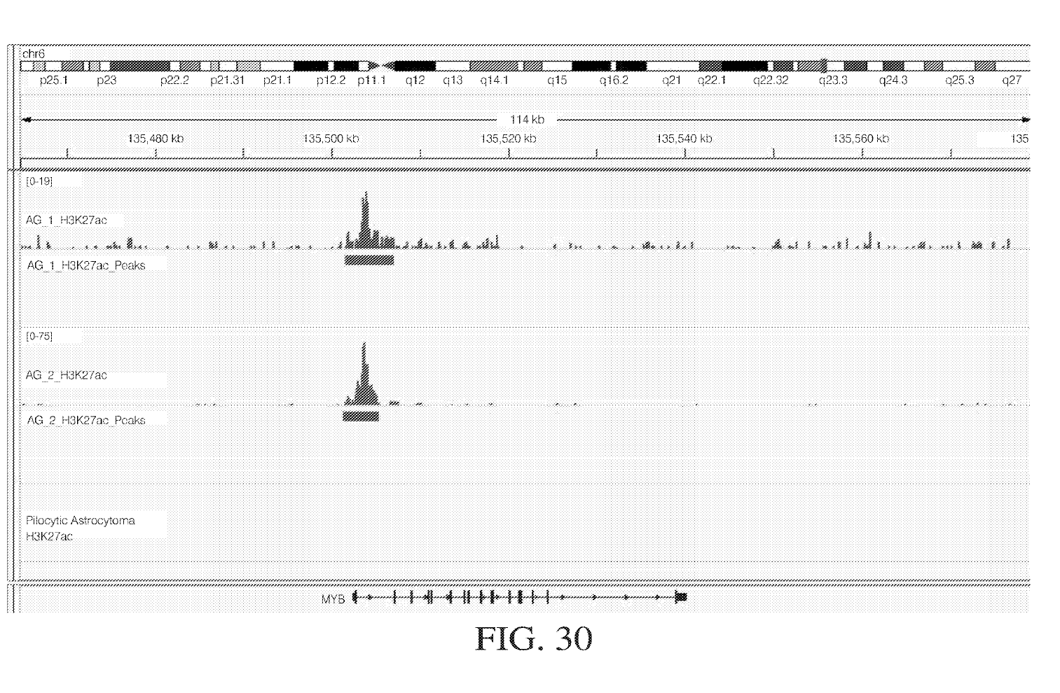

An aberrant enhancer associated with the MYB promoter in MYB-QKI-defined angiocentric glioma was observed (FIG. 27). Normal human cortical brain does not show evidence of MYB-related enhancers associated with H3K27ac43, and, indeed, we did not observe the formation of H3K27ac enhancer peaks at MYB in the pilocytic astrocytoma (FIG. 30). However, in both angiocentric gliomas, a large H3K27ac peak associated with the MYB promoter (M5E1) was observed. RNA-seq showed expression of the first nine exons of MYB corresponding to those retained in the rearrangement, suggesting that the aberrant M5E1 enhancer is regulating expression of truncated MYB from the rearranged allele. The lack of expression of full-length MYB indicates that the aberrant enhancer does not regulate the expression of the remaining wild-type MYB allele.

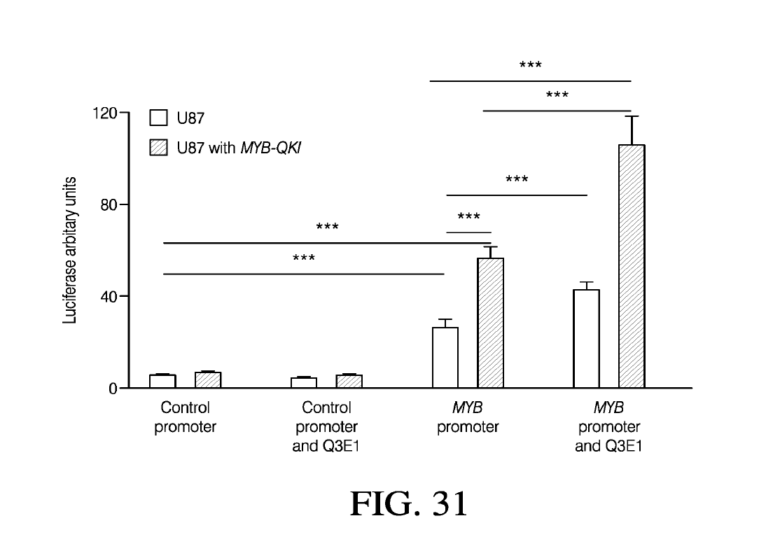

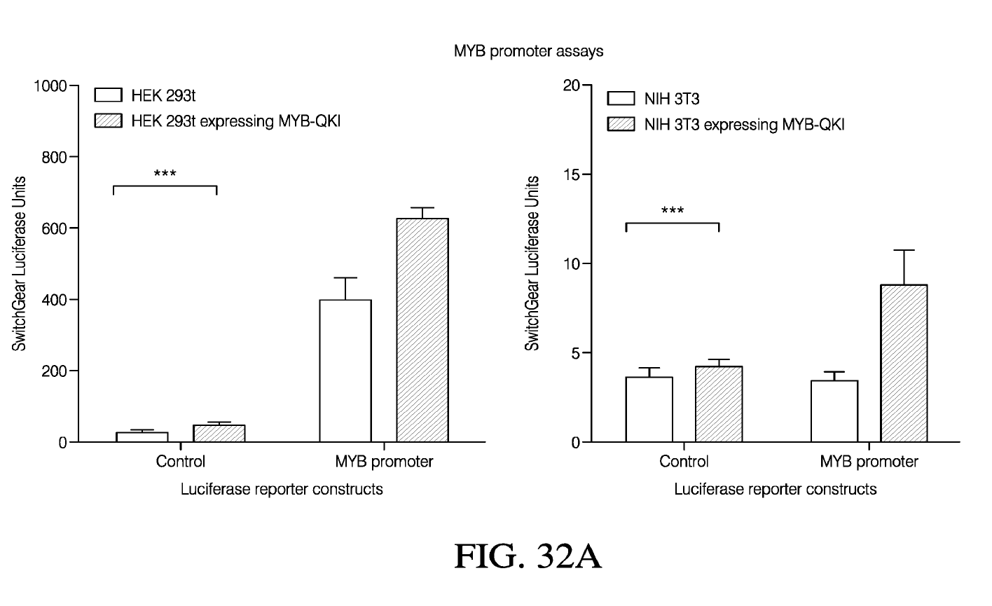

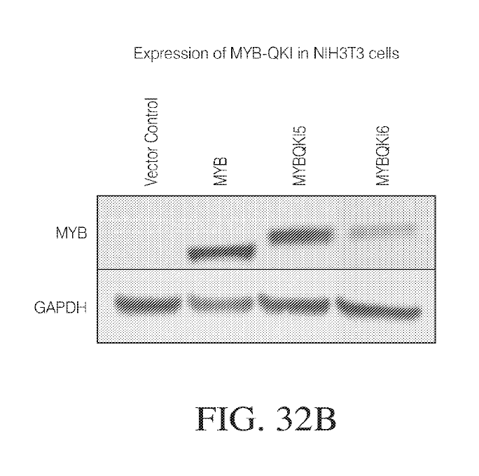

Whether MYB-QKI fusion protein was able to functionally activate the MYB promoter was examined by creating a luciferase reporter construct with the human MYB promoter upstream of the luciferase gene (MYB-luc). Significant induction of MYB promoter activity in human U87 glioma cells stably expressing MYB-QKI with the MYB-luc reporter as compared to U87 cells with the MYB-luc or promoterless control luciferase construct alone was observed (FIG. 31). This suggests that MYB-QKI contributes to an autoregulatory feedback loop, possibly by binding to the MYB promoter. MYB-QKI activated the MYB promoter in two additional cellular contexts (HEK293T and NIH3T3 cells; FIGS. 32A, 32B).

Enhancers in the 3′ UTR of QKI could aberrantly activate the MYB promoter when translocated, thereby further driving MYB-QKI expression. The proximal enhancer sequence from the QKI 3′ UTR (Q3E1) was cloned upstream of the human MYB promoter in the MYB-luc reporter construct. The baseline activity of the Q3E1-MYB-luc construct was higher than that of the MYB-luc construct in U87 cells, with activation increased by approximately 1.5-fold, a level of activation shown to have biological relevance in other diseases. Expression of MYB-QKI with the Q3E1-MYB-luc reporter led to even higher activity, again consistent with an autoregulatory feedback loop in the presence of the fusion protein (FIG. 31).

### Example 10

### MYB-QKI Fusion Protein is Oncogenic

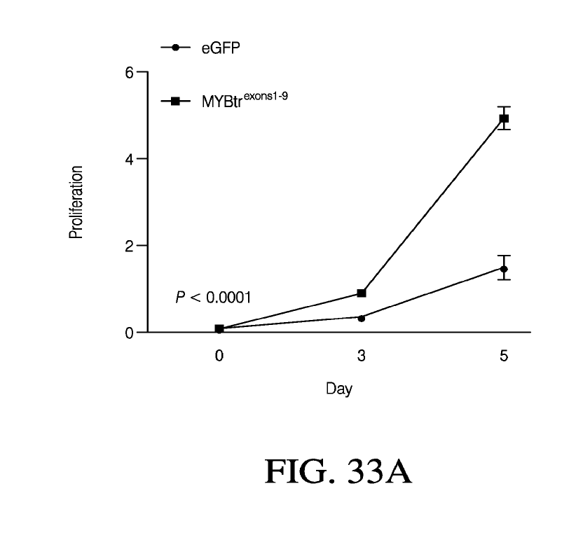

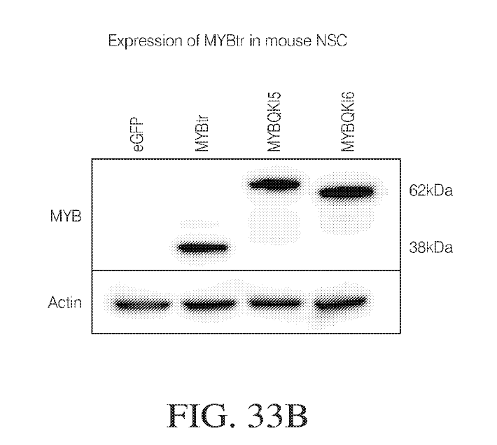

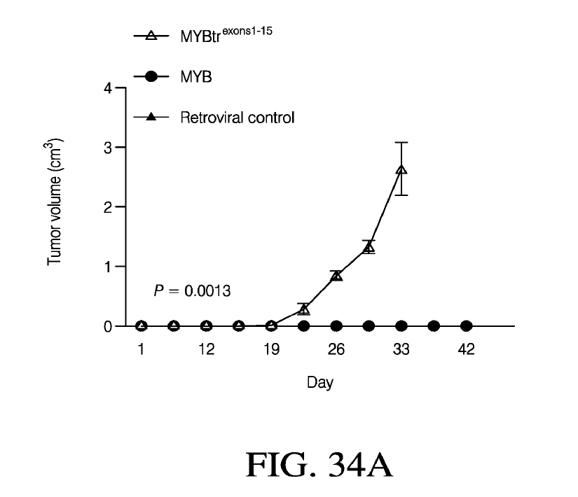

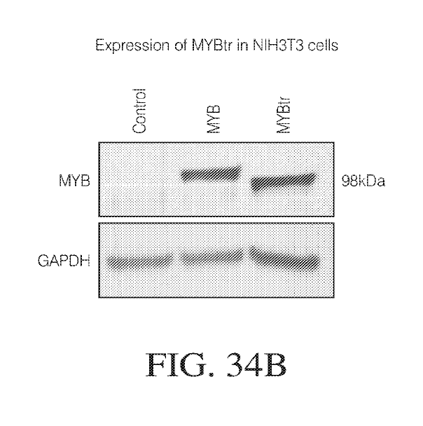

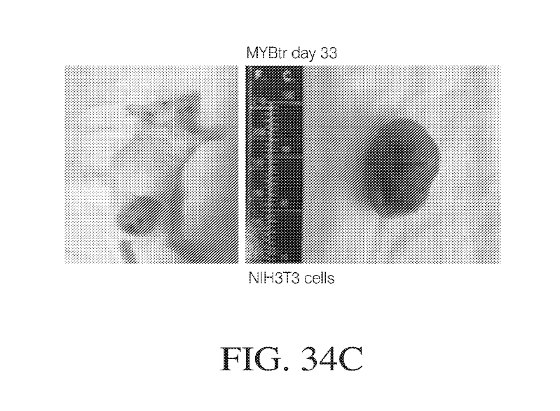

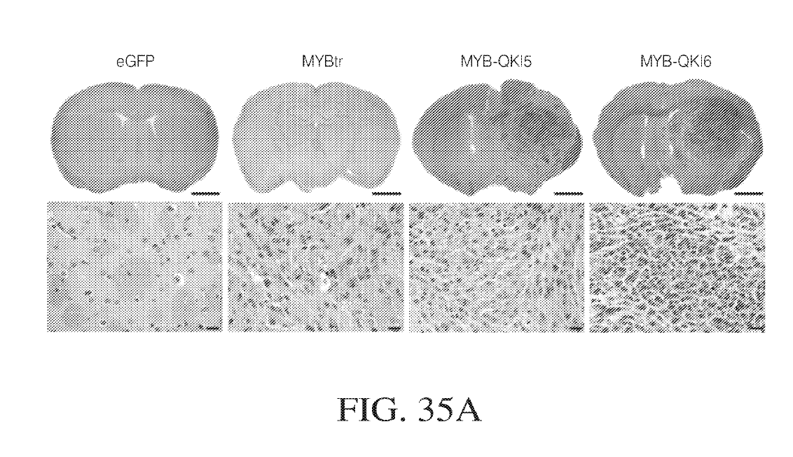

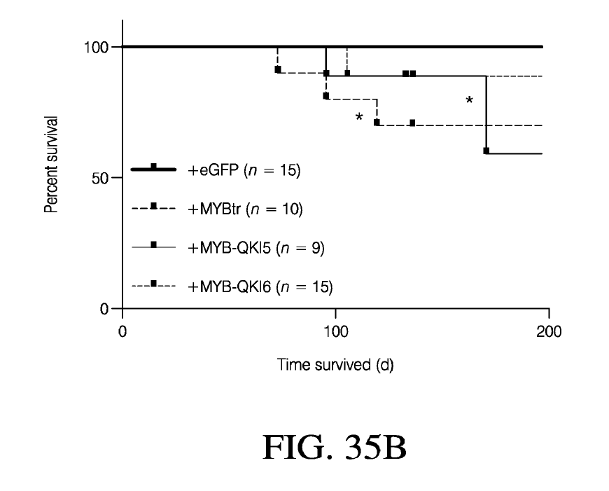

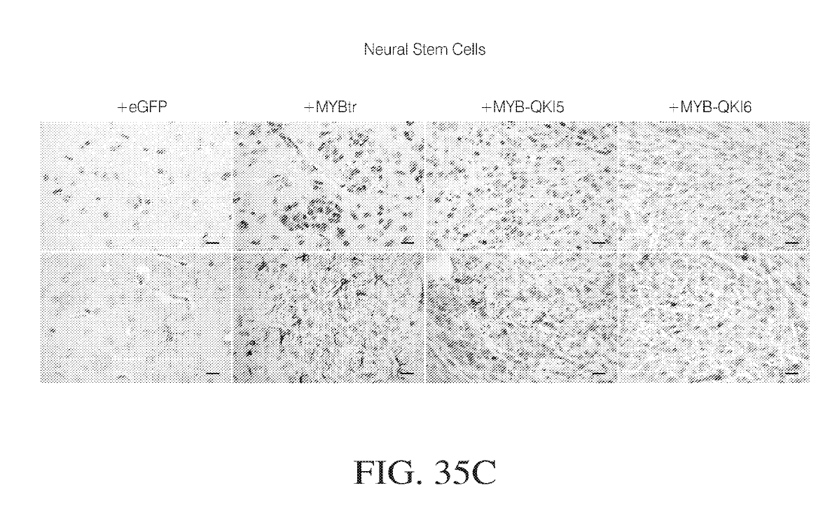

Expression of truncated MYB has previously been reported to be oncogenic. In mNSCs, overexpression of truncated MYB from exons 1-9 (short variant) increased the cell proliferation rate in comparison to eGFP control (FIGS. 33A, 33B), and in NIH3T3 cells overexpression of human MYBtr^{exons1-15} but not full-length MYB induced tumor formation when cells were injected into mouse flanks (FIGS. 34A, 34B, 34C). Furthermore, mNSCs expressing human truncated MYB induced diffuse glioma formation 100 days, on average, after intracranial injection in mice (FIGS. 35A, 35B). These tumors expressed OLIG2 and GFAP in a subset of tumor cells, a pattern similar to that observed in human diffuse gliomas (FIG. 35C).

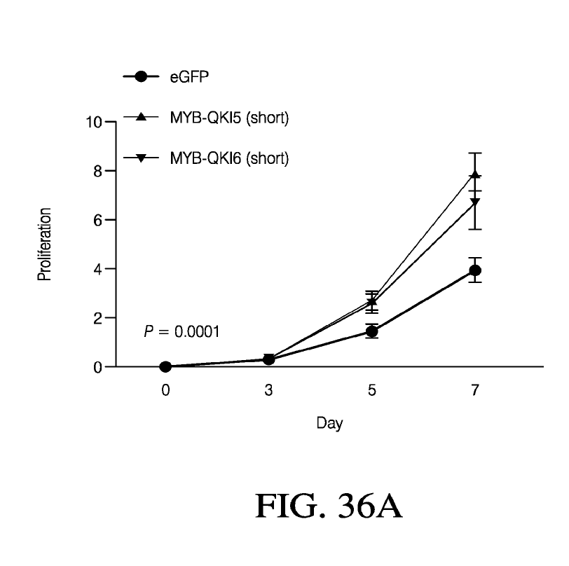

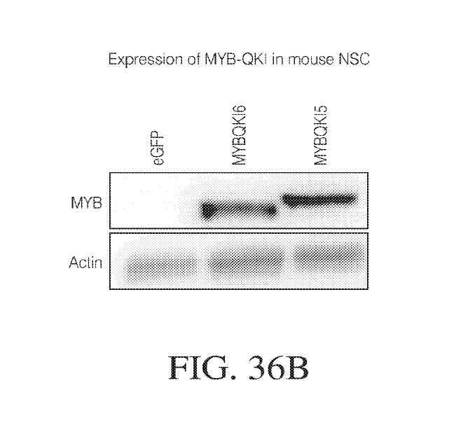

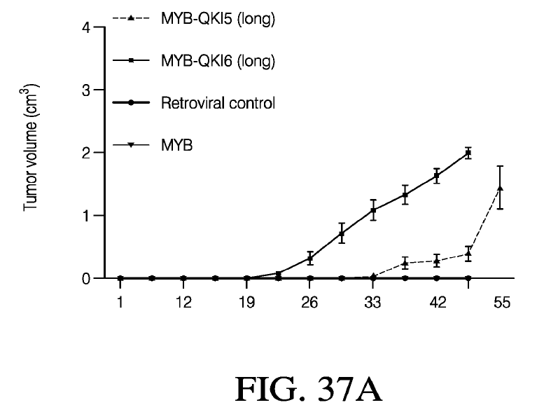

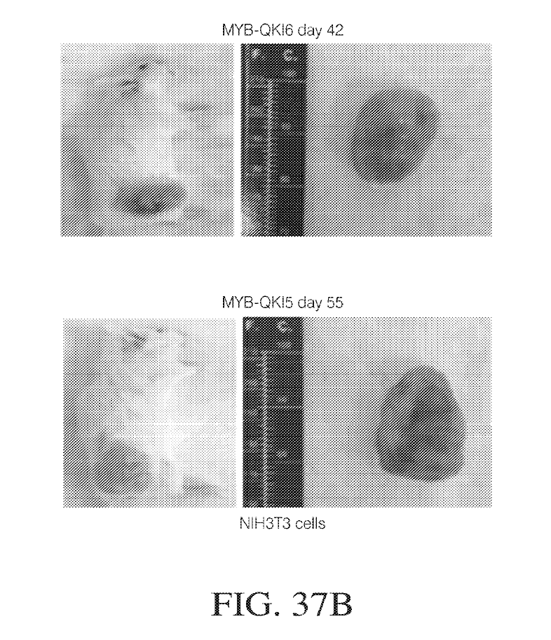

To test whether MYB-QKI fusions are oncogenic, human MYB-QKI5 and MYB-QKI6 were stably expressed in mNSCs and NIH3T3 cells. In mNSCs, overexpression of either isoform led to a significantly increased proliferation rate as compared to expression of eGFP (p<0.0001; FIGS. 36A, 36B). Similarly, both isoforms induced anchorage-independent growth when overexpressed in NIH3T3 cells. In vivo overexpression of both MYB-QKI5 and MYB-QKI6 in NIH3T3 cells but not of full-length MYB led to tumor formation as flank xenografts (FIGS. 37A, 37B). Intracranial injections with mNSCs overexpressing MYB-QKI5 or MYB-QKI6 resulted in glioma formation and infiltrating tumor cells, with some evidence of enhanced growth around vessels and a clustered growth pattern, features similar to those of angiocentric glioma and distinct from the histology seen in models of adult glioblastoma (for example, EGFRvIII overexpression in Ink4a/Arf−/− NSCs). However, these tumors differed from human angiocentric gliomas in that they had high-grade features with frequent mitoses and marked cytological atypia (FIGS. 35A, 35C). Immunohistochemical analysis showed diffuse GFAP expression and a subset of OLIG2-positive tumor cells, a pattern similar to that seen in human angiocentric gliomas (FIG. 35C).

In total, it was established that flank injections in 15 mice of NIH3T3 cells overexpressing either truncated MYB or MYB-QKI fusion protein (five vector controls) and intracranial injections in 29 mice of mNSCs expressing truncated MYB or MYB-QKI fusion protein (15 vector controls). Flank tumors were observed in all 15 mice injected with NIH3T3 cells overexpressing either truncated MYB or MYB-QKI and five intracranial tumors from mice injected with mNSCs expressing truncated MYB or MYB-QKI. Tumor formation was not observed from any cells expressing vector control. These data represent a significant enrichment of tumor formation in cells expressing truncated MYB or MYB-QKI fusion protein (P<0.0001).

### Example 11

### MYB-QKI Rearrangement Disrupts QKI, a Tumor Suppressor

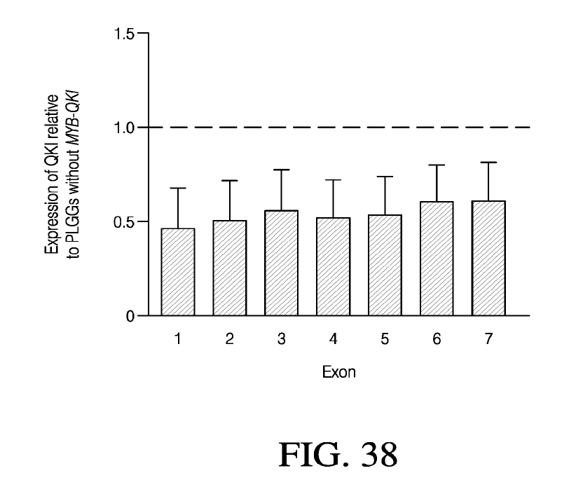

Next, how the disruption of QKI might contribute to oncogenicity in tumors that harbor MYB-QKI rearrangement was studied. Exon-specific RNA-seq analysis of angiocentric gliomas with MYB-QKI rearrangement (n=4) showed reduced expression of QKI as compared to PLGGs that harbored BRAF alterations (n=5) (FIG. 38). These data suggest that the MYB-QKI rearrangement might contribute to tumor formation through reduced expression of QKI, a tumor-suppressor gene.

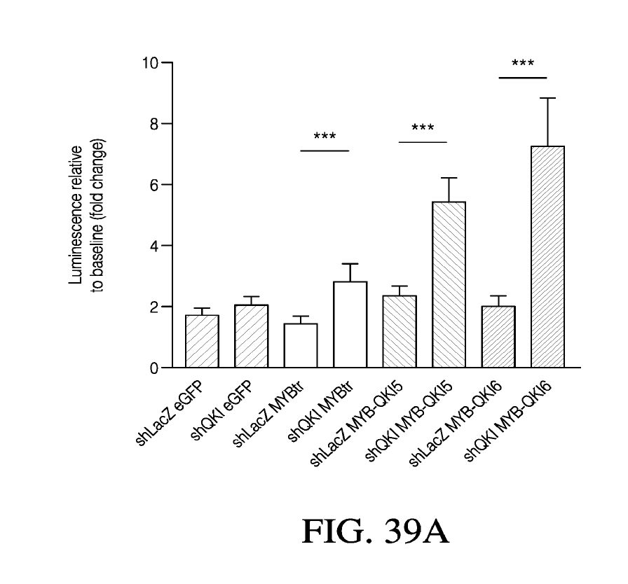

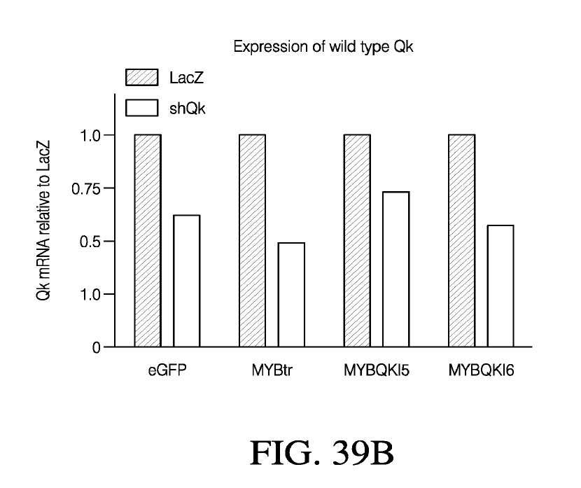

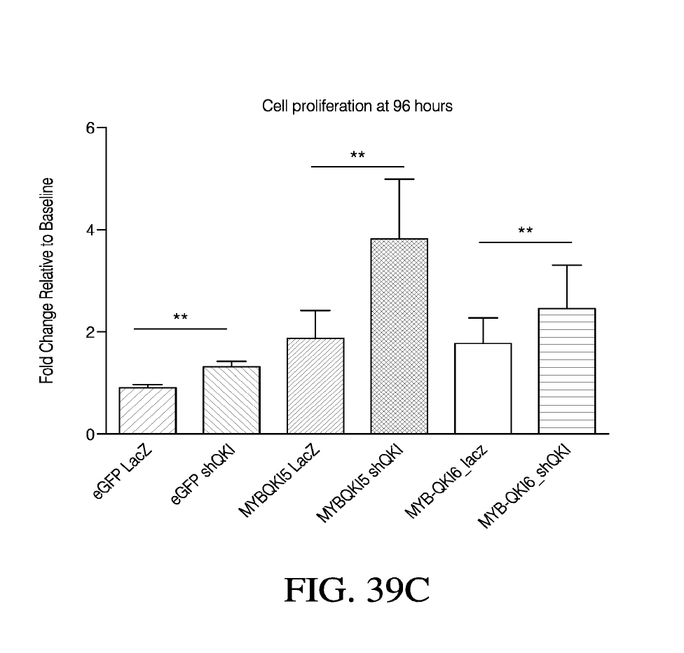

Indeed, suppressing wild-type Qk (a mouse homologue of human QKI) using short hairpin RNAs (shRNAs) that targeted the first four exons of Qk led to increased proliferation of mNSCs, with the greatest increase observed in the context of preexisting human MYB-QKI expression. In mNSCs overexpressing human truncated MYB, MYB-QKI5 or MYB-QKI6, suppression of wild-type Qk was sufficient to increase proliferation within only 3 days of suppression (FIGS. 39A, 39B). The greatest effect was observed in cells overexpressing MYB-QKI fusion protein, despite a similar or lower degree of suppression of Qk in these cells as compared to cells overexpressing eGFP or truncated MYB. Increased proliferation within 3 days of suppression in cells expressing eGFP was not observed, although a mild increase in proliferation on day 5 was observed (FIG. 39C). These data suggest that MYB-QKI overexpression and QKI suppression exert cooperative functional effects.

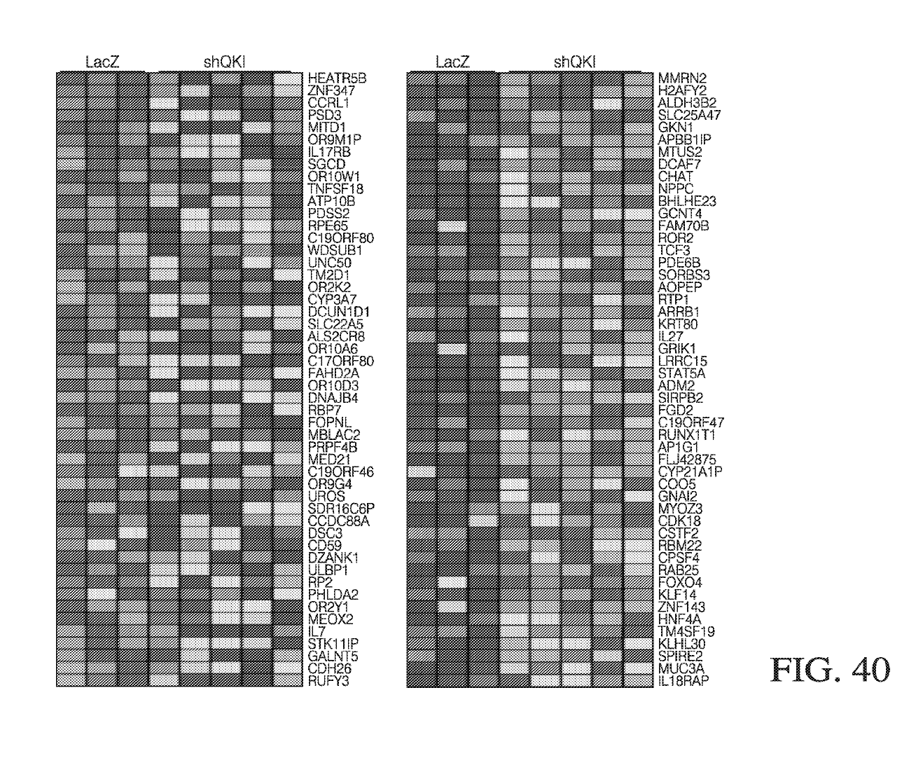

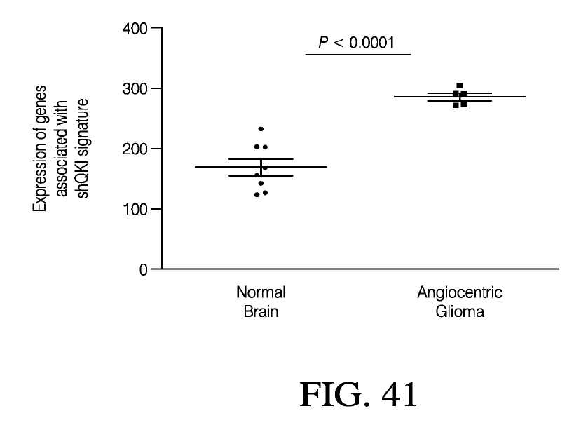

Suppression of Qk by shRNA in mNSCs expressing MYB-QKI6 led to differential expression of 309 genes relative to cells instead expressing control shRNA to lacZ (q<0.25). QKI has previously been reported to regulate the expression of microRNAs (miRNAs), and we also observed upregulation of ten miRNAs with suppression of wild-type Qk, including miR-717. Isoform 7 of mouse Qk is predicted to contain a miRNA regulatory element (MRE) for miR-717. Angiocentric gliomas exhibit molecular effects consistent with QKI suppression. In mNSCs, a signature consisting of the 50 genes whose expression was most correlated with Qk suppression was defined (FIG. 40). This signature was significantly enriched in angiocentric gliomas relative to normal human brain (P<0.0001; FIG. 41).

### Example 12

### Inhibition of Growth and Induction of Death of Cells Harboring MYB-QKI Rearrangement

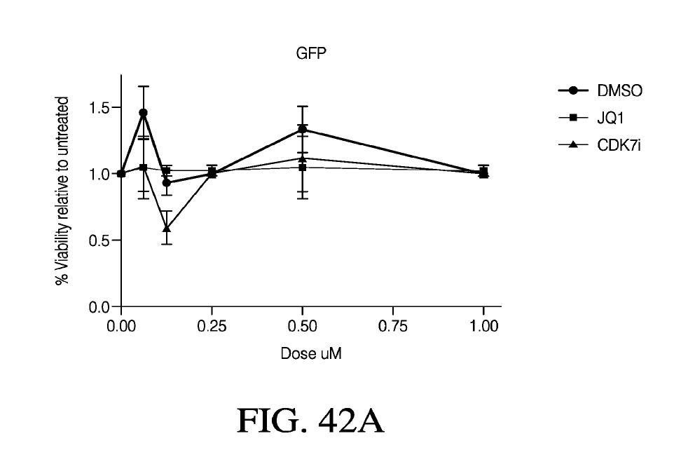

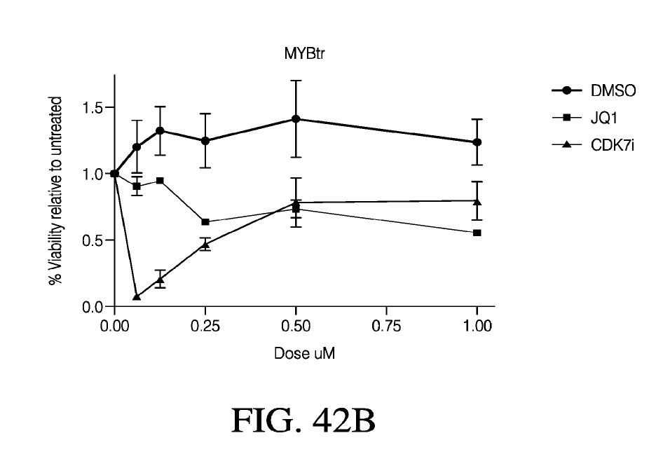

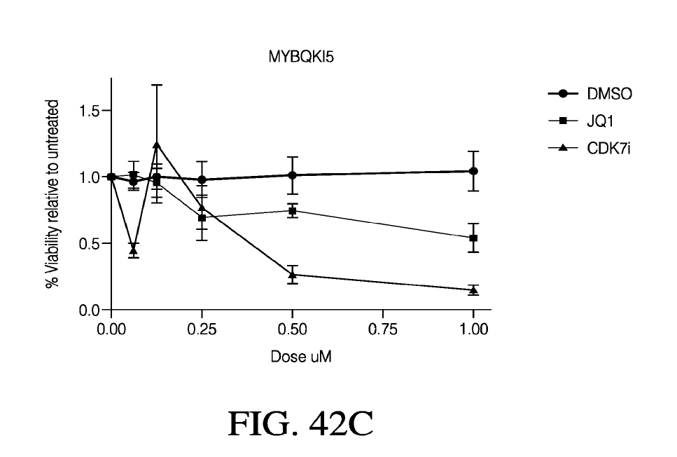

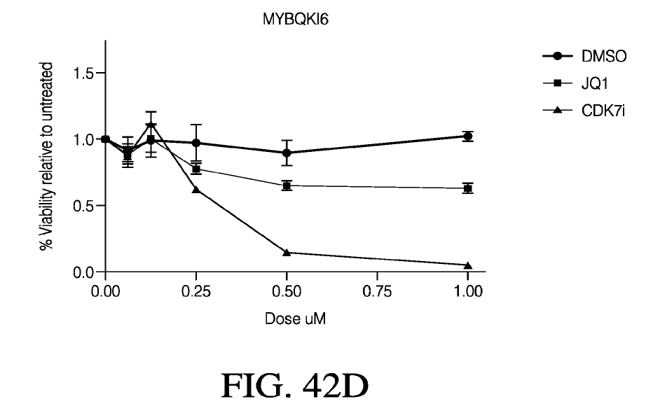

Modifiers of the MYB pathway and key upstream and downstream effectors were examined for their ability to inhibit growth and induce death of cells expressing MYB-QKI fusion protein. As shown in FIGS. 42A-D, BET inhibitor JQ1 significantly compromised the viability of mouse neural stem cells engineered to express truncated MYB (FIG. 42B), MYB-QKI5 (short variant) (FIG. 42C), and MYB-QKI6 (short variant) (FIG. 42D) in comparison with the viability of mouse neural stem cells engineered to express GFP as a negative control (FIG. 42A). Thus, cells expressing truncated MYB or MYB-QKI were specifically sensitive to inhibition of BET by JQ1.

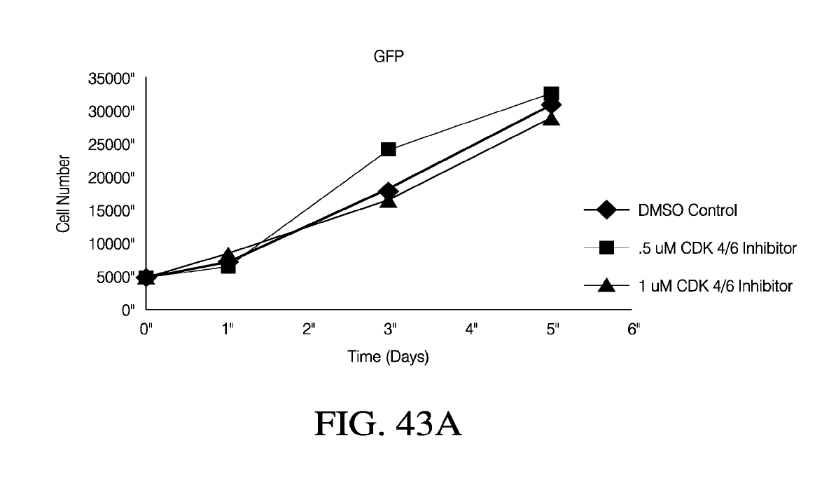

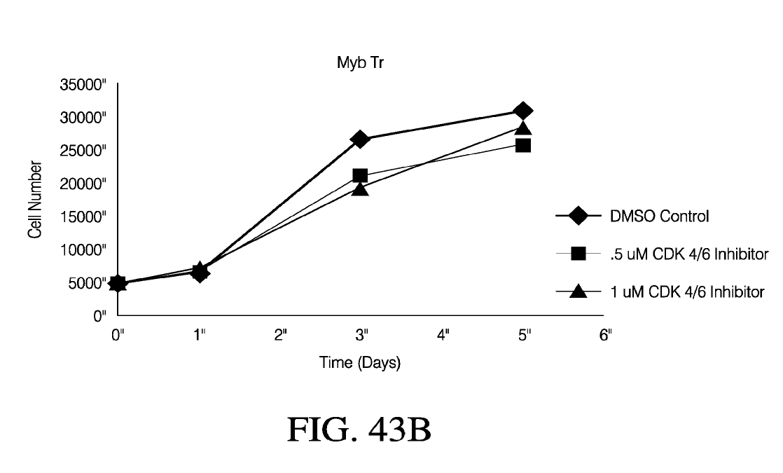

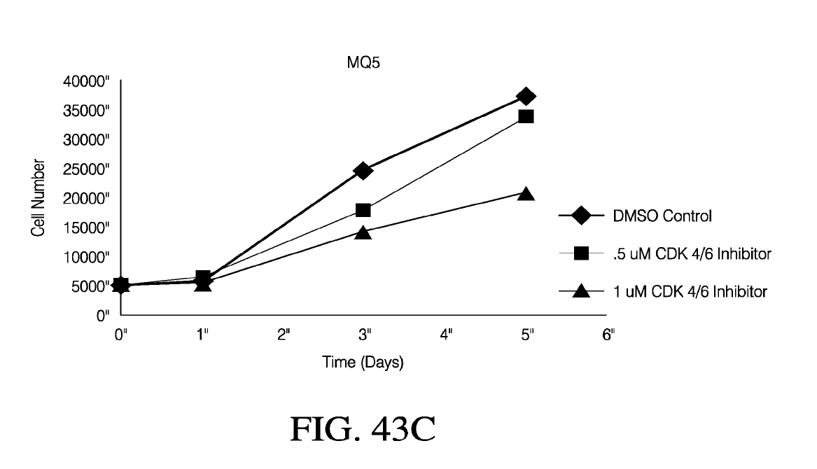

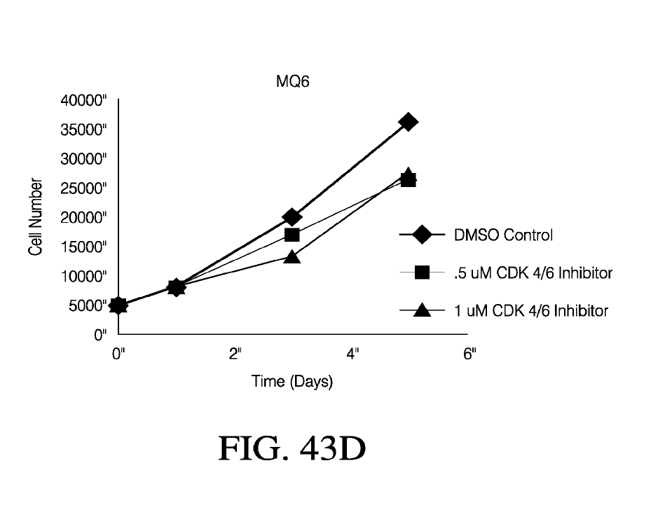

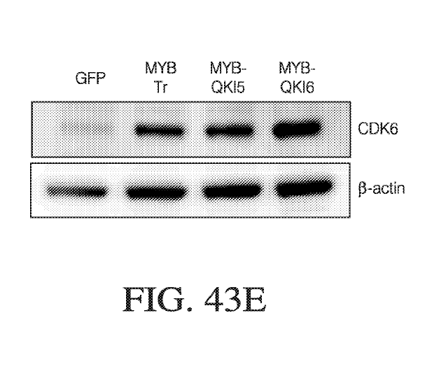

Inhibition of CDK7 was also examined using a specific CDK7 inhibitor THZ1. As shown in FIGS. 42A-D, THZ1 (“CDK7i”) significantly compromised the viability of mouse neural stem cells engineered to express truncated MYB (FIG. 42B), MYB-QKI5 (short variant) (FIG. 42C), and MYB-QKI6 (short variant) (FIG. 42D) in comparison with the viability of mouse neural stem cells engineered to express GFP as a negative control (FIG. 42A). Thus, cells expressing MYB-QKI were sensitive to inhibition of CDK7 by THZ1. This effect is specific with CDK7. As shown in FIGS. 43A-D, LY2835219, inhibitor of CDK4 and CDK6, did not strongly inhibit the growth of mouse neural stem cells engineered to express truncated MYB (FIG. 43B), MYB-QKI5 (short variant) (FIG. 43C), and MYB-QKI6 (short variant) (FIG. 43D) in comparison with the growth of mouse neural stem cells engineered to express GFP as a negative control (FIG. 43A), though the expression of CDK6 was increased in these cells (FIG. 43E). Thus, cells expressing truncated MYB or MYB-QKI were specifically sensitive to inhibition of CDK7 by THZ1.

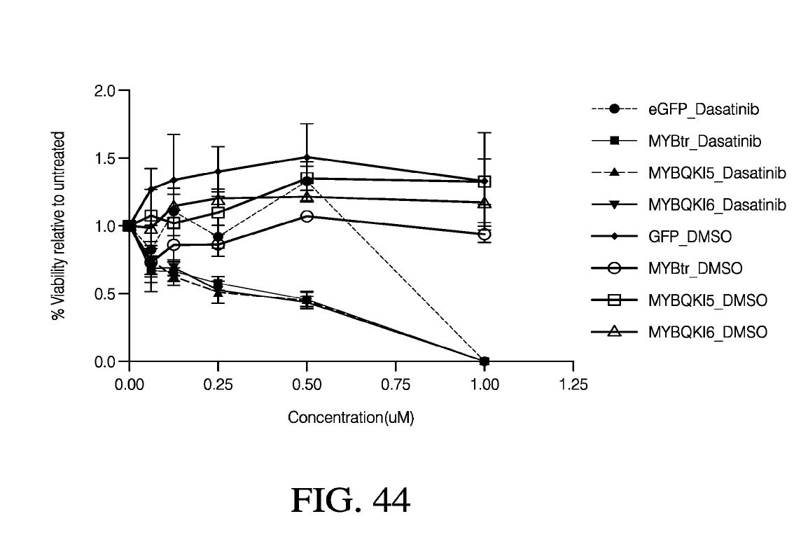

Inhibition of c-KIT was also examined using a c-KIT inhibitor dasatinib. As shown in FIG. 44, dasatinib at a concentration of 0.1-0.5 μM specifically compromised the viability of mouse neural stem cells engineered to express truncated MYB (“MYBtr”), MYB-QKI5 (short variant) (“MYBQKI5”), and MYB-QKI6 (short variant) (“MYBQKI6”) without causing significant cell death in mouse neural stem cells engineered to express GFP (“GFP”). Thus, cells expressing truncated MYB or MYB-QKI were specifically sensitive to inhibition of c-KIT by dasatinib.

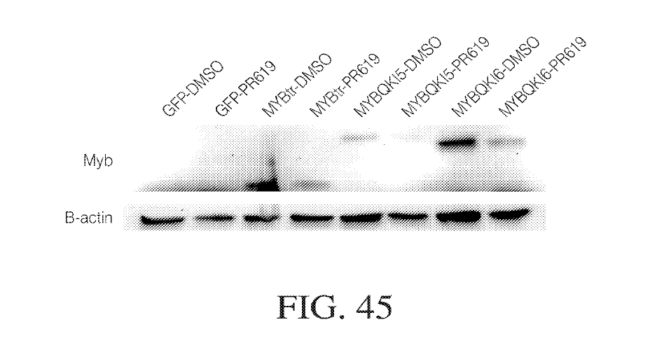

Another possible approach of suppressing cells expressing truncated MYB or MYB-QKI is reducing the amount of the truncated MYB or MYB-QKI protein, for example, by increasing the degradation of this protein. DUBs remove ubiquitination and thereby increase the half-life of proteins. Thus, DUB inhibitors may accelerate the degradation of truncated MYB or MYB-QKI. As shown in FIG. 45, a cell-permeable DUB inhibitor, PR619, reduced the amount of truncated MYB (“MYBtr”), MYB-QKI5 (short variant) (“MYBQKI5”), and MYB-QKI6 (short variant) (“MYBQKI6”). Thus, PR619 may also specifically inhibit the viability and growth of cells expressing truncated MYB or MYB-QKI.

### Example 13

### Methods

### Whole-Genome Sequencing and Processing

PLGGs and normal controls from CBTTC—Children's Hospital of Philadelphia and Dana-Farber/Harvard Cancer Center-Pediatric Low Grade Astrocytoma Consortium were sequenced at Beijing Genomics Institute at Children's Hospital of Philadelphia and the Broad Institute. DNA was randomly fragmented, and libraries were prepared for paired-end sequencing on an Illumina HiSeq 2000 instrument. Sequencing files from recently published PLGG data sets were accessed. Read pairs were aligned to reference genome hg19 (Build 37) using the Burrows-Wheeler Aligner (BWA) with options -q 5 -132 -k 2 -o 1. Reads were sorted by coordinates, normalized and cleaned, and duplicates were marked using SAMtools and Picard. Base quality score assignments were recalibrated to control for biases due to flow cell, lane, dinucleotide context and machine cycle using the Genome Analysis Toolkit (GATK). Copy number alterations were evaluated using SegSeq. GISTIC 2.0 was used to identify recurrent copy number alterations. Somatic point mutations and short indels were called using MuTect and IndelLocator and visual inspection in the Integrative Genomics Viewer (IGV). MutSig (version 2.0) was applied to detect significantly recurrent mutations. Rearrangements and breakpoints were identified using dRanger, BreakPointer and visual inspection. All analyses were performed within Firehose.

### RNA Sequencing and Analysis Pipeline

Following RNA extraction (RNeasy kit, Qiagen), library construction was performed using a non-strand-specific Illumina TruSeq protocol. Flow cell cluster amplification and sequencing were performed according to the manufacturer's protocols using HiSeq 2000 or 2500 instruments, with a 76-bp paired-end run including an 8-base index barcode read. RNA-seq files were downloaded from published data sets. RNA-seq BAM files were transformed to fastq files using the Picard SamToFastq algorithm. Raw paired-end reads were aligned to the hg19 reference genome and preprocessed using PRADA (pipeline for RNA sequencing data analysis). PRADA was used within Firehose to determine gene expression levels, exon expression levels and quality metrics and for the detection of fusion transcripts. BAM files were also assessed by visual inspection.

### Array CGH

DNA was extracted from archival formalin-fixed, paraffin embedded samples and array CGH was performed as previously described (Ramkissoon et al., 2013). GC content—normalized copy number data were cleaned of known germline copy number variations, and circular binary segmentation was used to segment the copy number data (α=0.001, undo.splits=sdundo, undo.s.d.=1.5, minimum width=5).

### Whole-Exome Sequencing

Whole-exome sequencing was performed from formalin-fixed, paraffin-embedded samples (without matched controls). These samples were used to confirm driver alterations identified by whole-genome sequencing. DNA was extracted using the Qiagen DNA Blood and Tissue kit. Libraries with a 250-bp average insert size were prepared by Covaris sonication, followed by two rounds of size selection (Agencourt AMPure XP beads) and ligation to specific barcoded adaptors (Illumina TruSeq) for multiplexed analysis. Exome hybrid capture was performed with the Agilent Human All Exon v2 (44 Mb) bait set.

Sequence data were aligned to the hg19 reference genome with BWA using parameters -q 5 -132 -k 2 -t 4 -o 1. Aligned data were sorted, marked for duplicates and indexed with Picard tools. Base quality score recalibration and local realignment around insertions and deletions was achieved with GATK.

Mutations were called with MuTect, filtered against a panel of normal samples and annotated to genes with Oncotator. Likely germline SNPs were removed by filtering against the ESP and Exome Aggregation Consortium (ExAC) databases.

### Histological Assignment

Histological subtype assignments were according to previously published data. Samples not previously published were centrally reviewed and classified by a board-certified neuropathologist (K. L. L., S. Santagata or S. H. R.) using WHO 2007 criteria.

### MYB FISH Analysis

FISH was performed using 5-μm formalin-fixed, paraffin-embedded tissue sections and Homebrew probes RP11-63K22 (5′ to MYB; directly labeled in SpectrumOrange) and RP11-170P19 (3′ to MYB; directly labeled in SpectrumGreen) that map to 6q23.3. MYB status was assessed in 50 tumor nuclei per sample. A CEP6 aqua probe (Invitrogen) mapping to the centromeric region of chromosome 6 was co-hybridized as a control.

### Immunohistochemistry

Diaminobenzidine (DAB), bright-field staining was performed according to standard protocols on 5-μm paraffin-embedded sections. Heat and 10 mM sodium citrate buffer (pH 6.0) were used for antigen retrieval for antibodies to MYB (Abcam for human tissue, Bethyl Laboratories for mouse tissue), OLIG2 (Chemicon) and GFAP (Millipore). Counterstaining for nuclei was performed using Mayer's hematoxylin stain, and coverslips were mounted with Permount (Fisher Scientific). Sections from the left occipital pole of a normal adult brain autopsy were used to assess MYB levels.

### Analysis of QKI Alterations in TCGA Samples

GISTIC 2.0 analyses were performed across 10,570 tumor samples from 31 lineages from TCGA, as previously described (Zack et al., 2013).

### Analysis of Gene Expression in Normal Tissues

RNA-seq data for normal pediatric brain samples were accessed from the BRAINSPAN Atlas of the developing human brain. MYB expression levels from RNA-seq obtained from normal autopsy tissues were downloaded from the GTEx Consortium. Expression levels were compared using ANOVA and t tests. P values <0.05 were considered to be significant.

### Vector Construction and Generation of NIH3T3 Stable Lines

MYB-QKI5 and MYB-QKI6 constructs were synthesized as Gateway-compatible entry clones. Truncated MYB constructs were generated via PCR mutagenesis using MYB-QKI fusions as templates. Full-length MYB and QKI constructs were purchased as Gateway entry clones from PlasmID/DF/HCC DNA Resource Core. MYB-QKI5 and MYB-QKI6, truncated MYB, full-length MYB and QKI constructs were subcloned into a Gateway-compatible N-Myc-tagged pMXs-Puro retroviral vector (Cell Biolabs). Platinum-E retroviral packaging cells (Cell BioLabs) were used to generate retrovirus according to the manufacturer's protocols. NIH3T3 cells were infected with retrovirus-containing medium for 6 h, and selection with puromycin was commenced 48 h after infection. Stable expression of Myc-tagged proteins was confirmed via immunoblot analysis with horseradish peroxidase (HRP)-conjugated antibody to Myc (Invitrogen R951-25; 1:5,000 dilution), antibody to MYB (Abcam; 1:5,000 dilution) and antibody to QKI (Bethyl Lab A300-183A; 1:1,000 dilution).

### Soft Agar Colony Formation Assays and Quantification

Anchorage-independent growth of NIH3T3 cells was assayed as previously described56 with the following modifications. NIH3T3 cells expressing each of the MYB-QKI15, MYB-QKI6, truncated MYB, full-length MYB and full-length QKI proteins and retroviral vector control were plated in 0.7% agar with DMEM and DBS in 96-well plates (in triplicate). Cell colonies were allowed to form for 2 weeks, and images were acquired. Images were analyzed using ImageJ software, and colonies with an area greater than 500 pixels were quantified.

### Generation of Reporter Constructs Containing the MYB Promoter and Enhancer Constructs

To assess the effect of candidate enhancer regions on MYB promoter activity, the human MYB promoter sequence was cloned into the pLightSwitch_Prom vector (Active Motif) that contains a multiplecloning site upstream of a Renilla luciferase reporter gene (RenSP) without a promoter. The MluI-BglII site in the pLightSwitch_Prom vector was used to clone the MYB promoter sequence, and the MluI site was further used to clone candidate enhancer regions upstream of the MYB promoter. The human QKI 3′ UTR enhancer sequences (hg18, chr. 6: 163,920,360-163,920,809 and chr. 6: 163,921,548-163,921,972) were synthesized by Invitrogen and cloned into the reporter constructs as described above. LightSwitch Random Promoter Control 1 (Active Motif) containing a 1-kb non-conserved, non-genic and non-repetitive fragment from the human genome cloned upstream of the RenSP luciferase reporter gene was used as a negative control. A reporter vector with a housekeeping gene promoter, LightSwitch ACTB Promoter Control, was used as a positive control for all assays. The luciferase reporter constructs containing the MYB promoter alone or together with enhancers were transfected into the U87 glioma line (or a U87 line stably expressing MYB-QKI) using Lipofectamine 3000 (n=5) or cotransfected with MYB-QKI or vector control into HEK293 cells via Lipofectamine 2000 (Invitrogen) or into NIH3T3 lines stably expressing MYB-QKI using PolyFect (Qiagen). Luciferase activity was quantified 24 h after transfection using LightSwitch Luciferase Assay Reagent (Active Motif) according to the manufacturer's protocol.

### Mim-1 Reporter Construct Generation and MYB Transactivation Assays

Luciferase reporter constructs containing a consensus DNA-binding sequence for c-MYB were generated. The reporter construct was designed using the core MYB recognition element (MRE) consensus sequence PyAAC(G/T)G, which is present in the mim-1 gene promoter, a previously described MYB target. Double-stranded oligonucleotides were generated by annealing primers mim-1 forward and mim-1 reverse. The annealed oligonucleotide was ligated into pGL4.10[luc2] vector (Promega) digested with XhoI and HindIII. The pRL Renilla luciferase reporter vector (Promega, E2261) served as an internal control in all assays. The mim-1 reporter construct and pRL Renilla vector (at a ratio of 30:1) were cotransfected into HEK293 cells along with indicated fusions or controls via Lipofectamine 2000. Luciferase activity was quantified 24 h after transfection using the Dual-Luciferase Reporter Assay System (Promega) according to the manufacturer's protocol.

### Cell Lines

NIH3T3, HEK293T and U87 MG cell lines were obtained directly from ACTT and were not reauthenticated. All cell lines were routinely tested (at least every 3 months) for mycoplasma infection.

### Generation of Neural Stem Cells

Embryonic mNSCs were derived from C57BL/6 wild-type E14.5 mouse embryos (purchased from Taconic) as previously described. mNSCs were maintained in culture medium with 1:1 ratio of DMEM (Gibco) and neural stem cell medium (Gibco) supplemented with B27 (Gibco), epidermal growth factor (EGF; 02653, Stem Cell), fibroblast growth factor (FGF; GF003, Millipore) and heparin (07980, Stem Cell).

### Overexpression of Transcripts in Mouse Neural Stem Cells

HEK293T cells were transfected with 10 μg of pLEX307 lentiviral expression vectors (a gift from D. Root; Addgene plasmid 41392) with packaging plasmids encoding PSPAX2 and VSVG using Lipofectamine. Lentivirus-containing supernatant was collected 48 h after transfection, pooled and concentrated (ultrafiltration). Target mNSCs underwent infection using a spin protocol (2,000 rpm for 120 min at 30° C. with no polybrene). Puromycin selection (0.5 μg/ml) commenced 48 h after infection.

Qk shRNA Experiments and Proliferation Assays

Lentiviral vectors (pLKO) encoding shRNAs specific for mouse Qk, targeting sequences in the first four exons of Qk, and control shLacZ were obtained from the RNAi Consortium. Lentivirus was produced by transfection of HEK293T cells with vectors encoding each shRNA (10 μg) with packaging plasmids encoding PSPAX2 and VSVG using Lipofectamine (Invitrogen, 56532). Lentivirus-containing supernatant was collected 48 h after transfection and concentrated. Target mNSCs underwent infection using a spin protocol (2,000 rpm for 120 min at 30° C. with no polybrene). Cells were placed into proliferation assays 48 h after infection.

### Cell Proliferation Assays

1,000 cells/well were plated in 96-well plates, with five replicates. Cell viability was measured by assessing ATP content using CellTiter-Glo (Promega). Means±s.e.m. were calculated.

### Immunoblotting

Cells were lysed and subjected to SDS-PAGE gradient gels as previously described3l. Blots were probed with antibodies against MYB (ab45150, Abcam at 1:500 dilution), QKI (A300-183A, Bethyl Laboratories at 1:1,000 dilution) and actin (sc-1615, Santa Cruz Biotechnology at 1:2,000 dilution).

### RNA Extraction and Real-Time RT-PCR

RNA was extracted with the RNeasy kit (Qiagen). cDNA was synthesized from 1 μg of RNA using High-Capacity RNA-to-cDNA kits (Applied Biosystems). Samples were amplified in triplicate, and the data were analyzed using the ΔΔCt method.

### Gene Expression Analysis of Neural Stem Cells Expressing MYB-QKI

RNA was extracted from three independently generated pools of mNSCs expressing one of eGFP, truncated MYB, MYB-QKI5, MYB-QKI6 or truncated QKI. Gene expression profiles were assayed using Affymetrix Mouse Gene 2.0 ST microarrays. CEL files were RMA normalized58. Comparative marker selection analysis59 was performed in GenePattern using default settings. Genes with P<0.05 and q<0.35 were considered to have significant changes in expression. GSEA was performed using the C2 (CP) gene sets (MSigDB). Gene sets with nominal P<0.05 were considered to be significantly altered. The MYB-QKI signature was defined using the ClassNeighbors module of GenePattern (default settings).

### Antibody Optimization and ChIP-Seq Analysis

The antibody and concentrations that produced the highest signal-to-noise ratio for MYB ChIP-seq was systematically determined using automated ChIP-seq methodology. Two antibodies for MYB, Abcam ab45150 and Sigma SAB4501936, were tested. Abcam 45150 had previously been used for ChIP of MYB. The sheared chromatin was split among three ratios of antibody/chromatin (0.5 μl, 1 μl and 5 μl of each antibody/1,000,000 cells) and ChIP-seq was performed. An antibody targeting H3K27ac (Cell Signaling Technology, D5E4; optimized at 1 μl/1,000,000 cells) was included as a positive control. We found 1 μl of ab45150/1,000,000 cells to be optimal. Results from the MYB ChIP-seq analysis were validated in three ways. First, MYB ChIP-seq was performed in K562 cells and enrichment at genes reported to be target genes in a previous study of these cells was confirmed. Second, HOMER was used to perform an unbiased motif analysis across the peaks identified (peak detection threshold of 1×10-7) in mNSCs overexpressing MYB-QKI and MYB motifs were identified to be the most enriched motifs across all peaks (P=1-681). 92% of all peaks (P-value threshold=1×10-7; 3,392/3,672 peaks) contained a MYB motif. Enrichment of MYB motifs was significantly higher in data generated with the antibody to MYB (P=1-681) as compared to those generated from enrichment with antibody to H3K27ac (P=1-25). Third, our results from mNSCs were compared to other published MYB ChIP-seq results. Significant enrichment of target genes reported in these studies (P<0.0001) was observed in the MYB-bound genes identified in our study (MYB peaks containing a MYB motif) using a χ2 test with Yates correction.

ChIP libraries were indexed, pooled and sequenced on Illumina HiSeq 2000 sequencers. Raw data were aligned to the mm9 mouse reference genome using Picard tools. Raw sequencing data were mapped to the reference genome using Bowtie2 version 2.2.1 with parameters -p 4 -k 1. Peaks were called using MACS version 1.4.2 relative to an input control. A P-value threshold for enrichment of 1×10⁻⁷ was used. The density of genomic regions was calculated using bamliquidator batch, version 1.1.0. Reads were extended 200 bp and normalized to read density in units of RPM/bp. To calculate genome-wide overlap, all enriched H3K27ac peaks were extended 5 kb in each direction and divided into 50 bins, and read density was calculated in each bin. Density was normalized to the largest value observed in each experiment across the genome and plotted as a heat map. Peaks and alignments were converted to TDFs by IGV tools and visualized with IGV. BED files of published ChIP-seq data for H3K27ac chromatin maps from normal brain66 were downloaded and visualized in IGV.

ChIP-seq enrichment for H3K27ac marks was performed on human PLGGs by Active Motif Analysis was performed as above using a P-value threshold for enrichment of 1×10⁻⁵. Super-enhancer analysis was performed as previously described (Chapuy et al., 2013).

### In Vivo Experiments

Mouse flank tumor studies with NIH3T3 stable cell lines. NIH3T3 cell lines were injected subcutaneously into the flanks of NSG mice (five mice for each cell line). Mice were 6-10 weeks of age, with equal representation of male and female mice. Tumor growth was measured biweekly. Ellipsoid tumor volume was calculated using the formula volume=½(length×width2).

For intracranial mouse injections, neurospheres were dissociated and resuspended at 100,000 viable cells/pl. One microliter was injected into the right striatum of immunocompromised ICR-SCID mice. Mice were monitored and euthanized at the onset of neurological symptoms. Brains were subjected to routine histological analysis. Tumors were scored as present on the basis of identification of atypical cells by a neuropathologist. Four- to six-week-old male IcrTac:ICRPrkdc-Scid mice from Taconic were used. A total of 44 mice were used.

Mouse injections were not randomized nor were experimenters blinded to mouse identity. Sample size was not predetermined. Qualitative assessment of tumorigenicity was the primary outcome measured. Neuropathologists were blinded to group allocation.

### Statistical Analysis

For statistical analysis (unless otherwise described), P values were calculated using Fisher's exact tests, t tests or Pearson's tests as appropriate. ANOVA with correction was used for comparison of multiple groups. Log-rank (Mantel-Cox) survival analysis was performed for mouse studies, and Kaplan-Meier curves were generated. The error bars shown depict s.e.m.

### Sequence Listing

SEQ ID NO: 1—genomic sequence of human QKI gene
SEQ ID NO: 2—genomic sequence of human MYB gene
SEQ ID NO: 3—intron 4 of human QKI gene
SEQ ID NO: 4—exon 4 of human QKI gene
SEQ ID NO: 5—intron 9 of human MYB gene
SEQ ID NO: 6—intron 11 of human MYB gene
SEQ ID NO: 7—intron 15 of human MYB gene
SEQ ID NO: 8—exon 16 of human MYB gene

### CITATIONS

Beroukhim, R. et al. Assessing the significance of chromosomal aberrations in cancer: methodology and application to glioma. Proc. Natl. Acad. Sci. USA 104, 20007-20012 (2007).
Chapuy, B. et al. Discovery and characterization of super-enhancer-associated dependencies in diffuse large B cell lymphoma. Cancer Cell 24, 777-790 (2013).
Jones, D. T. W. et al. Recurrent somatic alterations of FGFR1 and NTRK2 in pilocytic strocytoma. Nat. Genet. 45, 927-932 (2013).
Ramkissoon, L. A. et al. Genomic analysis of diffuse pediatric low-grade gliomas identifies recurrent oncogenic truncating rearrangements in the transcription factor MYBL1. Proc. Natl. Acad. Sci. USA 110, 8188-8193 (2013).
Zack, T. I. et al. Pan-cancer patterns of somatic copy number alteration. Nat. Genet. 45, 1134-1140 (2013).
Zhang, J. et al. Whole-genome sequencing identifies genetic alterations in pediatric low-grade gliomas. Nat. Genet. 45, 602-612 (2013).

1. A method of determining an increased likelihood that a pediatric glioma is an angiocentric glioma, the method comprising
a. obtaining a sample of the pediatric glioma;
b. isolating genomic DNA, RNA or protein from the pediatric glioma; and
c. screening the genomic DNA, RNA or protein for the presence of an MYB-QKI rearrangement in the pediatric glioma,
wherein the likelihood that a pediatric glioma is an angiocentric glioma is increased if the MYB-QKI rearrangement is identified in the pediatric glioma.

2. A method of identifying incidence of MYB-QKI rearrangement in a pediatric glioma comprising screening a genomic DNA, RNA or protein from at least one pediatric glioma cell for the presence of an MYB-QKI rearrangement.

3. The method of claim 1 or 2, wherein the pediatric glioma is a pediatric low-grade glioma.

4. The method of claim 1 or 2, wherein the MYB-QKI rearrangement comprises a fusion of a MYB gene and a QKI gene.

5. The method of claim 4, wherein the MYB-QKI rearrangement comprises a rearrangement breakpoint, wherein the rearrangement breakpoint is located in intron 4 of the QKI gene.

6. The method of claim 4, wherein the MYB-QKI rearrangement comprises a rearrangement breakpoint, wherein the rearrangement breakpoint is located in intron 9, intron 11 or intron 15 of the MYB gene.

7. The method of claim 4, wherein the fusion of the MYB gene and the QKI gene is an in-frame fusion.

8. The method of claim 1 or 2, wherein the screening of the genomic DNA comprises use of a cytogenetic technique.

9. The method of claim 8, wherein the cytogenetic technique is fluorescence in situ hybridization (FISH).

10. The method of claim 9, wherein the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 4 of a QKI gene, wherein the second region is located within 100 kb from exon 4 of the QKI gene.

11. The method of claim 1 or 2, wherein the screening of the genomic DNA comprises use of a molecular inversion probe.

12. The method of claim 1 or 2, wherein the screening of the genomic DNA comprises use of genomic DNA sequencing.

13. The method of claim 12, wherein the genomic DNA sequencing comprises whole-genome sequencing.

14. The method of claim 12, wherein the genomic DNA sequencing comprises whole-exome sequencing.

15. The method of claim 1 or 2, wherein screening of the RNA comprises screening for the presence of a MYB-QKI fusion RNA.

16. The method of claim 15, wherein the screening of the RNA comprises use of RNA sequencing.

17. The method of claim 16, wherein the mRNA sequencing comprises whole-transcriptome sequencing.

18. The method of claim 1 or 2, wherein the screening of the protein comprises screening for the presence of a MYB-QKI fusion protein.

19. The method of claim 18, wherein the screening of the protein comprises use of immunohistochemistry.

20. The method of claim 18, wherein the screening of the proteins comprises use of an anti-MYB antibody and an anti-QKI antibody.

21. The method of claim 18, wherein the anti-MYB antibody and the anti-QKI antibody are each conjugated to a fluorescent moiety and fluorescence resonance energy transfer (FRET) between the two fluorescent moieties occurs if the two moieties are in proximity.

22. The method of claim 20, wherein the screening of the protein comprises use of a proximity ligation assay.

23. The method of claim 18, wherein the screening of the protein comprises use of an antibody that binds specifically to a joint region of the fusion protein wherein the joint region comprises at least one amino acid of the MYB protein sequence and at least one amino acid of the QKI protein sequence.

24. The method of claim 18, wherein the screening of the protein comprises use of mass spectrometry.

25. A kit for detecting MYB-QKI rearrangement, comprising at least a first FISH probe that hybridizes to a first region 5′ to exon 16 of a MYB gene, wherein the first region is located within 100 kb from exon 16 of the MYB gene.

26. The kit of claim 25, further comprising a second FISH probe that hybridizes to a second region 3′ to exon 4 of a QKI gene, wherein the second region is located within 100 kb from exon 4 of the QKI gene.

27. A method of treating a pediatric glioma in a subject in need thereof comprising:
a. obtaining a sample of the pediatric glioma;
b. isolating genomic DNA, RNA or protein from the pediatric glioma;
c. screening the genomic DNA, RNA or protein for the presence of an MYB-QKI rearrangement in the pediatric gliomas, wherein the likelihood that a pediatric glioma is an angiocentric glioma is increased if MYB-QKI rearrangement is identified in the pediatric glioma; and
d. performing surgical resection on the subject if the MYB-QKI rearrangement is present,
thereby treating the pediatric glioma in the subject.

28. A method of treating a pediatric glioma in a subject in need thereof, the method comprising:
a. screening a genomic DNA, RNA or protein from the pediatric glioma for the presence of an MYB-QKI rearrangement; and
b. performing surgical resection on the subject if the MYB-QKI rearrangement is present,
thereby treating the pediatric glioma in the subject.

29. The method of claim 27 or 28, wherein the pediatric glioma is a pediatric low-grade glioma.

30. The method of claim 27 or 28, wherein the MYB-QKI rearrangement comprises a fusion of a MYB gene and a QKI gene.

31. The method of claim 30, wherein the MYB-QKI rearrangement comprises a rearrangement breakpoint, wherein the rearrangement breakpoint is located in intron 4 of the QKI gene.

32. The method of claim 30, wherein the MYB-QKI rearrangement comprises a rearrangement breakpoint, wherein the rearrangement breakpoint is located in intron 9, intron 11 or intron 15 of the MYB gene.

33. The method of claim 30, wherein the fusion of the MYB gene and the QKI gene is an in-frame fusion.

34. The method of claim 27 or 28, wherein the screening of the genomic DNAs comprises use of a cytogenetic technique.

35. The method of claim 34, wherein the cytogenetic technique is fluorescence in situ hybridization (FISH).

36. The method of claim 35, wherein the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 4 of a QKI gene, wherein the second region is located within 100 kb from exon 4 of the QKI gene.

37. The method of claim 27 or 28, wherein the screening of the genomic DNA comprises use of a molecular inversion probe.

38. The method of claim 27 or 28, wherein the screening of the genomic DNA comprises use of genomic DNA sequencing.

39. The method of claim 38, wherein the genomic DNA sequencing comprises whole-genome sequencing.

40. The method of claim 38, wherein the genomic DNA sequencing comprises whole-exome sequencing.

41. The method of claim 27 or 28, wherein the screening of the RNA comprises screening for the presence of a MYB-QKI fusion RNA.

42. The method of claim 41, wherein the screening of the RNA comprises use of mRNA sequencing.

43. The method of claim 41, wherein the mRNA sequencing comprises whole-transcriptome sequencing.

44. The method of claim 27 or 28, wherein the screening of the protein comprises screening for the presence of a MYB-QKI fusion protein.

45. The method of claim 44, wherein the screening of the protein comprises use of immunohistochemistry.

46. The method of claim 44, wherein the screening of the protein comprises use of an anti-MYB antibody and an anti-QKI antibody.

47. The method of claim 46, wherein the anti-MYB antibody and the anti-QKI antibody are each conjugated to a fluorescent moiety and fluorescence resonance energy transfer (FRET) between the two fluorescent moieties occurs if the two moieties are in proximity.

48. The method of claim 46, wherein the screening of the protein comprises use of a proximity ligation assay.

49. The method of claim 44, wherein the screening of the protein comprises use of an antibody that binds specifically to a joint region of the fusion protein wherein the joint region comprises at least one amino acid of the MYB protein sequence and at least one amino acid of the QKI protein sequence.

50. The method of claim 44, wherein the screening of the protein comprises use of mass spectrometry.

51. The method of claim 27 or 28, wherein radiation therapy is not provided to the subject if an MYB-QKI rearrangement is identified.

52. The method of claim 27 or 28, wherein chemotherapy is not provided to the subject if the MYB-QKI rearrangement is identified.

53. A method for treating a pediatric glioma having an MYB-QKI rearrangement in a subject in need thereof, wherein the MYB-QKI rearrangement comprises an MYB-QKI fusion DNA, the method comprising administering to the subject an agent that deletes at least a 100 bp portion of the MYB-QKI fusion DNA.

54. A method for treating a pediatric glioma having an MYB-QKI rearrangement in a subject in need thereof, wherein the MYB-QKI rearrangement comprises an MYB-QKI fusion DNA, the method comprising administering to the subject an agent that generates a frame-shifting mutation of the MYB-QKI fusion DNA.

55. The method of claim 53 or 54, wherein the agent comprises:
a. a Cas9 protein or a polynucleotide encoding a Cas9 protein; and
b. a CRISPR-Cas system guide RNA polynucleotide targeting the MYB-QKI genomic locus.

56. A method for treating a pediatric glioma having a MYB-QKI rearrangement in a subject in need thereof, wherein the MYB-QKI rearrangement comprises an MYB-QKI fusion DNA which is transcribed to an MYB-QKI fusion RNA, the method comprising administering to the subject an agent that reduces the amount of the MYB-QKI fusion RNA in a cell in the subject.

57. The method of claim 56, wherein the agent comprises an interfering RNA that targets an MYB-QKI mRNA.

58. The method of claim 56, wherein the agent inhibits the activity of at least one enhancer in the region within or 3′ to the genomic location of the MYB-QKI fusion gene that is operably linked to the genomic sequence of the MYB-QKI.

59. The method of claim 58, wherein the enhancer is located within 15 kb from the genomic location of the 5′ end of the MYB portion of the MYB-QKI fusion DNA.

60. The method of claim 58, wherein the enhancer is located between 100 kb and 500 kb from the genomic location of the 3′ end of the MYB-QKI fusion DNA.

61. The method of any one of claims 54, 55, and 58-60, wherein the agent comprises an antagonist of BET.

62. The method of claim 61, wherein the antagonist of BET is selected from the group consisting of JQ1, GSK1210151A, GSK525762, OTX-015, TEN-010, CPI-203, CPI-0610, RVX-208, and LY294002.

63. The method of claim 62, wherein the antagonist of BET is JQ1.

64. The method of any one of claims 54, 55, and 58-60, wherein the agent comprises an antagonist of CDK7.

65. The method of claim 64, wherein the antagonist of CDK7 is selected from the group consisting of THZ1, BS-181, flavopiridol, P276-00, R-roscovitine, R547, SNS-032, and ZK 304709.

66. The method of claim 65, wherein the antagonist of CDK7 is THZ1.

67. The method of claim 56, wherein the agent inhibits H3K27 acetylation at the MYB-QKI locus.

68. A method for treating a pediatric glioma having an MYB-QKI rearrangement in a subject, wherein the MYB-QKI rearrangement comprises an MYB-QKI fusion DNA which is transcribed to an MYB-QKI fusion RNA, which RNA is translated to an MYB-QKI fusion protein, the method comprising administering to the subject an agent that reduces the amount or activity of the MYB-QKI fusion protein.

69. The method of claim 68, wherein the agent increases the rate of MYB-QKI protein degradation.

70. The method of claim 69, wherein the agent comprises an antagonist of at least one deubiquitinating enzyme (DUB).

71. The method of claim 70, wherein the antagonist of DUB is selected from the group consisting of PR619, VLX1570, b-AP15, PX-478, and WPI 130.

72. The method of claim 71, wherein the antagonist of DUB is PR619.

73. A method for treating a pediatric glioma having a MYB-QKI rearrangement in a subject in need thereof, the method comprising administering to the subject an antagonist of c-Kit.

74. The method of claim 73, wherein the antagonist of c-Kit is selected from the group consisting of axitinib, dovitinib, dasatinib, imatinib, motesanib, pazopanib, masitinib, vatalanib, cabozantinib, tivozanib, OSI-930, Ki8751, telatinib, pazopanib, and tyrphostin AG 1296.

75. The method of claim 74, wherein the antagonist of c-Kit is dasatinib.

76. The method of any of the claims 53-75, wherein the pediatric glioma is a pediatric low-grade glioma.

77. The method of claim 76, wherein the pediatric low-grade glioma is an angiocentric glioma.

78. A method of determining an increased likelihood that a pediatric glioma is an angiocentric glioma, the method comprising
a. obtaining a sample of the pediatric glioma;
b. isolating genomic DNA from the pediatric glioma; and
c. screening the genomic DNA for the presence of an MYB alteration in the pediatric glioma,
wherein the likelihood that a pediatric glioma is an angiocentric glioma is increased if an MYB alteration is identified in the pediatric glioma.

79. A method of classifying a pediatric glioma, the method comprising screening a genomic DNA from the pediatric glioma for the presence of an MYB alteration.

80. The method of claim 78 or 79, wherein the pediatric glioma is a pediatric low-grade glioma.

81. The method of claim 80, wherein the MYB alteration comprises one or more of: a copy number alteration, a truncation, a fusion, a rearrangement, a 5′ deletion, a 3′deletion, a breakpoint, a translocation, an inversion, and an insertion.

82. The method of claim 81, wherein the MYB alteration comprises a 3′ deletion of MYB.

83. The method of claim 82, wherein the 3′ deletion of MYB comprises a deletion of a portion of the MYB gene comprising all exons and introns 3′ to MYB intron 15.

84. The method of claim 82, wherein the 3′ deletion of MYB comprises a deletion of a portion of the MYB gene comprising all exons and introns 3′ to MYB intron 11.

85. The method of claim 82, wherein the 3′ deletion of MYB comprises a deletion of a portion of the MYB gene comprising all exons and introns 3′ to MYB intron 9.

86. The method of claim 82, wherein the expression level of MYB exons 1-9 is higher in the pediatric glioma with the MYB alteration than in a control sample.

87. The method of claim 86, wherein the control sample comprises a sample of pediatric glioma wherein MYB alteration does not occur.

88. The method of claim 86, wherein the control sample comprises a sample of normal brain or spine tissue.

89. The method of claim 78 or 79, wherein the screening of the genomic DNA comprises use of a cytogenetic technique.

90. The method of claim 89, wherein the cytogenetic technique is fluorescence in situ hybridization (FISH).

91. The method of claim 90, wherein the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 15 of a MYB gene, wherein the second region is located within 100 kb from exon 15 of the MYB gene.

92. The method of claim 78 or 79, wherein the screening of the genomic DNA comprises use of genomic DNA sequencing.

93. The method of claim 92, wherein the genomic DNA sequencing comprises whole-genome sequencing.

94. The method of claim 92, wherein the genomic DNA sequencing comprises whole-exome sequencing.

95. A kit for detecting MYB alteration, comprising at least a first FISH probe that hybridizes to a first region 5′ to exon 16 of a MYB gene, wherein the first region is located within 100 kb from exon 16 of the MYB gene.

96. The kit of claim 95, further comprising a second FISH probe that hybridizes to a second region 3′ to exon 15 of a MYB gene, wherein the second region is located within 100 kb from exon 15 of the MYB gene.

97. A method of treating a pediatric glioma in a subject in need thereof, the method comprising:
a. obtaining a sample of the pediatric glioma;
b. isolating genomic DNA from the pediatric glioma;
c. screening the genomic DNA for the presence of an MYB alteration in the pediatric gliomas, wherein the likelihood that a pediatric glioma is an angiocentric glioma is increased if an MYB alteration is identified in the pediatric glioma; and
d. performing surgical resection on the subject if the MYB alteration is present,
thereby treating the pediatric glioma in the subject.

98. A method of treating a pediatric glioma in a subject in need thereof, the method comprising:
a. screening a genomic DNA from the pediatric glioma for the presence of an MYB alteration; and
b. performing surgical resection on the subject if the MYB alteration is present,
thereby treating the pediatric glioma in the subject.

99. The method of claim 97 or 98, wherein the pediatric glioma is a pediatric low-grade glioma.

100. The method of claim 97 or 98, wherein the MYB alteration comprises one or more of: a copy number alteration, a truncation, a fusion, a rearrangement, a 5′ deletion, a 3′deletion, a breakpoint, a translocation, an inversion, and an insertion.

101. The method of claim 100, wherein the MYB alteration comprises a 3′ deletion of MYB.

102. The method of claim 101, wherein the 3′ deletion of MYB comprises a deletion of a portion of the MYB gene comprising all exons and introns 3′ to MYB intron 15.

103. The method of claim 101, wherein the 3′ deletion of MYB comprises a deletion of a portion of the MYB gene comprising all exons and introns 3′ to MYB intron 11.

104. The method of claim 101, wherein the 3′ deletion of MYB comprises a deletion of a portion of the MYB gene comprising all exons and introns 3′ to MYB intron 9.

105. The method of claim 101, wherein the expression level of MYB exons 1-9 is higher in the pediatric glioma with the MYB alteration than in a control sample.

106. The method of claim 105, wherein the control sample comprises a sample of pediatric glioma wherein MYB alteration does not occur.

107. The method of claim 105, wherein the control sample comprises a sample of normal brain or spine tissue.

108. The method of claim 97 or 98, wherein the screening of the genomic DNAs comprises use of a cytogenetic technique.

109. The method of claim 108, wherein the cytogenetic technique is fluorescence in situ hybridization (FISH).

110. The method of claim 109, wherein the FISH comprises use of at least a first FISH probe that hybridizes to a first region 5′ to exon 16 of a MYB gene, wherein the first region is located within 100 kb from exon 16 of the MYB gene, and at least a second FISH probe that hybridizes to the a second region 3′ to exon 15 of a MYB gene, wherein the second region is located within 100 kb from exon 15 of the MYB gene.

111. The method of claim 97 or 98, wherein the screening of the genomic DNA comprises use of genomic DNA sequencing.

112. The method of claim 111, wherein the genomic DNA sequencing comprises whole-genome sequencing.

113. The method of claim 111, wherein the genomic DNA sequencing comprises whole-exome sequencing.

114. The method of claim 97 or 98, wherein radiation therapy is not provided to the subject if an MYB rearrangement is identified.

115. The method of claim 97 or 98, wherein chemotherapy is not provided to the subject if the MYB rearrangement is identified.

116. A method for treating a pediatric glioma having an MYB alteration in a subject in need thereof, the method comprising administering to the subject an agent that deletes at least a 100 bp portion of the MYB DNA from the genomic location of MYB alteration.

117. A method for treating a pediatric glioma having an MYB alteration in a subject in need thereof, the method comprising administering to the subject an agent that generates a frame-shifting mutation of the MYB DNA from the genomic location of MYB alteration.

118. The method of claim 116 or 117, wherein the MYB alteration comprises one or more of:
a copy number alteration, a truncation, a fusion, a rearrangement, a 5′ deletion, a 3′ deletion, a breakpoint, a translocation, an inversion, and an insertion.

119. The method of claim 118, wherein the MYB alteration comprises a 3′ deletion of MYB.

120. The method of claim 119, wherein the 3′ deletion of MYB comprises a deletion of a portion of the MYB gene comprising all exons and introns 3′ to MYB intron 15.

121. The method of claim 119, wherein the 3′ deletion of MYB comprises a deletion of a portion of the MYB gene comprising all exons and introns 3′ to MYB intron 11.

122. The method of claim 119, wherein the 3′ deletion of MYB comprises a deletion of a portion of the MYB gene comprising all exons and introns 3′ to MYB intron 9.

123. The method of claim 116 or 117, wherein the agent comprises:
a. a Cas9 protein or a polynucleotide encoding a Cas9 protein; and
b. a CRISPR-Cas system guide RNA polynucleotide targeting the MYB genomic locus.

124. A method for treating a pediatric glioma having a MYB alteration in a subject in need thereof, the method comprising administering to the subject an agent that reduces the amount of the MYB RNA in a cell in the subject.

125. The method of claim 124, wherein the MYB alteration comprises one or more of: a copy number alteration, a truncation, a fusion, a rearrangement, a 5′ deletion, a 3′deletion, a breakpoint, a translocation, an inversion, and an insertion.

126. The method of claim 125, wherein the MYB alteration comprises a 3′ deletion of MYB.

127. The method of claim 126, wherein the 3′ deletion of MYB comprises a deletion of a portion of the MYB gene comprising all exons and introns 3′ to MYB intron 15.

128. The method of claim 126, wherein the 3′ deletion of MYB comprises a deletion of a portion of the MYB gene comprising all exons and introns 3′ to MYB intron 11.

129. The method of claim 126, wherein the 3′ deletion of MYB comprises a deletion of a portion of the MYB gene comprising all exons and introns 3′ to MYB intron 9.

130. The method of claim 124, wherein the agent comprises an interfering RNA that targets a MYB mRNA.

131. The method of claim 130, wherein the agent inhibits the activity of at least one enhancer that is operably linked to the genomic sequence of MYB.

132. The method of claim 123, 124, or 131, wherein the agent comprises an antagonist of BET.

133. The method of claim 132, wherein the antagonist of BET is selected from the group consisting of JQ1, GSK1210151A, GSK525762, OTX-015, TEN-010, CPI-203, CPI-0610, RVX-208, and LY294002.

134. The method of claim 133, wherein the antagonist of BET is JQ1.

135. The method of claim 123, 124, or 131, wherein the agent comprises an antagonist of CDK7.

136. The method of claim 135, wherein the antagonist of CDK7 is selected from the group consisting of THZ1, BS-181, flavopiridol, P276-00, R-roscovitine, R547, SNS-032, and ZK 304709.

137. The method of claim 136, wherein the antagonist of CDK7 is THZ1.

138. The method of claim 124, wherein the agent inhibits H3K27 acetylation at the MYB locus where MYB alteration occurs.

139. A method for treating a pediatric glioma having an MYB alteration in a subject, comprising reducing the amount or activity of the MYB protein.

140. The method of claim 139, wherein the MYB alteration comprises one or more of: a copy number alteration, a truncation, a fusion, a rearrangement, a 5′ deletion, a 3′deletion, a breakpoint, a translocation, an inversion, and an insertion.

141. The method of claim 140, wherein the MYB alteration comprises a 3′ deletion of MYB.

142. The method of claim 141, wherein the 3′ deletion of MYB comprises a deletion of a portion of the MYB gene comprising all exons and introns 3′ to MYB intron 15.

143. The method of claim 142, wherein the 3′ deletion of MYB comprises a deletion of a portion of the MYB gene comprising all exons and introns 3′ to MYB intron 11.

144. The method of claim 142, wherein the 3′ deletion of MYB comprises a deletion of a portion of the MYB gene comprising all exons and introns 3′ to MYB intron 9.

145. The method of claim 139, wherein the agent increases the rate of MYB protein degradation.

146. The method of claim 140, wherein the agent comprises an antagonist of at least one DUB.

147. The method of claim 146, wherein the antagonist of DUB is selected from the group consisting of PR619, VLX1570, b-AP15, PX-478, and WPI 130.

148. The method of claim 147, wherein the antagonist of DUB is PR619.

149. A method for treating a pediatric glioma having a MYB alteration in a subject in need thereof, the method comprising administering to the subject an antagonist of c-Kit.

150. The method of claim 149, wherein the antagonist of c-Kit is selected from the group consisting of axitinib, dovitinib, dasatinib, imatinib, motesanib, pazopanib, masitinib, vatalanib, cabozantinib, tivozanib, OSI-930, Ki8751, telatinib, pazopanib, and tyrphostin AG 1296.

151. The method of claim 150, wherein the antagonist of c-Kit is dasatinib.

152. The method of any of the claims 116-151, wherein the pediatric glioma is a pediatric low-grade glioma.

153. The method of claim 152, wherein the pediatric low-grade glioma is an angiocentric glioma.
